(19) United States
(12) Patent Application Publication
(10) Pub. No.: US 2011/0144032 A1
(43) Pub. Date: Jun. 16, 2011

(54) BIOLOGICAL APPLICATIONS OF STEROID BINDING DOMAINS

(76) Inventors: Christopher Hovens, Surrey Hills (AU); Niall Corcoran, Flemington (IE); Anthony Costello, Port Melbourne (AU)

(21) Appl. No.: 13/057,927

(22) PCT Filed: Aug. 7, 2009
(86) PCT No.: PCT/AU09/01008

§ 371 Date: Feb. 7, 2011

Related U.S. Application Data
(60) Provisional application No. 61/103,420, filed on Oct. 7, 2008.
(60) Provisional application No. 61/103,442, filed on Oct. 7, 2008.
(60) Provisional application No. 61/087,285, filed on Aug. 8, 2008.
(60) Provisional application No. 61/103,446, filed on Oct. 7, 2008.

(30) Foreign Application Priority Data
Sep. 10, 2008 (AU) PCT/AU2008/001338
Oct. 6, 2008 (AU) 2008905185
Oct. 6, 2008 (AU) 2008905186
Oct. 6, 2008 (AU) 2008905187

(51) Int. Cl. A61K 38/00; C07K 2/00; A61P 35/00

(52) U.S. Cl. 514/19.5; 530/300; 514/19.3

(57) ABSTRACT

A polypeptide comprising an androgen binding region, the androgen binding region capable of binding to an androgen at a sufficient affinity or avidity such that upon administration of the polypeptide to a mammalian subject the level of biologically available androgen is decreased.

### FIELD OF THE INVENTION

The present invention relates to the use of certain nuclear receptor ligand binding domain fusion proteins to extend serum half life of a nuclear hormone receptor binding region.

### BACKGROUND TO THE INVENTION

Nuclear hormone receptors are a class of proteins found within the interior of cells that are responsible for sensing the presence of hormones. In response, hormone activated nuclear receptors work in concert with other proteins to increase the expression of specific genes.

Nuclear receptors have the ability to directly bind to DNA and regulate the expression of adjacent genes, hence these receptors are classified as transcription factors. The regulation of gene expression by nuclear receptors is ligand dependent. In other words, nuclear receptors normally are only active in the presence of ligand. More specifically, ligand binding to a nuclear receptor results in a conformational change in the receptor which in turn activates the receptor resulting in up-regulation of gene expression.

A unique property of nuclear receptors which differentiate them from other classes of receptors is their ability to directly interact with and control the expression of genomic DNA. Consequently nuclear receptors play key roles in development and homeostasis of organisms.

One class of important ligands of the nuclear hormone receptors are the steroid hormones. The steroid hormones are all derived from cholesterol. Moreover, with the exception of vitamin D, they all contain the same cyclopentanophenanthrene ring and atomic numbering system as cholesterol. The conversion of C27 cholesterol to the 18-, 19-, and 21-carbon steroid hormones (designated by the nomenclature C with a subscript number indicating the number of carbon atoms, e.g. C19 for androstanes) involves the rate-limiting, irreversible cleavage of a 6-carbon residue from cholesterol, producing pregnenolone (C21) plus isocaproaldehyde. Steroids with 21 carbon atoms are known systematically as pregnanes, whereas those containing 19 and 18 carbon atoms are known as androstanes and estranes, respectively.

All the steroid hormones exert their action by passing through the plasma membrane and binding to intracellular receptors. Steroid hormone-receptor complexes exert their action by binding to specific nucleotide sequences in the DNA of responsive genes. These DNA sequences are identified as hormone response elements, HREs. The interaction of steroid-receptor complexes with DNA leads to altered rates of transcription of the associated genes.

Steroid hormones have defined roles in many biological processes in the body, with the regulation of steroid level being important in maintaining health. Pathological conditions related to abnormally low levels of steroid hormone are often treated simply by administering exogenous hormone to return serum levels to normal. For example, a male with low levels of testosterone may be treated with synthetic testosterone by way of intramuscular injection or a transdermal gel.

However, conditions relating to excess levels of steroid hormone (hypersteroidal conditions) are more difficult to treat. Cushing's syndrome and Cushing's disease are hormonal disorders caused by an abnormally high circulating level of corticosteroid hormones. These conditions are suffered by humans, and other animals such as dogs, cats and horses. Surgical treatment is possible, often involving the removal of one or both of the adrenal glands. While this may be effective, the patient must often take daily supplements of adrenal cortex hormones for the rest of his or her life. However, if the patient cannot safely undergo surgery or if surgery fails, medical therapy may have either a primary or adjunctive role.

Compounds used for the treatment of Cushing's disease work via three broad mechanisms of action. Neuromodulatory compounds modulate corticotropin (ACTH) release from the pituitary, steroidogenesis inhibitors reduce cortisol levels by adrenolytic activity and/or direct enzymatic inhibition and glucocorticoid antagonists block cortisol action at its receptor. In general, neuromodulatory compounds (bromocriptine, cyproheptidine, somatostatin and valproic acid) are not very effective agents for Cushing's disease. Steroidogenesis inhibitors, including mitotane, metyrapone, ketoconazole, and aminoglutethimide, are the agents of choice for medical therapy of Cushing's disease. In general, ketoconazole is the best tolerated of these agents and may be effective as monotherapy in approximately 70% of patients. Mitotane and metyrapone may be effective as single agents, while aminoglutethimide generally must be given in combination. The intravenously-administered etomidate may be used when patients cannot take medications by mouth. These agents have various undesired side-effects, and some such as ketoconazole are very expensive.

Another hypersteroidal condition is polycystic ovary syndrome (PCOS). This is a hormone imbalance in women that can cause irregular periods, unwanted hair growth, and acne. PCOS begins during the teenage years and can be mild or severe. This disorder is characterized by changes to the ovaries such that multiple follicles accumulate in the ovaries without ovulation. The ovary secretes higher levels of testosterone and estrogens. A goal of therapy is to reduce testosterone levels in the serum. Antiandrogen medications in current use include birth control hormones, spironolactone, flutamide and finasteride. These agents have many side effects. For example, while flutamide is an excellent antiandrogen (typically curing hirsuitism) it is hepatatoxic. Spironolactone is safer than flutamide, but somewhat less effective as an antiandrogen. Estrogens suppress testosterone production by inhibiting the release of LHRH from the hypothalamus, but are now rarely used because of concerns about cardiovascular toxicity.

Adrenal gland disorders are another group of conditions relating to increased steroid levels. The adrenal cortex produces glucocorticoids (primarily cortisol), mineralocorticoids (primarily aldosterone), and androgens (primarily dehydroepiandrosterone and androstenedione). Glucocorticoids promote and inhibit gene transcription in many cells and organ systems. Prominent effects include anti-inflammatory actions and increased hepatic gluconeogenesis. Mineralocorticoids regulate electrolyte transport across epithelial surfaces, particularly renal conservation of Na in exchange for K. Adrenal androgens' chief physiologic activity occurs after conversion to testosterone and dihydrotestosterone.

The adrenal medulla is composed of chromaffin cells, which synthesize and secrete catecholamines (mainly epinephrine and lesser amounts of norepinephrine). Chromaffin cells also produce bioactive amines and peptides (eg, histamine, serotonin, chromogranins, neuropeptide hormones). Epinephrine and norepinephrine, the major effector amines of the sympathetic nervous system, are responsible for the “flight or fight” response (ie, chronotropic and inotropic effects on the heart; bronchodilation; peripheral and splanchnic vasoconstriction with skeletal muscular vasodilation; metabolic effects including glycogenolysis, lipolysis, and renin release).

Hyperfunction of the adrenal gland produces distinct clinical syndromes. Hypersecretion of androgens results in adrenal virilism; of glucocorticoids, Cushing's syndrome; and of aldosterone, hyperaldosteronism (aldosteronism). These syndromes frequently have overlapping features. Hyperfunction may be compensatory, as in congenital adrenal hyperplasia, or due to acquired hyperplasia, adenomas, or adenocarcinomas.

Adrenal virilism is a syndrome in which excessive adrenal androgens cause virilization. Diagnosis is clinical and confirmed by elevated androgen levels with and without dexamethasone suppression; determining the underlying cause may involve adrenal imaging, with needle biopsy if a mass lesion is found. Treatment depends on the cause.

Adrenal virilism is caused by an androgen-secreting adrenal tumor or by adrenal hyperplasia. Sometimes, the tumor secretes both excess androgens and cortisol, resulting in Cushing's syndrome (discussed infra) with suppression of ACTH secretion and atrophy of the contralateral adrenal. Adrenal hyperplasia is usually congenital; delayed virilizing adrenal hyperplasia is a variant of congenital adrenal hyperplasia. Both are caused by a defect in hydroxylation of cortisol precursors; cortisol precursors accumulate and are shunted into the production of androgens. The defect is only partial in delayed virilizing adrenal hyperplasia, so clinical disease may not develop until adulthood.

Cushing's syndrome is a constellation of clinical abnormalities caused by chronic high blood levels of cortisol or related corticosteroids. Cushing's disease is Cushing's syndrome that results from excess pituitary production of ACTH, usually secondary to a pituitary adenoma. Typical symptoms include “moon” faces and truncal obesity with thin arms and legs. Diagnosis is by history of receiving corticosteroids or by elevated serum cortisol.

Hyperfunction of the adrenal cortex can be ACTH-dependent or ACTH-independent. ACTH-dependent hyperfunction may result from hypersecretion of ACTH by the pituitary gland; secretion of ACTH by a nonpituitary tumor, such as small cell carcinoma of the lung or a carcinoid tumor (ectopic ACTH syndrome); or administration of exogenous ACTH. ACTH-independent hyperfunction usually results from therapeutic administration of corticosteroids or from adrenal adenomas or carcinomas; rare causes include primary pigmented nodular adrenal dysplasia (usually in adolescents) and macronodular dysplasia (in older patients).

Whereas the term Cushing's syndrome denotes the clinical picture resulting from cortisol excess from any cause, Cushing's disease refers to hyperfunction of the adrenal cortex from pituitary ACTH excess. Patients with Cushing's disease usually have a small adenoma of the pituitary gland.

Primary aldosteronism (Conn's syndrome) is aldosteronism caused by autonomous production of aldosterone by the adrenal cortex (due to hyperplasia, adenoma, or carcinoma). Symptoms and signs include episodic weakness, elevated BP, and hypokalemia. Diagnosis includes measurement of plasma aldosterone levels and plasma renin activity. Treatment depends on cause. A tumor is removed if possible; in hyperplasia, spironolactone or related drugs may normalize BP and eliminate other clinical features.

Aldosterone is the most potent mineralocorticoid produced by the adrenals. It causes Na retention and K loss. In the kidney, aldosterone causes transfer of Na from the lumen of the distal tubule into the tubular cells in exchange for K and hydrogen. The same effect occurs in salivary glands, sweat glands, cells of the intestinal mucosa, and in exchanges between ICFs and ECFs.

Aldosterone secretion is regulated by the renin-angiotensin system and, to a lesser extent, by ACTH. Renin, a proteolytic enzyme, is stored in the juxtaglomerular cells of the kidney. Reduction in blood volume and flow in the afferent renal arterioles induces secretion of renin. Renin transforms angiotensinogen from the liver to angiotensin I, which is transformed by ACE to angiotensin II. Angiotensin II causes secretion of aldosterone and, to a much lesser extent, secretion of cortisol and deoxycorticosterone; it also has pressor activity. Na and water retention resulting from increased aldosterone secretion increases the blood volume and reduces renin secretion.

Primary aldosteronism is caused by an adenoma, usually unilateral, of the glomerulosa cells of the adrenal cortex or, more rarely, by adrenal carcinoma or hyperplasia. Adenomas are extremely rare in children, but the syndrome sometimes occurs in childhood adrenal carcinoma or hyperplasia. In adrenal hyperplasia, which is more common in elderly men, both adrenals are overactive, and no adenoma is present.

Secondary aldosteronism is increased adrenal production of aldosterone in response to nonpituitary, extra-adrenal stimuli, including renal artery stenosis and hypovolemia. Symptoms are those of primary aldosteronism. Treatment involves correcting the underlying cause.

Secondary aldosteronism is caused by reduced renal blood flow, which stimulates the renin-angiotensin mechanism with resultant hypersecretion of aldosterone. Causes of reduced renal blood flow include obstructive renal artery disease (eg, atheroma, stenosis), renal vasoconstriction (as occurs in accelerated hypertension), and edematous disorders (eg, heart failure, cirrhosis with ascites, nephrotic syndrome). Secretion may be normal in heart failure, but hepatic blood flow and aldosterone metabolism are reduced, so circulating levels of the hormone are high.

Like steroids, the thyroid hormones are important ligands of nuclear hormone receptors. The thyroid gland, located in the anterior neck just below the cricoid cartilage, consists of 2 lobes connected by an isthmus. Follicular cells in the gland produce the 2 main thyroid hormones, tetraiodothyronine (thyroxine, T₄), and triiodothyronine (T₃). These hormones act on cells in virtually every body tissue by combining with nuclear receptors and altering expression of a wide range of gene products. Thyroid hormone is required for normal brain and somatic tissue development in the fetus and newborn, and, in all ages, regulates protein, carbohydrate, and fat metabolism.

T₃ is the most active form; T₄ has only minimal hormonal activity. However, T₄ is much longer lasting and can be converted to T₃ (in most tissues) and thus serves as a reservoir for T₃. A 3rd form of thyroid hormone, reverse T₃ (rT₃), has no metabolic activity; levels of rT₃ increase in certain diseases.

Hyperthyroidism (thyrotoxicosis) is characterized by hypermetabolism and elevated serum levels of free thyroid hormones. Symptoms are many but include tachycardia, fatigue, weight loss, and tremor. Diagnosis is clinical and with thyroid function tests. Treatment depends on cause.

Graves' disease (toxic diffuse goiter), the most common cause of hyperthyroidism, is characterized by hyperthyroidism and one or more of the following: goiter, exophthalmos, and pretibial myxedema. It is caused by an autoantibody against the thyroid TSH receptor; unlike most autoantibodies, which are inhibitory, this autoantibody is stimulatory, thus producing continuous synthesis and secretion of excess T₄ and T₃. Graves' disease (like Hashimoto's thyroiditis) sometimes occurs with other autoimmune disorders, including type 1 diabetes, vitiligo, premature graying of hair, pernicious anemia, connective tissue diseases, and polyglandular deficiency syndrome.

Thus, the prior art describes a number of treatment modalities that either inhibit the production of steroid hormones or block the action of circulating hormone. From the foregoing description of the prior art, it is clear that prior art treatments for hormonal conditions have at least one problem, and any given treatment may therefore be unsuitable for certain classes of patient. It is an aspect of the present invention to overcome or alleviate a problem of the prior art by providing alternative treatments for hormonal conditions.

Serum is commonly used as a supplement to basal growth medium in cell culture. The most common type of serum used for cell growth is foetal bovine serum (FBS), also known as foetal calf serum (FCS). Fetal bovine serum is obtained from fetuses harvested in abattoirs from healthy dams fit for human consumption. Occasionally, there may be use of other bovine sera, such as newborn calf serum or donor bovine serum. In cell culture, serum provides a wide variety of macromolecular proteins, low molecular weight nutrients, carrier proteins for water—insoluble components, and other compounds necessary for in vitro growth of cells, such as hormones and attachment factors. Serum also adds buffering capacity to the medium and binds or neutralizes toxic components. Attempts to replace serum entirely with serum-free medium have met only with limited success.

While serum includes many beneficial biologically active molecules necessary for successful cell culture, it also contains actives that are undesirable in some applications. Hormones are one class of active molecule that must often be removed from serum before use. For example, in the study of hormone-dependent model cancer cell lines it is typically required to study the cell both in the presence and absence of hormone. At present, activated charcoal is used to deplete serum of hormones. Charcoal-stripping reduces the concentration of steroid hormones in serum, for example estradiol, progesterone, cortisol, and testosterone.

Charcoal-stripped FBS is used to elucidate the effects of hormones in a variety of in vitro systems. Studies include steroid-receptor binding, steroid regulation of cellular receptors, hormone secretion of various tissues and the function of thyroid hormones.

Charcoal stripped serum is treated by filtering chilled serum through an activated carbon adsorbent filter to remove non-polar material. This treatment removes lipophilic material but has little effect on the concentration of salts dissolved in the serum. However, Charcoal stripping is non-specific, removing a range of actives (both desirable and undesirable) from serum. For example, charcoal stripping does not allow for specific steroid hormones to remain in the serum.

Of particular concern where the serum is used for tissue culture, a study by HyClone Inc (a manufacturer of charcoal treated serum) showed significant alteration in levels of insulin, some vitamins and many peptide growth factors. Moreover, charcoal/dextran treatment was shown to deplete different steroid species to very different extents. For example, testosterone levels were more than halved, while the level of progesterone remained substantially unchanged. (“Art To Science in Tissue Culture”, Hyclone Laboratories, Inc., Vol 12 No. 3/4, Summer/Fall 1993). The same study found that charcoal/dextran treatment may unmask endotoxin activity in serum, with a doubling in endotoxin activity as measured by Limulus Amoebocyte Lysate assay being noted after treatment.

In support of the abbve findings, a study by Lamarre et al (Urology 69(1), 2007) found that charcoal stripping significantly reduced the level of vascular endothelial growth factor. Patel et al (J Urol 164: 1420-1425, 2000) showed that charcoal-stripped serum is highly toxic to LNCaP cells, leading to a selective outgrowth of anaplastic androgen-insensitive cells.

Another study demonstrated that charcoal-stripping of serum was shown to remove stimulators of the MAPK signalling pathway and in turn led to downregulation of osteogenesis and upregulation of adipogenesis in a model cell line (Dang and Lowik, Molecular and Cellular Biochemistry, Volume 268, Numbers 1-2, January 2005, pp. 159-167(9)).

In addition to the aforementioned problems, the process of charcoal stripping is expensive and labor intensive. Typically, it is necessary to prepare an activated charcoal/dextran suspension in buffer requiring overnight stirring in a refrigerated environment. The next day the suspension is autoclaved for sterility. After cooling, an equal volume of serum is added to the charcoal/dextran suspension, and stirred at 45° C. for 1 hour. This mixture is then aliquotted into smaller volumes and centrifuged to remove the charcoal. Often, multiple rounds of centrifugation are required to completely remove the charcoal.

It is an aspect of the present invention to overcome or alleviate the prior art by providing methods and compositions for selectively depleting a biological fluid of an active.

A reference herein to a patent document or other matter which is given as prior art is not to be taken as an admission that that document or matter was, in Australia, known or that the information it contains was part of the common general knowledge as at the priority date of any of the claims.

Throughout the description and claims of the specification, the word “comprise” and variations of the word, such as “comprising” and “comprises”, is not intended to exclude other additives, components, integers or steps.

Breast cancer is the most-frequently diagnosed cancer and the second most common cause of death from cancer in women, exceeded only by lung cancer. Breast cancer is a disease causing significant morbidity and mortality throughout the world. There are many different types of breast cancer, and it is not uncommon for a single breast tumor to be a combination of types and to have a mixture of invasive and in situ cancer (cancer that has not spread nor invaded surrounding tissue, and remains confined to the ducts or lobules of the breast).

The two main types of breast adenocarcinomas are ductal carcinomas (also known as intraductal carcinoma), which is the most common non-invasive breast cancer, and lobular carcinomas. Ductal carcinoma in situ (also known as intraductal carcinoma) is the most common type of noninvasive breast cancer. Lobular carcinoma in situ (LOIS, also called lobular neoplasia), while not regarded as a true cancer, is sometimes classified as a type of noninvasive breast cancer, and women with this condition have a higher risk of developing an invasive breast cancer.

The most common breast cancer is invasive (or infiltrating) ductal carcinoma (IDC)—about 80% of invasive breast cancers are IDC. This cancer originates in a duct of the breast, and has progressed past the wall of the duct and invaded the'fatty tissue of the breast. At this point, it can metastasize, or spread to other parts of the body via the lymphatic system and bloodstream. About 10% of invasive breast cancers are invasive (or infiltrating) lobular carcinoma (ILC), which starts in the lobules of the breast, which can metastasize to other parts of the body.

In addition to the above breast cancers, there are uncommon types of breast cancer such as inflammatory breast cancer and medullary cancer, which account for about 1-3% and 5% of all of breast cancers, respectively, metaplastic tumors and tubular carcinomas (both rare variants of invasive ductal cancer), mucinous carcinoma (also known as colloid carcinoma), Paget disease of the nipple, phylloides tumor, and tubular carcinoma.

Women living in Australia, North America and Western Europe have the highest rates of breast cancer in the world. The chance of developing invasive breast cancer at some time in a woman's life is about 1 in 8 (13% of women). World-wide, about 1,150,000 people are diagnosed with breast cancer each year, and of those diagnosed about 410,000 die each year, In Australia, 11,866 new cases were diagnosed in 2001, with the incidence rising from 100.4 cases per 100,000 population in 1991 to 117.2 cases per 100,000 population in 2001. Furthermore, it is estimated that in 2007 about 178,480 new cases of invasive breast cancer will be diagnosed among women in the United States.

In Australia, about 1 in 70 women will develop ovarian cancer during their lifetime—every year around 1200 women will be diagnosed with ovarian cancer and nearly 800 women will die of the disease. Ovarian cancer is the sixth most common cause of cancer death in women—in Australia it is now more common than cervical cancer and it kills many more women. Of the 1200 cases diagnosed each year, about 75% will be advanced stage, and a staggering 75% will not survive past 5 years. In the United States, ovarian cancer is the leading cause of death from gynecologic malignancies and is the fourth most common cause of cancer mortality in women. During 2006, there were projected to be over 20,180 new cases of ovarian cancer in the US resulting in 15,310 deaths (as estimated by the American Cancer Society).

Given the prevalence and seriousness of these diseases, significant research has been directed to achieving control or cures for breast and ovarian cancer. There are a number of treatments known in the art, all of which have at least one adverse side effect.

For breast cancer, primary treatment is surgery for most patients, often with combined with radiation therapy. Chemotherapy, hormone therapy, or both may also be used, depending on tumor and patient characteristics. For inflammatory or advanced breast cancer, primary treatment is systemic therapy, which, for inflammatory breast cancer, is usually followed by surgery and radiation therapy. Surgery is usually not helpful for advanced cancer. Paget's disease of the nipple is treated as for other forms of breast cancer, although a very few patients can be treated successfully with local excision only.

Localised therapies are intended to treat a tumor at the site without affecting the rest of the body, and include surgery and radiation therapy. Mastectomy, championed by William Halstead more than 100 years ago has saved the lives of millions of women with advanced breast cancer, and involves removal of the entire breast, (or both breasts). Radical mastectomy, which involved removal of the breast, axillary lymph nodes and the pectoral muscles, has largely been replaced by a less-disfiguring approach, known as modified radical mastectomy, which involves removal of the axillary nodes and the breast.

The complications of such radical surgery have resulted in the push towards alternative treatments that do not involve loss of the breast. In the 1980s, breast-conserving surgery (BCS) with a 6-week protracted course of whole-breast irradiation (WBI) became popular. In breast conserving surgery, removal of only the breast lump and a surrounding margin of normal tissue is conducted in lumpectomy, and radiation therapy and/or chemotherapy may be conducted subsequent to surgery. Partial (or segmental) mastectomy (often referred to quadrantectomy) removes more breast tissue (up to a quarter of the breast) than a lumpectomy (up to one-quarter of the breast). Similarly, radiation therapy and/or chemotherapy is usually given after surgery.

Possible side effects of mastectomy and lumpectomy include wound infection, hematoma (accumulation of blood in the wound), and seroma (accumulation of clear fluid in the wound). If axillary lymph nodes are also removed, swelling of the arm (lymphedema) is common—about 25% to 30% of women who had underarm lymph nodes removed develop lymphedema. Lymphedema also occurs in up to 5% of women who have sentinel lymph node biopsy; a surgical breast cancer treatment involving removing the sentinel node (the first lymph node into which a tumor drains) and establishing whether further lymph nodes need to be surgically removed. This swelling may last for only a few weeks but may also be long lasting. Other side effects of surgery include temporary or permanent limitations in arm and shoulder movement, numbness of the upper-inner arm skin, tenderness of the area, and hardness due to scar tissue that forms in the surgical site. If upon lumpectomy there is cancer at the margin of biopsied tissue, additional surgery (re-excision) may be required to remove further tissue.

External beam radiation therapy, treatment with high-energy rays or particles that destroy cancer cells, may be used to destroy cancer cells that remain in the breast, chest wall, or underarm area after surgery. The area treated by radiation therapy may also include supraclavicular lymph nodes (nodes above the collarbone) and internal mammary lymph nodes (nodes beneath the sternum or breast bone in the center of the chest). More recently, a new paradigm of partial-breast treatment with breast conserving surgery and partial-breast irradiation (PBI) has been proposed which administers radiation over a much shorter period, and to only the part of the breast with the cancer. It is hoped that partial breast irradiation, which is currently being done in clinical research trials, will prove to be equal to the current, standard whole breast irradiation. Nonetheless, the complications of external beam radiation therapy are swelling and heaviness in the breast, sunburn-like skin changes in the treated area which can last for 6 to 12 months, and fatigue. A further, albeit rare, complication is the development of another cancer called angiosarcoma, which can be treated with mastectomy but can be fatal. Brachytherapy, also known as internal or interstitial radiation, involves the placement of radioactive seeds or pellets directly into breast tissue next to the cancer. Another form of brachytherapy, MammoSite, consists of a balloon attached to a thin tube which is inserted into the lumpectomy space and filled with a saline solution into which a radioactive source is then temporarily placed (through the tube), and following treatment the balloon is then deflated and removed. Complications of brachytherapy include seroma, balloon rupture and wound infections.

Following axillary dissection or radiation therapy, lymphatic drainage of the ipsilateral arm can be impaired, sometimes resulting in significant swelling due to lymphedema. The magnitude of this effect may be proportional to the number of nodes removed. A specially trained therapist must treat lymphedema—special massage techniques once or twice daily may help drain fluid from congested areas toward functioning lymph basins; low-stretch bandaging is applied immediately after manual drainage. After the lymphedema resolves, patients require daily exercise and overnight bandaging of the affected limb indefinitely.

In most cases, chemotherapy is most effective, either as an adjuvant or neoadjuvant therapy, when combinations of more than one chemotherapy drug are used together. The most effective cytotoxic drugs for treatment of metastatic breast cancer are capecitabine, doxorubicin (including its liposomal formulation), gemcitabine, the taxanes paclitaxel and docetaxel, and vinorelbine. Response rate to a combination of drugs is higher than that to a single drug, but survival is not improved and toxicity is increased. Thus, some oncologists use single drugs sequentially. Combination chemotherapy regimens (eg, cyclophosphamide, methotrexate, plus 5-fluorouracil doxorubicin, plus cyclophosphamide) are more effective than a single drug. Acute adverse effects depend on the regimen, but usually include nausea, vomiting, mucositis, fatigue, alopecia, myelosuppression, and thrombocytopenia. The most commonly used combinations include; Cyclophosphamide (Cytoxan), methotrexate (Amethopterin, Mexate, Folex), and fluorouracil (Fluorouracil, 5-FU, Adrucil) [abbreviated CMF]; Cyclophosphamide, doxorubicin (Adriamycin), and fluorouracil [abbreviated CAF]; Doxorubicin (Adriamycin) and cyclophosphamide [abbreviated AC]; Doxorubicin (Adriamycin) and cyclophosphamide followed by paclitaxel (Taxol) or docetaxel (Taxotere) [abbreviated AC->T] or docetaxel concurrent with AC [abbreviated TAC]; Doxorubicin (Adriamycin), followed by CMF; Cyclophosphamide, epirubicin (Ellence), and fluorouracil [abbreviated CEF] with or without docetaxel; Cyclophosphamide and Docetaxel (TC); and Gemcitabine (Gemzar) and paclitaxel (Taxol) [abbreviated GT].

These drugs often have severe toxicity and their use often requires close supervision. For instance, the complications of cyclophosphamide therapy can include aemorrhagic cystitis; gonadal suppression; pigmentation, rash; cardiotoxicity; fluid retention; poor wound healing; hyperuricaemia; gastrointestinal upset; nephrotoxicity; hepatotoxicity; pulmonary fibrosis; sec malignancy, infection; alopecia; haematological effects; and veno-occlusive disease.

The complications of methotrexate therapy can include CNS toxicity; hepato- and nephro-toxicity; gastrointestinal toxicity including ulcerative stomatitis; bone marrow depression; immunosuppression; opportunistic infection especially P. carinii pneumonia; lymphatic, proliferative disorders; fatigue, malaise; infertility; pulmonary toxicity; rash; fever; cardiovascular, and ophthalmic effects.

The complications of fluorouracil therapy can include local pain, pruritus; pigmentation, burning, dermatitis, and scarring.

The complications of doxorubicin therapy can include cardiotoxicity, mucositis; myelosuppression, leucopenia, haemorrhage; injection site reaction; red urine; male infertility; premature menopause; thromboembolism; alopecia; anorexia; gastrointestinal upset, abdominal pain; hyperpigmentation; dehydration; and flushing.

The complications of docetaxel therapy can include rash, sensitivity phenomena; alopecia; hand foot syndrome; haematological effects; oedema; gastrointestinal upset; hypertension, hypotension; neurosensory symptoms; injection site reaction; lacrimation both with and without conjunctivitis; visual effects; ear, and labyrinth disorders.

The complications of epirubicin therapy can include cardiotoxicity; extravasation; vesication; myelosuppression; CNS, cardiovascular, haematological, gastrointestinal, ocular, hepatic disturbances; dehydration; alopecia; hyperuricaemia; red urine; thromboembolism; amenorrhoea, and premature menopause.

The complications of gemcitabine therapy can include flu-like syndrome; oedema; hepatic, cardiac, blood disorders; somnolence; gastrointestinal upset; pulmonary effects; proteinuria, haematuria; rash (severe skin reactions, rare); pruritus; alopecia; and mouth ulceration.

The complications of taxol therapy can include hypersensitivity including anaphylactoid reactions; cardiovascular effects incl hypotension, arrhythmia; bone marrow suppression; peripheral neuropathy; arthralgia, myalgia; raised LFTs; gastrointestinal upset, perforation; alopecia; and injection site reactions.

A problem of multi-targeted agents is that the clinical effects of these drugs most likely result from both their on-target, and off target, effects. The toxicities mentioned above can be off-target effects, resulting from unintended and unknown functions, however it has been proposed that clinicians prefer multi-targeted drugs since they aim to maximize the chance for antitumor activity. Changes in dose (to increase efficacy) may amplify these off-target effects.

Choice of therapy depends on the hormone-receptor status of the tumor, length of the disease-free interval (from diagnosis to manifestation of metastases), number of metastatic sites and organs affected, and patient's menopausal status. Most patients with symptomatic metastatic disease are treated with systemic hormone therapy or chemotherapy. Radiation therapy alone may be used to treat isolated, symptomatic bone lesions or local skin recurrences not amenable to surgical resection. Radiation therapy is the most effective treatment for brain metastases, occasionally achieving long-term control. Patients with multiple metastatic sites outside the CNS should initially be given systemic therapy. There is no proof that treatment of asymptomatic metastases substantially increases survival, and it may reduce quality of life.

Hormone therapy is another form of adjuvant systemic therapy. The hormone estrogen is produced mainly by a woman's ovaries until menopause, after which it is made mostly in the body's fat tissue where a testosterone-like hormone (androstenedione) made by the adrenal gland is converted into estrogen by the enzyme aromatase. Estrogen promotes the growth of about two thirds of breast cancers (those containing estrogen or progesterone receptors and called hormone receptor positive cancers). Because of this, several approaches to blocking the effect of estrogen or lowering estrogen levels are used to treat breast cancer, including selective estrogen receptor modulators (SERMS) and aromatase inhibitors.

Hormone therapy is preferred over chemotherapy for patients with estrogen receptor-positive (ER+) tumors, a disease-free interval of greater than 2 years, or disease that is not life threatening. Tamoxifen is often used first in premenopausal women. Ovarian, ablation by surgery, radiation therapy, or use of a luteinizing-releasing hormone agonist (eg, buserelin, goserelin, leuprolide) is a reasonable alternative. Combination therapy of ovarian ablation with tamoxifen therapy is another alternative. If the cancer initially responds to hormone therapy but progresses months or years later, additional forms of hormone therapy may be used sequentially until no further response is seen.

SERMS are a class of compounds that exert various levels of anti-estrogenic activity in the breast and uterus while showing variable estrogenic effects in other tissues. These tissue-specific effects depend upon the level of interaction of the co-activators and co-repressors and other associated proteins with the estrogen receptor. There are currently two major SERMS are currently in use in the clinic and clinical trials; tamoxifen, and raloxifene.

Tamoxifen has been shown to improve survival at all stages of breast cancer, and adjuvant tamoxifen for about 5 years reduces the annual breast cancer death rate by 31% in women with cancers expressing the estrogen receptor. However, the complications of tamoxifen therapy can include hot flushes; vaginal bleeding, discharge; pruritus vulvae; headache; fluid retention; uterine fibroids, endometriosis; endometrial changes including cancer, uterine sarcoma (mostly malignant, mixed Mullerian tumours); cystic ovarian swellings; haematological changes; hypercalcaemia; thromboembolic phenomena; gastrointestinal intolerance; bone, tumour pain; ocular changes; lightheadedness; rash; alopecia; liver enzyme changes; raised triglycerides, pancreatitis; and in rare cases severe hepatic abnormalities and interstitial pneumonitis. Despite approval by the US FDA, only 5-30% of high-risk women agree to take tamoxifen as a preventive agent because of these reported side effects (in particular endometrial cancer, thromboembolic events, and hot flashes).

Raloxifene has been demonstrated to reduce the risk of invasive breast cancer by 44% in women, however in the same study, the risk of fatal stroke was increased by 49%, and complications of raloxifene therapy may include hot flushes; leg cramps; and thromboembolism. Importantly, half of breast cancers are not prevented or delayed by tamoxifen or raloxifene.

Aromatase inhibitors are compounds that inhibit the transformation of androstenedione and testosterone into estrone and estradiol, respectively. There are two classes of aromatase inhibitors, namely steroidal (e.g. exemestane) and nonsteroidal (e.g. anastrazole and letrozole) available. The complications of exemestane therapy can include hot flushes; fatigue; pain including joint pain, musculoskeletal; oedema; gastrointestinal upset; sweating; headache; dizziness; carpal tunnel syndrome; insomnia; depression; rash; alopecia; lymphopenia; thrombocytopenia; and leucopenia. The complications of anastrazole therapy can include hot flushes; asthenia; joint pain, stiffness; vaginal dryness, bleeding; hair thinning; rash; gastrointestinal upset; headache; carpal tunnel syndrome; hypercholesterolaemia; anorexia (mild); somnolence; severe skin reactions; hypersensitivity including anaphylaxis among others. The complications of letrozole therapy can include hot flushes; gastorintestinal upset; fatigue; anorexia; increased appetite, sweating, weight; hypercholesterolaemia; depression; headache; dizziness; alopecia; rash; arthralgia; myalgia; bone pain, fracture; osteoporosis; and peripheral oedema. Aromatase inhibitors are more effective than tamoxifen as first-line therapy for postmenopausal women with advanced breast cancer or as adjuvant therapy in preventing recurrence of breast cancer however, in addition to the possible side effects listed above, the long-term effects of aromatase inhibitors remain to be evaluated.

Fulvestrant, a steroidal ‘pure’ antiestrogen (i.e. it is free of any estrogen-like activity in the absence of estrogens), exerts its action by blocking the binding of estrogens to the estrogen receptor in all tissues—causing generalized estrogen deprivation. The complications of fulvestrant therapy can include hot flushes; nausea; injection site reaction; asthenia; pain; headache; vasodilatation; bone pain; pharyngitis; dyspnoea; raised liver function tests; and less commonly hypersensitivity. While fulvestrant has been shown to be equivalent to tamoxifen as a primary treatment of advanced breast cancer, no difference was observed in median time to progression compared with anastrazole (in patients who had progressed despite prior endocrine therapy).

A significant problem with the anti-estrogen therapies discussed infra is that patients may demonstrate signs of resistance to the drug at first instance, or may develop resistance in the course of therapy. While the cause of anti-estrgoen resistance has not been definitively elucidated, one theory is that mutation(s) in the target (i.e. the estrogen receptor or aromatase molecule) result in a lower affinity of the drug for the target.

Ovarian cancer primarily affects peri- and post-menopausal women. Nulliparity, delayed childbearing, and delayed menopause increase risk, as does a personal or family history of endometrial, breast, or colon cancer. Ovarian cancers are histologically diverse, with at least 80% originating in the epithelium, and of these 75% of these cancers are serous cystadenocarcinoma and the rest include mucinous, endometrioid, transitional cell, clear cell, unclassified carcinomas, and Brenner tumor. The remaining 20% of ovarian cancers originate in primary ovarian germ cells or in sex cord and stromal cells or are metastases to the ovary (most commonly, from the breast or gastrointestinal tract). Germ cell cancers usually occur in women <30 and include dysgerminomas, immature teratomas, endodermal sinus tumors, embryonal carcinomas, choriocarcinomas, and polyembryomas. Stromal (sex cord-stromal) cancers include granulosa-theca cell tumors and Sertoli-Leydig cell tumors.

Ovarian cancer spreads by direct extension, exfoliation of cells into the peritoneal cavity (peritoneal seeding), lymphatic dissemination to the pelvis and around the aorta, or, less often, hematogenously to the liver or lungs. Surgery (hysterectomy and bilateral salpingo-oophorectomy (removal of the ovaries and fallopian tupes) is usually indicated. An exception is nonepithelial or low-grade unilateral epithelial cancer in young patients; fertility can be preserved by not removing the unaffected ovary and uterus. All visibly involved tissue is surgically removed if possible.

Following surgery, changes in sex drive are common. Other complications may include hot flashes and other symptoms of menopause, if both ovaries are removed, increased risk of heart disease and osteoporosis; depression and other forms of psychological distress, blood clots in veins of the legs, risk of infection, internal bleeding, and in the case of hysterectomy, urinary incontinence. Radiation therapy is used infrequently. Chemotherapy may involve topotecan, liposomal doxorubicin, docetaxel, vinorelbine, gemcitabine, hexamethylmelamine, and oral etoposide, and bleomycin.

The complications of topotecan therapy may include haematological and CNS disturbances; fever; infection, sepsis including fatalities; gastrointestinal upset; fatigue; asthenia; alopecia; anorexia; increased liver function tests; dyspnoea and cough among others.

The complications of doxorubicin therapy may include myelosuppression; cardiomyopathy, congestive heart failure; gastrointestinal upset; rash; opportunistic infections; palmar plantar erythrodysaesthesia; severe skin, infusion reactions; extravasation injury; alopecia; myalgia and neuropathy among others.

The complications of vinorelbine therapy may include haematological toxicity; neurological disturbances; gastrointestinal upset; fatigue, fever, arthralgia, myalgia; ischaemic cardiac disease; respiratory distress especially with concomitant mitomycin; and alopecia.

The complications of etoposide therapy may include myelosuppression; gastrointestinal upset; alopecia; and hypotension among others.

The complications of bleomycin therapy may include pulmonary, mucocutaneous toxicity; dermatological changes; renal and hepatic toxicity; hypersensitivity reactions; fever; chills; headache; tiredness; GI upset and anorexia among others.

Cancer of the endometrium is another gynecological cancer that causes significant morbidity and mortality. Endometrial cancer refers to several types of malignancy which arise from the endometrium, or lining of the uterus. Endometrial cancers are the most common gynecologic cancers in the United States, with over 35,000 women diagnosed each year in the U.S. The most common subtype, endometrioid adenocarcinoma, typically occurs within a few decades of menopause, is associated with excessive estrogen exposure, often develops in the setting of endometrial hyperplasia, and presents most often with vaginal bleeding. Because symptoms usually bring the disease to medical attention early in its course, endometrial cancer is only the third most common cause of gynecologic cancer death (behind ovarian and cervical cancer).

Endometrial cancer may sometimes be referred to as uterine cancer. However, different cancers may develop from other tissues of the uterus, including cervical cancer, sarcoma of the myometrium, and trophoblastic disease.

The primary treatment is surgical, typically involving abdominal hysterectomy, and removal of both ovaries and any suspicious pelvic and para-aortic lymph nodes,

Women who are at increased risk for recurrence are often offered surgery in combination with radiation therapy. Chemotherapy may also be considered in some cases such as cisplatin, carboplatin, doxorubicin, and paclitaxel. The side effects of Doxorubicin and Paclitaxel have been considered supra, while those for cisplatin and carboplating include nephrotoxicity, ototoxicity, vestibular toxicity, myelosuppression, anemia, nausea and vomiting, diarrhea, neurotoxicity, muscle cramps, ocular toxicity, anaphylactic-like reactions, and hepatotoxicity,

Thus, the prior art describes many treatment modalities that either physically remove or destroy cells involved in gynecological cancers. Other approaches concentrate on blocking the estrogen receptor by chemical means and by inhibition of the production of estrone and estradiol. From the foregoing description of the prior art, it is clear that every treatment has at least one problem, and may therefore be unsuitable for certain classes of patient. It is an aspect of the present invention to overcome or alleviate a problem of the prior art by providing alternative treatments for breast cancer.

A reference herein to a patent document or other matter which is given as prior art is not to be taken as an admission that that document or matter was, known or that the information it contains was part of the common general knowledge as at the priority date of any of the claims.

A reference herein to a patent document or other matter which is given as prior art is not to be taken as an admission that that document or matter was known or that the information it contains was part of the common general knowledge as at the priority date of any of the claims.

Throughout the description and claims of the specification, the word “comprise” and variations of the word, such as “comprising” and “comprises”, is not intended to exclude other additives, components, integers or steps.

Prostate cancer is a disease causing significant morbidity and mortality throughout the world. The most prevalent form, prostatic adenocarcinoma, arises from the malignant transformation and clonal expansion of epithelial cells lining the secretory acini of the prostate gland. Cancers arising from other prostatic cells types, including transitional cell carcinoma, mesenchymal tumours and lymphomas are much less common.

Prostate adenocarcinoma is the most commonly diagnosed internal malignancy in men in North America, Northern and Western Europe, Australia and New Zealand, as well as parts of Africa. Over 650,000 new cases were diagnosed worldwide in the year 2002, with a mortality rate of over 30%. In Australia, 11,191 new cases were diagnosed in 2001 (age standardized incidence of 128.5 per 100,000) and 2,718 men died of the disease. The incidence is higher in the United States of America (173.8 per 100,000 per year) where in 2005 it is estimate there were over 230,000 new cases diagnosed, and over 30,000 deaths.

Given the prevalence and seriousness of the disease, significant research has been directed to achieving control or a cure for prostate cancer. There are a number of treatments known in the art, all of which have at least one adverse side effect.

Surgical removal of the prostate by radical prostatectomy with or without a regional lymph node dissection is the yardstick against which all other therapies are measured. The standard retropubic approach was repopularised in the 1980s and has been refined into a procedure with a high cure rate and low morbidity. With careful patient selection, 10 year biochemical free recurrence rates of 75% are reported. Improved understanding of pelvic anatomy, particularly at the prostatic apex and the course of the neurovascular bundles has reduced the two most common complications, incontinence and impotence, however these side effects remain significant problems.

External beam radiotherapy can achieve long-term survival in some patients, with success being proportional the total dose delivered to the prostate tumour. In early series where median dose was limited due to rectal and urinary toxicity, biochemical failure occurred in over 50% of patients. Improvements in radiation planning and delivery such as using conformal or intensity-modulated protocols increase the precision by which the target volume corresponds to the tumour volume, allowing higher doses of radiotherapy to be delivered without an increase in complications. Modern series have a similar 10 year biochemical recurrence free survival to radical prostatectomy. The main difference is in the side effect profile, with radiotherapy being associated with a lower risk of urinary incontinence and impotence, at least in the short term, though potency rates do not differ greatly from those achieved with nerve sparing surgery. Severe toxicity such as chronic radiation cystitis or proctitis can be particularly difficult to manage if they occur.

Brachytherapy involves the placement of radioactive seeds transperineally directly into the prostate gland, and has reported biochemical-recurrence free survival rates similar to radical prostatectomy for highly selected cases. Two types of radioactivity sources are used, both of which have a short distance of action: low energy sources, typically iodine-125 or palladium-103 seeds which are placed permanently in the prostate, and high energy sources such as iridium-192 seeds which are placed temporarily. The main advantage of this technique over external beam radiotherapy is that with accurate preoperative computed tomography planning and appropriate seed placement under transrectal ultrasound control, a highly conformal dose distribution can be achieved which results in the delivery of much higher radiation doses with a lower incidence of rectal and neurovascular side-effects. One of the main difficulties even with modern practice is mismatch in dosimetry between planned implantation and the actual implantation because of seed migration, anisotropy of the individual seeds and inaccurate needle placement. In cases where inadequate dosimetry is suspected on postoperative imaging addition implants, or for high risk cases, adjuvant low dose external-beam radiotherapy may be added. The predominant complication is obstructive urinary symptoms due to gland oedema which may precipitate acute urinary retention. There is also a high risk of urinary incontinence following a formal transurethral resection.

Once cancerous cells have metastasized to areas remote from the prostate, removal of the gland becomes redundant. Despite the opportunity for early diagnosis with PSA testing, it is estimated that in the United States at least 14% of patients still present with disease that has spread outside the prostate gland and is no longer amenable to curative therapy. In addition, 30-40% of patients treated initially with curative intent will ultimately fail. Androgen deprivation therapy (ADT) is the usual first line treatment for patients with metastatic disease. Early randomised trials established that treatment of advanced prostate cancer with ADT improves symptoms, delays progression, and probably prolongs survival, with reported remission rates of 85-95%.

The growth of prostate cancer cells at some stages of disease can be reliant on the presence of androgen. Methods for altering the levels of androgen in the blood have been the subject of intensive investigation for many years, revealing a number of sites in the androgen endocrine axis that may be targeted, the most drastic method being bilateral orchidectomy, or surgical castration. For many years, this procedure was the ‘gold standard’ for achieving androgen deprivation. Following removal of the testes, serum testosterone falls rapidly to reach castrate levels (<50 ng/ml) within 9 hours. Side effects are secondary to this fall in testosterone and include hot flushes, reduced libido, fatigue and erectile dysfunction. Increasingly recognised are the medium to long term complications which include osteoporosis, weight gain, loss of muscle mass, anaemia, and a decline in cognitive function. Despite its relatively low cost, surgical castration has fallen from favour due to its irreversible nature and adverse psychological impact on the patient.

Androgen levels may be lowered using LHRH agonists and antagonists. These agents, including leuprolide, goserelin and triptorelin, are peptide analogues of LHRH, and are given as a subcutaneous depot injection every 1-4 months. When released in a pulsatile manner from the hypothalamus, LHRH stimulates the release of LH from the anterior pituitary, and thus testicular production of testosterone. Chronic administration of supraphysiological levels however, after an initial increase in testosterone secretion, leads to downregulation of its cognate receptor and suppression of LH release. Castrate levels of testosterone are seen within 3 to 4 weeks. Because of the initial ‘testosterone flare reaction’, patients with critical tumour deposits must be covered with an antiandrogen when initially commencing a LHRH agonist. The side effects of treatment with LHRH agonists and antagonists are identical to those seen post bilateral orchidectomy.

Another class of drug are the antiandrogens. These agents compete with testosterone and dihydrotestosterone (DHT) for androgen receptor (AR) binding but do not themselves activate the receptor. Non-steroidal antiandrogens such as bicalutamide, flutamide and nilutamide act only at the level of the androgen receptor, including in the hypothalamus where testosterone inhibits LHRH secretion in a classical negative feedback loop. LH secretion, and thus serum testosterone, remains high, so the sexual side effects experienced with castration are reduced. However, due to the peripheral aromatization of testosterone to oestradiol, gynecomastia and breast pain are both common and troublesome. Steroidal antiandrogens, such as the progestin cyproterone acetate, also inhibit LH secretion, but are associated with the sexual side effects of surgical and medical castration. At least in metastatic disease, antiandrogen monotherapy has been shown to be inferior to castration and it's use is therefore limited to patients unable or unwilling to tolerate the side effects of androgen suppression

Prolonged combination of an antiandrogen with an LHRH agonist is termed maximum androgen blockade as the regimen inhibits the effects of the remaining 5-10% of testosterone derived from the adrenal gland. Although an improvement in survival compared to castration alone is reported in some studies, routine use as a first line hormonal treatment is not recommended by most due to increased cost and side effect profile.

Estrogens are also known in the art for their ability to deplete androgen. Although initially the hormonal treatment of choice, diethylstilbestrol, which suppresses testosterone production by inhibiting the release of LHRH from the hypothalamus, is now rarely used as a first line agent because of concerns about cardiovascular toxicity.

Thus, the prior art describes many treatment modalities that either physically remove or destroy prostate cancer cells. Other approaches concentrate on limiting the amount of circulating testosterone by surgical or chemical means. From the foregoing description of the prior art, it is clear that every treatment has at least one problem, and may therefore be unsuitable for certain classes of patient. It is an aspect of the present invention to overcome or alleviate a problem of the prior art by providing alternative treatments for prostate cancer.

A reference herein to a patent document or other matter which is given as prior art is not to be taken as an admission that that document or matter was, in Australia, known or that the information it contains was part of the common general knowledge as at the priority date of any of the claims.

Throughout the description and claims of the specification, the word “comprise” and variations of the word, such as “comprising” and “comprises”, is not intended to exclude other additives, components, integers or steps.

Much research has been devoted to fertility control in economically important animals, companion animals, and pests. For many reasons it is desired to alter the reproductive physiology of an animal to control parameters such as fertility, lactation, and behavior. The ability to control animals in this way is important in the management not only of single animals but also groups of animals.

There are many reasons why it is desirable to control the reproductive physiology of an animal, or a group of animals. For example, racing animals such as greyhound bitches or mares may be excluded from racing or show given that males may be distracted by a female in estrus. In situations where a mare or greyhound bitch in estrus is allowed to race, her performance is typically below that when in anestrus. It would therefore be desirable to control the timing of the estrus such that a female animals is able to race on a specified date. Sex steroid hormones are known to affect the meat characteristics of certain livestock animals. A well known example is ‘Boar taint’ which is a particular taste of pork from male pigs which have been slaughtered at an age when the levels of circulating androgens have reached a certain level.

Two different strategies are often used to the control of estrus in horses. One strategy simply controls when the mare will come into estrus, while the other strategy prevents estrus entirely. Prostaglandin (PGF2α; Lutalyse**) can control the onset of estrus by causing regression of the mature corpus luteum on the ovary. A mature corpus luteum is present on the ovary about 5 days after the mare goes out of heat. When the corpus luteum regresses, the mare returns to estrus. This strategy takes considerable planning and the mare's estrous cycle must be completely understood and monitored closely to be successful.

To use this method, a mare must be out of estrus at least 5 days before receiving prostaglandin. The injection will cause regression of the mature corpus luteum so the mare will come into estrus in 1-7 days, be in estrus 5-7 days, and then be out of estrus for around 14 days. A disadvantage of this method is that a significant amount of information and planning are needed for success.

An alternative strategy prevents estrus by administering progesterone, which prevents the mare from entering estrus as long as it is administered. Progesterone, if given at the proper dosage, will prevent estrus, but will not stop estrus very well once it has already begun. Several different types of progesterone are available. The oldest form is the injectable progesterone in oil. Progesterone in oil must be administered daily to prevent signs of estrus. While effective, daily injections may not be tolerated well for prolonged periods by either mare or owner.

Progesterone-like cattle implants have been used in an attempt to prevent estrus in mares. These progesterone-like implants are surgically placed just under the skin and theoretically should prevent estrus. However, in scientific studies there have been no effects of the subcutaneous implants on changing the mare's estrous cycle. Failure of these implants to prevent estrus is probably due to the type of progesterone they contain, insufficient release of progesterone, and other hormones that are present in the implants.

The only drug that is approved for preventing estrus in the mare is a progestogen called Regu-Mate**. A progestogen is a progesterone-like compound that mimics progesterone, but is not actually progesterone. Regu-Mate** is given orally, and must be given everyday to prevent estrus in the mare. A significant disadvantage in using Regu-Mate** is the expense, as much as $3.70 per day. Another drawback is that some women, who may be medicating the horses, may suffer menstrual-like cramps if the Regu-Mate** contacts the skin.

It is often desired to control the estrus of companion breeding animals to accommodate owners schedules, the availability of stud animals, or the shipments of chilled or frozen semen, or for purposes of increasing the number, frequency or size of litters in such animals. In dogs, one approach involves the use of exogenous estrogen to prime the hypothalamic-pituitary-ovarian axis so as to either induce a false pro-estrus that is expected to be followed by a normal proestrus or induce a proestrus that will progress in a fertile estrus when supplemented with a subsequent gonadotrophin administration. Alternatively, one or more exogenous gonadotropic hormone preparations may be administered to stimulate an ovarian response that results in proestrus followed by a fertile estrus with either spontaneous ovulation or ovulation induced by additional hormone (hCG or GnRH) administration. Another approach is to administer GnRH or a GnRH-agonist in a manner that elicits pituitary release of endogenous gonadotrophins LH and FSH sufficient to provoke an ovarian response that produces normal proestrus and subsequent fertile estrus and spontaneous ovulations. Yet a further approach is the administration of a dopamine agonist that provokes hypothalamic or pituitary hormone responses that lead in time to a premature but otherwise apparently natural proestrus and fertile estrus. All of the methods reported, when assessed in repeated or large studies have a significant failure rate and involve one or more of the following drawbacks: smaller than normal litters in a significant percentage of successful attempts; disruption and possible prolongation of the normal cycle; and, theoretically a possibly increased risk of reproductive tract disease due to premature and possibly excessive stimulation of the reproductive tract by the administered hormones or changes in endogenous hormones provoked by the treatment.

It is further desirable to be able to control the reproductive physiologies of a number of animals in a herd. Typically, the aim is to synchronize reproductive cycles such that all animals are processed through the various phases of husbandry such as conception, gestation, parturition, management of neonates and the like. Processing animals as a group is clearly more cost effective than dealing with individual animals across a longer period of time in an unsynchronized herd. Furthermore, less non-productive days (for example when the animal is not gestating or lactating) are encountered where a herd is reproductively synchronized.

Reproductive synchronization is also desirable in milk-producing animals such that lactation occurs at predetermined times of the year.

Synchronization of reproductive physiologies is also required in some breeding programs. For example, where an embryo transfer is part of the program, it is necessary to synchronize the reproductive cycles of the donor and recipient animals. It is also desirable to control estrus in animals for artificial insemination programs. For example, a single aliquot of frozen semen may contain sufficient material to inseminate several females. However, the cycles of the females may not be synchronized such that the thawed semen would need to be refrozen and thawed when each female came into estrus. This can be avoided by artificially synchronizing the cycles of all females to be inseminated.

Parturition is another reproductive event for which a level of control is often required. For example, it may be necessary to induce labor for the convenience of the animal's owner, or to synchronise labor with one or more other animals such that all animals can receive veterinary attention in a single visit by the veterinarian. Similarly, the onset of lactation for a dairy herd (for example, of cows, or goats) is set by the date of parturition. Timing of conception (and therefore parturition) can also be useful in breeding racing horses. The age of a racing horse is taken from 1 January, and so it is desirable for a foal to be born as soon as possible after that date. This may translate to improved performance of the horse as a two year old.

It is desirable to advance or delay natural breeding seasons in animals. For example, it is known that photoperiod and the timing of an animal's breeding season are related. Photoperiodism ensures in nature that offspring are born at a time of year when food is plentiful. For example, it has been shown in ewes that increasing photoperiod in the late winter-spring leads to an obligatory reproductive onset in the autumn. While these mechanisms have a function in nature, they can be problematic for animals used for production.

Given that male animals do not exhibit a reproductive cycle, fertility control in males more often relates to sterilization. While surgical castration is a commonly used form of sterilization, it is not reversible. The prior art has provided many methods for the non-surgical sterilization of animals. One approach has been to vaccinate the animal against an endogenous molecule involved in the process of conception. For example, a number of studies of female cats fed orally either of engineered strains of Salmonella expressing zona pellucida (ZP) protein concluded that neither vaccine induced sufficient immune responses to effect contraception. This was in spite of showing that there were specific antibodies produced that recognized the ZP antigen or the microbe expressing the ZP, i.e. antibodies against Salmonella. Even though the dosing was performed under highly controlled laboratory conditions, individual cats responded very differently ranging from little to moderate responses.

Other vaccination strategies target the hormone GnRH, that controls the level of sex hormones. In one study, cats were vaccinated with GnRH in an effort to sterilize the animals. While some animals showed decreased levels of fertility, some of the cats did not respond to the vaccine in a significant way (Levy et al, Theriogenology 62 (2004) 1116-1130).

Reversible chemical castration may also be desirable for “teaser” animals which are used to test whether or not a female animal is on heat. In the event that actual copulation occurs between the male and female, conception is prevented where the male is chemically castrated. Furthermore, reversible castration could be advantageous in racing animals to improve performance.

It is an aspect of the present invention to overcome or alleviate a problem of the prior art by providing compositions and methods for regulation of a reproductive physiology of a non-human animal in a non-surgical manner that is also reversible.

A reference herein to a patent document or other matter which is given as prior art is not to be taken as an admission that that document or matter was, in Australia, known or that the information it contains was part of the common general knowledge as at the priority date of any of the claims.

Throughout the description and claims of the specification, the word “comprise” and variations of the word, such as “comprising” and “comprises”, is not intended to exclude other additives, components, integers or steps.

### SUMMARY OF THE INVENTION

In one aspect of the invention there is provided a polypeptide comprising an androgen binding region, the androgen binding region capable of binding to an androgen at a sufficient affinity or avidity such that upon administration of the polypeptide to a mammalian subject the level of biologically available androgen is decreased.

Some embodiments comprise such a polypeptide wherein the level of biologically available androgen is measured in the blood of the subject. Some embodiments comprise such a polypeptide wherein the level of biologically available androgen is measured in a prostate cell of the subject. Some embodiments comprise such a polypeptide wherein the prostate cell is a prostate epithelial cell. Some embodiments comprise such a polypeptide wherein the level of biologically available androgen is decreased such that the growth of a prostate cancer cell in the subject is decreased or substantially arrested. Some embodiments comprise such a polypeptide having an affinity for an androgen that is equal to or greater than the affinity between the androgen and a protein that naturally binds to the androgen.

Some embodiments comprise such a polypeptide having an affinity for testosterone that is equal to or greater than the affinity between testosterone and sex hormone binding globulin. Some embodiments comprise such a polypeptide having an affinity for testosterone that is equal to or greater than the affinity between testosterone and the 5-alpha-reductase enzyme present in a prostate epithelial cell. Some embodiments comprise such a polypeptide having an affinity for testosterone that is equal to or greater than for the affinity between testosterone and the androgen receptor present in a prostate epithelial cell. Some embodiments comprise such a polypeptide having an affinity for dihydrotestosterone that is equal to or greater than for the affinity between dihydrotestosterone and the androgen receptor present in a prostate epithelial cell.

Some embodiments comprise such a polypeptide wherein the androgen binding region includes the androgen binding domain from the human androgen receptor. Some embodiments comprise such a polypeptide wherein the androgen binding region includes the androgen binding domain from the sex hormone binding globulin. Some embodiments comprise such a polypeptide having a single androgen binding region. Some embodiments comprise such a polypeptide comprising a carrier region. Some embodiments comprise such a polypeptide wherein the carrier is the Fc region of human IgG. Some embodiments comprise such a polypeptide capable of entering a prostate cell. Some embodiments comprise such a polypeptide wherein the prostate cell is a prostate epithelial cell. Some embodiments comprise such a polypeptide that is selected from the group consisting of a fusion protein, a monoclonal antibody, a polyclonal antibody, and a single chain antibody. Some embodiments comprise such a polypeptide comprising a multimerisation domain.

Some embodiments comprise a nucleic acid molecule capable of encoding such a polypeptide. Some embodiments comprise a vector comprising such a nucleic acid molecule. Some embodiments comprise a composition comprising a such polypeptide.

In one aspect of the invention, there is provided a method for treating or preventing prostate cancer in a subject, the method comprising administering to a subject in need thereof an effective amount of a ligand capable of binding androgen in the subject, such that the level of biologically available androgen in the subject is decreased as compared with the level of biologically available androgen present in the subject prior to administration of the polypeptide.

Some embodiments comprise such a method wherein the level of biologically available androgen is measured in the blood of the subject. Some embodiments comprise such a method wherein the level of biologically available androgen is measured in a prostate cell of the subject. Some embodiments comprise such a method wherein the prostate cell is a prostate epithelial cell. Some embodiments comprise such a method wherein the prostate cancer is in the androgen dependent phase. Some embodiments comprise such a method wherein the ligand is a polypeptide as described herein.

Some embodiments comprise a method for treating or preventing prostate cancer, the method comprising administering to a subject in need thereof an effective amount of a nucleic acid molecule or a vector as described herein.

Some embodiments comprise a method for treating or preventing testosterone flare in the treatment of a subject with an LHRH agonist or antagonist comprising administering to a subject in need thereof an effective amount of a polypeptide as described herein

Some embodiments comprise use of a polypeptide as described herein in the manufacture of a medicament for the treatment or prevention of prostate cancer. Some embodiments comprise use of a polypeptide as described herein in the manufacture of a medicament for the treatment or prevention of testosterone flare.

Some embodiments comprise use of a nucleic acid molecule according to the invention in the manufacture of a medicament for the treatment or prevention of prostate cancer. Some embodiments comprise use of a nucleic acid molecule according to the invention in the manufacture of a medicament for the treatment or prevention of testosterone flare.

Some embodiments comprise use of a vector according to the invention in the manufacture of a medicament for the treatment or prevention of prostate cancer. Some embodiments comprise use of a vector according to the invention in the manufacture of a medicament for the treatment or prevention of testosterone flare

In one embodiment there is provided a polypeptide comprising an estrogen or androgen binding region, the binding region capable of binding to an estrogen or androgen at a sufficient affinity or avidity such that upon administration of the polypeptide to a mammalian subject the level of biologically available estrogen or androgen is decreased.

Some embodiments comprise such a polypeptide wherein the level of biologically available estrogen or androgen is measured in the blood of the subject. Some embodiments comprise such a polypeptide wherein the level of biologically available estrogen is measured in a breast cell or an ovarian cell of the subject, or the level of biologically available androgen is measured in an endometrial cell of the subject. Some embodiments comprise such a polypeptide wherein the level of biologically available estrogen or androgen is decreased such that the growth of a breast cancer cell, an ovarian cancer cell or an endometrial cancer cell in the subject is decreased or substantially arrested.

Some embodiments comprise such a polypeptide having an affinity or avidity for an estrogen or androgen that is equal to or greater than the affinity or avidity between the estrogen or the androgen and a protein that naturally binds to the estrogen or the androgen. Some embodiments comprise such a polypeptide having an affinity or avidity for estradiol or testosterone that is equal to or greater than the affinity or avidity between estradiol and sex hormone binding globulin, or testosterone and sex hormone binding globulin. Some embodiments comprise such a polypeptide having an affinity or avidity for estradiol or testosterone that is equal to or greater than the affinity or avidity between estradiol and the estrogen receptor, or testosterone and the androgen receptor. Some embodiments comprise such a polypeptide wherein the estrogen binding region comprises the estrogen binding domain from the human estrogen receptor, or a functional equivalent thereof.

Some embodiments comprise such a polypeptide wherein the androgen binding region comprises the androgen binding domain from the human androgen receptor, or a functional equivalent thereof. Some embodiments comprise such a polypeptide wherein the estrogen or androgen binding region comprises the estrogen or androgen binding domain from sex hormone binding globulin, or a functional equivalent thereof. Some embodiments comprise such a polypeptide having a single estrogen or androgen binding region. Some embodiments comprise such a polypeptide comprising a carrier region. Some embodiments comprise such a polypeptide wherein the carrier region is the Fc region of human IgG, or a functional equivalent thereof. Some embodiments comprise such a polypeptide capable of entering a breast cell, an ovarian cell, or an endometrial cell. Some embodiments comprise such a polypeptide that is selected from the group consisting of a fusion protein, a monoclonal antibody, a polyclonal antibody, and a single chain antibody. Some embodiments comprise such a polypeptide comprising a multimerisation domain.

Some embodiments comprise a nucleic acid molecule capable of encoding a polypeptide according to the invention Some embodiments comprise a vector comprising a nucleic acid molecule according to the invention. Some embodiments comprise a composition comprising a polypeptide according to the invention and a pharmaceutically acceptable carrier.

In one aspect of the invention there is provided a method for treating or preventing an estrogen-related cancer or an androgen-related cancer in a subject, the method comprising administering to a subject in need thereof an effective amount of a ligand capable of binding estrogen or androgen in the subject, such that the level of biologically available estrogen or androgen in the subject is decreased as compared with the level of biologically available estrogen or androgen present in the subject prior to administration of the ligand.

Some embodiments comprise such a method wherein the estrogen-related cancer is selected from the group consisting of breast cancer and ovarian cancer. Some embodiments comprise such a method wherein the androgen-related cancer is endometrial cancer. Some embodiments comprise such a method wherein the level of biologically available estrogen is measured in a breast cell or an ovarian cell. Some embodiments comprise such a method wherein the level of biologically available androgen is measured in an endometrial cell. Some embodiments comprise such a method wherein the level of biologically available estrogen or androgen is measured in the blood of the subject. Some embodiments comprise such a method wherein the ligand is a polypeptide according to the invention. Some embodiments comprise such a method for treating or preventing an estrogen-related cancer or an androgen-related cancer, the method comprising administering to a subject in need thereof an effective amount of a nucleic acid molecule according to the invention, or a vector according to claim 18.

Some embodiments comprise such a method wherein the estrogen-related cancer is selected from the group consisting of breast cancer and ovarian cancer. Some embodiments comprise such a method wherein the androgen-related cancer is endometrial cancer. Some embodiments comprise such a method for treating or preventing estrogen flare or testosterone flare in the treatment of a subject having estrogen-related cancer with an LHRH agonist or antagonist comprising administering to a subject in need thereof an effective amount of a polypeptide according to the invention.

Some embodiments comprise use of a polypeptide of the invention in the manufacture of a medicament for the treatment or prevention of an estrogen-related cancer or an androgen-related cancer. Some embodiments comprise such a method according to claim 31 wherein the estrogen-related cancer is selected from the group consisting of breast cancer and ovarian cancer. Some embodiments comprise such a method wherein the androgen-related cancer is endometrial cancer.

Some embodiments comprise use of a polypeptide of the invention in the manufacture of a medicament for the treatment or prevention of estrogen flare or testosterone flare.

In one aspect of the invention there is provided a polypeptide comprising a nuclear hormone receptor agonist binding region, the nuclear hormone receptor agonist binding region capable of binding to a nuclear hormone receptor agonist at a sufficient affinity or avidity such that upon administration of the polypeptide to a mammalian subject the level of biologically available nuclear hormone receptor agonist is decreased.

Some embodiments comprise use of a polypeptide of the invention wherein the level of biologically available nuclear hormone receptor agonist is measured in the blood of the subject. Some embodiments comprise use of a polypeptide of the invention having an affinity or avidity for the nuclear hormone receptor agonist that is equal to or greater than the affinity or avidity between the nuclear hormone receptor agonist and a natural carrier of the nuclear hormone receptor agonist. Some embodiments comprise use of a polypeptide of the invention wherein the natural carrier is selected from the group consisting of SHBG, albumin, transcortin and thyroid hormone binding globulin. Some embodiments comprise use of a polypeptide of the invention wherein the nuclear hormone receptor agonist binding region includes a sequence from the ligand binding region of a nuclear hormone receptor, or functional equivalent thereof. Some embodiments comprise use of a polypeptide of the invention wherein the nuclear hormone receptor is selected from the group consisting of an androgen receptor, a glucocorticoid receptor, a mineralocorticoid receptor, a progestin receptor, a progesterone receptor, an estrogen receptor, and a thyroid hormone receptor.

Some embodiments comprise use of a polypeptide of the invention wherein the nuclear hormone receptor agonist is selected from the group consisting of corticosterone (11beta,21-dihydroxy-4-pregnene-3,20-dione); deoxycorticosterone (21-hydroxy-4-pregnene-3,20-dione); cortisol (11beta,17,21-trihydroxy-4-pregnene-3,20-dione); 11-deoxycortisol (17,21-dihydroxy-4-pregnene-3,20-dione); cortisone (17,21-dihydroxy-4-pregnene-3,11,20-trione); 18-hydroxycorticosterone (11beta,18,21-trihydroxy-4-pregnene-3,20-dione); 1α-hydroxycorticosterone (1alpha,11beta,21-trihydroxy-4-pregnene-3,20-dione); aldosterone 18,11-hemiacetal of 11beta,21-dihydroxy-3,20-dioxo-4-pregnen-18-al, androstenedione (4-androstene-3,17-dione); 4-hydroxy-androstenedione; 11β-hydroxyandrostenedione (11 beta-4-androstene-3,17-dione); androstanediol (3-beta,17-beta-Androstanediol); androsterone (3alpha-hydroxy-5alpha-androstan-17-one); epiandrosterone (3beta-hydroxy-5alpha-androstan-17-one); adrenosterone (4-androstene-3,11,17-trione); dehydroepiandrosterone (3beta-hydroxy-5-androsten-17-one); dehydroepiandrosterone sulphate (3beta-sulfoxy-5-androsten-17-one); testosterone (17beta-hydroxy-4-androsten-3-one); epitestosterone (17alpha-hydroxy-4-androsten-3-one); 5α-dihydrotestosterone (17beta-hydroxy-5alpha-androstan-3-one 5β-dihydrotestosterone; 5-beta-dihydroxy testosterone (17beta-hydroxy-5beta-androstan-3-one); 11β-hydroxytestosterone (11beta,17beta-dihydroxy-4-androsten-3-one); 11-ketotestosterone (17beta-hydroxy-4-androsten-3,17-dione), estrone (3-hydroxy-1,3,5(10)-estratrien-17-one); estradiol (1,3,5(10)-estratriene-3,17beta-diol); estriol 1,3,5(10)-estratriene-3,16alpha,17beta-triol; pregnenolone (3-beta-hydroxy-5-pregnen-20-one); 17-hydroxypregnenolone (3-beta,17-dihydroxy-5-pregnen-20-one); progesterone (4-pregnene-3,20-dione); 17-hydroxyprogesterone (17-hydroxy-4-pregnene-3,20-dione); progesterone (pregn-4-ene-3,20-dione); T3 and T4.

Some embodiments comprise use of a polypeptide of the invention wherein the nuclear hormone receptor agonist binding region includes the androgen binding domain from the sex hormone binding globulin, or functional equivalent thereof. Some embodiments comprise use of a polypeptide of the invention having a single nuclear hormone receptor agonist binding region. Some embodiments comprise use of a polypeptide of the invention comprising a carrier region. Some embodiments comprise use of a polypeptide of the invention wherein the carrier is the Fc region of human IgG, or functional equivalent thereof.

Some embodiments comprise use of a polypeptide of the invention that is selected from the group consisting of a fusion protein, a monoclonal antibody, a polyclonal antibody, and a single chain antibody. Some embodiments comprise use of a polypeptide of the invention comprising a multimerisation domain.

Some embodiments comprise a nucleic acid molecule capable of encoding a polypeptide according to the invention. Some embodiments comprise a vector comprising a nucleic acid molecule according to the invention. Some embodiments comprise a composition comprising a polypeptide according to the invention and a pharmaceutically acceptable carrier.

In one aspect of the invention there is provided a method for treating or preventing a condition related to excess nuclear hormone receptor agonist in a subject, the method comprising administering to a subject in need thereof an effective amount of a ligand capable of binding a nuclear hormone receptor agonist in the subject, such that the level of biologically available nuclear hormone receptor agonist in the subject is decreased as compared with the level of biologically available nuclear hormone receptor agonist present in the subject prior to administration of the polypeptide.

Some embodiments comprise such a method wherein the level of biologically available nuclear hormone receptor agonist is measured in the blood of the subject. Some embodiments comprise such a method wherein the ligand is a polypeptide according to the invention. Some embodiments comprise such a method wherein the ligand is in the form of a composition according to the invention.

Some embodiments comprise such a method method for treating or preventing a condition related to excess nuclear hormone receptor agonist, the method comprising administering to a subject in need thereof an effective amount of a nucleic acid molecule or a vector according to the invention. Some embodiments comprise such a method wherein the condition related to excess nuclear hormone receptor agonist is selected from the group consisting of congenital adrenal hyperplasia (CAH), apparent mineralocorticoid excess (AME), hypertension, Cushing syndrome, Cushing disease, an excess androgen disorder in a female, polycystic ovary syndrome (PCOS), hirsutism, menstrual irregularity, dysfunctional uterine bleeding, amenorrhea, infertility, ovarian enlargement or frequent ovarian cysts, endometrial hyperplasia, fibrocystic breasts, adult virilization, an excess androgen disorder in a male, hypofertility, infertility, acne, premature balding, pediatric virilization, precocious puberty, clitoral enlargement, undesired increased muscle strength, frontal hair thinning, undesired deepening of the voice, menstrual disruption, anovulation, adrenal virilism, hyperaldosteronism, thyrotoxicosis, hypermetabolism, tachycardia, fatigue, weight loss, tremor, Graves' disease, goiter, exophthalmos, and pretibial myxedema.

Some embodiments comprise use of a polypeptide according to the invention in the manufacture of a medicament for the treatment or prevention of a condition related to excess nuclear hormone receptor agonist. Some embodiments comprise use of a nucleic acid molecule according to the invention in the manufacture of a medicament for the treatment or prevention of a condition related to excess nuclear hormone receptor agonist.

In one aspect of the invention there is provided a polypeptide for regulating a reproductive physiology of an animal, the polypeptide comprising a steroid sex hormone binding region, the steroid sex hormone binding region capable of binding to a steroid sex hormone at a sufficient affinity or avidity such that upon administration of the polypeptide to the animal the level of biologically available steroid sex hormone is decreased.

Some embodiments comprise such a polypeptide wherein the level of biologically available steroid sex hormone is measured in the blood of the animal. Some embodiments comprise such a polypeptide having an affinity or avidity for the steroid sex hormone that is equal to or greater than the affinity or avidity between the steroid sex hormone and a natural carrier of the steroid sex hormone. Some embodiments comprise such a polypeptide wherein the natural carrier is selected from the group consisting of SHBG and albumin. Some embodiments comprise such a polypeptide wherein the steroid sex hormone binding region comprisesa sequence from the binding region of a steroid sex hormone receptor. Some embodiments comprise such a polypeptide wherein the steroid sex hormone receptor is selected from the group consisting of an androgen receptor, a progesterone receptor, and an estrogen receptor.

Some embodiments comprise such a polypeptide wherein the steroid sex hormone is selected from the group consisting of androstenedione (4-androstene-3,17-dione); 4-hydroxy-androstenedione; 11β-hydroxyandrostenedione (11beta-4-androstene-3,17-dione); androstanediol (3-beta,17-beta-Androstanediol); androsterone (3alpha-hydroxy-5alpha-androstan-17-one); epiandrosterone (3beta-hydroxy-5alpha-androstan-17-one); adrenosterone (4-androstene-3,11,17-trione); dehydroepiandrosterone (3beta-hydroxy-5-androsten-17-one); dehydroepiandrosterone sulphate (3beta-sulfoxy-5-androsten-17-one); testosterone (17beta-hydroxy-4-androsten-3-one); epitestosterone (17alpha-hydroxy-4-androsten-3-one); 5α-dihydrotestosterone (17beta-hydroxy-5alpha-androstan-3-one 5β-dihydrotestosterone; 5-beta-dihydroxy testosterone (17beta-hydroxy-5beta-androstan-3-one); 11β-hydroxytestosterone (11beta,17beta-dihydroxy-4-androsten-3-one); 11-ketotestosterone (17beta-hydroxy-4-androsten-3,17-dione), estrone (3-hydroxy-1,3,5(10)-estratrien-17-one); estradiol (1,3,5(10)-estratriene-3,17beta-diol); estriol 1,3,5(10)-estratriene-3,16alpha,17beta-triol; pregnenolone (3-beta-hydroxy-5-pregnen-20-one); 17-hydroxypregnenolone (3-beta,17-dihydroxy-5-pregnen-20-one); progesterone (4-pregnene-3,20-dione); 17-hydroxyprogesterone (17-hydroxy-4-pregnene-3,20-dione) and progesterone (pregn-4-ene-3,20-dione).

Some embodiments comprise such a polypeptide having a single steroid sex hormone binding region. Some embodiments comprise such a polypeptide comprising a carrier region. Some embodiments comprise such a polypeptide wherein the carrier region comprisesa sequence of the IgG Fc region. Some embodiments comprise such a polypeptide that is selected from the group consisting of a fusion protein, a monoclonal antibody, a polyclonal antibody, and a single chain antibody. Some embodiments comprise such a polypeptide comprising a multimerisation domain. Some embodiments comprise such a polypeptide in combination with a pharmaceutically acceptable carrier.

Some embodiments comprise a nucleic acid molecule capable.of encoding a polypeptide according to the invention. Some embodiments comprise a vector comprising a nucleic acid molecule according to the invention.

Some embodiments comprise a method for regulating a reproductive physiology of an animal, the method comprising administering to a subject in need thereof an effective amount of a polypeptide according to the invention. Some embodiments comprise such a method wherein the level of biologically available steroid is measured in the blood of the subject. Some embodiments comprise such a method wherein the level of biologically available steroid is measured in the blood of the subject wherein the polypeptide is in the form of a composition according to the invention.

Some embodiments comprise a method forregulating a reproductive physiology, the method comprising administering to a subject in need thereof an effective amount of a nucleic acid molecule or a vector according to the invention. Some embodiments comprise such a method wherein the reproductive physiology is selected from the group consisting of ovulation, conception, parturition, commencement of estrus, maintenance of estrus, termination of estrus, commencement of pregnancy, maintenance of pregnancy, termination of pregnancy, erection, semen production, spermatogenesis, or a behaviour selected from the group consisting of restlessness, agitation, hyperactivity, frequent urination, sniffing or licking a stallion, straddling posture, clitoral “winking”, raising the tail, dominance, aggression, Flehmen response, impatience, alertness, hyperactivity, restlessness, vocalization, nudging or smelling or biting a mare. Some embodiments comprise such a method wherein the animal is selected from the group consisting of a horse, a pig, a cow, a goat, a sheep, an alpaca, a dog, and a cat.

Some embodiments comprise use of a polypeptide according to the invention in the manufacture of a medicament for regulating a reproductive physiology in an animal. Some embodiments comprise use of a nucleic acid molecule according to the invention in the manufacture of a medicament for regulating a reproductive physiology in an animal. Some embodiments comprise use of a vector according to the invention in the manufacture of a medicament for regulating a reproductive physiology in an animal. Some embodiments comprise use according to the invention wherein the animal is selected from the group consisting of a horse, a pig, a cow, a goat, a sheep, an alpaca, a dog, and a cat.

The polypeptides of the present invention may comprise a carrier region which in one embodiment may be the Fc region of human IgG or an anologue thereof. The disclosure in the present specification uses immunoglobulins and IgG in particular to illustrate certain principals. Equally, however, the polypeptide may comprise any suitable carrier region or use any other suitable method of prolonging serum half life. In some embodiments, it may comprise one or more of a common plasma protein, human serum albumin, an immunoglobulin, a human domain antibody, an immunoglobulin heavy chain variable domain, a highly solvated, physiologically inert chemicall polymer (such as a polyethylene glycol), a transferin, an arabinogalactan fusion protein, through site-specific incorporation of a glycosylation site, a protein of long plasma half life, a high molecular weight protein, or a biological equivalent thereof.

Common plasma proteins such as human serum albumin (HSA) and immunoglobulins (Igs), including humanized antibodies, show long half-lives, typically of 2-3 weeks, which is attributable to their specific interaction with the neonatal Fc receptor (FcRn) and endosomal recycling (Ghetie and Ward, 2002). In contrast, most other proteins of pharmaceutical interest, in particular recombinant antibody fragments, hormones, interferons, etc., suffer from rapid clearance. This is particularly true for proteins whose size is below the threshold value for kidney filtration of about 70 kDa (Caliceti and Veronese, 2003). In these cases, the plasma half-life of an unmodified pharmaceutical protein may be considerably less than an hour, thus rendering it essentially useless for most therapeutic applications. In order to achieve sustained pharmacological action and also improved patient compliance, with required dosing intervals extending to several days or even weeks, two major strategies have been established for the purposes of biopharmaceutical drug development.

The half-life in vivo of a biologically active protein or peptide can be substantially prolonged by covalently coupling such protein or peptide to a polypeptide fragment capable of binding to a serum protein. Thus, according to one aspect of the invention, there is provided a process for extending the half-life in vivo of a biologically active protein or peptide, such process comprising the steps of covalently coupling the protein or peptide to a polypeptide fragment which is capable of binding to a serum protein. When administering the protein or peptide conjugate resulting from such process the binding thereof to the serum protein results in substantially extended biological activity due to increased half-life thereof.

According to a preferred embodiment of this aspect of the invention said polypeptide fragment is capable of binding to serum albumin, such as a serum albumin of mammal origin, for example human serum albumin.

The binding polypeptide fragment of the conjugate can for example originate from streptococcal protein G.

Another aspect of the invention is constituted by the use of the protein or peptide conjugate as defined above for the manufacture of a drug or medicament which, when administered to a mammal including man, shows extended half life in vivo thus prolonging the biological activity of the conjugate.

Alternatively Human domain antibodies (dAbs) that bind to mouse, rat and/or human serum albumin (SA) can be fused to the Ligand binding domains of Nuclear receptors (NR-LBD) and these fusion AlbudAbs could potentially be used to generate a range of long half-life versions of the Nuclear receptor ligand binding domains (NR-LBD) in order to improve their dosing regimen and/or clinical effect.

In some embodiments, the polypeptide binding moiety has binding specificity for serum albumin. For example, the polypeptide binding moiety can be an antigen-binding fragment of an antibody that has binding specificity for serum albumin.

In some embodiments the carrier protein is an immunoglobulin heavy chain variable domain that has binding specificity for serum albumin, or an immunoglobulin light chain variable domain that has binding specificity for serum albumin. In such embodiments, the nuclear receptor ligand binding domain (NR-LBD) can be located amino terminally to the carrier protein moiety, or can be located amino terminally to NR-LBD. Preferably, the heavy chain variable domain and light chain variable domain have binding specificity for human serum albumin.

PEGylation, a fundamentally different methodology for prolonging the plasma half-life of biopharmaceuticals is the conjugation with highly solvated and physiologically inert chemical polymers, thus effectively enlarging the hydrodynamic diameter of the therapeutic protein beyond the glomerular pore size of 3-5 nm (Caliceti and Veronese, 2003). Covalent coupling under biochemically mild conditions with activated derivatives of polyethylene glycol (PEG), either randomly via Lys side chains (Clark et al., 1996↓) or by means of specifically introduced Cys residues (Rosendahl et al., 2005↓), has been tremendously successful in yielding several approved drugs. Corresponding advantages have been achieved especially in conjunction with small proteins possessing specific pharmacological activity, for example Pegasys®, a chemically PEGylated recombinant IFN-α-2a (Harris and Chess, 2003↓; Walsh, 2003↓).

Many PEG derivatives, covering a range of sizes and including branched versions, with differing reactive groups and spacers, are currently available, thus making PEGylation the method of choice for tailoring the plasma half-life of biopharmaceuticals in the range from days to weeks. This offers advantages also for the clinical application of bacterially produced antibody fragments instead of costly full size Igs. Although the plasma half-life of an Fab′ fragment is usually shorter than 1 h, its area under the curve (AUC) can be dramatically increased 13.5-fold by site-specific conjugation with a single 40 kDa PEG chain (Chapman, 2002↓).

Polyethylene glycol (PEG) is a substance that can be attached to a protein, resulting in longer-acting, sustained activity of the protein. If the activity of a protein is prolonged by the attachment to PEG, the frequency that the protein needs to be administered may be decreased. PEG attachment, however, often decreases or destroys the protein's therapeutic activity. While in some instance PEG attachment can reduce immunogenicity of the protein, in other instances it may increase immunogenicity.

PEG is a highly flexible and soluble polymer that has gained widespread scientific and regulatory acceptance as a chemical modification for therapeutic proteins. PEGylation improves PK predominantly by increasing the effective size of a protein, with most significant effects for proteins smaller than 70 kDa [24 and 25]. PEGylation can also reduce immunogenicity and aggregation [26]. Although a variety of chemistries exist [27 and 28] for coupling PEGs of various sizes to proteins, the greatest attachment specificity generally arises from PEGylation at the N-terminus or unpaired cysteines.

Another serum protein, glycosylated human transferrin (Tf) has also been used to make fusions with therapeutic proteins to target delivery to the interior of cells or to carry agents across the blood-brain barrier. These fusion proteins comprising glycosylated human Tf have been used to target nerve growth factor (NGF) or ciliary neurotrophic factor (CNTF) across the blood-brain barrier by fusing full-length Tf to the agent. See U.S. Pat. Nos. 5,672,683 and 5,977,307. In these fusion proteins, the Tf portion of the molecule is glycosylated and binds to two atoms of iron, which is required for Tf binding to its receptor on a cell and, according to the inventors of these patents, to target delivery of the NGF or CNTF moiety across the blood-brain barrier. Transferrin fusion proteins have also been produced by inserting an HIV-1 protease, target sequence into surface exposed loops of glycosylated transferrin to investigate the ability to produce another form of Tf fusion for targeted delivery to the inside of a cell via the Tf receptor (Ali et al. (1999) J. Biol. Chem. 274(34):24066-24073).

Serum transferrin (Tf) is a monomeric glycoprotein with a molecular weight of 80,000 daltons that binds iron in the circulation and transports it to various tissues via the transferrin receptor (TfR) (Aisen et al. (1980) Ann. Rev. Biochem. 49: 357-393; MacGillivray et al. (1981) J. Biol. Chem. 258: 3543-3553, U.S. Pat. No. 5,026,651). Tf is one of the most common serum molecules, comprising up to about 5-10% of total serum proteins. Carbohydrate deficient transferrin occurs in elevated levels in the blood of alcoholic individuals and exhibits a longer half life (approximately 14-17 days) than that of glycosylated transferrin (approximately 7-10 days). See van Eijk et al. (1983) Clin. Chim. Acta 132:167-171, Stibler (1991) Clin. Chem. 37:2029-2037 (1991), Arndt (2001) Clin. Chem. 47(1):13-27 and Stibler et al. in “Carbohydrate-deficient consumption”, Advances in the Biosciences, (Ed Nordmann et al.), Pergamon, 1988, Vol. 71, pages 353-357).

The structure of Tf has been well characterized and the mechanism of receptor binding, iron binding and release and carbonate ion binding have been elucidated (U.S. Pat. Nos. 5,026,651, 5,986,067 and MacGillivray et al. (1983) J. Biol. Chem. 258(6):3543-3546).

Transferrin and antibodies that bind the transferrin receptor have also been used to deliver or carry toxic agents to tumor cells as cancer therapy (Baselga and Mendelsohn, 1994), and transferrin has been used as a non-viral gene therapy vector to deliver DNA to cells (Frank et al., 1994; Wagner et al., 1992). The ability to deliver proteins to the central nervous system (CNS) using the transferrin receptor as the entry point has been demonstrated with several proteins and peptides including CD4 (Walus et al., 1996), brain derived neurotrophic factor (Pardridge et al., 1994), glial derived neurotrophic factor (Albeck et al.), a vasointestinal peptide analogue (Bickel et al., 1993), a beta-amyloid peptide (Saito et al., 1995), and an antisense oligonucleotide (Pardridge et al., 1995).

Therapeutic proteins like human interferon alpha2 generally possess short serum half-lives due to their small size, hence rapid renal clearance, and susceptibility to serum proteases. Chemical derivatization, such as addition of polyethylene glycol (PEG) groups overcomes both problems, but at the expense of greatly decreased bioactivity. One method yields biologically potent interferon alpha2b (IFNalpha2) in high yields and with increased serum half-life when expressed as arabinogalactan-protein (AGP) chimeras in cultured tobacco cells. Thus IFNalpha2-AGPs targeted for secretion typically gave 350-1400-fold greater secreted yields than the non-glycosylated IFNalpha2 control. The purified AGP domain itself was not immunogenic when injected into mice and only mildly so when injected as a fusion glycoprotein. Importantly, the AGP-IFNalpha2 chimeras showed up to a 13-fold increased in vivo serum half-life while the biological activity remained similar to native IFNalpha2. The use of arabinogalactan glycomodules may provide a general approach to the enhanced production of therapeutic proteins by plants.

GlycosylationSite-specific incorporation of glycosylation sites serves as an additional approach for improving PK. A notable example is Amgen's hyperglycosylated erythropoietin (Epo) variant Aranesp® (darbepoetin alfa), engineered to contain two additional N-linked glycosylation sites. The additional glycosylation increases the serum half-life threefold while reducing in vitro binding roughly fourfold [31]. Thus, Aranesp® is another example of how modification can improve in vivo efficacy, despite reducing specific activity. Accordingly, future efforts could benefit from using rational methods to identify N-linked or O-linked glycosylation sites that best maintain the structural and functional properties of the protein.

The carrier protein can be any polypeptide fused to an NR-LBD protein. Examples of carrier proteins include those proteins with a long plasma half-life. Preferred carrier proteins are at least 50 amino acids, at least 100 amino acids, or at least 200 amino acids in length. Typically, proteins that exhibit an extended serum half-life are those proteins which have a high molecular weight, e.g., greater than 50,000 Daltons. Preferably, the carrier protein limits the proteolytic cleavage of the fusion protein. The circulating half-life of the NR-LBD fusion protein can be measured by assaying the serum level of the fusion protein as a function of time.

In one embodiment, the carrier protein can also contain an alteration in its sequence, for example, preferably in the C-terminal portion of the carrier protein, e.g., within about 100 residues, more preferably within about 50 residues, or about 25 residues, and even more preferably within about 10 residues from the C-terminus of the carrier protein.

In one embodiment, the carrier protein is albumin, for example, human serum albumin (HSA). The genes coding for HSA are highly polymorphic and more than 30 different genetic alleles have been reported (Weitkamp L. R. et al., Ann. Hum. Genet. 37 (1973) 219-226, the teachings of which are hereby incorporated by reference). Alternatively, the albumin can be from any animal such as dog, chicken, duck, mouse or rat.

In another embodiment the carrier protein is an antibody. In general, an antibody-based NR-LBD fusion protein of the invention comprises a portion of an immunoglobulin (Ig) protein joined to an NR-LBD protein. Examples of immunoglobulins include IgG, IgM, IgA, IgD, and IgE.

The immunoglobulin protein or a portion of an immunoglobulin protein can include a variable or a constant domain. An immunoglobulin (Ig) chain preferably includes a portion of an immunoglobulin heavy chain, for example, an immunoglobulin variable region capable of binding a preselected cell-type. In a preferred embodiment, the Ig chain comprises a variable region specific for a target antigen as well as a constant region. The constant region may be the constant region normally associated with the variable region, or a different one, e.g., variable and constant regions from different species. In a more preferred embodiment, an Ig chain includes a heavy chain. The heavy chain may include any combination of one or more CH1, CH2, or CH3 domains. Preferably, the heavy chain includes CH1, CH2, and CH3 domains, and more preferably only CH2 and CH3 domains. In one embodiment, the portion of the immunoglobulin includes an Fv region with fused heavy and light chain variable regions.

In one embodiment, the carrier protein comprises an Fc portion of an immunoglobulin protein. As used herein, “Fc portion” encompasses domains derived from the constant region of an immunoglobulin, preferably a human immunoglobulin, including a fragment, analog, variant, mutant or derivative of the constant region. Suitable immunoglobulins include IgG1, IgG2, IgG3, IgG4, and other classes. The constant region of an immunoglobulin is defined as a naturally-occurring or synthetically-produced polypeptide homologous to the immunoglobulin C-terminal region, and can include a CH1 domain, a hinge, a CH2 domain, a CH3 domain, or a CH4 domain, separately or in combination.

In the present invention, the Fc portion typically includes at least a CH2 domain. For example, the Fc portion can include, from N-terminus to C-terminus, hinge, CH2, and CH3 domains. Alternatively, the Fc portion can include all or a portion of the hinge region, the CH2 domain and/or the CH3 domain.

The constant region of an immunoglobulin is responsible for many important antibody functions including Fc receptor (FcR) binding and complement fixation. There are five major classes of heavy chain constant region, classified as IgA, IgG, IgD, IgE, IgM, each with characteristic effector functions designated by isotype. For example, IgG is separated into four y subclasses: .gamma.1, .gamma.2, .gamma.3, and .gamma.4, also known as IgG1, IgG2, IgG3, and IgG4, respectively.

IgG molecules interact with multiple classes of cellular receptors including three classes of Fc.gamma. receptors (Fc.gamma.R) specific for the IgG class of antibody, namely Fc.gamma.RI, Fc.gamma.RII, and Fc.gamma.RIII. The important sequences for the binding of IgG to the Fc.gamma.R receptors have been reported to be located in the CH2 and CH3 domains. The serum half-life of an antibody is influenced by the ability of that antibody to bind to an Fc receptor (FcR). Similarly, the serum half-life of immunoglobulin fusion proteins is also influenced by the ability to bind to such receptors (Gillies S D et al., (1999) Cancer Res. 59:2159-66, the teachings of which are hereby incorporated by reference). Compared to those of IgG1, CH2 and CH3 domains of IgG2 and IgG4 have biochemically undetectable or reduced binding affinity to Fc receptors. It has been reported that immunoglobulin fusion proteins containing CH2 and CH3 domains of IgG2 or IgG4 had longer serum half-lives compared to the corresponding fusion proteins containing CH2 and CH3 domains of IgG1 (U.S. Pat. No. 5,541,087; Lo et al., (1998) Protein Engineering, 11:495-500, the teachings of which are hereby incorporated by reference). Accordingly, preferred CH2 and CH3 domains for the present invention are derived from an antibody isotype with reduced receptor binding affinity and effector functions, such as, for example, IgG2 or IgG4. More preferred CH2 and CH3 domains are derived from IgG2.

The hinge region is normally located C-terminal to the CH1 domain of the heavy chain constant region. In the IgG isotypes, disulfide bonds typically occur within this hinge region, permitting the final tetrameric molecule to form. This region is dominated by prolines, serines and threonines. When included in the present invention, the hinge region is typically at least homologous to the naturally-occurring immunoglobulin region that includes the cysteine residues to form disulfide bonds linking the two Fc moieties. Representative sequences of hinge regions for human and mouse immunoglobulins can be found in Borrebaeck, C. A. K., ed., (1992) ANTIBODY ENGINEERING, A PRACTICAL GUIDE, W.H. Freeman and Co., the teachings of which are hereby incorporated by reference. Suitable hinge regions for the present invention can be derived from IgG1, IgG2, IgG3, IgG4, and other immunoglobulin classes. The IgG1 hinge region has three cysteines, two of which are involved in disulfide bonds between the two heavy chains of the immunoglobulin. These same cysteines permit efficient and consistent disulfide bonding formation between Fc portions. Therefore, a preferred hinge region of the present invention is derived from IgG1, more preferably from human IgG1. In some embodiments, the first cysteine within the human IgG1 hinge region is mutated to another amino acid, preferably serine. The IgG2 isotype hinge region has four disulfide bonds that tend to promote oligomerization and possibly incorrect disulfide bonding during secretion in recombinant systems. A suitable hinge region can be derived from an IgG2 hinge; the first two cysteines are each preferably mutated to another amino acid. The hinge region of IgG4 is known to form interchain disulfide bonds inefficiently. However, a suitable hinge region for the present invention can be derived from the IgG4 hinge region, preferably containing a mutation that enhances correct formation of disulfide bonds between heavy chain-derived moieties (Angal S, et al. (1993) Mol. Immunol., 30:105-8, the teachings of which are hereby incorporated by reference).

In accordance with the present invention, the Fc portion can contain CH2 and/or CH3 domains and a hinge region that are derived from different antibody isotypes, i.e., a hybrid Fc portion. For example, in one embodiment, the Fc portion contains CH2 and/or CH3 domains derived from IgG2 or IgG4 and a mutant hinge region derived from IgG1. Alternatively, a mutant hinge region from another IgG subclass is used in a hybrid Fc portion. For example, a mutant form of the IgG4 hinge that allows efficient disulfide bonding between the two heavy chains can be used. A mutant hinge can also be derived from an IgG2 hinge in which the first two cysteines are each mutated to another amino acid. Such hybrid Fc portions facilitate high-level expression and improve the correct assembly of the Fc fusion proteins. Assembly of such hybrid Fc portions has been described in U.S. Patent Application Publication No. 20030044423, the disclosure of which is hereby incorporated by reference.

In some embodiments, the Fc portion contains amino acid modifications that generally extend the serum half-life of an Fc fusion protein. Such amino acid modifications include mutations substantially decreasing or eliminating Fc receptor binding or complement fixing activity. For example, the glycosylation site within the Fc portion of an immunoglobulin heavy chain can be removed. In IgG1, the glycosylation site is Asn297. In other immunoglobulin isotypes, the glycosylation site corresponds to Asn297 of IgG1. For example, in IgG2 and IgG4, the glycosylation site is the asparagine within the amino acid sequence Gln-Phe-Asn-Ser. Accordingly, a mutation of Asn297 of IgG1 removes the glycosylation site in an Fc portion derived from IgG1. In one embodiment, Asn297 is replaced with Gln. Similarly, in IgG2 or IgG4, a mutation of asparagine within the amino acid sequence Gln-Phe-Asn-Ser removes the glycosylation site in an Fc portion derived from IgG2 or IgG4 heavy chain. In one embodiment, the asparagine is replaced with a glutamine. In other embodiments, the phenylalanine within the amino acid sequence Gln-Phe-Asn-Ser is further mutated to eliminate a potential non-self T-cell epitope resulting from asparagine mutation. For example, the amino acid sequence Gln-Phe-Asn-Ser within an IgG2 or IgG4 heavy chain can be replaced with a Gln-Ala-Gln-Ser amino acid sequence.

It has also been observed that alteration of amino acids near the junction of the Fc portion and the non-Fc portion can dramatically increase the serum half-life of the Fc fusion protein (PCT publication WO 01/58957, the disclosure of which is hereby incorporated by reference). Accordingly, the junction region of an Fc-NR-LBD fusion protein of the present invention can contain alterations that, relative to the naturally-occurring sequences of an immunoglobulin heavy chain and an NR-LBD protein, preferably lie within about 10 amino acids of the junction point. These amino acid changes can cause an increase in hydrophobicity by, for example, changing the C-terminal lysine of the Fc portion to a hydrophobic amino acid such as alanine or leucine.

In other embodiments, the Fc portion contains amino acid alterations of the Leu-Ser-Leu-Ser segment near the C-terminus of the Fc portion of an immunoglobulin heavy chain. The amino acid substitutions of the Leu-Ser-Leu-Ser segment eliminate potential junctional T-cell epitopes. In one embodiment, the Leu-Ser-Leu-Ser amino acid sequence near the C-terminus of the Fc portion is replaced with an Ala-Thr-Ala-Thr amino acid sequence. In other embodiments, the amino acids within the Leu-Ser-Leu-Ser segment are replaced with other amino acids such as glycine or proline. Detailed methods of generating amino acid substitutions of the Leu-Ser-Leu-Ser segment near the C-terminus of an IgG1, IgG2, IgG3, IgG4, or other immunoglobulin class molecule have been described in U.S. Patent Application Publication No. 20030166877, the disclosure of which is hereby incorporated by reference.

According to the invention, an antibody-based fusion protein with an enhanced in vivo circulating half-life can be further enhanced by modifying within the Fc portion itself. These may be residues including or adjacent to Ile 253, His 310 or His 435 or other residues that can affect the ionic environments of these residues when the protein is folded in its 3-dimensional structure. The resulting proteins can be tested for optimal binding at pH 6 and at pH 7.4-8 and those with high levels of binding at pH 6 and low binding at pH 8 are selected for use in vivo. Such mutations can be usefully combined with the junction mutations of the invention.

In another embodiment of the invention, the binding affinity of fusion proteins for FcRp is optimized by alteration of the interaction surface of the Fc moiety that contacts FcRp. The important sequences for the binding of IgG to the FcRp receptor have been reported to be located in the CH2 and CH3 domains. According to the invention, alterations of the fusion junction in a fusion protein are combined with alterations of the interaction surface of Fc with FcRp to produce a synergistic effect. In some cases it may be useful to increase the interaction of the Fc moiety with FcRp at pH 6, and it may also be useful to decrease the interaction of the Fc moiety with FcRp at pH 8. Such modifications include alterations of residues necessary for contacting Fc receptors or altering others that affect the contacts between other heavy chain residues and the FcRp receptor through induced conformational changes. Thus, in a preferred embodiment, an antibody-based fusion protein with enhanced in vivo circulating half-life is obtained by first linking the coding sequences of an Ig constant region and a second, non-immunoglobulin protein and then introducing a mutation (such as a point mutation, a deletion, an insertion, or a genetic rearrangement) in an IgG constant region at or near one or more amino acid selected from Ile.sub.253, His.sub.310 and His.sub.435. The resulting antibody-based fusion proteins have a longer in vivo circulating half-life than the unmodified fusion proteins.

In certain circumstances it is useful to mutate certain effector functions of the Fc moiety. For example, complement fixation may be eliminated. Alternatively or in addition, in another set of embodiments the Ig component of the fusion protein has at least a portion of the constant region of an IgG that has reduced binding affinity for at least one of Fc.gamma.RI, Fc.gamma.RII or Fc.gamma.RIII. For example, the gamma4 chain of IgG may be used instead of gamma1. The alteration has the advantage that the gamma4 chain results in a longer serum half-life, functioning synergistically with one or more mutations at the fusion junction. Similarly, IgG2 may also be used instead of IgG1. In an alternative embodiment of the invention, a fusion protein includes a mutant IgG1 constant region, for example an IgG1 constant region having one or more mutations or deletions of Leu.sub.234, Leu.sub.235, Gly.sub.236, Gly.sub.237, Asn.sub.297, or Pro.sub.331. In a further embodiment of the invention, a fusion protein includes a mutant IgG3 constant region, for example an IgG3 constant region having one or more mutations or deletions of Leu.sub.281, Leu.sub.282, Gly283, Gly.sub.284, Asn.sub.344, or Pro.sub.378. However, for some applications, it may be useful to retain the effector function that accompanies Fc receptor binding, such as ADCC.

In some embodiments, the carrier protein of the fusion protein is a hormone, neurotrophin, body-weight regulator, serum protein, clotting factor, protease, extracellular matrix component, angiogenic factor, anti-angiogenic factor, or another secreted protein or secreted domain. For example, CD26, IgE receptor, polymeric IgA receptor, other antibody receptors, Factor VIII, Factor IX, Factor X, TrkA, PSA, PSMA, Flt-3 Ligand, endostatin, angiostatin, and domains of these proteins.

In other embodiments, the carrier protein is a non-human or non-mammalian protein. For example, HIV gp120, HIV Tat, surface proteins of other viruses such as adenovirus, and RSV, other HIV components, parasitic surface proteins such as malarial antigens, and bacterial surface proteins are preferred. These non-human proteins may be used, for example, as antigens, or because they have useful activities. For example, the carrier polypeptide may be streptokinase, staphylokinase, urokinase, tissue plasminogen activator, or other proteins with useful enzymatic activities.

In certain embodiments, the carrier protein is a cytokine. The term “cytokine” is used herein to describe naturally occurring or recombinant proteins, analogs thereof, and fragments thereof which elicit a specific biological response in a cell which has a receptor for that cytokine. Preferably, cytokines are proteins that may be produced and excreted by a cell. Preferred cytokines include interleukins such as IL-2, IL-4, IL-5, IL-6, IL-7, IL-10, IL-12, IL-13, IL-14, IL-15, IL-16 and IL-18, hematopoietic factors such as granulocyte-macrophage colony stimulating factor (GM-CSF), granulocyte colony stimulating factor (G-CSF) and erythropoietin, tumor necrosis factors (TNF) such as TNF.alpha., lymphokines such as lymphotoxin, regulators of metabolic processes such as leptin, interferons such as interferon .alpha., interferon .beta., and interferon .gamma., and chemokines.

In one aspect the present invention provides a polypeptide comprising a nuclear hormone receptor agonist binding region, the nuclear hormone receptor agonist binding region capable of binding to a nuclear hormone receptor agonist at a sufficient affinity or avidity such that upon administration of the polypeptide to a mammalian subject the level of biologically available nuclear hormone receptor agonist is decreased. The level of biologically available nuclear hormone receptor agonist may be measured in the blood of the subject.

In one embodiment, the polypeptide has an affinity or avidity for the nuclear hormone receptor agonist that is equal to or greater than the affinity or avidity between the nuclear hormone receptor agonist and a natural carrier of the nuclear hormone receptor agonist, such as SHBG, albumin, transcortin and thyroid hormone binding globulin.

In one embodiment of the polypeptide, the nuclear hormone receptor agonist binding region includes a sequence from the ligand binding region of a nuclear hormone receptor, or functional equivalent thereof. The nuclear hormone receptor may be an androgen receptor, a glucocorticoid receptor, a mineralocorticoid receptor, a progestin receptor, a progesterone receptor, an estrogen receptor, or a thyroid hormone receptor. In one embodiment, the polypeptide has a single nuclear hormone receptor agonist binding region.

In one embodiment of the polypeptide the nuclear hormone receptor agonist is corticosterone (11beta,21-dihydroxy-4-pregnene-3,20-dione); deoxycorticosterone (21-hydroxy-4-pregnene-3,20-dione); cortisol (11beta,17,21-trihydroxy-4-pregnene-3,20-dione); 11-deoxycortisol (17,21-dihydroxy-4-pregnene-3,20-dione); cortisone (17,21-dihydroxy-4-pregnene-3,11,20-trione); 18-hydroxycorticosterone (11beta,18,21-trihydroxy-4-pregnene-3,20-dione); 1α-hydroxycorticosterone (1alpha,11beta,21-trihydroxy-4-pregnene-3,20-dione); aldosterone 18,11-hemiacetal of 11beta,21-dihydroxy-3,20-dioxo-4-pregnen-18-al, androstenedione (4-androstene-3,17-dione); 4-hydroxy-androstenedione; 11p-hydroxyandrostenedione (11beta-4-androstene-3,17-dione); androstanediol (3-beta,17-beta-Androstanediol); androsterone (3alpha-hydroxy-5alpha-androstan-17-one); epiandrosterone (3beta-hydroxy-5alpha-androstan-17-one); adrenosterone (4-androstene-3,11,17-trione); dehydroepiandrosterone (3beta-hydroxy-5-androsten-17-one); dehydroepiandrosterone sulphate (3beta-sulfoxy-5-androsten-17-one); testosterone (17beta-hydroxy-4-androsten-3-one); epitestosterone (17alpha-hydroxy-4-androsten-3-one); 5α-dihydrotestosterone (17beta-hydroxy-5alpha-androstan-3-one 5β-dihydrotestosterone; 5-beta-dihydroxy testosterone (17beta-hydroxy-5beta-androstan-3-one); 11β-hydroxytestosterone (11beta,17beta-dihydroxy-4-androsten-3-one); 11-ketotestosterone (17beta-hydroxy-4-androsten-3,17-dione), estrone (3-hydroxy-1,3,5(10)-estratrien-17-one); estradiol (1,3,5(10)-estratriene-3,17beta-diol); estriol 1,3,5(10)-estratriene-3,16alpha,17beta-triol; pregnenolone (3-beta-hydroxy-5-pregnen-20-one); 17-hydroxypregnenolone (3-beta,17-dihydroxy-5-pregnen-20-one); progesterone (4-pregnene-3,20-dione); 17-hydroxyprogesterone (17-hydroxy-4-pregnene-3,20-dione); progesterone (pregn-4-ene-3,20-dione); T₃ or T₄.

In another embodiment of the polypeptide the nuclear hormone receptor agonist binding region includes the androgen binding domain from the sex hormone binding globulin, or functional equivalent thereof.

In one embodiment, the polypeptide comprises a carrier region such as the Fc region of human IgG. The polypeptide may be in the form of a fusion protein, a monoclonal antibody, a polyclonal antibody, or a single chain antibody, and may comprise comprising a multimerisation domain.

In another aspect the present invention provides a nucleic acid molecule capable of encoding a polypeptide as described herein, and also a vector comprising that nucleic acid.

In a further aspect the present invention provides a composition comprising a polypeptide as described herein and a pharmaceutically acceptable carrier.

Yet a further aspect of the present invention provides a method for treating or preventing a condition related to excess nuclear hormone receptor agonist in a subject, the method comprising administering to a subject in need thereof an effective amount of a ligand capable of binding a nuclear hormone receptor agonist in the subject, such that the level of biologically available nuclear hormone receptor agonist in the subject is decreased as compared with the level of biologically available nuclear hormone receptor agonist present in the subject prior to administration of the polypeptide. The level of biologically available nuclear hormone receptor agonist may be measured in the blood of the subject.

In one embodiment of the method, the ligand is a polypeptide as described herein. In another embodiment the ligand is in the form of a composition as described herein.

The present invention provides in a further aspect a method for treating or preventing a condition related to excess nuclear hormone receptor agonist, the method comprising administering to a subject in need thereof an effective amount of a nucleic acid molecule as described herein, or a vector as described herein.

Also provided is use of a polypeptide, nucleic acid molecule or vector as described herein in the manufacture of a medicament for the treatment or prevention of a condition related to excess nuclear hormone receptor agonist.

Conditions related to excess nuclear hormone receptor agonist amenable to treatment or prevention with the polypeptides, compositions, nucleic acid molecules and vectors congenital adrenal hyperplasia (CAH), apparent mineralocorticoid excess (AME), hypertension, Cushing syndrome, Cushing disease, an excess androgen disorder in a female, polycystic ovary syndrome (PCOS), hirsutism, menstrual irregularity, dysfunctional uterine bleeding, amenorrhea, infertility, ovarian enlargement or frequent ovarian cysts, endometrial hyperplasia, fibrocystic breasts, adult virilization, an excess androgen disorder in a male, hypofertility, infertility, acne, premature balding, pediatric virilization, precocious puberty, clitoral enlargement, undesired increased muscle strength, frontal hair thinning, undesired deepening of the voice, menstrual disruption, anovulation, adrenal virilism, hyperaldosteronism, thyrotoxicosis, hypermetabolism, tachycardia, fatigue, weight loss, tremor, Graves' disease, goiter, exophthalmos, and pretibial myxedema.

In one aspect, the present invention provides a polypeptide comprising an estrogen or androgen binding region, the binding region capable of binding to an estrogen or androgen at a sufficient affinity or avidity such that upon administration of the polypeptide to a mammalian subject the level of biologically available estrogen or androgen is decreased. The level of biologically available estrogen or androgen may be measured in the blood of the subject. The level of biologically available estrogen may also be measured in a breast cell or an ovarian cell of the subject, or the level of biologically available androgen is measured in an endometrial cell of the subject.

In one form of the invention the polypeptide is such that upon administration of the polypeptide the level of biologically available estrogen or androgen is decreased such that the growth of a breast cancer cell, an ovarian cancer cell or an endometrial cancer cell in the subject is decreased or substantially arrested.

In one embodiment, the polypeptide has an affinity or avidity for an estrogen or androgen that is equal to or greater than the affinity or avidity between the estrogen or the androgen and a protein that naturally binds to the estrogen or the androgen.

In another embodiment, the polypeptide has an affinity or avidity for estradiol or testosterone that is equal to or greater than the affinity or avidity between estradiol and sex hormone binding globulin, or testosterone and sex hormone binding globulin.

In a further embodiment the polypeptide has an affinity or avidity for estradiol or testosterone that is equal to or greater than the affinity or avidity between estradiol and the estrogen receptor, or testosterone and the androgen receptor.

In one form of the polypeptide the estrogen binding region comprises the estrogen binding domain from the human estrogen receptor, or a functional equivalent thereof, or the androgen binding region comprises the androgen binding domain from the human androgen receptor, or a functional equivalent thereof. The estrogen or androgen binding region may also comprise the estrogen or androgen binding domain from sex hormone binding globulin, or a functional equivalent thereof.

In one embodiment, the polypeptide has a single estrogen or androgen binding region.

In one form of the polypeptide, the polypeptide is capable of entering a breast cell, an ovarian cell, or an endometrial cell.

The polypeptide may be in the form of a fusion protein, a monoclonal antibody, a polyclonal antibody, or a single chain antibody. The polypeptide may also comprise a multimerisation domain.

In another aspect the present invention provides a nucleic acid molecule capable of encoding a polypeptide as described herein, and also a vector comprising that nucleic acid.

In a further aspect the present invention provides a composition comprising a polypeptide as described herein and a pharmaceutically acceptable carrier.

In yet a further aspect the present invention provides a method for treating or preventing an estrogen-related cancer or an androgen-related cancer in a subject, the method comprising administering to a subject in need thereof an effective amount of a ligand capable of binding estrogen or androgen in the subject, such that the level of biologically available estrogen or androgen in the subject is decreased as compared with the level of biologically available estrogen or androgen present in the subject prior to administration of the ligand. The estrogen-related cancer may be breast cancer or ovarian cancer, while the androgen-related cancer may be endometrial cancer. In one form of the method, the ligand is a polypeptide as described herein.

In one embodiment of the method the level of biologically available estrogen is measured in a breast cell or an ovarian cell. In another embodiment the level of biologically available androgen is measured in an endometrial cell. The level of biologically available estrogen or androgen may be measured in the blood of the subject.

In a first aspect the present invention provides a bi-functional molecule comprising (i) a first region capable of binding to a steroid hormone and/or steroid hormone associated molecule in solution and (ii) a second region having means for removing the bi-functional molecule and any bound steroid hormone and/or steroid hormone associated molecule from solution. The first region may be substantially specific for a steroid hormone and/or steroid hormone associated molecule.

The first region of the bi-functional molecule may be capable of binding a steroid including corticosterone (11beta,21-dihydroxy-4-pregnene-3,20-dione); deoxycorticosterone (21-hydroxy-4-pregnene-3,20-dione); cortisol (11beta,17,21-trihydroxy-4-pregnene-3,20-dione); 11-deoxycortisol (17,21-dihydroxy-4-pregnene-3,20-dione); cortisone (17,21-dihydroxy-4-pregnene-3,11,20-trione); 18-hydroxycorticosterone (11beta,18,21-trihydroxy-4-pregnene-3,20-dione); 1α-hydroxycorticosterone (1alpha,11beta,21-trihydroxy-4-pregnene-3,20-dione); aldosterone 18,11-hemiacetal of 11beta,21-dihydroxy-3,20-dioxo-4-pregnen-18-al, androstenedione (4-androstene-3,17-dione); 4-hydroxy-androstenedione; 11β-hydroxyandrostenedione (11beta-4-androstene-3,17-dione); androstanediol (3-beta,17-beta-Androstanediol); androsterone (3alpha-hydroxy-5alpha-androstan-17-one); epiandrosterone (3beta-hydroxy-5alpha-androstan-17-one); adrenosterone (4-androstene-3,11,17-trione); dehydroepiandrosterone (3beta-hydroxy-5-androsten-17-one); dehydroepiandrosterone sulphate (3beta-sulfoxy-5-androsten-17-one); testosterone (17beta-hydroxy-4-androsten-3-one); epitestosterone (17alpha-hydroxy-4-androsten-3-one); 5α-dihydrotestosterone (17beta-hydroxy-5alpha-androstan-3-one 5β-dihydrotestosterone; 5-beta-dihydroxy testosterone (17beta-hydroxy-5beta-androstan-3-one); 11β-hydroxytestosterone (11beta,17beta-dihydroxy-4-androsten-3-one); 11-ketotestosterone (17beta-hydroxy-4-androsten-3,17-dione), estrone (3-hydroxy-1,3,5(10)-estratrien-17-one); estradiol (1,3,5(10)-estratriene-3,17beta-diol); estriol 1,3,5(10)-estratriene-3,16alpha,17beta-triol; pregnenolone (3-beta-hydroxy-5-pregnen-20-one); 17-hydroxypregnenolone (3-beta,17-dihydroxy-5-pregnen-20-one); progesterone (4-pregnene-3,20-dione); 17-hydroxyprogesterone (17-hydroxy-4-pregnene-3,20-dione) and progesterone (pregn-4-ene-3,20-dione).

The first region of the bi-functional molecule may also be capable of binding to a molecule associated with a steroid hormone including sex hormone binding globulin (SHBG) and albumin. The first region may also be directed to a site formed on the binding of a steroid hormone with an associated molecule.

In one embodiment, the bi-functional molecule is a polypeptide. Where the molecule is a polypeptide the first region may comprises the steroid binding region of a steroid receptor, or functional equivalent thereof. The steroid receptor may be an androgen receptor, a glucocorticoid receptor, a mineralocorticoid receptor, a progestin receptor, a progesterone receptor, or an estrogen receptor.

In one form of the bi-functional molecule, the first region has an affinity for a steroid hormone that is equal to or greater than the affinity between the steroid hormone and a natural carrier of the steroid hormone, such as sex hormone binding globulin (SHBG) or albumin.

In one embodiment, the means for removing the bi-functional molecule and any bound steroid hormone and/or steroid hormone associated molecule from solution comprises means for decreasing the solubility of the bi-functional molecule and any bound steroid hormone and/or steroid hormone associated molecule. In one form of the bi-functional molecule the means for decreasing solubility is aggregation. The decrease in solubility may occur in response to an environmental stimulus such as a change in temperature.

In one embodiment, the second region of the bi-functional molecule comprises the motif Val-Pro-Gly-X-Gly, and wherein X is any amino acid. In another embodiment X is any amino acid except Pro. The motif may be repeated in the second region.

In one embodiment the second region is an elastin-like polypeptide.

In another aspect the present invention provides a method for depleting a solution of a steroid hormone, the method comprising the steps of exposing the serum to a bi-functional molecule as described herein, allowing the steroid hormone and/or steroid hormone associated molecule to bind to the bi-functional molecule, and removing the bi-functional molecule and any bound steroid hormone and/or steroid hormone associated molecule from the solution. In one form of the method the solution is a serum.

Where the bi-functional molecule comprises means for decreasing solubility in response to an environmental stimulus, the method comprises the step of exposing the solution to the environmental stimulus after the step of allowing the steroid hormone and/or associated molecule to bind to the bi-functional molecule.

In one form of the method, the insoluble complex is separated from the biological fluid by a method selected from the group consisting of microfiltration, centrifugation, and decanting.

In another aspect of the present invention there is provided a serum that is depleted in only 1, 2, 3, 4 or 5 steroid hormone species.

In another aspect the present invention provides a serum that is depleted in a steroid hormone, the serum comprising one or more non-steroidal biologically active molecules at normal concentration. The non-steroidal biologically active molecule may be an antibody (such as IgA, IgE, IgG, IgM), a clotting factor (such as Factor I, Factor II, Factor III, Factor IV, Factor V, Factor VI, Factor VII, Factor VIII, Factor IX, Factor X, Factor XI, Factor XII, Factor XIII), a transport protein (such as transferrin, sex hormone binding globulin), a cytokine (such as PDGF, EGF, TGF-alpha, TGF-beta, FGF, NGF, any one of IL-1 to IL-13, interferon), a colony stimulating factor (such as G-CSF, M-CSF, GM-CSF), a basophilic mediator molecule (such as histamine, serotonin, prostaglandins, leukotrienes), a protein hormone (such as thyroid-stimulating hormone (TSH), follicle-stimulating hormone (FSH), Luteinizing hormone, Prolactin (PRL), Growth hormone (OH), Parathyroid hormone, Human chorionic gonadotropin (HCG), Insulin, Erythropoietin, Insulin-like growth factor-1 (IGF-1) Angiotensinogen, Thrombopoietin Leptin, Retinol Binding Protein 4, Adiponectin), a peptide hormone (such as Adrenocorticotropic hormone (ACTH), Antidiuretic hormone (ADH)(vasopressin), Oxytocin, Thyrotropin-releasing hormone (TRH), Gonadotropin-releasing hormone (GnRH) peptide, Growth hormone-releasing hormone (GHRH), Corticotropin-releasing hormone (CRH), Glucagon Somatostatin Amylin Atrial-natriuretic peptide (ANP) Gastrin, Secretin Neuropeptide Y, Ghrelin, PYY3-36), a tyrosine derivative hormone (including Dopamine, Melatonin, Thyroxine (T4), Adrenaline (epinephrine), Noradrenaline (norepinephrine), Cholecystokinin (CCK), a vitamin and an endotoxin.

Yet a further aspect of the present invention provides a steroid hormone depleted serum product produced according to a method as described herein.

In another aspect the present invention provides a method for treating or preventing an estrogen-related cancer or an androgen-related cancer, the method comprising administering to a subject in need thereof an effective amount of a nucleic acid molecule or a vector as described herein. The estrogen-related cancer may be breast cancer or ovarian cancer, while the androgen-related cancer may be endometrial cancer.

In a further aspect the present invention provides a method for treating or preventing estrogen flare or testosterone flare in the treatment of a subject having estrogen-related cancer with an LHRH agonist or antagonist comprising administering to a subject in need thereof an effective amount of a polypeptide, nucleic acid or vector as described herein.

A further aspect of the present invention provides use of a polypeptide, nucleic acid molecule or vector as described herein in the manufacture of a medicament for the treatment or prevention of an estrogen-related cancer or an androgen-related cancer. The estrogen-related cancer may be breast cancer or ovarian cancer, while the androgen-related cancer may be endometrial cancer.

Yet a further aspect of the present invention provides use of a polypeptide, nucleic acid or vector as described herein in the manufacture of a medicament for the treatment or prevention of estrogen flare or testosterone flare.

In one aspect, the present invention provides a polypeptide comprising an androgen binding region, the androgen binding region capable of binding to an androgen at a sufficient affinity or avidity such that upon administration of the polypeptide to a mammalian subject the level of biologically available androgen is decreased. Applicant proposes that the administration of a polypeptide capable of sequestering androgen (for example testosterone or dihydrotestosterone) in the body may have efficacy in the treatment of prostate cancer.

In the context of the invention, the level of biologically available androgen may be measured in the blood of the subject, or within a prostate cell, and especially a prostate epithelial cell. In one form of the invention the polypeptide is capable of decreasing the level of biologically available androgen such that the growth of a prostate cancer cell in the subject is decreased or substantially arrested.

The polypeptide may have an affinity for testosterone that is equal to or greater than the affinity between the androgen and a protein that naturally binds to testosterone such as the sex hormone binding globulin. The polypeptide may have an affinity for testosterone that is equal to or greater than the affinity between, testosterone and the 5-alpha-reductase enzyme present in a prostate epithelial cell, or the androgen receptor present in a prostate epithelial cell.

In another form of the invention the polypeptide has an affinity for dihydrotestosterone that is equal to or greater than the affinity between dihydrotestosterone and the androgen receptor present in a prostate epithelial cell.

In one form of the polypeptide, the androgen binding region includes the androgen binding domain from the human androgen receptor, or the androgen binding domain from the sex hormone binding globulin.

In one form of the invention the polypeptide has a single androgen binding region. In another form, the polypeptide includes a carrier region such as the Fc region of human IgG. A further form of the polypeptide includes a multimerisation domain. The polypeptide may take the form of a fusion protein, a monoclonal antibody, a polyclonal antibody, or a single chain antibody.

The polypeptide may be capable of entering a prostate cell, and especially a prostate epithelial cell.

In another aspect, the present invention provides a nucleic acid molecule capable of encoding a polypeptide as described herein. A further aspect of the present invention provides a vector including a nucleic acid molecule as described herein.

In another aspect the present invention provides a composition comprising a polypeptide as described herein and a pharmaceutically acceptable carrier.

Yet a further aspect of the invention provides a method for treating or preventing prostate cancer in a subject, the method including administering to a subject in need thereof an effective amount of a ligand capable of binding androgen in the subject, such that the level of biologically available androgen in the subject is decreased. In one embodiment of the method, the ligand is a polypeptide as described herein.

Another aspect of the invention provides a method for treating or preventing prostate cancer, the method including administering to a subject in need thereof an effective amount of a nucleic acid molecule as described herein, or a vector as described herein.

In yet a further aspect, the present invention provides a method for treating or preventing testosterone flare including administering to a subject in need thereof an effective amount of a polypeptide as described herein.

Still a further aspect of the invention provides that use of a polypeptide as described herein in the manufacture of a medicament for the treatment or prevention of prostate cancer or testosterone flare.

In another aspect, the present invention provides the use of a nucleic acid molecule as described herein in the manufacture of a medicament for the treatment or prevention of prostate cancer or testosterone flare.

Still a further aspect provides the use of a vector as described herein in the manufacture of a medicament for the treatment or prevention of prostate cancer or testosterone flare.

In one aspect, the present invention provides a polypeptide for regulating a reproductive physiology of an animal, the polypeptide comprising a steroid sex hormone binding region, the steroid sex hormone binding region capable of binding to a steroid sex hormone at a sufficient affinity or avidity such that upon administration of the polypeptide to the animal the level of biologically available steroid sex hormone is decreased. The level of biologically available steroid sex hormone may be measured in the blood of the animal.

The polypeptide may have an affinity or avidity for the steroid sex hormone that is equal to or greater than the affinity or avidity between the steroid sex hormone and a natural carrier of the steroid sex hormone such as SHBG or albumin.

The steroid sex hormone binding region of the polypeptide may comprise a sequence from the binding region of a steroid sex hormone receptor, such as an androgen receptor, a progesterone receptor, or an estrogen receptor.

The steroid sex hormone may be androstenedione (4-androstene-3,17-dione); 4-hydroxy-androstenedione; 11β-hydroxyandrostenedione (11beta-4-androstene-3,17-dione); androstanediol (3-beta,17-beta-Androstanediol); androsterone (3alpha-hydroxy-5alpha-androstan-17-one); epiandrosterone (3beta-hydroxy-5alpha-androstan-17-one); adrenosterone (4-androstene-3,11,17-trione); dehydroepiandrosterone (3beta-hydroxy-5-androsten-17-one); dehydroepiandrosterone sulphate (3beta-sulfoxy-5-androsten-17-one); testosterone (17beta-hydroxy-4-androsten-3-one); epitestosterone (17alpha-hydroxy-4-androsten-3-one); 5α-dihydrotestosterone (17beta-hydroxy-5alpha-androstan-3-one 5β-dihydrotestosterone; 5-beta-dihydroxy testosterone (17beta-hydroxy-5beta-androstan-3-one); 11β-hydroxytestosterone (11beta,17beta-dihydroxy-4-androsten-3-one); 11-ketotestosterone (17beta-hydroxy-4-androsten-3,17-dione), estrone (3-hydroxy-1,3,5(10)-estratrien-17-one); estradiol (1,3,5(10)-estratriene-3,17beta-diol); estriol 1,3,5(10)-estratriene-3,16alpha,17beta-triol; pregnenolone (3-beta-hydroxy-5-pregnen-20-one); 17-hydroxypregnenolone (3-beta,17-dihydroxy-5-pregnen-20-one); progesterone (4-pregnene-3,20-dione); 17-hydroxyprogesterone (17-hydroxy-4-pregnene-3,20-dione), or progesterone (pregn-4-ene-3,20-dione).

The polypeptide may have a single steroid sex hormone binding region. The polypeptide may comprise a carrier region such as a sequence of the IgG Fc region. The polypeptide may be in the form of a fusion protein, a monoclonal antibody, a polyclonal antibody, or a single chain antibody, and may comprise a multimerisation domain. Another aspect the present invention provides a composition comprising a polypeptide as described herein in combination with a pharmaceutically acceptable carrier.

In other aspects, the present invention provides a nucleic acid molecule capable of encoding a polypeptide as described herein, and a vector comprising that nucleic acid molecule.

In a further aspect, the present invention provides a method for regulating a reproductive physiology of an animal, the method comprising administering to a subject in need thereof an effective amount of a polypeptide as described herein. The level of biologically available steroid may be measured in the blood of the subject. The polypeptide may be administered in the form of a composition as described herein.

Another aspect of the invention provides a method for regulating a reproductive physiology, the method comprising administering to a subject in need thereof an effective amount of a nucleic acid molecule as described herein or a vector as described herein.

In another aspect, the present invention provides use of a polypeptide, nucleic acid molecule or vector as described herein in the manufacture of a medicament for the regulating a reproductive physiology in an animal.

The reproductive physiologies for which the present polypeptides and methods may be applicable include ovulation, conception, parturition, commencement of estrus, maintenance of estrus, termination of estrus, commencement of pregnancy, maintenance of pregnancy, termination of pregnancy, erection, semen production, spermatogenesis, or a behaviour selected from the group consisting of restlessness, agitation, hyperactivity, frequent urination, sniffing or licking a stallion, straddling posture, clitoral “winking”, raising the tail, dominance, aggression, Flehmen response, impatience, alertness, hyperactivity, restlessness, vocalization, nudging or smelling or biting a mare.

The polypeptides and methods are applicable to any non-human animal, but particularly to mammals and preferably important agriculturally and economically important animals for example from the equid, porcine, bovine, caprine, ovine, canine, feline, deer and alpaca families, as well as companion animals such as dogs and cats.

### BRIEF DESCRIPTION OF THE FIGURES

FIG. 1 shows a map of pFUSE-hIgG1-Fc2.

FIG. 2 shows a map of pFUSE-hIgG1e2-Fc2.

FIG. 3 shows a map of pFUSE-mIgG1-Fc2.

FIG. 4 shows a Western blot of AR IgG1 Fc, and IgG1 Fc control fusion proteins. Western blot of AR IgG1 Fc, and IgG1 Fc control fusion proteins. 8 μl of concentrated AR-IgG Fc and 1 μl of concentrated IgG Fc CHO cell supernatants were loaded on to a 12% SDS PAGE gel and separated at 170V for 70 min. Proteins were transferred onto nitrocellulose membrane (100V for 90 min) using standard techniques. The blot was then probed with an anti-human IgG Fc-HRP conjugated antibody (Pierce, cat no:31413) at 1:20,000 dilution and developed using the Super Signal West femto developing kit (Pierce, cat no:34094) according to the manufacturers specifications. Clearly detectable bands of the expected sizes were observed of approx 55 kD for the AR IgG1 Fc fusion protein and 28 kD for the control IgG1 Fc protein.

FIG. 5 is a bar graph showing growth of human prostate cancer cell line LNCaP in the presence of various media and treatments over 5 days as assessed by the calcein fluorescence assay. The results depict the means of six independent wells with error bars representing the SEM values.

Table 1. Results of the LNCaP growth experiments (FIG. 5) in tabular form.

FIG. 6A is a graph depicting standard curve of known free testosterone concentrations (blue dots) versus free testosterone concentration of control mouse serum (red dot) and free testosterone concentration of serum from mice injected with the AR-IgG1 Fc fusion protein (green dot).

FIG. 6B is a bar graph showing mean values of free testosterone levels in serum of mice either injected or not with AR IgG Fc fusion protein (25 ng).

Table 2. Results of the in vivo free testosterone levels experiments (FIG. 6) in tabular form.

FIG. 6C is a bar graph showing average values of free testosterone levels in serum of SCID/NOD mice either injected with AR-LBD IgG1 Fc fusion protein (200 μl of 1 ng/μl) or with control IgG1 Fc protein (200 μl of 1 ng/μl).

FIG. 6D is a bar graph showing average percentage values of free testosterone levels in serum of SCID/NOD mice either injected with AR-LBD IgG1 Fc fusion protein (200 μl of 1 ng/μl) or with control IgG1 Fc protein (200 μl of 1 ng/μl). Values are depicted as percentage of control IgG1 Fc group.

FIG. 7A depicts representative images of final prostate tumour sizes of NUDE mice either injected twice with either A:control IgG1 Fc protein (200 μl of 1 ng/μl) or B: AR-LBD IgG1 Fc fusion protein (200 μl of 1 ng/μl).

FIG. 7B is a graphical depiction of prostate tumour volumes throughout timecourse of the experiment of male NUDE mice, injected twice in the tail vein with either control IgG1 Fc protein (200 μl of 1 ng/μl), or with AR-LBD IgG1 Fc fusion protein (200 μl of 1 ng/μl).

FIG. 7C is a graphical depiction of final average prostate tumour weights (mg) of male NUDE mice either injected twice with either control IgG1 Fc protein (IgG) (200 μl of 1 ng/μl), or with AR-LBD IgG1 Fc fusion protein (AR) (200 μl of 1 ng/μl). Numbers represent the mean tumour weights of the respective groups.

FIG. 11 is a graphical depiction of the domain structure and restriction map of the AR-ELP ORF nucleotide sequence. The His-tag, AR LBD (ligand binding domain), ELP (Elastin like peptide) domain and S-tag regions are depicted.

FIG. 12 is a graphical depiction of the domain structure of the AR-ELP polypeptide. The His-tag, AR LBD (ligand binding domain), ELP (Elastin like peptide) domain and S-tag regions are depicted.

FIG. 13 is a graphical depiction of the domain structure of the ER-ELP ORF nucleotide sequence. The His-tag, ER LBD (ligand binding domain), ELP (Elastin like peptide) domain and S-tag regions are depicted.

FIG. 14 is a graphical depiction of the domain structure of the ER-ELP polypeptide. The His-tag, ER LBD (ligand binding domain), ELP (Elastin like peptide) domain and S-tag regions are depicted.

### DETAILED DESCRIPTION OF THE INVENTION

In a first aspect the present invention provides a polypeptide comprising a nuclear hormone receptor agonist binding region, the nuclear hormone receptor agonist binding region capable of binding to a nuclear hormone receptor agonist at a sufficient affinity or avidity such that upon administration of the polypeptide to a mammalian subject the level of biologically available nuclear hormone receptor agonist is decreased. It is proposed that polypeptides having the ability to bind to a nuclear hormone receptor agonist are useful in decreasing the level of steroid hormones such as progestins, androgens, estrogens, corticosteroids, and thyroid hormones. Without wishing to be limited by theory, the polypeptides may bind nuclear hormone receptor agonist molecules thereby decreasing the level of agonist available to bind the cognate nuclear hormone receptor. Accordingly, the polypeptides will find use in the treatment of conditions relating to an excess of steroid hormones and thyroid hormones in the body.

Typically, the polypeptide has an affinity or avidity for a nuclear hormone receptor agonist that is sufficiently high such that upon administration of the polypeptide to a mammalian subject, the polypeptide is capable of decreasing biologically available nuclear hormone receptor agonist in the blood or a cell of the subject to a level lower than that demonstrated in the subject prior to administration of the polypeptide. As used herein, the term “biologically available nuclear hormone receptor agonist” means an agonist that is capable of exerting its biological activity. As will be understood, the present invention is directed to polypeptides that are capable of decreasing the level of nuclear hormone receptor agonist available to bind to its cognate receptor in the subject. For example, in the context of the present invention where the nuclear hormone receptor agonist is testosterone, the term “biologically available” means that the testosterone is free for conversion to dihydrotestosterone, which subsequently binds to the androgen receptor. Where the agonist is dihydrotestosterone (typically located intracellularly) the term “biologically available” means that the dihydrotestosterone is free to bind to an androgen receptor.

The present invention is distinct from approaches of the prior art that aim to decrease the production of steroid hormones and thyroid hormones, by surgically removing the source of the hormone (for example, the adrenal glands).

The present invention is also distinguished from prior art treatments that act to block 5-alpha-reductase, the enzyme that converts testosterone to dihydrotestosterone. While both testosterone and dihydrotestosterone are able to bind the androgen receptor, dihydrotestosterone is the more potent ligand. Thus, while compounds such as finasteride can limit the level of dihydrotestosterone in a cell, they are unable to affect the binding of testosterone directly to the androgen receptor.

The polypeptides of the present invention are also different to compounds of the prior art such as flutamide and spirinolactone that bind to the androgen receptor. While these compounds have some efficacy in blocking the receptor they are incapable (as a monotherapy) to sufficiently limit androgen signaling. In addition, some patients have one or more mutations in the androgen receptor gene such that compounds of the prior art may act only as partial agonists of the androgen receptor. By contrast, the polypeptides of the present invention bind to molecules that have a set chemical structure, and “escape” variants do not need to be accounted for.

In the context of the present invention, the term “nuclear hormone receptor agonist” is intended to include any naturally occurring or synthetic steroid hormone, thyroid hormone or any functionally equivalent molecule that is present in a subject. Thus, the invention includes polypeptides that bind to hormones that are endogenous, and also those that have been administered to a patient in the course of medical treatment.

In one form of the invention, the nuclear hormone receptor agonist is a corticosteroid. Corticosteroids are a group of natural and synthetic analogues of the hormones secreted by the hypothalamic-anterior pituitary-adrenocortical (HPA) axis. These include glucocorticoids, which are anti-inflammatory agents with a large number of other functions; mineralocorticoids, which control salt and water balance primarily through action on the kidneys. Exemplary corticosteroids include corticosterone (11beta,21-dihydroxy-4-pregnene-3,20-dione); deoxycorticosterone (21-hydroxy-4-pregnene-3,20-dione); cortisol (11beta,17,21-trihydroxy-4-pregnene-3,20-dione); 11-deoxycortisol (17,21-dihydroxy-4-pregnene-3,20-dione); cortisone (17,21-dihydroxy-4-pregnene-3,11,20-trione); 18-hydroxycorticosterone (11beta,18,21-trihydroxy-4-pregnene-3,20-dione); 1α-hydroxycorticosterone (1alpha,11beta,21-trihydroxy-4-pregnene-3,20-dione); and aldosterone 18,11-hemiacetal of 11beta,21-dihydroxy-3,20-dioxo-4-pregnen-18-al.

In one form of the invention, the nuclear hormone receptor agonist is an androgen. Androgens stimulate or control the development and maintenance of masculine characteristics in vertebrates by binding to androgen receptors. This includes the activity of the accessory male sex organs and development of male secondary sex characteristics. Exemplary androgens include androstenedione (4-androstene-3,17-dione); 4-hydroxy-androstenedione; 11β-hydroxyandrostenedione (11beta-4-androstene-3,17-dione); androstanediol (3-beta,17-beta-Androstanediol); androsterone (3alpha-hydroxy-5alpha-androstan-17-one); epiandrosterone (3beta-hydroxy-5alpha-androstan-17-one); adrenosterone (4-androstene-3,11,17-trione); dehydroepiandrosterone (3beta-hydroxy-5-androsten-17-one); dehydroepiandrosterone sulphate (3beta-sulfoxy-5-androsten-17-one); testosterone (17beta-hydroxy-4-androsten-3-one); epitestosterone (17alpha-hydroxy-4-androsten-3-one); 5α-dihydrotestosterone (17beta-hydroxy-5alpha-androstan-3-one 5β-dihydrotestosterone; 5-beta-dihydroxy testosterone (17beta-hydroxy-5beta-androstan-3-one); 11β-hydroxytestosterone (11beta,17beta-dihydroxy-4-androsten-3-one); and 11-ketotestosterone (17beta-hydroxy-4-androsten-3,17-dione).

In one form of the invention, the nuclear hormone receptor agonist is an estrogen. Estrogens are a group of steroid compounds, named for their importance in the estrous cycle, and functioning as the primary female sex hormone. Exemplary estrogens include estrone (3-hydroxy-1,3,5(10)-estratrien-17-one); estradiol (1,3,5(10)-estratriene-3,17beta-diol); and estriol 1,3,5(10)-estratriene-3,16alpha,17beta-triol.

In one form of the invention, the nuclear hormone receptor agonist is a progestin. Progestins are a synthetic progestogen that has some biological activity similar to progesterone and is most well known for the applications in hormonal contraception, but progestins (and progesterone) also have applications in the treatment of dysmenorrhea, endometriosis, functional uterine bleeding, and amenorrhea. Exemplary progestins include pregnenolone (3-beta-hydroxy-5-pregnen-20-one); 17-hydroxypregnenolone (3-beta,17-dihydroxy-5-pregnen-20-one); progesterone (4-pregnene-3,20-dione); and 17-hydroxyprogesterone (17-hydroxy-4-pregnene-3,20-dione). Progesterone (pregn-4-ene-3,20-dione) can be considered a natural progestin, and is included in the scope of the present invention.

In another form of the invention the nuclear hormone receptor agonist is a thyroid hormone, including T₃ and T₄.

Steroid hormones and thyroid hormones exert their biological activities via a common mechanism, both agonizing members of the nuclear hormone receptor superfamily. All members of the superfamily function as transcription factors. The members are highly related in both primary amino acid sequence and the organization of functional domains suggesting that many aspects of their mechanism of action are conserved. Indeed, progress in understanding of steroid hormone action has been facilitated by studies of many nuclear receptor family members.

Steroid hormone receptors share a modular structure in which six distinct structural and functional domains, A to F, are displayed (Evans, Science 240, 889-895, 1988, the contents of which is herein incorporated by reference). A nuclear hormone receptor is characterized by a variable N-terminal region (domain A/B), followed by a centrally located, highly conserved DNA-binding domain (hereinafter referred to as DBD; domain C), a variable hinge region (domain D), a conserved hormone binding domain; domain E) and a variable C-terminal region (domain F).

The N-terminal region, which is highly variable in size and sequence, is poorly conserved among the different members of the superfamily. This part of the receptor is involved in the modulation of transcription activation (Bocquel et al, Nucl. Acid Res., 17, 2581-2595, 1989; Tora et al, Cell 59, 477-487, 1989, the contents of which are herein incorporated by reference).

The DBD consists of approximately 66 to 70 amino acids and is responsible for DNA-binding activity: it targets the receptor to specific DNA sequences called hormone responsive elements within the transcription control unit of specific target genes on the chromatin (Martinez and Wahli; In “Nuclear Hormone Receptors”, Acad. Press, 125-153, 1991, the contents of which is herein incorporated by reference).

The hormone binding domain is located in the C-terminal part of the receptor and is primarily responsible for agonist binding activity. This domain is therefore required for recognition and binding of the agonist thereby determining the specificity and selectivity of the hormone response of the receptor. In the context of the present invention, the hormone binding domain is the most important region since it affords the polypeptides of the present invention the ability to effectively sequester biologically available hormone.

In the absence of hormone, steroid hormone receptors exist as inactive oligomeric complexes with a number of other proteins including chaperon proteins, namely the heat shock proteins Hsp90 and Hsp70 and cyclophilin-40 and p23. The role of Hsp90 and other chaperons is to maintain the receptors folded in an appropriate conformation to respond rapidly to hormonal signals. Following hormone binding, the oligomeric complex dissociates allowing the receptors to function either directly as transcription factors by binding to DNA in the vicinity of target genes or indirectly by modulating the activity of other transcription factors.

As discussed supra receptors for thyroid hormones are members of the same family of nuclear receptors agonized, by steroid hormones. They also function as hormone-activated transcription factors and thereby act by modulating gene expression. In contrast to steroid hormone receptors, thyroid hormone receptors bind DNA in the absence of hormone, usually leading to transcriptional repression. Hormone binding is associated with a conformational change in the receptor that causes it to function as a transcriptional activator. However, it will be appreciated that as members of the same receptor family, thyroid hormone and steroid hormone receptors display many structural and functional similarities.

Mammalian thyroid hormone receptors are encoded by two genes, designated alpha and beta. Further, the primary transcript for each gene can be alternatively spliced, generating different alpha and beta receptor isoforms. Currently, four different thyroid hormone receptors are recognized: alpha-1, alpha-2, beta-1 and beta-2.

Like other members of the nuclear hormone receptor superfamily, thyroid hormone receptors encapsulate three functional domains: a transactivation domain at the amino terminus that interacts with other transcription factors to form complexes that repress or activate transcription, DNA-binding domain that binds to sequences of promoter DNA, and a ligand-binding and dimerization domain at the carboxy-terminus.

In light of the above, it will be appreciated that all steroid hormones and thyroid hormones have a cognate receptor which includes sequences capable of binding a steroid or thyroid hormone molecule. The present invention provides polypeptides capable of binding to a steroid or thyroid hormone such that the ability of the hormone to agonize the cognate nuclear hormone receptor is decreased, or even completely inhibited. In one embodiment of the polypeptide, the nuclear hormone receptor agonist binding region includes sequences from the hormone binding domain of the mineralocorticoid receptor, or functional equivalent thereof. The sequence for the human mineralocorticoid receptor is known:

METKGYHSLPEGLDMERRWGQVSQAVERSSLGPTERTDENNYMEIVNVS
CVSGAIPNNSTQGSSKEKQELLPCLQQDNNRPGILTSDIKTELESKELS
ATVAESMGLYMDSVRDADYSYEQQNQQGSMSPAKIYQNVEQLVKFYKGN
GHRPSTLSCVNTPLRSFMSDSGSSVNGGVMRAVVKSPIMCHEKSPSVCS
PLNMTSSVCSPAGINSVSSTTASFGSFPVHSPITQGTPLTCSPNVENRG
SRSHSPAHASNVGSPLSSPLSSMKSSISSPPSHCSVKSPVSSPNNVTLR
SSVSSPANINNSRCSVSSPSNTNNRSTLSSPAASTVGSICSPVNNAFSY
TASGTSAGSSTLRDVVPSPDTQEKGAQEVPFPKTEEVESAISNGVTGQL
NIVQYIKPEPDGAFSSSCLGGNSKINSDSSFSVPIKQESTKHSCSGTSF
KGNPTVNPFPFMDGSYFSFMDDKDYYSLSGILGPPVPGFDGNCEGSGFP
VGIKQEPDDGSYYPEASIPSSAIVGVNSGGQSFHYRIGAQGTISLSRSA
RDQSFQHLSSFPPVNTLVESWKSHGDLSSRRSDGYPVLEYIPENVSSST
LRSVSTGSSRPSKICLVCGDEASGCHYGVVTCGSDKVFFKRAVEGQHNY
LCAGRNDCIIDKIRRKNCPACRLQKCLQAGMNLGARKSKKLGKLKGIHE
EQPQQQQPPPPPPPPQSPEEGTTYIAPAKEPSVNTALVPQLSTISRALT
PSPVMVLENIEPEIVYAGYDSSKPDTAENLLSTLNRLAGKQMIQVVKWA
KVLPGFKNLPLEDQITLIQYSWMCLSSFALSWRSYKHTNSQFLYFAPDL
VFNEEKMHQSAMYELCQGMHQISLQFVRLQLTFEEYTIMKVLLLLSTIP
KDGLKSQAAFEEMRTNYIKELRKMVTKCPNNSGQSWQRFYQLTKLLDSM
HDLVSDLLEFCFYTFRESHALKVEFPAMLVEIISDQLPKVESGNAKPLY
FHRK

The hormone binding region has been identified by Jalaguier et at (Journal of Steroid Biochemistry and Molecular Biology, Volume 57, Number 1, January 1996, pp. 43-50(8), the contents of which is herein incorporated by reference), as including the residues of approximately 727-984. To improve the solubility of polypeptide (and therefore improve pharmacokinetic properties), a C808S mutation may be introduced into the above sequence.

In one form of the polypeptide, the mineralocorticoid receptor hormone binding domain is produced in accordance with the method of Fraser et al (J Biol Chem, Vol. 274, Issue 51, 36305-36311, Dec. 17, 1999, the contents of which is herein incorporated by reference). In that publication, the binding domain is amplified by PCR from the plasmid pRShMRNX, as described by Arriza et al (Science (1987) 237, 268-275, the contents of which is herein incorporated by reference).

In another embodiment of the polypeptide, the nuclear hormone receptor agonist binding region includes sequences from the hormone binding domain of the glucocorticoid receptor, or functional equivalent thereof. Given its biological and pharmaceutical importance, there has been enormous interest in elucidating the hormone binding domain of this receptor. Bledsoe et al (Cell 110(1)2002, 93-105, the contents of which is herein incorporated by reference) describe the expression, purification, crystallization, and structure determination of the binding domain in complex with ligand. The full wild type sequence of the human glucocorticoid receptor is known:

MDSKESLTPG REENPSSVLA QERGDVMDFY KTLRGGATVK VSASSPSLAV
ASQSDSKQRR LLVDFPKGSV SNAQQPDLSK AVSLSMGLYM GETETKVMGN
DLGFPQQGQI SLSSGETDLK LLEESIANLN RSTSVPENPK SSASTAVSAA PTEKEFPKTH
SDVSSEQQHL KGQTGTNGGN VKLYTTDQST FDILQDLEFS SGSPGKETNE SPWRSDLLID
ENCLLSPLAG EDDSFLLEGN SNEDCKPLIL PDTKPKIKDN GDLVLSSPSN VTLPQVKTEK
EDFIELCTPG VIKQEKLGTV YCQASFPGAN IIGNKMSAIS VHGVSTSGGQ MYHYDMNTAS
LSQQQDQKPI FNVIPPIPVG SENWNRCQGS GDDNLTSLGT LNFPGRTVFS NGYSSPSMRP
DVSSPPSSSS TATTGPPPKL CLVCSDEASG CHYGVLTCGS CKVFFKRAVE
GQHNYLCAGR NDCIIDKIRR KNCPACRYRK CLQAGMNLEA RKTKKKIKGI QQATTGVSQE
TSENPGNKTI VPATLPQLTP TLVSLLEVIE PEVLYAGYDS SVPDSTWRIM TTLNMLGGRQ
VIAAVKWAKA IPGFRNLHLD DQMTLLQYSW
MFLMAFALGW RSYRQSSANL LCFAPDLIIN EQRMTLPCMY DQCKHMLYVS SELHRLQVSY
EEYLCMKTLL LLSSVPKDGL KSQELFDEIR MTYIKELGKA IVKREGNSSQ NWQRFYQLTK
LLDSMHEVVE NLLNYCFQTF LDKTMSIEFP EMLAEIITNQ IPKYSNGNIK KLLFHQK

The structure reveals a distinct steroid binding pocket with features that explain ligand binding and selectivity. In one embodiment of the polypeptide, the nuclear hormone receptor agonist binding region includes residues approximately 521 to 777 of the glucocorticoid receptor. In one form of the polypeptide a F602S mutation is introduced into the above sequence. This mutation improves solubility and has been shown to effectively bind glucocorticoid (Bledsoe et al 2002)

In one form of the polypeptide, the glucocorticoid receptor hormone binding domain is produced in accordance with the method of Fraser et al (J Biol Chem, Vol. 274, Issue 51, 36305-36311, Dec. 17, 1999, the contents of which is herein incorporated by reference). Briefly, The OR LBD was derived from the plasmid pRShGRBX (Keightley, M.-C., and Fuller, P. J. (1994) Mol. Endocrinol. 8, 431-439, the contents of which is herein incorporated by reference). This construct was derived from pRShGRNX as described by Rupprecht et al (Mol. Endocrinol. (1993) 7, 597-603, the contents of which is herein incorporated by reference).

In another form of the polypeptide, the nuclear hormone receptor agonist binding region includes sequences from the hormone binding domain of the progesterone receptor, or functional equivalent thereof. Like all nuclear hormone receptors, the progesterone receptor has a regulatory domain, a DNA binding domain, a hinge section, and a hormone binding domain. The progesterone receptor has two isoforms (A and B). The single-copy human (hPR) gene uses separate promoters and translational start sites to produce the two isoforms. Both are included in the scope of this invention:

Williams and Sigler have solved the atomic structure of progesterone complexed with its receptor (Nature. 1998 May 28; 393(6683):392-6, the contents of which is herein incorporated by reference). The authors report the 1.8 A crystal structure of a progesterone-bound ligand-binding domain of the human progesterone receptor. The nature of this structure explains the receptor's selective affinity or avidity for progestins and establishes a common mode of recognition of 3-oxy steroids by the cognate receptors. The wild type sequence of the human progesterone sequence is known:

MTELKAKGPRAPHVAGGPPSPEVGSPLLCRPAAGPFPGSQTSDTLPEVSAIPISLDGL
LFPRPCQGQDPSDEKTQDQQSLSDVEGAYSRAEATRGAGGSSSSPPEKDSGLLDSV
LDTLLAPSGPGQSQPSPPACEVTSSWCLFGPELPEDPPAAPATQRVLSPLMSRSGCK
VGDSSGTAAAHKVLPRGLSPARQLLLPASESPHWSGAPVKPSPQAAAVEVEEEDGS
ESEESAGPLLKGKPRALGGAAAGGGAAAVPPGAAAGGVALVPKEDSRFSAPRVALV
EQDAPMAPGRSPLATTVMDFIHVPILPLNHALLAARTRQLLEDESYDGGAGAASAFAP
PRSSPCASSTPVAVGDFPDCAYPPDAEPKDDAYPLYSDFQPPALKIKEEEEGAEASA
RSPRSYLVAGANPAAFPDFPLGPPPPLPPRATPSRPGEAAVTAAPASASVSSASSSG
STLECILYKAEGAPPQQGPEAPPPCKAPGASGCLLPRDGLPSTSASAAAAGAAPALYP
ALGLNGLPQLGYQAAVLKEGLPQVYPPYLNYLRPDSEASQSPQYSFESLPQKICLICG
DEASGCHYGVLTCGSCKVFFKRAMEGQHNYLCAGRNDCIVDKIRRKNCPACRLRKC
CQAGMVLGGRKFKKFNKVRVVRALDAVALPQPVGVPNESQALSQRFTFSPGQDIQLI
PPLINLLMSIEPDVIYAGHDNTKPDTSSSLLTSLNQLGERQLLSVVKWSKSLPGFRNLHI
DDQITLIQYSWMSLMVFGLGWRSYKHVSGQMLYFAPDLILNEQRMKESSFYSLCLTM
WQIPQEFVKLQVSQEEFLCMKVIILLNTIPLEGLRSQTQFEEMRSSYIRELIKAIGLRQK
GVVSSSQRFYQLTKLLDNLHDLVKQLHLYCLNTFIQSRALSVEFPEMMSEVIAAQLPKI
LAGMVKPLLFHKK

In one embodiment of the polypeptide, the nuclear hormone receptor agonist binding region includes residues approximately 676 to 693 of the progesterone receptor.

In another embodiment of the polypeptide, the nuclear hormone receptor agonist binding region includes sequences from the hormone binding domain of the estrogen receptor, or functional equivalent thereof. Wurtz et al (J Med. Chem. 1998 May 21; 41(11), the contents of which is herein incorporated by reference) published a three-dimensional model of the human estrogen receptor hormone binding domain. The quality of the model was tested against mutants, which affect the binding properties. A thorough analysis of all published mutants was performed with Insight II to elucidate the effect of the mutations. 45 out of 48 mutants can be explained satisfactorily on the basis of the model. After that, the natural ligand estradiol was docked into the binding pocket to probe its interactions with the protein. Energy minimizations and molecular dynamics calculations were performed for various ligand orientations with Discover 2.7 and the CFF91 force field. The analysis revealed two favorite estradiol orientations in the binding niche of the binding domain forming hydrogen bonds with Arg394, Glu353 and His524. The crystal structure of the ER LBD in complex with estradiol has been published (Brzozowski et al. Nature 389, 753-758, 1997, the contents of which is herein incorporated by reference). The amino acid sequence of the human estrogen receptor is as follows:

MTMTLHTKASGMALLHQIQGNELEPLNRPQLKIPLERPLGEVYLDSSK
PAVYNYPEGAAYEFNAAAAANAQVYGQTGLPYGPGSEAAAFGSNGLGG
FPPLNSVSPSPLMLLHPPPQLSPFLQPHGQQVPYYLENEPSGYTVREA
GPPAFYRPNSDNRRQGGRERLASTNDKGSMAMESAKETRYCAVCNDYA
SGYHYGVWSCEGCKAFFKRSIQGHNDYMCPATNQCTIDKNRRKSCQAC
RLRKCYEVGMMKGGIRKDRRGGRMLKHKRQRDDGEGRGEVGSAGDMRA
ANLWPSPLMIKRSKKNSLALSLTADQMVSALLDAEPPILYSEYDPTRP
FSEASMMGLLTNLADRELVHMINWAKRVPGFVDLTLHDQVHLLECAWL
EILMIGLVWRSMEHPGKLLFAPNLLLDRNQGKCVEGMVEIFDMLLATS
SRFRMMNLQGEEFVCLKSIILLNSGVYTFLSSTLKSLEEKDHIHRVLD
KITDTLIHLMAKAGLTLQQQHQRLAQLLLILSHIRHMSNKGMEHLYSM
KCKNVVPLYDLLLEMLDAHRLHAPTSRGGASVEETDQSHLATAGSTSS
HSLQKYYITGEAEGFPATV

In another embodiment of the polypeptide, the nuclear hormone receptor agonist binding region includes sequences from the hormone binding domain of the androgen receptor, or functional equivalent thereof. The gene encoding the receptor is more than 90 kb long and codes for a protein that has 3 major functional domains. The N-terminal domain, which serves a modulatory function, is encoded by exon 1 (1,586 bp). The DNA-binding domain is encoded by exons 2 and 3 (152 and 117 bp, respectively). The steroid-binding domain is encoded by 5 exons which vary from 131 to 288 bp in size. The amino acid sequence of the human androgen receptor protein is described by the following sequence.

MEVQLGLGRVYPRPPSKTYRGAFQNLFQSVREVIQNPGPRHPEAASAA
PPGASLLLLQQQQQQQQQQQQQQQQQQQQQETSPRQQQQQQGEDGSPQ
AHRRGPTGYLVLDEEQQPSQPQSALECHPERGCVPEPGAAVAASKGLP
QQLPAPPDEDDSAAPSTLSLLGPTFPGLSSCSADLKDILSEASTMQLL
QQQQQEAVSEGSSSGRAREASGAPTSSKDNYLGGTSTISDNAKELCKA
VSVSMGLGVEALEHLSPGEQLRGDCMYAPLLGVPPAVRPTPCAPLAEC
KGSLLDDSAGKSTEDTAEYSPFKGGYTKGLEGESLGCSGSAAAGSSGT
LELPSTLSLYKSGALDEAAAYQSRDYYNFPLALAGPPPPPPPPHPHAR
IKLENPLDYGSAWAAAAAQCRYGDLASLHGAGAAGPGSGSPSAAASSS
WHTLFTAEEGQLYGPCGGGGGGGGGGGGGGGGGGGGGGGGEAGAVAPY
GYTRPPQGLAGQESDFTAPDVWYPGGMVSRVPYPSPTCVKSEMGPWMD
SYSGPYGDMRLETARDHVLPIDYYFPPQKTCLICGDEASGCHYGALTC
GSCKVFFKRAAEGKQKYLCASRNDCTIDKFRRKNCPSCRLRKCYEAGM
TLGARKLKKLGNLKLQEEGEASSTTSPTEETTQKLTVSHIEGYECQPI
FLNVLEAIEPGVVCAGHDNNQPDSFAALLSSLNELGERQLVHVVKWAK
ALPGFRNLHVDDQMAVIQYSWMGLMVFAMGWRSFTNVNSRMLYFAPDL
VFNEYRMHKSRMYSQCVRMRHLSQEFGWLQITPQEFLCMKALLLFSII
PVDGLKNQKFFDELRMNYIKELDRIIACKRKNPTSCSRRFYQLTKLLD
SVQPIARELHQFTFDLLIKSHMVSVDFPEMMAEIISVQVPKILSGKVK
PIYFHTQ

The identity of the steroid binding domain has been the subject of considerable research (Ai at al, Chem Res Toxicol 2003, 16, 1652-1660; Bohl et al, J Biol Chem 2005, 280(45) 37747-37754; Duff and McKewan, Mol Endocrinol 2005, 19(12) 2943-2954; Ong et al, Mol Human Reprod 2002, 8(2) 101-108; Poujol et al, J Biol Chem 2000, 275(31) 24022-24031; Rosa at al, J Clin Endocrinol Metab 87(9) 4378-4382; Marhefka et al, J Med Chem 2001, 44, 1729-1740; Matias at al, J Biol Chem 2000, 275(34) 26164-26171; McDonald et al, Cancer Res 2000, 60, 2317-2322; Sack et al, PNAS 2001, 98(9) 4904-4909; Steketee et al, Int J Cancer 2002, 100, 309-317; the contents of which are all herein incorporated by reference). While the exact residues essential for steroid binding are not known, it is generally accepted that the region spanning the approximately 250 amino acid residues in the C-terminal end of the molecule is involved (Trapman et at (1988). Biochem Biophys Res Commun 153, 241-248, the contents of which is herein incorporated by reference).

In one embodiment of the polypeptide the androgen binding region includes or consists of the sequence defined approximately by the 230 C-terminal amino acids of the sequence dnnqpd . . . iyfhtq.

Some studies have considered the crystal structure of the steroid binding domain of the human androgen receptor in complex with a synthetic steroid. For example, Sack et at (ibid) propose that the 3-dimensional structure of the receptor includes a typical nuclear receptor ligand binding domain fold. Another study proposes that the steroid binding pocket consists of approximately 18 (noncontiguous) amino acid residues that interact with the ligand (Matias et al, ibid). It is emphasized that this study utilized a synthetic steroid ligand (R1881) rather than actual dihydrotestosterone. The binding pocket for dihydrotestosterone may include the same residues as that shown for R1181 or different residues.

Further crystallographic data on the steroid binding domain complexed with agonist predict 11 helices (no helix 2) with two anti-parallel β-sheets arranged in a so-called helical sandwich pattern. In the agonist-bound conformation the carboxy-terminal helix 12 is positioned in an orientation allowing a closure of the steroid binding pocket. The fold of the ligand binding domain upon hormone binding results in a globular structure with an interaction surface for binding of interacting proteins like co-activators.

In one embodiment, the androgen binding region includes or consists of the steroid hormone binding domain of the cognate receptor, but is devoid of regions of the receptor that are not involved in steroid hormone binding.

In one embodiment of the polypeptide the nuclear hormone receptor agonist binding region includes a thyroid hormone binding domain of a thyroid hormone receptor, or functional equivalent thereof. In one embodiment of the polypeptide, the nuclear hormone receptor agonist binding region includes residues of a C-terminal region of a thyroid hormone receptor. In one embodiment of the polypeptide the C-terminus region includes residues included in the region from approximately residue 227 to the C-terminus. The thyroid receptor providing sequences for the nuclear hormone receptor agonist binding region may be alpha-1, alpha-2, beta-1 or beta-2.

From the above, it will be understood that the identity of the minimum residues required for binding any given steroid hormone or thyroid hormone may not have been settled at the filing date of this application. Accordingly, the present invention is not limited to polypeptides comprising any specific region of the receptor. It is therefore to be understood that the scope of the present invention is not necessarily limited to any specific residues as detailed herein.

In any event, the skilled person understands that various alterations may be made to the nuclear hormone receptor agonist binding sequence without completely ablating the ability of the sequence to bind steroid or thyroid hormone. Indeed it may be possible to alter the sequence to improve the ability of the domain to bind a steroid or thyroid hormone. Therefore, the scope of the invention extends to functional equivalents of the binding domain of the cognate receptor. It is expected that certain alterations could be made to the ligand binding domain sequence of the receptor without substantially affecting the ability of the domain to bind steroid. For example, the possibility exists that certain amino acid residues may be deleted, substituted, or repeated. Furthermore, the sequence may be truncated at the C-terminus and/or the N-terminus. Furthermore additional bases may be introduced within the sequence. Indeed, it may be possible to achieve a sequence having an increased affinity or avidity for a hormone by trialing a number of alterations to the amino acid sequence. The skilled person will be able to ascertain the effect (either positive or negative) on the binding by way of standard association assay with hormone, as described herein.

A proportion of hormone circulating in the blood is not biologically available. For example, the vast majority of testosterone circulating in the blood is not biologically available in that about 98% is bound to serum protein. In men, approximately 40% of serum protein bound testosterone is associated with sex hormone binding globulin (SHBG),which has an association constant (Ka) of about 1×10⁹ L/mol. The remaining approximately 60% is bound weakly, to albumin with a Ka of approximately 3×10⁴ L/mol. Estradiol also binds to SHBG to a significant extent. Other steroid hormones such as progesterone, cortisol, and other corticosteroids are bound by transcortin in the serum. Thyroid hormones (thyroxines) may be bound in the circulation to thyroxine-binding globulin (approximately 70%), transthyretin (10-15%) or albumin (15-20%).

As discussed supra, the polypeptide is capable, of decreasing biologically available steroid or thyroid hormone. In this regard, assays that measure levels of total hormone in the blood (i.e. free hormone in addition to bound hormone) may not be relevant to an assessment of whether a polypeptide is capable of decreasing biologically available hormone. A more relevant assay would be one that measures free hormone. These assays require determination of the percentage of unbound hormone by a dialysis procedure, estimation of total hormone, and the calculation of free hormone. For example, free steroid hormone can also be calculated if total steroid, SHBG, and albumin concentrations are known (Sødergard et al, Calculation of free and bound fractions of testosterone and estradiol-17β to human plasma proteins at body temperature. J Steroid Biochem. 16:801-810; the contents of which is herein incorporated by reference). Methods are also available for determination of free steroid without dialylis. These measurements may be less accurate than those including a dialysis step, especially when the steroid hormone levels are low and SHBG levels are elevated (Rosner W. 1997, J Clin Endocrinol Metabol. 82:2014-2015; the contents of which is herein incorporated by reference; Giraudi et al. 1988. Steroids. 52:423-424; the contents of which is herein incorporated by reference). However, these assays may nevertheless be capable of determining whether or not a polypeptide is capable of decreasing biologically available steroid hormone.

Another method of measuring biologically available steroid is disclosed by Nankin et al 1986 (J Clin Endocrinol Metab. 63:1418-1423; the contents of which is herein incorporated by reference. This method determines the amount of steroid not bound to SHBG and includes that which is nonprotein bound and weakly bound to albumin. The assay method relies on the fact SHBG is precipitated by a lower concentration of ammonium sulfate, 50%, than albumin. Thus by precipitating a serum sample with 50% ammonium sulfate and measuring the steroid value in the supernate, non-SHBG bound or biologically available steroid is measured. This fraction of steroid can also be calculated if total steroid, SHBG, and albumin levels are known.

Further exemplary methods of determining levels of biologically available testosterone are disclosed in de Ronde et al., 2006 (Clin Chem 52(9):1777-1784; the contents of which is herein incorporated by reference). Methods for assaying free dihydrotestosterone (Horst et al Journal of Clinical Endocrinology and Metabolism 45: 522, 1977, the contents of which is herein incorporated by reference), dihydroepiandosterone (Parker and O'Dell Journal of Clinical Endocrinology and Metabolism 47: 600, 1978, the contents of which is herein incorporated by reference), estrogen (Blondeau and Robel (1975) Eur. J. Biochem. 55, 375-384, the contents of which is herein incorporated by reference), estradiol (Mounib et al Journal of Steroid Biochemistry 31: 861-865, 1988), cortisol (Celerico et al, Clinical Chemistry, Vol 28, 1343-1345, 1982, the contents of which is herein incorporated by reference), cortisone (Meulenberg and Hofman. Clinical Chemistry 36: 70-75, 1990, the contents of which is herein incorporated by reference) aldosterone (Deck, et al J Clin Endocrinol Metab 36: 756, 1973, the contents of which is herein incorporated by reference), progesterone (Batra et al Journal of Clinical Endocrinology and Metabolism 42: 1041, 1976, the contents of which is herein incorporated by reference), and thyroxine (Fritz et al Clin Chem. 2007 May; 53(5):911-5).

In determining whether or not a polypeptide is capable of decreasing biologically available androgen, the skilled person will understand that it may be necessary to account for the natural variability of androgen levels that occur in an individual. It is known that androgen levels fluctuate in an individual according to many factors, including the time of day and the amount of exercise performed. For example, it is typically observed that testosterone levels are higher in the morning as compared with a sample taken in the evening. Even in consideration of these variables, by careful planning of sample withdrawal, or by adjusting a measurement obtained from the individual, it will be possible to ascertain whether the level of biologically available androgen in an individual (and the resultant effect on prostate cancer growth) has been affected by the administration of a polypeptide as described herein. Cortisol levels are known to fluctuate throughout the day, and also in response to environmental stress.

In one form of the invention the polypeptide has an affinity or avidity for hormone that is equal to or greater than that noted for natural carriers of hormone in the body. As discussed supra, natural carriers in the blood include SHBG, serum albumin, transcortin and thyroxine binding globulin. It will be appreciated that the binding of hormone to these natural carriers is reversible, and an equilibrium exists between the bound and unbound form of the hormone. In one form of the invention, to decrease the level of biologically available hormone to below that normally present (for example less than 1-2% in the case of testosterone) the polypeptide has an affinity or avidity for the hormone that is greater than that between the cognate binding protein and the hormone. Thus in one embodiment of the invention, the polypeptide has an association constant for the hormone that is greater than that for a natural carrier such as SHBG, albumin, transcortin or thyroid hormone binding globulin.

In another form of the invention the polypeptide has an association constant for the hormone that is approximately equal or less than that for the cognate natural carrier. In this embodiment, while free hormone may bind to the natural carrier in preference to the polypeptide, addition of polypeptide to the circulation may still be capable of decreasing the level of biologically available steroid hormone. Where the polypeptide has a low affinity or avidity for hormone, it may be necessary to administer the polypeptide in larger amounts to ensure that the level of hormone is sufficiently depleted.

In another form of the invention the polypeptide has an affinity or avidity for the hormone that is sufficiently high such that it is capable of maintaining decreased levels of hormone levels within a cell. Administration of the polypeptide can achieve this result by depleting the level of hormone in the circulation such that little or no hormone can therefore enter the cell. Additionally, or alternatively, the polypeptide is capable of entering the cell and binding to intracellular hormone.

Where the hormone is dihydrotestosterone, another form of the invention provides that the polypeptide has an affinity or avidity for dihydrotestosterone that is sufficiently high such that it is capable of maintaining decreased levels of dihydrotestosterone levels within a cell. These forms of the polypeptide interfere with the binding of testosterone and/or dihydrotestosterone to the androgen receptor within the cell. Testosterone and dihydrotestosterone are capable of binding to common targets (for example, the androgen receptor) and it is therefore proposed that the polypeptides described herein are capable of binding to both testosterone and dihydrotestosterone.

In a further form of the invention the polypeptide has an affinity or avidity for the steroid hormone that is equal to or greater than that between the steroid and any enzyme that can catalyze the steroid into a new active form of the hormone. An exemplary enzyme is that of 5-alpha-reductase. Upon entry of testosterone into the cell, the steroid is typically converted to dihydrotestosterone by the enzyme 5-alpha-reductase. In order to decrease the opportunity for intracellular testosterone to associate with the enzyme the polypeptide has a greater affinity or avidity than the enzyme for testosterone. By virtue of the superior binding of testosterone with the polypeptide, the opportunity for conversion of testosterone to dihydrotestosterone is limited. However, given the potential for a reversible association of testosterone with the polypeptide, all testosterone may eventually be converted to the dihydro form. In that case it is desirable for the polypeptide to be capable of binding to testosterone and dihydrotestosterone, or for two polypeptide species to be used (one for binding testosterone, and the other for binding dihydrotestosterone). In this embodiment of the invention, the precursor and product of the 5-alpha-reductase catalyzed reaction are liable to be bound to polypeptide the end result being lowered concentrations of both molecules available for binding to the androgen receptor.

In a further embodiment, the polypeptide has an affinity or avidity for dihydrotestosterone that is equal to or greater than the affinity or avidity of the androgen receptor for dihydrotestosterone. In another embodiment, the polypeptide has an affinity or avidity for testosterone that is equal to or greater than the affinity or avidity of the androgen receptor for testosterone.

In one form of the invention the nuclear hormone receptor agonist binding region of the polypeptide includes a sequence or sequences derived from the steroid binding domain of the human sex hormone binding protein, or functional equivalent thereof. The sequence of human SHBG is described by the following sequence:

ESRGPLATSRLLLLLLLLLLRHTRQGWALRPVLPTQSAHDPPAVHLSN
GPGQEPIAVMTFDLTKITKTSSSFEVRTWDPEGVIFYGDTNPKDDWFM
LGLRDGRPEIQLHNHWAQLTVGAGPRLDDGRWHQVEVKMEGDSVLLEV
DGEEVLRLRQVSGPLTSKRHPIMRIALGGLLFPASNLRLPLVPALDGC
LRRDSWLDKQAEISASAPTSLRSCDVESNPGIFLPPGTQAEFNLRDIP
QPHAEPWAFSLDLGLKQAAGSGHLLALGTPENPSWLSLHLQDQKVVLS
SGSGPGLDLPLVLGLPLQLKLSMSRVVLSQGSKMKALALPPLGLAPLL
NLWAKPQGRLFLGALPGEDSSTSFCLNGLWAQGQRLDVDQALNRSHEI
WTHSCPQSPGNGTDASH

The scope of the invention extends to fragments and functional equivalents of the above protein sequence.

As discussed supra, SHBG is responsible for binding the vast majority of sex hormones in the serum. Accordingly, in one embodiment of the invention the nuclear hormone receptor agonist binding region of the polypeptide includes the steroid binding domain of SHBG, or functional equivalent thereof. This domain comprises the region defined approximately by amino acid residues 18 to 177.

While the polypeptide may have more than one nuclear hormone receptor agonist binding region, in one form of the invention the polypeptide has only a single nuclear hormone receptor agonist binding region. This form of the polypeptide may be advantageous due to the potentially small size of the molecule. A smaller polypeptide may have a longer half life in the circulation, or may elicit a lower level of immune response in the body. A smaller polypeptide may also have a greater ability to enter a cell to neutralize an intracellular steroid hormone receptor agonist.

It is emphasized that the nuclear hormone receptor agonist binding region of the polypeptide is not restricted to any specific sequence or sequences described herein. The domain may be determined by reference to any other molecule (natural or synthetic) capable of binding androgen including any carrier protein, enzyme, receptor, or antibody.

In one form of the invention, the polypeptide includes a carrier region. The role of the carrier region is to perform any one or more of the following functions: to generally improve a pharmacological property of the polypeptide including bioavailability, toxicity, and half life; limit rejection or destruction by an immune response; facilitate the expression or purification of the polypeptide when produced in recombinant form; all as compared with a polypeptide that does not include a carrier region.

In one form of the invention, the carrier region comprises sequence(s) of the Fc region of an IgG molecule. Methods are known in the art for generating Fc-fusion proteins, with a number being available in kit form by companies such as Invivogen (San Diego Calif.). The Invivogen system is based on the pFUSE-Fc range of vectors which include a collection of expression plasmids designed to facilitate the construction of Fc-fusion proteins. The plasmids include wild-type Fc regions from various species and isotypes as they display distinct properties

The plasmids include sequences from human wild type Fc regions of IgG1, IgG2, IgG3 and IgG4. Furthermore, engineered human Fc regions are available that exhibit altered properties.

pFUSE-Fc plasmids feature a backbone with two unique promoters: EF1 prom/HTLV 5′UTR driving the Fc fusion and CMV enh/FerL prom driving the selectable marker Zeocin. The plasmid may also contain an IL2 signal sequence for the generation of Fc-Fusions derived from proteins that are not naturally secreted.

The Fc region binds to the salvage receptor FcRn which protects the fusion protein from lysosomal degradation giving increased half-life in the circulatory system. For example, the serum half-life of a fusion protein including the human IgG3 Fc region is around one week. In another form of the invention the Fc region includes human IgG1, IgG2 or IgG4 sequence which increases the serum half-life to around 3 weeks. Serum half-life and effector functions (if desired) can be modulated by engineering the Fc region to increase or reduce its binding to FcRn, FcγRs and C1q respectively.

Increasing the serum persistence of a therapeutic antibody is one way to improve efficacy, allowing higher circulating levels, less frequent administration and reduced doses. This can be achieved by enhancing the binding of the Fc region to neonatal FcR (FcRn). FcRn, which is expressed on the surface of endothelial cells, binds the IgG in a pH-dependent manner and protects it from degradation. Several mutations located at the interface between the CH2 and CH3 domains have been shown to increase the half-life of IgG1 (Hinton P R. et al., 2004. J Biol. Chem. 279(8):6213-6; the contents of which is herein incorporated by reference, Vaccaro C. et al., 2005. Nat. Biotechnol. 23(10):1283-8; the contents of which is herein incorporated by reference).

In one form of the invention, the carrier region comprises sequence(s) of the wild type human Fc IgG1 region, as described by the following sequence, or functional equivalents thereof

THTCPPCPAPELLGGPSVFLFPPKPKDTLMISRTPEVTCVVVDVSHED
PQVKFNWYVDGVQVHNAKTKPREQQYNSTYRVVSVLTVLHQNWLDGKE
YKCKVSNKALPAPIEKTISKAKGQPREPQVYTLPPSREEMTKNQVSLT
CLVKGFYPSDIAVEWESNGQPENNYKTTPPVLDSDGSFFLYSKLTVDK
SRWQQGNVFSCSVMHEALHNHYTQKSLSLSPG

While the polypeptide may be a fusion protein such as that described supra, it will be appreciated that the polypeptide may take any form that is capable of achieving the aim of binding a steroid hormone such that the level of steroid hormone in the blood or a cell is decreased.

For example, the polypeptide may be a therapeutic antibody. Many methods are available to the skilled artisan to design therapeutic antibodies that are capable of binding to a predetermined target, persist in the circulation for a sufficient period of time, and cause minimal adverse reaction on the part of the host (Carter, Nature Reviews (Immunology) Volume 6, 2006; the contents of which is herein incorporated by reference).

In one embodiment, the therapeutic antibody is a single clone of a specific antibody that is produced from a cell line, including a hybridoma cell. There are four classifications of therapeutic antibodies: murine antibodies; chimeric antibodies; humanized antibodies; and fully human antibodies. These different types of antibodies are distinguishable by the percentage of mouse to human parts making up the antibodies. A murine antibody contains 100% mouse sequence, a chimeric antibody contains approximately 30% mouse sequence, and humanized and fully human antibodies contain only 5-10% mouse residues.

Fully murine antibodies have been approved for human use on transplant rejection and colorectal cancer. However, these antibodies are seen by the human immune system as foreign and may need further engineering to be acceptable as a therapeutic.

Chimeric antibodies are a genetically engineered fusion of parts of a mouse antibody with parts of a human antibody. Generally, chimeric antibodies contain approximately 33% mouse protein and 67% human protein. They combine the specificity of the murine antibody with the efficient human immune system interaction of a human antibody. Chimeric antibodies can trigger an immune response and may require further engineering before use as a therapeutic. In one form of the invention, the polypeptides include approximately 67% human protein sequences.

Humanized antibodies are genetically engineered such that the minimum mouse part from a murine antibody is transplanted onto a human antibody. Typically, humanized antibodies are 5-10% mouse and 90-95% human. Humanized antibodies counter adverse immune responses seen in murine and chimeric antibodies. Data from marketed humanized antibodies and those in clinical trials show that humanized antibodies exhibit minimal or no response of the human immune system against them. Examples of humanized antibodies include Enbrel® and Remicade®. In one form of the invention, the polypeptides are based on the non-ligand specific sequences included in the Enbrel® or Remicade® antibodies.

Fully human antibodies are derived from transgenic mice carrying human antibody genes or from human cells. An example of this is the Humira® antibody. In one form of the invention, the polypeptide of the present invention is based on the non-ligand specific sequences included in the Humira® antibody.

The polypeptide may be a single chain antibody (scFv), which is an engineered antibody derivative that includes heavy- and lightchain variable regions joined by a peptide linker. ScFv antibody fragments are potentially more effective than unmodified IgG antibodies. The reduced size of 27-30 kDa allows penetration of tissues and solid tumors more readily (Huston et al. (1993). Int. Rev. Immunol. 10, 195-217; the contents of which is herein incorporated by reference). Methods are known in the art for producing and screening scFv libraries for activity, with exemplary methods being disclosed in is disclosed by Walter et al 2001, Comb Chem High Throughput Screen; 4(2):193-205; the contents of which is herein incorporated by reference.

The polypeptide may have greater efficacy as a therapeutic if in the form of a multimer. The polypeptide may be effective, or have improved efficacy when present as a homodimer, homotrimer, or homotetramer; or as a heterodimer, heterotrimer, or heterotetramer. In these cases, the polypeptide may require multimerisation sequences to facilitate the correct association of the monomeric units. Thus, in one embodiment the polypeptide includes a multimerisation region. It is anticipated that where the steroid binding region of the polypeptide includes sequences from SHBG, a multimerisation region may be included.

In another aspect, the present invention provides a composition comprising a polypeptide of the present invention in combination with a pharmaceutically acceptable carrier. The skilled person will be enabled to select the appropriate carrier(s) to include in the composition. Potentially suitable carriers include a diluent, adjuvant, excipient, or vehicle with which the polypeptide is administered. Diluents include sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin.

The polypeptides of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Furthermore, aqueous compositions useful for practicing the methods of the invention have physiologically compatible pH and osmolality. One or more physiologically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers are included in an amount required to maintain pH of the composition in a physiologically acceptable range. One or more physiologically acceptable salts can be included in the composition in an amount sufficient to bring osmolality of the composition into an acceptable range. Such salts include those having sodium, potassium or ammonium cations and chloride, citrate, ascorbate, borate, phosphate, bicarbonate, sulfate, thiosulfate or bisulfite anions.

In another aspect, the present invention provides a method for treating or preventing a condition related to excess nuclear hormone receptor agonist in a subject, the method comprising administering to a subject in need thereof an effective amount of a ligand capable of binding a nuclear hormone receptor agonist in the subject, such that the level of biologically available nuclear hormone receptor agonist in the subject is decreased as compared with the level of biologically available nuclear hormone receptor agonist present in the subject prior to administration of the polypeptide.

The present invention includes the treatment and prevention of all conditions related to the presence of excess nuclear hormone receptor agonist. For example congenital adrenal hyperplasia (CAH) refers to a family of inherited disorders in which defects occur in one of the enzymatic steps required to synthesize cortisol from cholesterol in the adrenal gland. Because of the impaired cortisol secretion, adrenocorticotropic hormone (ACTH) levels rise via a negative feedback system, which results in hyperplasia of the adrenal cortex. In the vast majority of cases, there is an accumulation of the precursors immediately proximal to the 21-hydroxylation step in the pathway of cortisol synthesis. These excess precursors are converted to potent androgens, which cause in utero virilization of the external genitalia of the female fetus in the classical form of CAH. Newborn males have normal genitalia although, as with females, they may develop other signs of androgen excess in childhood.

Another condition is apparent mineralocorticoid excess (AME). AME is a genetic disorder that typically causes severe hypertension in children, pre- and postnatal growth failure, low to undetectable levels of potassium, renin, and aldosterone levels, and is caused by a deficiency of 11b-hydroxysteroid dehydrogenase type 2 (11b-HSD2). This potentially fatal disease is caused by autosomal recessive mutations in the HSD11B2 gene.

Cushing syndrome is a disorder caused by prolonged exposure of the body's tissues to high levels of corticosteroids (glucocorticoids). Corticosteroids are powerful steroid hormones produced by the adrenal glands, located above each kidney. They regulate the metabolism of proteins, carbohydrates, and fats. They reduce the immune system's inflammatory responses and regulated maintain blood pressure and cardiac function. A vital function of corticosteroids is to assist the body respond to stress.

Corticosteroid production by the adrenal glands follows a sequence of events. The hypothalamus (see Anatomy of the Endocrine System) releases corticotropin-releasing hormone (CRH), which causes the pituitary gland to secrete adrenocorticotropic hormone (ACTH), which in turn stimulates the adrenal glands to produce corticosteroid. When the corticosteroid level is low, more CRH and ACTH are produced; when the corticosteroid level is high, less CRH and ACTH are produced. Under normal conditions, the corticosteroid level and CRH/ACTH levels are in dynamic balance; Cushing disease occurs when that balance is disturbed.

Excess corticosteroids have detrimental effects on many of the tissues and organs of the body. All of these effects together are called Cushing syndrome.

Overproduction of corticosteroids can be caused by a tumor in the pituitary gland, which produces excess ACTH, thereby stimulating the adrenal gland to produce excess corticosteroids. This condition is called Cushing disease because the origin is in the hypothalamic pituitary system. Cushing syndrome is a collection of symptoms which are similar to Cushing disease but is not the result of pituitary ACTH overproduction.

Endogenous Cushing syndrome is the result of autonomous, unregulated production of corticosteroids by a tumor within one or both of the adrenal glands themselves. The most common cause of Cushing syndrome, however, is exogenous Cushing syndrome, which results from taking excessive amounts of corticosteroid drugs for the treatment of long-term diseases such as asthma, arthritis, and lupus.

Cushing syndrome is also a relatively common condition in domestic dogs and horses where it is almost invariably caused by pituitary neoplasia, characterised by abnormal fat deposition. The syndrome in horses leads to weight loss, polyuria and polydipsia and may cause laminitis. It is emphasized that the present methods of treatment and prevention include non-human subjects.

Excess androgen disorders occur in approximately 10% to 20% of all women and usually start during puberty. Many of the women consider themselves to be normal but some may have polycystic ovary syndrome (PCOS) or hirsutism. Women with androgen disorders frequently present with gynecological problems including menstrual irregularity, dysfunctional uterine bleeding, amenorrhea, infertility, ovarian enlargement or frequent ovarian cysts, endometrial hyperplasia, fibrocystic breasts, or even virilization.

Excess androgen can also lead to morbidity in males, in conditions such as hypofertility, infertility, acne and premature balding.

Virilization can occur in childhood in either boys or girls due to excessive amounts of androgens. Typical effects of virilization in children are pubic hair, accelerated growth and bone maturation, increased muscle strength, acne, adult body odor, and sometimes growth of the penis. In a boy, virilization may signal precocious puberty, while congenital adrenal hyperplasia and androgen producing tumors (usually) of the gonads or adrenals are occasional causes in both sexes.

Virilization in a woman can manifest as clitoral enlargement, increased muscle strength, acne, hirsutism, frontal hair thinning, deepening of the voice, and menstrual disruption due to anovulation. Some of the possible causes of virilization in women are Polycystic ovary syndrome, Androgen-producing tumors of the ovaries, adrenal glands, or pituitary gland, hypothyroidism, anabolic steroid exposure, congenital adrenal hyperplasia due to 21-hydroxylase deficiency (late-onset).

Conditions related to adrenal dysfunction such as adrenal virilism, and hyperaldosteronism are also included in the scope of the invention. Conditions related to hyperthyroidism (thyrotoxicosis), are also contemplated including hypermetabolism, tachycardia, fatigue, weight loss, tremor, Graves' disease, goiter, exophthalmos, and pretibial myxedema.

In one form of the invention, the ligand is a polypeptide as described herein.

The amount of the polypeptide that will be effective for its intended therapeutic use can be determined by standard techniques well known to clinicians. Generally, suitable dosage ranges for intravenous administration are generally approximately 20 to 500 micrograms of active compound per kilogram body weight. Effective doses may be extrapolated from dose-response curves derived from in vitro or animal model test systems.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. In cases of local administration or selective uptake, the effective local concentration of the compounds may not be related to plasma concentration. One having skill in the art will be able to optimize therapeutically effective local dosages without undue experimentation.

The dosage regime could be arrived at by routine experimentation on the part of the clinician. Generally, the aim of therapy would be to bind all, or the majority of free steroid in the blood to the polypeptide. In deciding an effective dose, the amount of polypeptide could be titrated from a low level up to a level whereby the level of biologically available nuclear hormone receptor agonist is undetectable. Methods of assaying biologically available nuclear hormone receptor agonists are known in the art, as discussed elsewhere herein. Alternatively, it may be possible to theoretically estimate (for example on a molar basis) the amount of polypeptide required to neutralize substantially all free nuclear hormone receptor agonist. Alternatively, the amount could be ascertained empirically by performing a trial comparing the dosage with clinical effect. This may give an indicative mg/kg body weight dosage for successful therapy.

The duration of treatment and regularity of dosage could also be arrived at by theoretical methods, or by reference to the levels of biologically available nuclear hormone receptor agonist in the patient and/or clinical effect.

In one form of the invention, the level of biologically available nuclear hormone receptor agonist is measured in the blood of the subject, and/or in a cell of the subject.

The methods of treatment will be most efficacious where the hormonal condition has been diagnosed. However, it will be appreciated that the polypeptides may be used prophylactically before a hormonal condition has been diagnosed. Polypeptide may be administered in this way to a person with a predisposition to a relevant disease to prevent damaging effect of excess nuclear hormone receptor agonist.

In another aspect, the present invention provides a method for treating or preventing a condition related to excess nuclear hormone receptor agonist in a subject, the method comprising administering to a subject in need thereof an effective amount of a nucleic acid molecule or vector encoding a polypeptide as disclosed herein. The present invention encompasses the use of nucleic acids encoding the polypeptides of the invention for transfection of cells in vitro and in vivo. These nucleic acids can be inserted into any of a number of well-known vectors for transfection of target cells and organisms. The nucleic acids are transfected into cells ex vivo and in vivo, through the interaction of the vector and the target cell. The compositions are administered (e.g., by injection into a muscle) to a subject in an amount sufficient to elicit a therapeutic response. An amount adequate to accomplish this is defined as “a therapeutically effective dose or amount.” For gene therapy procedures in the treatment or prevention of human disease, see for example, Van Brunt (1998) Biotechnology 6:1149 1154, the contents of which is incorporated herein by reference. Methods of treatment or prevention including the aforementioned nucleic acid molecules and vectors may include treatment with other compounds useful in the treatment of hormonal conditions.

In yet a further aspect, the present invention provides the use of a polypeptide as described herein in the manufacture of a medicament for the treatment or prevention of a condition related to excess nuclear hormone receptor agonist in a subject.

In another aspect, the present invention provides the use of a nucleic acid molecule as described herein in the manufacture of a medicament for the treatment or prevention of a condition related to excess nuclear hormone receptor agonist in a subject.

Still a further aspect provides the use of a vector as described herein in the manufacture of a medicament for the treatment or prevention of a condition related to excess steroid in a subject

The present invention will now be more fully described by reference to the following non-limiting Examples.

In a first aspect, the present invention provides a bi-functional molecule comprising (i) a first region capable of binding to a steroid hormone and/or steroid hormone associated, molecule in solution and (ii) a second region having means for removing the bi-functional molecule and any bound steroid hormone and/or steroid hormone associated molecule from solution. Applicant has found that bi-functional molecules such as those described herein may be used for depleting a steroid hormone from a solution, including biological fluids such as serum.

It is proposed that the use of these bi-functional molecules are advantageous in the production of steroid-depleted sera due to the greater specificity of depletion. The specificity (or lack of specificity) of target steroid hormone can be accurately controlled by altering the binding region of the bi-functional molecule. Accordingly, in one form of the bi-functional molecule the first region is substantially specific for a steroid hormone and/or steroid hormone-associated molecule. By contrast, prior art methods such as charcoal stripping indiscriminately adsorb any lipophilic molecule. As discussed in the Background section herein, the prior art substantially alter levels of non-steroidal components in serum leading to a wide range of problems in tissue culture. Furthermore, such methods are labor intensive and time consuming.

The first region may be capable of binding to a free steroid hormone or a steroid hormone bound to another molecule. In the context of the present invention, the term “steroid hormone” is intended to include any naturally occurring or synthetic steroid hormone, or any functionally equivalent molecule. Thus, the invention includes bi-functional molecules that bind to steroid hormones that are naturally produced by an animal (and therefore potentially present in serum), and also steroids that have been administered to an animal prior to collection of serum.

Steroid hormones can be classified into the following groups: corticosteroids, androgens, estrogens, progestins, and progesterone. Corticosteroids are a group of natural and synthetic analogues of the hormones secreted by the hypothalamic-anterior pituitary-adrenocortical (HPA) axis. These include glucocorticoids, which are anti-inflammatory agents with a large number of other functions; mineralocorticoids, which control salt and water balance primarily through action on the kidneys. Exemplary corticosteroids include corticosterone (11beta,21-dihydroxy-4-pregnene-3,20-dione); deoxycorticosterone (21-hydroxy-4-pregnene-3,20-dione); cortisol (11beta,17,21-trihydroxy-4-pregnene-3,20-dione); 11-deoxycortisol (17,21-dihydroxy-4-pregnene-3,20-dione); cortisone (17,21-dihydroxy-4-pregnene-3,11,20-trione); 18-hydroxycorticosterone (11beta,18,21-trihydroxy-4-pregnene-3,20-dione); 1α-hydroxycorticosterone (1alpha,11beta,21-trihydroxy-4-pregnene-3,20-dione); and aldosterone 18,11-hemiacetal of 11beta,21-dihydroxy-3,20-dioxo-4-pregnen-18-al.

Androgens stimulate or control the development and maintenance of masculine characteristics in vertebrates by binding to androgen receptors. This includes the activity of the accessory male sex organs and development of male secondary sex characteristics. Exemplary androgens include androstenedione (4-androstene-3,17-dione); 4-hydroxy-androstenedione; 11β-hydroxyandrostenedione (11beta-4-androstene-3,17-dione); androstanediol (3-beta,17-beta-Androstanediol); androsterone (3alpha-hydroxy-5alpha-androstan-17-one); epiandrosterone (3beta-hydroxy-5alpha-androstan-17-one); adrenosterone (4-androstene-3,11,17-trione); dehydroepiandrosterone (3beta-hydroxy-5-androsten-17-one); dehydroepiandrosterone sulphate (3beta-sulfoxy-5-androsten-17-one); testosterone (17beta-hydroxy-4-androsten-3-one); epitestosterone (17alpha-hydroxy-4-androsten-3-one); 5α-dihydrotestosterone (17beta-hydroxy-5alpha-androstan-3-one 5β-dihydrotestosterone; 5-beta-dihydroxy testosterone (17beta-hydroxy-5beta-androstan-3-one); 11β-hydroxytestosterone (11beta,17beta-dihydroxy-4-androsten-3-one); and 1′-ketotestosterone (17beta-hydroxy-4-androsten-3,17-dione).

Estrogens are a group of steroid compounds, named for their importance in the estrous cycle, and functioning as the primary female sex hormone. Exemplary estrogens include estrone (3-hydroxy-1,3,5(10)-estratrien-17-one); estradiol (1,3,5(10)-estratriene-3,17beta-diol); and estriol 1,3,5(10)-estratriene-3,16alpha,17beta-triol.

Progestins are a synthetic progestogen that has some biological activity similar to progesterone and is most well known for the applications in hormonal contraception, but progestins (and progesterone) also have applications in the treatment of dysmenorrhea, endometriosis, functional uterine bleeding, and amenorrhea. Exemplary progestins include pregnenolone (3-beta-hydroxy-5-pregnen-20-one); 17-hydroxypregnenolone (3-beta,17-dihydroxy-5-pregnen-20-one); progesterone (4-pregnene-3,20-dione); and 17-hydroxyprogesterone (17-hydroxy-4-pregnene-3,20-dione). Progesterone (pregn-4-ene-3,20-dione) can be considered a natural progestin, and is included in the scope of the present invention.

The aforementioned steroid hormones are those found in the human. Where the serum is from other species (for example cows), it is intended that the equivalent steroid hormones are substituted.

The bi-functional molecules of the present invention may also be capable of binding to a molecule associated with a steroid hormone. While a proportion of steroid hormones in the blood occur freely in solution, the majority is typically bound to serum proteins such as albumin and sex hormone binding globulin (SHBG). It is therefore possible to deplete steroid using bi-functional molecules directed to a protein that is associated with a steroid hormone. Alternatively, the binding region could be directed to a target formed by components of the steroid hormone and associated protein in combination.

The bi-functional molecule may be an organic or inorganic compound, or a combination thereof. Where the bi-functional molecule is organic, the molecule may be predominantly or completely in the form of DNA or RNA. As nuclear hormones, steroid hormones may be capable of binding to nucleotide sequences.

In another form of the invention the bi-functional molecule is a predominantly or completely in the form of a polypeptide. One advantage of using a polypeptide is that toxicity issues are lessened. Sera produced by the present methods will be useful in tissue culture where it is necessary to limit any exposure of the cells to substances that may affect their viability. Peptides are of a biological origin and therefore typically of lower toxicity than many molecules that are not of biological origin. Furthermore, peptides can be produced by large scale fermentation of genetically modified bacteria, such as E. Coli. The use of polypeptides also allows fine control over the binding properties of the first region, with further details being provided infra.

The first region of the bi-functional molecule is capable of binding to the steroid hormone molecule and/or a molecule associated with the hormone. For the efficient depletion of steroid hormone, the binding must be of sufficient strength such that the binding is not materially disrupted by the process used to remove the bi-functional protein from solution. Thus, when choosing a first region for the bi-functional molecule the skilled person may take into account the full range of physicochemical changes that take place in the course of a proposed depletion method.

The binding between the first region and the steroid molecule may be substantially specific or substantially non-specific in nature. Where the binding is substantially specific, it is possible to very selectively remove certain steroid hormones, while leaving other molecules (and even other steroid hormone species) in solution. Where the binding is non-specific, the steroid hormone and/or steroid hormone associated molecule binds to the binding region, however other molecule(s) may also bind. This may be advantageous in situations where a binding region can bind to a range of molecules and it is desired to remove all species from the serum. In one form of the method, the binding region is substantially specific for a given steroid molecule such that the biological fluid is depleted only of that species of molecule and is otherwise substantially unchanged.

Where the bi-functional molecule is a polypeptide, in one embodiment, the first region comprises the steroid binding region of a steroid receptor, or functional equivalent thereof.

In another embodiment of a polypeptide bi-functional molecule, the first region includes sequences from the hormone binding domain of the mineralocorticoid receptor, or functional equivalent thereof. The sequence for the human mineralocorticoid receptor is known:

METKGYHSLPEGLDMERRWGQVSQAVERSSLGPTERTDENNYMEIVNV
SCVSGAIPNNSTQGSSKEKQELLPCLQQDNNRPGILTSDIKTELESKE
LSATVAESMGLYMDSVRDADYSYEQQNQQGSMSPAKIYQNVEQLVKFY
KGNGHRPSTLSCVNTPLRSFMSDSGSSVNGGVMRAVVKSPIMCHEKSP
SVCSPLNMTSSVCSPAGINSVSSTTASFGSFPVHSPITQGTPLTCSPN
VENRGSRSHSPAHASNVGSPLSSPLSSMKSSISSPPSHCSVKSPVSSP
NNVTLRSSVSSPANINNSRCSVSSPSNTNNRSTLSSPAASTVGSICSP
VNNAFSYTASGTSAGSSTLRDVVPSPDTQEKGAQEVPFPKTEEVESAI
SNGVTGQLNIVQYIKPEPDGAFSSSCLGGNSKINSDSSFSVPIKQEST
KHSCSGTSFKGNPTVNPFPFMDGSYFSFMDDKDYYSLSGILGPPVPGF
DGNCEGSGFPVGIKQEPDDGSYYPEASIPSSAIVGVNSGGQSFHYRIG
AQGTISLSRSARDQSFQHLSSFPPVNTLVESWKSHGDLSSRRSDGYPV
LEYIPENVSSSTLRSVSTGSSRPSKICLVCGDEASGCHYGVVTCGSCK
VFFKRAVEGQHNYLCAGRNDCIIDKIRRKNCPACRLQKCLQAGMNLGA
RKSKKLGKLKGIHEEQPQQQQPPPPPPPPQSPEEGTTYIAPAKEPSVN
TALVPQLSTISRALTPSPVMVLENIEPEIVYAGYDSSKPDTAENLLST
LNRLAGKQMIQVVKWAKVLPGFKNLPLEDQITLIQYSWMCLSSFALSW
RSYKHTNSQFLYFAPDLVFNEEKMHQSAMYELCQGMHQISLQFVRLQL
TFEEYTIMKVLLLLSTIPKDGLKSQAAFEEMRTNYIKELRKMVTKCPN
NSGQSWQRFYQLTKLLDSMHDLVSDLLEFCFYTFRESHALKVEFPAML
VEIISDQLPKVESGNAKPLYFHRK

The hormone binding region has been identified by Jalaguier et al (Journal of Steroid Biochemistry and Molecular Biology, Volume 57, Number 1, January 1996, pp. 43-50(8), the contents of which is herein incorporated by reference), as including the residues of approximately 727-984. To improve the solubility of polypeptide (and therefore improve pharmacokinetic properties), a C808S mutation may be introduced into the above sequence.

In one form of the bifunctional molecule, the mineralocorticoid receptor hormone binding domain is produced in accordance with the method of Fraser et al (J Biol Chem, Vol. 274, Issue 51, 36305-36311, Dec. 17, 1999, the contents of which is herein incorporated by reference). In that publication, the binding domain is amplified by PCR from the plasmid pRShMRNX, as described by Arriza et al (Science (1987) 237, 268-275, the contents of which is herein incorporated by reference).

In another embodiment of the bi-functional molecule, the first region includes sequences from the hormone binding domain of the glucocorticoid receptor, or functional equivalent thereof. Given its biological and pharmaceutical importance, there has been enormous interest in elucidating the hormone binding domain of this receptor. Bledsoe et al (Cell 110(1)2002, 93-105, the contents of which is herein incorporated by reference) describe the expression, purification, crystallization, and structure determination of the binding domain in complex with ligand. The full wild type sequence, of the human glucocorticoid receptor is known:

MDSKESLTPG REENPSSVLA QERGDVMDFY KTLRGGATVK
VSASSPSLAV ASQSDSKQRR LLVDFPKGSV SNAQQPDLSK
AVSLSMGLYM GETETKVMGN DLGFPQQGQI SLSSGETDLK
LLEESIANLN RSTSVPENPK SSASTAVSAA PTEKEFPKTH
SDVSSEQQHL KGQTGTNGGN VKLYTTDQST FDILQDLEFS
SGSPGKETNE SPWRSDLLID ENCLLSPLAG EDDSFLLEGN
SNEDCKPLIL PDTKPKIKDN GDLVLSSPSN VTLPQVKTEK
EDFIELCTPG VIKQEKLGTV YCQASFPGAN IIGNKMSAIS
VHGVSTSGGQ MYHYDMNTAS LSQQQDQKPI FNVIPPIPVG
SENWNRCQGS GDDNLTSLGT LNFPGRTVFS NGYSSPSMRP
DVSSPPSSSS TATTGPPPKL CLVCSDEASG CHYGVLTCGS
CKVFFKRAVE GQHNYLCAGR NDCIIDKIRR KNCPACRYRK
CLQAGMNLEA RKTKKKIKGI QQATTGVSQE TSENPGNKTI
VPATLPQLTP TLVSLLEVIE PEVLYAGYDS SVPDSTWRIM
TTLNMLGGRQ VIAAVKWAKA IPGFRNLHLD DQMTLLQYSW
MFLMAFALGW RSYRQSSANL LCFAPDLIIN EQRMTLPCMY
DQCKHMLYVS SELHRLQVSY EEYLCMKTLL LLSSVPKDGL
KSQELFDEIR MTYIKELGKA IVKREGNSSQ NWQRFYQLTK
LLDSMHEVVE NLLNYCFQTF LDKTMSIEFP EMLAEIITNQ
IPKYSNGNIK KLLFHQK

The structure reveals a distinct steroid binding pocket with features that explain ligand binding and selectivity. In one embodiment of the polypeptide, the first region includes residues approximately 521 to 777 of the glucocorticoid receptor. In one form of the polypeptide a F602S mutation is introduced into the above sequence. This mutation improves solubility and has been shown to effectively bind glucocorticoid (Bledsoe et al 2002).

In one form of the bifunctional molecule, the glucocorticoid receptor hormone binding domain is produced in accordance with the method of Fraser et al (J Biol Chem, Vol. 274, Issue 51, 36305-36311, Dec. 17, 1999, the contents of which is herein incorporated by reference). Briefly, The GR LBD was derived from the plasmid pRShGRBX (Keightley, M.-C., and Fuller, P. J. (1994) Mol. Endocrinol. 8, 431-439, the contents of which is herein incorporated by reference). This construct was derived from pRShGRNX as described by Rupprecht et al (Mol. Endocrinol. (1993) 7, 597-603, the contents of which is herein incorporated by reference).

In another form of the bi-functional molecule, the first region includes sequences from the hormone binding domain of the progesterone receptor, or functional equivalent thereof. Like all nuclear hormone receptors, the progesterone receptor has a regulatory domain, a DNA binding domain, a hinge section, and a hormone binding domain. The progesterone receptor has two isoforms (A and B). The single-copy human (hPR) gene uses separate promoters and translational start sites to produce the two isoforms. Both are included in the scope of this invention:

Williams and Sigler have solved the atomic structure of progesterone complexed with its receptor (Nature. 1998 May 28; 393(6683):392-6, the contents of which is herein incorporated by reference). The authors report the 1.8 A crystal structure of a progesterone-bound ligand-binding domain of the human progesterone receptor. The nature of this structure explains the receptor's selective affinity or avidity for progestins and establishes a common mode of recognition of 3-oxy steroids by the cognate receptors. The wild type sequence of the human progesterone sequence is known:

MTELKAKGPRAPHVAGGPPSPEVGSPLLCRPAAGPFPGSQTSDTLPEV
SAIPISLDGLLFPRPCQGQDPSDEKTQDQQSLSDVEGAYSRAEATRGA
GGSSSSPPEKDSGLLDSVLDTLLAPSGPGQSQPSPPACEVTSSWCLFG
PELPEDPPAAPATQRVLSPLMSRSGCKVGDSSGTAAAHKVLPRGLSPA
RQLLLPASESPHWSGAPVKPSPQAAAVEVEEEDGSESEESAGPLLKGK
PRALGGAAAGGGAAAVPPGAAAGGVALVPKEDSRFSAPRVALVEQDAP
MAPGRSPLATTVMDFIHVPILPLNHALLAARTRQLLEDESYDGGAGAA
SAFAPPRSSPCASSTPVAVGDFPDCAYPPDAEPKDDAYPLYSDFQPPA
LKIKEEEEGAEASARSPRSYLVAGANPAAFPDFPLGPPPPLPPRATPS
RPGEAAVTAAPASASVSSASSSGSTLECILYKAEGAPPQQGPFAPPPC
KAPGASGCLLPRDGLPSTSASAAAAGAAPALYPALGLNGLPQLGYQAA
VLKEGLPQVYPPYLNYLRPDSEASQSPQYSFESLPQKICLICGDEASG
CHYGVLTCGSCKVFFKRAMEGQHNYLCAGRNDCIVDKIRRKNCPACRL
RKCCQAGMVLGGRKFKKFNKVRVVRALDAVALPQPVGVPNESQALSQR
FTFSPGQDIQLIPPLINLLMSIEPDVIYAGHDNTKPDTSSSLLTSLNQ
LGERQLLSVVKWSKSLPGFRNLHIDDQITLIQYSWMSLMVFGLGWRSY
KHVSGQMLYFAPDLILNEQRMKESSFYSLCLTMWQIPQEFVKLQVSQE
EFLCMKVLLLLNTIPLEGLRSQTQFEEMRSSYIRELIKAIGLRQKGVV
SSSQRFYQLTKLLDNLHDLVKQLHLYCLNTFIQSRALSVEFPEMMSEV
IAAQLPKILAGMVKPLLFHKK

In one embodiment of the bi-functional molecule, the first region includes residues approximately 676 to 693 of the progesterone receptor.

In another embodiment of the bi-functional molecule, the first region includes sequences from the hormone binding domain of the estrogen receptor, or functional equivalent thereof. Wurtz et al (J Med. Chem. 1998 May 21; 41(11), the contents of which is herein incorporated by reference) published a three-dimensional model of the human estrogen receptor hormone binding domain. The quality of the model was tested against mutants, which affect the binding properties. A thorough analysis of all published mutants was performed with Insight II to elucidate the effect of the mutations. 45 out of 48 mutants can be explained satisfactorily on the basis of the model. After that, the natural ligand estradiol was docked into the binding pocket to probe its interactions with the protein. Energy minimizations and molecular dynamics calculations were performed for various ligand orientations with Discover 2.7 and the CFF91 force field. The analysis revealed two favorite estradiol orientations in the binding niche of the binding domain forming hydrogen bonds with Arg394, Glu353 and His524. The crystal structure of the ER LBD in complex with estradiol has been published (Brzozowski et al. Nature 389, 753-758, 1997, the contents of which is herein incorporated by reference). The amino acid sequence of the human estrogen receptor is as follows:

MTMTLHTKASGMALLHQIQGNELEPLNRPQLKIPLERPLGEVYLDSSK
PAVYNYPEGAAYEFNAAAAANAQVYGQTGLPYGPGSEAAAFGSNGLGG
FPPLNSVSPSPLMLLHPPPQLSPFLQPHGQQVPYYLENEPSGYTVREA
GPPAFYRPNSDNRRQGGRERLASTNDKGSMAMESAKETRYCAVCNDYA
SGYHYGVWSCEGCKAFFKRSIQGHNDYMCPATNQCTIDKNRRKSCQAC
RLRKCYEVGMMKGGIRKDRRGGRMLKHKRQRDDGEGRGEVGSAGDMRA
ANLWPSPLMIKRSKKNSLALSLTADQMVSALLDAEPPILYSEYDPTRP
FSEASMMGLLTNLADRELVHMINWAKRVPGFVDLTLHDQVHLLECAWL
EILMIGLVWRSMEHPGKLLFAPNLLLDRNQGKCVEGMVEIFDMLLATS
SRFRMMNLQGEEFVCLKSIILLNSGVYTFLSSTLKSLEEKDHIHRVLD
KITDTLIHLMAKAGLTLQQQHQRLAQLLLILSHIRHMSNKGMEHLYSM
KCKNVVPLYDLLLEMLDAHRLHAPTSRGGASVEETDQSHLATAGSTSS
HSLQKYYITGEAEGFPATV

In another embodiment of the bi-functional molecule, the first region includes sequences from the hormone binding domain of the androgen receptor, or functional equivalent thereof. The gene encoding the receptor is more than 90 kb long and codes for a protein that has 3 major functional domains. The N-terminal domain, which serves a modulatory function, is encoded by exon 1 (1,586 bp). The DNA-binding domain is encoded by exons 2 and 3 (152 and 117 bp, respectively). The steroid-binding domain is encoded by 5 exons which vary from 131 to 288 bp in size. The amino acid sequence of the human androgen receptor protein is described by the following sequence.

MEVQLGLGRVYPRPPSKTYRGAFQNLFQSVREVIQNPGPRHPEAASAA
PPGASLLLLQQQQQQQQQQQQQQQQQQQQQETSPRQQQQQQGEDGSPQ
AHRRGPTGYLVLDEEQQPSQPQSALECHPERGCVPEPGAAVAASKGLP
QQLPAPPDEDDSAAPSTLSLLGPTFPGLSSCSADLKDILSEASTMQLL
QQQQQEAVSEGSSSGRAREASGAPTSSKDNYLGGTSTISDNAKELCKA
VSVSMGLGVEALEHLSPGEQLRGDCMYAPLLGVPPAVRPTPCAPLAEC
KGSLLDDSAGKSTEDTAEYSPFKGGYTKGLEGESLGCSGSAAAGSSGT
LELPSTLSLYKSGALDEAAAYQSRDYYNFPLALAGPPPPPPPPHPHAR
IKLENPLDYGSAWAAAAAQCRYGDLASLHGAGAAGPGSGSPSAAASSS
WHTLFTAEEGQLYGPCGGGGGGGGGGGGGGGGGGGGGGGGEAGAVAPY
GYTRPPQGLAGQESDFTAPDVWYPGGMVSRVPYPSPTCVKSEMGPWMD
SYSGPYGDMRLETARDHVLPIDYYFPPQKTCLICGDEASGCHYGALTC
GSCKVFFKRAAEGKQKYLCASRNDCTIDKFRRKNCPSCRLRKCYEAGM
TLGARKLKKLGNLKLQEEGEASSTTSPTEETTQKLTVSHIEGYECQPI
FLNVLEAIEPGVVCAGHDNNQPDSFAALLSSLNELGERQLVHVVKWAK
ALPGFRNLHVDDQMAVIQYSWMGLMVFAMGWRSFTNVNSRMLYFAPDL
VFNEYRMHKSRMYSQCVRMRHLSQEFGWLQITPQEFLCMKALLLFSII
PVDGLKNQKFFDELRMNYIKELDRIIACKRKNPTSCSRRFYQLTKLLD
SVQPIARELHQFTFDLLIKSHMVSVDFPEMMAEIISVQVPKILSGKVK
PIYFHTQ

The identity of the steroid binding domain has been the subject of considerable research (Ai et al, Chem Res Toxicol 2003, 16, 1652-1660; Bohl et al, J Biol Chem 2005, 280(45) 37747-37754; Duff and McKewan, Mol Endocrinol 2005, 19(12) 2943-2954; Ong et al, Mol Human Reprod 2002, 8(2) 101-108; Poujol et al, J Biol Chem 2000, 275(31) 24022-24031; Rosa et al, J Clin Endocrinol Metab 87(9) 4378-4382; Marhefka et al, J Med Chem 2001, 44, 1729-1740; Matias et al, J Biol Chem 2000, 275(34) 26164-26171; McDonald et al, Cancer Res 2000, 60, 2317-2322; Sack et al, PNAS 2001, 98(9) 4904-4909; Steketee et al, Int J Cancer 2002, 100, 309-317; the contents of which are all herein incorporated by reference). While the exact residues essential for steroid binding are not known, it is generally accepted that the region spanning the approximately 250 amino acid residues in the C-terminal end of the molecule is involved (Trapman et al (1988). Biochem Biophys Res Commun 153, 241-248, the contents of which is herein incorporated by reference).

In one embodiment of the bi-functional molecule the androgen binding region includes or consists of the sequence defined approximately by the 230 C-terminal amino acids of the sequence dnnqpd . . . iyfhtq.

Some studies have considered the crystal structure of the steroid binding domain of the human androgen receptor in complex with a synthetic steroid. For example, Sack et at (ibid) propose that the 3-dimensional structure of the receptor includes a typical nuclear receptor ligand binding domain fold. Another study proposes that the steroid binding pocket consists of approximately 18 (noncontiguous) amino acid residues that interact with the ligand (Matias et al, ibid). It is emphasized that this study utilized a synthetic steroid ligand (R1881) rather than actual dihydrotestosterone. The binding pocket for dihydrotestosterone may include the same residues as that shown for 81181 or different residues.

Further crystallographic data on the steroid binding domain complexed with agonist predict 11 helices (no helix 2) with two anti-parallel β-sheets arranged in a so-called helical sandwich pattern. In the agonist-bound conformation the carboxy-terminal helix 12 is positioned in an orientation allowing a closure of the steroid binding pocket. The fold of the ligand binding domain upon hormone binding results in a globular structure with an interaction surface for binding of interacting proteins like co-activators.

In one embodiment, the first region includes or consists of the steroid hormone binding domain of the cognate receptor, but is devoid of regions of the receptor that are not involved in steroid hormone binding.

From the above, it will be understood that where the bi-functional molecule is a polypeptide the identity of the minimum residues required for binding any given steroid hormone and/or steroid hormone associated molecule may not have been settled at the filing date of this application. Accordingly, the present invention is not limited to polypeptides comprising any specific region of the receptor. It is therefore to be understood that the scope of the present invention is not necessarily limited to any specific residues as detailed herein.

In any event, the skilled person understands that various alterations may be made to the sequence of the first region without completely ablating the ability of the sequence to bind steroid hormone and/or steroid hormone associated molecules. Indeed it may be possible to alter the sequence to improve the ability of the domain to bind a steroid hormone and/or steroid hormone associated molecule. Therefore, the scope of the invention extends to functional equivalents of the binding domain of the cognate receptor. It is expected that certain alterations could be made to the ligand binding domain sequence of the receptor without substantially affecting the ability of the domain to bind steroid. For example, the possibility exists that certain amino acid residues may be deleted, substituted, or repeated. Furthermore, the sequence may be truncated at the C-terminus and/or the N-terminus. Furthermore additional bases may be introduced within the sequence. Indeed, it may be possible to achieve a sequence having an increased affinity or avidity for a hormone by trialling a number of alterations to the amino acid sequence. The skilled person will be able to ascertain the effect (either positive or negative) on the binding by way of standard association assay with hormone, as described herein.

As for all amino acid and nucleotide sequences disclosed herein, the scope of the invention extends to fragments and functional equivalents of those sequences.

In one form of the invention the first region has an affinity or avidity for steroid hormone that is equal to or greater than that noted for natural carriers of steroid hormone. Steroid hormones are known to bind to carrier proteins in the serum, such as sex hormone binding globulin (SHBG) and serum albumin. It will be appreciated that the binding of steroid to these natural carriers is reversible, and an equilibrium exists between the bound and unbound form of the steroid hormone. Thus, in some circumstances it may desirable to deplete all hormone (i.e. bound and unbound) by using a bi-functional protein having a very high affinity or avidity for steroid. In this way, substantially all steroid is dissociated from its cognate carrier protein and transferred to the bi-functional protein. Accordingly, in one form of the invention, the first region has an affinity or avidity for steroid hormone that is greater than that between the cognate binding protein and the steroid hormone.

In another form of the invention the first region has an association constant for the steroid hormone that is about equal or less than that for the cognate natural carrier. In this embodiment, while free steroid may bind to the natural carrier in preference to the first region, addition of polypeptide to the circulation may still be capable of decreasing the level of steroid hormone. Where the polypeptide has a low affinity or avidity for steroid hormone, it may be necessary to use larger amounts of the bi-functional protein to ensure that the level of steroid is sufficiently depleted. Accordingly, in one form of the invention the first region of the bi-function protein includes a sequence of the steroid binding domain of the human sex hormone binding protein. The sequence of human SHBG is described by the following sequence

ESRGPLATSRLLLLLLLLLLRHTRQGWALRPVLPTQSAHDPPAVHLSN
GPGQEPIAVMTFDLTKITKTSSSFEVRTWDPEGVIFYGDTNPKDDWFM
LGLRDGRPEIQLHNHWAQLTVGAGPRLDDGRWHQVEVKMEGDSVLLEV
DGEEVLRLRQVSGPLTSKRHPIMRIALGGLLFPASNLRLPLVPALDGC
LRRDSWLDKQAEISASAPTSLRSCDVESNPGIFLPPGTQAEFNLRDIP
QPHAEPWAFSLDLGLKQAAGSGHLLALGTPENPSWLSLHLQDQKVVLS
SGSGPGLDLPLVLGLPLQLKLSMSRVVLSQGSKMKALALPPLGLAPLL
NLWAKPQGRLFLGALPGEDSSTSFCLNGLWAQGQRLDVDQALNRSHEI
WTHSCPQSPGNGTDASH

The role of the second region of the bi-functional molecule is to facilitate separation of the steroid hormone and/or steroid hormone associated molecule bound to the first region from the serum. The means for removing the bi-functional molecule and any bound steroid hormone and/or steroid hormone associated molecule from solution of the second region of the bi-functional molecule may be any suitable means known to the skilled person. One suitable means is a magnetic tag such that application of a magnetic field localizes the bi-functional molecule allowing hormone-depleted solution to be decanted. Another suitable means is a polyhistidine tag, which is attracted toward certain affinity, media. Further details of methods using tagged molecules are described infra.

Another suitable means for removing the bi-functional molecule and any bound steroid hormone and/or steroid hormone associated molecule from solution relies on a change in solubility of the bi-functional molecule. For example where the bi-functional molecule is a polypeptide, binding of a steroid to the first region could trigger a conformational change in the second region such that the polypeptide becomes substantially insoluble and precipitates. The precipitate (including bound steroid) could then be removed from the solvent.

It is further contemplated that binding of a plurality of bi-functional molecules to a plurality of steroid hormone and/or steroid hormone associated molecules could resulting in the formation of a large cross-linked molecule maintained by a network of non-covalent interactions. Such a large network of molecules would lead to a decrease in solubility, in turn leading to removal of the bi-functional molecule and bound steroid hormone and/or steroid hormone associated molecule from solution. In this form of the invention, the first region and second region of the bi-functional molecule are structurally indistinct, with both regions being found in the one portion of the bi-functional molecule.

The second region may comprise a multimerisation domain, such that the bi-functional molecules naturally assemble into large, insoluble particles. In this embodiment, it may be necessary for the first region to have a particularly strong affinity for steroid hormone and/or steroid hormone associated molecule given that binding would need to be rapid given the spontaneous mulitmerisation of the bifunctional molecules.

In one form of the bi-functional molecule, the second region comprises a sequence of an elastin-like polypeptide (ELP). ELPs are soluble in aqueous solution below their transition temperature. However, when the temperature is raised above the transition temperature, they undergo a phase transition, become insoluble, and form aggregates.

ELPs are biopolymers derived from a structural motif found in the mammalian elastin protein. An ELP molecule is composed of a Val-Pro-Gly-X-Gly (VPGXG) pentapeptide repeated from 1 up to 180 times, where X, the “guest residue,” is any amino acid that does not eliminate the phase transition characteristics of the ELP. The guest residue may be a naturally occurring or non-naturally occurring amino acid. For example, the residue may be selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine. The guest residue may be a non-classical amino acid. Examples of non-classical amino acids include: D-isomers of the common amino acids, 2,4-diaminobutyric acid, alpha-amino isobutyric acid, 4-aminobutyric acid, Abu, 2-amino butyric acid, gamma-Abu, epsilon-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, beta-alanine, fluoro-amino acids, designer amino acids such as beta-methyl amino acids, C-alpha-methyl amino acids, N-alpha-methyl amino acids, and amino acid analogs in general.

In some circumstances, the insertion of Pro as the guest residue causes loss of the phase transition characteristic. Accordingly, in one aspect of the invention X is not proline.

It will be appreciated by those of skill in the art that the ELPs need not consist of only Val-Pro-Gly-X-Gly in order to exhibit the desired phase transition. The oligomeric repeats may be separated by one or more amino acid residues that do not eliminate the phase transition characteristic. In a preferred aspect of the invention, the ratio of Val-Pro-Gly-X-Gly oligomeric repeats to other amino acid residues of the ELP is greater than about 75%, more preferably, the ratio is greater than about 85%, still more preferably, the ratio is greater than about 95%, and most preferably, the ratio is greater than about 99%.

Preferred ELPs are those that provide the bi-functional protein with a transition temperature that is within a range that permits the bi-functional protein to remain soluble while being produced in a recombinant organism. It will be understood by one of skill in the art that the preferred transition temperature will vary among organisms in respect of their temperature requirements for growth. For example, where the microbe used to culture the bi-functional protein is E. coli, the preferred transition temperature is from about 37.5 to about 42.5° C. in water, preferably about 40° C. in water. Useful and preferred temperatures can be readily determined by one of skill in the art for any organism.

Preferred transition temperatures are those that permit solubility in the recombinant organism during culturing and permit aggregation of the bi-functional protein by a small increase in temperature following cell lysis. For example, a preferred difference between the culture temperature and the transition temperature is in the range of about 30 to about 40° C. In another aspect, the temperature increase is in the range of about 1 to about 7.5° C.; more preferably, the required temperature increase is in the range of about 1 to about 5° C.

Studies have shown that the fourth residue (X) in the elastin pentapeptide sequence, VPGXG, can be altered without eliminating the formation of the beta-turn. These studies also showed that the transition temperature is a function of the hydrophobicity of the guest residue. By varying the identity of the guest residue(s) and their mole fraction(s), ELPs can be synthesized that exhibit an inverse transition over a 0-100° C. range.

The transition temperature for an ELP of given length can be decreased by incorporating a larger fraction of hydrophobic guest residues in the ELP sequence. Examples of suitable hydrophobic guest residues include valine, leucine, isoleucine, phenylalanine, tryptophan and methionine. Tyrosine, which is moderately hydrophobic, may also be used. Conversely, the transition temperature can be increased by incorporating residues, such as those selected from the group consisting of: glutamic acid, cysteine, lysine, aspartate, alanine, asparagine, serine, threonine, glysine, arginine, and glutamine; preferably selected from alanine, serine, threonine and glutamic acid.

The transition temperature can also be varied by altering the ELP chain length. The Transition temperature increases dramatically with decreasing molecular weight. In low ionic strength buffers, the transition temperatures of the lower molecular weight ELPs may be too high for protein purification. This may be addressed by altering ionic strength of the solution. For example, increasing ionic strength may be used to decrease the transition temperature if necessary. In some circumstances, altering ionic strength will cause difficulties (for example, where a serum is being treated for use in tissue culture), in which case recourse could be made to alterations in the ELP chain length.

In one embodiment of the invention, the ELP sequence and repeat length are such that a transition temperature between about 40° C. to 56° C. is obtained. Temperatures in the upper portion of that range are operable for the treatment of serum, for example, which may be heat inactivated at 56° for 30 minutes to remove complement proteins. However, for cost and simplicity purposes a transition temperature of between about 40° C. to 42° C.

For polypeptides having a molecular weight >100,000, the hydrophobicity Scale developed by Urry et al. (WO/1996/032406) is preferred for predicting the approximate transition temperature of a specific ELP sequence. For polypeptides having a molecular weight <100,000, the transition temperature is preferably determined by the following quadratic function:

Tₜ=M₀+M₁X+M₂X²

where X is the MW of the bi-functional moleclue, and M₀=116.21; M₁=−1.7499; M₂=0.010349.

The regression coefficient for this fit is 0.99793

ELP chain length may be important with respect to protein yields. In addition to the decreased total yield of expressed fusion protein observed with increasing ELP MW, the weight percent of target protein versus the ELP also decreases as the MW of the ELP carrier increases. In a preferred aspect of the invention, the ELP length is from 5 to about 500 amino acid residues, more preferably from about 10 to about 450 amino acid residues, and still more preferably from about 15 to about 150 amino acid residues. ELP length can be reduced while maintaining a target transition temperature by incorporating a larger fraction of hydrophobic guest residues in the ELP sequence.

Reduction of the size of the ELP sequences in the bi-functional molecule may be employed to substantially increase the yield of the target protein. Significant reduction of the ELP sequences may increase the expression yield of the bi-functional protein.

The skilled person will be familiar with methods for producing a bi-functional molecule as described herein. Where the bi-functional molecule is a protein, methods for the expression of fusion proteins in bacteria could be utilized. Polypeptides of the invention may be produced by known recombinant expression techniques. To recombinantly produce a bi-functional molecule according to the invention, a nucleic acid sequence encoding the polypeptide is operatively linked to a promoter such that the correct polypeptide sequence is produced. Preferred promoters are those useful for expression in E. coli, such as the T7 promoter. In a preferred embodiment, the nucleic acid is DNA.

Any commonly used expression system may be used, e.g., eukaryotic or prokaryotic systems. Specific examples include yeast, Pichia, mammalian, and bacterial systems, such as E. coli, and Caulobacter.

A vector comprising the correct nucleic acid sequence can be introduced into a cell for expression of the bi-functional polypeptide. The vector can remain episomal or become chromosomally integrated, as long as it can be transcribed to produce the desired RNA. Vectors can be constructed by standard recombinant DNA technology methods. Vectors can be plasmid, viral, or other types known in the art, used for replication and expression in eukaryotic or prokaryotic cells.

It will be appreciated by one of skill in the art that a wide variety of components known in the art may be included in the vectors of the present invention, including a wide variety of transcription signals, such as promoters and other sequences that regulate the binding of RNA polymerase to the promoter.

In another aspect, the present invention provides a method for depleting a solution of a steroid hormone molecule, the method comprising the steps of exposing the serum to a bi-functional molecule as described herein, allowing the steroid hormone and/or steroid hormone associated molecule to bind to the bi-functional molecule, and removing the bi-functional molecule and any bound steroid hormone and/or steroid hormone associated molecule from the solution.

In one form of the method the solution is a serum. As used herein the term “serum” includes any liquid that has been separated from clotted blood. Also included are any derivatives of serum, including one obtained by dilution, concentration, alteration to protein content, alteration to lipid content, alteration to nucleic acid content, alteration to pH, alteration to salt content, and the like.

In one form of the invention, the step of exposing the serum to the bi-functional molecule is carried out such that at least 50%, 60%, 70%, 80%, 95%, 96%, 97%, 98% or 99% of all steroid hormone in the serum becomes bound to bi-functional protein. Optimizing the conditions for binding is well within the skill of the ordinary person, and includes manipulation of parameters such as temperature and incubation time.

The method comprises the step of removing the bi-functional molecule and any bound steroid hormone and/or steroid hormone associated molecule separating the substantially insoluble bi-functional molecule in complex with the target molecule from the serum. The step of removal can be achieved by many methods known to the skilled person. For example, the bi-functional molecule could have incorporated a polyhistidine sequence (or “his tag”), allowing removal by exposure to affinity media such as NTA-agarose, HisPur resin or Talon resin. These resins could be used in a batch-wise method (as distinct from a column chromatography method) such that a batch of serum, for example, is incubated with an affinity medium in a stirred vessel. After the tagged bi-functional molecule has bound the majority of steroid hormone and/or steroid hormone associated molecules, the resin is left to settle and the steroid-depleted supernatant is removed.

A magnetic separation system may be useful in the removal step of the present methods. The bi-functional molecule may be coupled to a magnetic bead, with steroid hormone being depleted from the solution by the application of a magnetic field to the solution. Again, the steroid-depleted supernatant is harvested as the end product. Greater efficiencies in steroid removal may be gained by incorporating a steptavidin/biotin system into the magnetic purification protocol. BioCat GmbH (Heidelberg, Del.) offer commercial kits including reagents and instructions necessary for the implementation of a magnetic removal system.

In one form of the invention, this step includes simply waiting for the insoluble complex to settle in the reaction vessel. After settlement, the supernatant could be decanted. It is to be understood that the step of separating does not require absolute separation, and that only a proportion of the insoluble complex need be separated.

In one form of the invention, the step of separating provides substantial separation of the insoluble complex from the serum. The skilled person will be familiar with a range of methods suitable for effectively separating the insoluble complex, including filtration, centrifugation, flocculation, and the like. The skilled person is also familiar with such methods, and through trial and error arrive at a suitable protocol for the removal of the insoluble complex.

In another form of the invention, the removing method is one that is already utilized in the processing of serum for laboratory use. An example is sterilization using a filter capable of removing bacteria from the product. Such filters typically have a nominal pore size of 0.2 microns, and will perform the dual role of removing bacteria and the insoluble complex.

The present invention is capable of providing sera that are depleted in only specific steroid molecules, leaving other steroid molecules, and indeed all other molecules in serum at their normal concentrations. Accordingly, in a further aspect the present invention provides a serum that is depleted in only 1, 2, 3, 4 or 5 steroid hormone species.

Also provided is a serum that includes a non-steroidal biologically active molecule at its normal concentration. Carbon-stripping indiscriminately removes many lipophilic components of serum, however use of the present invention alleviates this problem due to the specific nature of depletion. In one embodiment of the invention, the biologically active molecule is any lipophilic molecule that it present in serum. In another embodiment the biologically active molecule is selected from the group consisting of an antibody (such as IgA, IgE, IgG, IgM), a clotting factor (such as Factor I, Factor II, Factor III, Factor IV, Factor V, Factor VI, Factor VII, Factor VIII, Factor IX, Factor X, Factor XI, Factor XII, Factor XIII), a transport protein (such as transferrin, sex hormone binding globulin), a cytokine (such as PDGF, EGF, TGF-alpha, TGF-beta, FGF, NGF, any one of IL-1 to IL-13, interferon), a colony stimulating factor (such as G-CSF, M-CSF, GM-CSF), a basophilic mediator molecule (such as histamine, serotonin, prostaglandins, leukotrienes), a protein hormone (such as thyroid-stimulating hormone (TSH), follicle-stimulating hormone (FSH), Luteinizing hormone, Prolactin (PRL), Growth hormone (GH), Parathyroid hormone, Human chorionic gonadotropin (HCG), Insulin, Erythropoietin, Insulin-like growth factor-1 (IGF-1) Angiotensinogen, Thrombopoietin Leptin, Retinol Binding Protein 4, Adiponectin), a peptide hormone (such as Adrenocorticotropic hormone (ACTH), Antidiuretic hormone (ADH)(vasopressin), Oxytocin, Thyrotropin-releasing hormone (TRH), Gonadotropin-releasing hormone (GnRH) peptide, Growth hormone-releasing hormone (GHRH), Corticotropin-releasing hormone (CRH), Glucagon Somatostatin Amylin Atrial-natriuretic peptide (ANP) Gastrin, Secretin Neuropeptide Y, Ghrelin, PYY3-36), a tyrosine derivative hormone (including Dopamine, Melatonin, Thyroxine (T4), Adrenaline (epinephrine), Noradrenaline (norepinephrine), Cholecystokinin (CCK).

The present invention also provides a serum product produced by a method described herein.

In a another aspect the present invention provides a polypeptide comprising an estrogen or androgen binding region, the binding region capable of binding to an estrogen or androgen at a sufficient affinity or avidity such that upon administration of the polypeptide to a mammalian subject the level of biologically available estrogen or androgen is decreased. Anti-estrogen or anti-androgen therapy in the form of a polypeptide capable of binding to and effectively sequestering estrogen or androgen molecules is effective in the treatment of cancers for which estrogen has an involvement (such as breast cancer and ovarian cancer), or where androgen levels are relevant (such as endometrial cancer). Without wishing to be limited by theory, it is thought that sequestration of estrogen or androgen prevents binding of the hormone to its cognate receptor in cancer cells, leading to a positive clinical effect.

This approach is fundamentally distinguished from other chemotherapeutic anti-estrogen modalities that either (i) compete with natural estrogens for the binding site on the estrogen receptor leading to the formation receptor complex that is converted incompletely to the fully activated form (e.g. tamoxifen), or (ii) competitively binding to an enzyme involved in estrogen production in the body (e.g. the aromatase inhibitor anastrazole). Given that the polypeptides of the present invention bind to hormones that have a set chemical structure “escape” variants do not pose any problem. By contrast, prior art therapies target protein molecules, which may mutate leading to a lowered affinity of the drug for the target.

Applicant further proposes that anti-androgen therapy in the form of a polypeptide capable of binding to and effectively sequestering androgen molecules is effective in the treatment of cancers for which androgen has an involvement, such as endometrial cancer. The present invention is distinct from approaches of the prior art that aim to surgically remove the cancer by way of hysterectomy, or the use of mitotic inhibitors such as paclitaxel. It is further proposed that the use of anti-androgen polypeptide may be useful in lowering the levels of estrogen in the blood, given that androgens are precursor molecules in the biosynthesis of estrogens.

Typically, the polypeptide has an affinity or avidity for art estrogen or androgen molecule that is sufficiently high such that upon administration of the polypeptide to a mammalian subject, the polypeptide is capable of decreasing biologically available estrogen or androgen hormone in the blood or a cell of the subject to a level lower than that demonstrated in the subject prior to administration of the polypeptide. As used herein, the term “biologically available estrogen or androgen” means an estrogen or androgen molecule that is capable of exerting its biological activity.

A large proportion of estrogen and androgen in the blood is not biologically available.

For example, the majority of estrogen and androgen circulating in the blood is not biologically available, with most (around 97%) bound to serum proteins such sex hormone binding globulin (SHBG) and albumin. Hormone binding to SHBG has an association constant (Ka) of about 1×10⁹ L/mol, while that bound to albumin has a much weaker association with a Ka of about 3×10⁴ L/mol.

As will be understood, the present invention is directed to polypeptides that are capable of decreasing the level of an estrogen or androgen hormone available to bind to its cognate receptor in the subject. For example, in the context of the present invention where the hormone is testosterone, the term “biologically available” means that the testosterone is free for conversion to dihydrotestosterone, which subsequently binds to the androgen receptor. Where the androgen is dihydrotestosterone (typically located intracellularly) the term “biologically available” means that the dihydrotestosterone is free to bind to an androgen receptor. Where the hormone is estradiol, the term “biologically available” means that the hormone is available to bind to the estrogen receptor.

In the context of the present invention, the term “estrogen” is intended to include any naturally occurring steroid compounds involved in the regulation of the estrous cycle, and functioning as the primary female sex hormone. Exemplary estrogens include estrone (3-hydroxy-1,3,5(10)-estratrien-17-one); estradiol (1,3,5(10)-estratriene-3,17beta-diol); and estriol (1,3,5(10)-estratriene-3,16alpha,17beta-triol).

As used herein, the term “androgen” is intended to include any natural occurring steroid compound Androgens involved in the development and maintenance of masculine characteristics in vertebrates by binding to androgen receptors. This includes the activity of the accessory male sex organs and development of male secondary sex characteristics. Exemplary androgens include androstenedione (4-androstene-3,17-dione); 4-hydroxy-androstenedione; 11β-hydroxyandrostenedione (11 beta-4-androstene-3,17-dione); androstanediol (3-beta,17-beta-Androstanediol); androsterone (3alpha-hydroxy-5alpha-androstan-17-one); epiandrosterone (3beta-hydroxy-5alpha-androstan-17-one); adrenosterone (4-androstene-3,11,17-trione); dehydroepiandrosterone (3beta-hydroxy-5-androsten-17-one); dehydroepiandrosterone sulphate (3beta-sulfoxy-5-androsten-17-one); testosterone (17beta-hydroxy-4-androsten-3-one); epitestosterone (17alpha-hydroxy-4-androsten-3-one); 5α-dihydrotestosterone (17beta-hydroxy-5alpha-androstan-3-one 5(3-dihydrotestosterone; 5-beta-dihydroxy testosterone (17beta-hydroxy-5beta-androstan-3-one); 116-hydroxytestosterone (11 beta,17beta-dihydroxy-4-androsten-3-one); and 11-ketotestosterone (17beta-hydroxy-4-androsten-3,17-dione).

Estrogens and androgens of the present invention include any functionally equivalent synthetic molecule. Thus, the invention includes polypeptides that bind to hormones that are endogenous, and also those that have been administered to a patient in the course of medical treatment.

In one form of the invention, the level of biologically available estrogen is measured in the blood of the subject, or in a breast or ovarian cell. In another form of the invention the level of biologically available estrogen is decreased such that the growth of a breast cancer cell in the subject is decreased or substantially arrested.

The polypeptide may be of high affinity or low affinity or high avidity or low avidity with respect to estrogen. In one embodiment, the polypeptide has an affinity or avidity for an estrogen that is equal to or greater than the affinity or avidity between the estrogen and a protein that naturally binds to the estrogen. As an example, the polypeptide may have an affinity or avidity for estradiol that is equal to or greater than the affinity or avidity between estradiol and sex hormone binding globulin. In another form of the invention the polypeptide has an affinity or avidity for estradiol that is equal to or greater than for the affinity or avidity between estrogen and the estrogen receptor.

The polypeptide may be of high affinity or low affinity or high avidity or low avidity with respect to androgen. In one embodiment, the polypeptide has an affinity or avidity for an androgen that is equal to or greater than the affinity or avidity between the androgen and a protein that naturally binds to the androgen. As an example, the polypeptide may have an affinity or avidity for testosterone that is equal to or greater than the affinity or avidity between testosterone and sex hormone binding globulin. In another form of the invention the polypeptide has an affinity or avidity for testosterone that is equal to or greater than for the affinity or avidity between testosterone and the androgen receptor.

In one embodiment of the polypeptide the estrogen binding region comprises the estrogen binding domain from the human estrogen receptor, or a functional equivalent thereof. Wurtz et al (J Med. Chem. 1998 May 21; 41(11), the contents of which is herein incorporated by reference) published a three-dimensional model of the human estrogen receptor hormone binding domain. The quality of the model was tested against mutants, which affect the binding properties. A thorough analysis of all published mutants was performed with Insight II to elucidate the effect of the mutations. 45 out of 48 mutants can be explained satisfactorily on the basis of the model. After that, the natural ligand estradiol was docked into the binding pocket to probe its interactions with the protein. Energy minimizations and molecular dynamics calculations were performed for various ligand orientations with Discover 2.7 and the CFF91 force field. The analysis revealed two favorite estradiol orientations in the binding niche of the binding domain forming hydrogen bonds with Arg394, Glu353 and His524. After our analysis, the crystal structure of the ER LBD in complex with estradiol was published (Brzozowski et al. Nature 389, 753-758, 1997, the contents of which is herein incorporated by reference). The amino acid sequence of the human estrogen receptor is as follows:

MTMTLHTKASGMALLHQIQGNELEPLNRPQLKIPLERPLGEVYLDSSK
PAVYNYPEGAAYEFNAAAAANAQVYGQTGLPYGPGSEAAAFGSNGLGG
FPPLNSVSPSPLMLLHPPPQLSPFLQPHGQQVPYYLENEPSGYTVREA
GPPAFYRPNSDNRRQGGRERLASTNDKGSMAMESAKETRYCAVCNDYA
SGYHYGVWSCEGCKAFFKRSIQGHNDYMCPATNQCTIDKNRRKSCQAC
RLRKCYEVGMMKGGIRKDRRGGRMLKHKRQRDDGEGRGEVGSAGDMRA
ANLWPSPLMIKRSKKNSLALSLTADQMVSALLDAEPPILYSEYDPTRP
FSEASMMGLLTNLADRELVHMINWAKRVPGFVDLTLHDQVHLLECAWL
EILMIGLVWRSMEHPGKLLFAPNLLLDRNQGKCVEGMVEIFDMLLATS
SRFRMMNLQGEEFVCLKSIILLNSGVYTFLSSTLKSLEEKDHIHRVLD
KITDTLIHLMAKAGLTLQQQHQRLAQLLLILSHIRHMSNKGMEHLYSM
KCKNVVPLYDLLLEMLDAHRLHAPTSRGGASVEETDQSHLATAGSTSS
HSLQKYYITGEAEGFPATV

In another form of the polypeptide, the androgen binding region comprises the androgen binding domain from the human androgen receptor, or a functional equivalent thereof. The gene encoding the receptor is more than 90 kb long and codes for a protein that has 3 major functional domains. The N-terminal domain, which serves a modulatory function, is encoded by exon 1 (1,586 bp). The DNA-binding domain is encoded by exons 2 and 3 (152 and 117 bp, respectively). The steroid-binding domain is encoded by 5 exons which vary from 131 to 288 bp in size. The amino acid sequence of the human androgen receptor protein is described by the following sequence.

MEVQLGLGRVYPRPPSKTYRGAFQNLFQSVREVIQNPGPRHPEAASAA
PPGASLLLLQQQQQQQQQQQQQQQQQQQQQETSPRQQQQQQGEDGSPQ
AHRRGPTGYLVLDEEQQPSQPQSALECHPERGCVPEPGAAVAASKGLP
QQLPAPPDEDDSAAPSTLSLLGPTFPGLSSCSADLKDILSEASTMQLL
QQQQQEAVSEGSSSGRAREASGAPTSSKDNYLGGTSTISDNAKELCKA
VSVSMGLGVEALEHLSPGEQLRGDCMYAPLLGVPPAVRPTPCAPLAEC
KGSLLDDSAGKSTEDTAEYSPFKGGYTKGLEGESLGCSGSAAAGSSGT
LELPSTLSLYKSGALDEAAAYQSRDYYNFPLALAGPPPPPPPPHPHAR
IKLENPLDYGSAWAAAAAQCRYGDLASLHGAGAAGPGSGSPSAAASSS
WHTLFTAEEGQLYGPCGGGGGGGGGGGGGGGGGGGGGGGGEAGAVAPY
GYTRPPQGLAGQESDFTAPDVWYPGGMVSRVPYPSPTCVKSEMGPWMD
SYSGPYGDMRLETARDHVLPIDYYFPPQKTCLICGDEASGCHYGALTC
GSCKVFFKRAAEGKQKYLCASRNDCTIDKFRRKNCPSCRLRKCYEAGM
TLGARKLKKLGNLKLQEEGEASSTTSPTEETTQKLTVSHIEGYECQPI
FLNVLEAIEPGVVCAGHDNNQPDSFAALLSSLNELGERQLVHVVKWAK
ALPGFRNLHVDDQMAVIQYSWMGLMVFAMGWRSFTNVNSRMLYFAPDL
VFNEYRMHKSRMYSQCVRMRHLSQEFGWLQITPQEFLCMKALLLFSII
PVDGLKNQKFFDELRMNYIKELDRIIACKRKNPTSCSRRFYQLTKLLD
SVQPIARELHQFTFDLLIKSHMVSVDFPEMMAEIISVQVPKILSGKVK
PIYFHTQ

The identity of the steroid binding domain has been the subject of considerable research (Ai et al, Chem Res Toxicol 2003, 16, 1652-1660; Bohl et al, J Biol Chem 2005, 280(45) 37747-37754; Duff and McKewan, Mol Endocrinol 2005, 19(12) 2943-2954; Ong et al, Mol Human Reprod 2002, 8(2) 101-108; Poujol et al, J Biol Chem 2000, 275(31) 24022-24031; Rosa et al, J Clin Endocrinol Metab 87(9) 4378-4382; Marhefka et al, J Med Chem 2001, 44, 1729-1740; Matias et al, J Biol Chem 2000, 275(34) 26164-26171; McDonald et al, Cancer Res 2000, 60, 2317-2322; Sack et al, PNAS 2001, 98(9) 4904-4909; Steketee et al, Int J Cancer 2002, 100, 309-317; the contents of which are all herein incorporated by reference). While the exact residues essential for steroid binding are not known, it is generally accepted that the region spanning the approximately 250 amino acid residues in the C-terminal end of the molecule is involved (Trapman et al (1988). Biochem Biophys Res Commun 153, 241-248, the contents of which is herein incorporated by reference).

In one embodiment of the invention the androgen binding region comprises or consists of the sequence approximately defined by the 230 C-terminal amino acids of the sequence dnnqpd . . . iyfhtq.

Some studies have considered the crystal structure of the steroid binding domain of the human androgen receptor in complex with a synthetic steroid. For example, Sack et al (ibid) propose that the 3-dimensional structure of the receptor includes a typical nuclear receptor ligand binding domain fold. Another study proposes that the steroid binding pocket has been consists of approximately 18 (noncontiguous) amino acid residues that interact with the ligand (Matias et al, ibid). It is emphasized that this study utilized a synthetic steroid ligand (R1881) rather than actual dihydrotestosterone. The binding pocket for dihydrotestosterone may include the same residues as that shown for R1181 or different residues.

Further crystallographic data on the steroid binding domain complexed with agonist predict 11 helices (no helix 2) with two anti-parallel n-sheets arranged in a so-called helical sandwich pattern. In the agonist-bound conformation the carboxy-terminal helix 12 is positioned in an orientation allowing a closure of the steroid binding pocket. The fold of the ligand binding domain upon hormone binding results in a globular structure with an interaction surface for binding of interacting proteins like co-activators.

In one embodiment, the estrogen or androgen binding region comprises or consists of the steroid hormone binding domain of the cognate receptor, but is devoid of regions of the receptor that are not involved in steroid hormone binding.

From the above, it will be understood that the identity of the minimum residues required for binding any given hormone may not have been settled at the filing date of this application. Accordingly, the present invention is not limited to polypeptides comprising any specific region of the receptor. It is therefore to be understood that the scope of the present invention is not necessarily limited to any specific residues as detailed herein.

In any event, the skilled person understands that various alterations may be made to the hormone binding sequence without completely ablating the ability of the sequence to bind estrogen or androgen. Indeed it may be possible to alter the sequence to improve the ability of the domain to bind an estrogen or androgen. Therefore, the scope of the invention extends to functional derivatives of the estrogen binding domain of the estrogen receptor, and to functional equivalents of the androgen binding domain of the androgen receptor. It is expected that certain alterations could be made to the hormone binding domain sequence of the relevant receptor without substantially affecting the ability of the domain to bind hormone. For example, the possibility exists that certain amino acid residues may be deleted, substituted, or repeated. Furthermore, the sequence may be truncated at the C-terminus and/or the N-terminus. Furthermore additional bases may be introduced within the sequence. Indeed, it may be possible to achieve a sequence having an increased affinity or avidity for estrogen or androgen by trialing a number of alterations to the amino acid sequence. The skilled person will be able to ascertain the effect (either positive or negative) on the binding by way of standard association assay with estrogen or androgen, as described herein.

In another form of the polypeptide the androgen or estrogen binding region comprises the estrogen binding domain from the sex hormone binding globulin, or a functional equivalent thereof.

In one form of the invention the steroid hormone binding region of the polypeptide comprises a sequence or sequences derived from the steroid binding domain of the human sex hormone binding protein, or a functional equivalent thereof. The sequence of human SHBG is described by the following sequence:

ESRGPLATSRLLLLLLLLLLRHTRQGWALRPVLPTQSAHDPPAVHLSN
GPGQEPIAVMTFDLTKITKTSSSFEVRTWDPEGVIFYGDTNPKDDWFM
LGLRDGRPEIQLHNHWAQLTVGAGPRLDDGRWHQVEVKMEGDSVLLEV
DGEEVLRLRQVSGPLTSKRHPIMRIALGGLLFPASNLRLPLVPALDGC
LRRDSWLDKQAEISASAPTSLRSCDVESNPGIFLPPGTQAEFNLRDIP
QPHAEPWAFSLDLGLKQAAGSGHLLALGTPENPSWLSLHLQDQKVVLS
SGSGPGLDLPLVLGLPLQLKLSMSRVVLSQGSKMKALALPPLGLAPLL
NLWAKPQGRLFLGALPGEDSSTSFCLNGLWAQGQRLDVDQALNRSHEI
WTHSCPQSPGNGTDASH

The scope of the invention extends to fragments and functional equivalents of the above protein sequence. As discussed supra, SHBG is responsible for binding the vast majority of sex hormones in the serum. Accordingly, in one embodiment of the invention the steroid hormone binding region of the polypeptide includes the steroid binding domain of SHBG, or a functional equivalent thereof. This domain comprises the region defined approximately by amino acid residues 18 to 177.

As discussed supra, the polypeptide is capable of decreasing biologically available estrogen. Exemplary methods for measuring of, estrogens, such as estradiol, include both indirect and direct immunoassays, and are discussed in Lee et al. 2006, J Clin Endocrinol Metab. 91(10):3791-7, Blondeau and Robel (1975) Eur. J. Biochem. 55, 375-384, and Mounib et al Journal of Steroid Biochemistry 31: 861-865, 1988) the contents of which are all herein incorporated by reference). Examining estradiol levels within the low postmenopausal range, 0-30 pg/ml (0 to 110 pmol/liter), requires more accurate and sensitive assays than the assay methods typically used to discriminate between postmenopausal and premenopausal levels in the 20- to 30-pg/ml range and were originally developed for use in younger women, with the range of interest exceeding 50 pg/ml (183 pmol/liter). Assays that measure levels of total estrogen in the blood (i.e. free hormone in addition to bound hormone) may not be relevant to an assessment of whether a polypeptide is capable of decreasing biologically available estrogen. A more relevant assay would be one that measures free estrogen. An indicator of free estrogen levels is the free estrogen index (FEI). The FEI may be calculated using total estradiol and SHBG values by the following equation: FEI=estradiol (pg/ml)×0.367/SHBG (nmol/l).

In another form of the invention the polypeptide is capable of decreasing the level of biologically available androgen. Free steroid hormone can also be calculated if total steroid, SHBG, and albumin concentrations are known (Sødergard et al, J Steroid Biochem. 16:801-810; the contents of which is herein incorporated by reference). Methods are also available for determination of free steroid without dialysis. These measurements may be less accurate than those including a dialysis step, especially when the steroid hormone levels are low and SHBG levels are elevated (Rosner W. 1997, J Clin Endocrinol Metabol. 82:2014-2015; the contents of which is herein incorporated by reference; Giraudi et al. 1988. Steroids. 52:423-424; the contents of which is herein incorporated by reference). However, these assays may nevertheless be capable of determining whether or not a polypeptide is capable of decreasing biologically available steroid hormone.

Another method of measuring biologically available androgen is disclosed by Nankin et al 1986 (J Clin Endocrinol Metab. 63:1418-1423; the contents of which is herein incorporated by reference. This method determines the amount of steroid not bound to SHBG and includes that which is nonprotein bound and weakly bound to albumin. The assay method relies on the fact SHBG is precipitated by a lower concentration of ammonium sulfate, 50%, than albumin. Thus by precipitating a serum sample with 50% ammonium sulfate and measuring the steroid value in the supernate, non-SHBG bound or biologically available steroid is measured. This fraction of steroid can also be calculated if total steroid, SHBG, and albumin levels are known.

Further exemplary methods of determining levels of biologically available testosterone are disclosed in de Ronde et al., 2006 (Clin Chem 52(9):1777-1784; the contents of which is herein incorporated by reference). Methods for assaying free dihydrotestosterone (Horst et al Journal of Clinical Endocrinology and Metabolism 45: 522,1977, the contents of which is herein incorporated by reference), dihydroepiandosterone (Parker and O'Dell Journal of Clinical Endocrinology and Metabolism 47: 600,1978, the contents of which is herein incorporated by reference).

In determining whether or not a polypeptide is capable of decreasing biologically available estrogen or androgen, the skilled person will understand that it may be necessary to account for the natural variability of estrogen and androgen levels that occur in an individual. It is known that estradiol and testosterone levels fluctuate in an individual according to many factors, including the time of day, the amount of exercise performed, and timing of the estrous cycle. Even in consideration of these variables, by careful planning of sample withdrawal, or by adjusting a measurement obtained from the individual, it will be possible to ascertain whether the level of biologically available estrogen or androgen in an individual (and the resultant effect on the growth of cancer cells) has been affected by the administration of a polypeptide as described herein.

In one form of the invention the polypeptide has an affinity or avidity for estrogen or androgen that is equal to or greater than that noted for natural carriers of estrogen in the body. As discussed supra, natural carriers in the blood include SHBG and serum albumin. It will be appreciated that the binding of estrogen to these natural carriers is reversible, and an equilibrium exists between the bound and unbound form of the hormone. In one form of the invention, to decrease the level of biologically available estradiol or testosterone to below that normally present (for example less than about 3% of total hormone in the blood) the polypeptide has an affinity or avidity for the hormone that is greater than that between the cognate binding protein and the hormone. Thus in one embodiment of the invention, the polypeptide has an association constant for the estrogen or androgen that is greater than that for a natural carrier of estrogen or androgen such as SHBG or albumin.

In one form of the polypeptide, the polypeptide has a single estrogen or androgen binding region. This embodiment of the polypeptide may be advantageous due to the potentially small size of the molecule. A smaller polypeptide may have a longer half life in the circulation, or may elicit a lower level of immune response in the body. A smaller polypeptide may also have a greater ability to enter a cell to neutralize intracellular hormone, such as dihydroxytestosterone.

One form of the invention provides a polypeptide with a carrier region. The role of the carrier region is to perform any one or more of the following functions: to generally improve a pharmacological property of the polypeptide including bioavailability, toxicity, and half life; limit rejection or destruction by an immune response; facilitate the expression or purification of the polypeptide when produced in recombinant form; all as compared with a polypeptide that does not include a carrier region.

In one form of the invention, the carrier region comprises sequence(s) of the Fc region of an IgG molecule. Methods are known in the art for generating Fc-fusion proteins, with a number being available in kit form by companies such as Invivogen (San Diego Calif.). The Invivogen system is based on the pFUSE-Fc range of vectors which include a collection of expression plasmids designed to facilitate the construction of Fc-fusion proteins. The plasmids include wild-type Fc regions from various species and isotypes as they display distinct properties

The plasmids include sequences from human wild type Fc regions of IgG1, IgG2, IgG3 and IgG4. Furthermore, engineered human Fc regions are available that exhibit altered properties.

pFUSE-Fc plasmids feature a backbone with two unique promoters: EF1 prom/HTLV 5′UTR driving the Fc fusion and CMV enh/FerL prom driving the selectable marker Zeocin. The plasmid may also contain an IL2 signal sequence for the generation of Fc-Fusions derived from proteins that are not naturally secreted.

The Fc region binds to the salvage receptor FcRn which protects the fusion protein from lysosomal degradation giving increased half-life in the circulatory system. For example, the serum half-life of a fusion protein including the human IgG3 Fc region is around one week. In another form of the invention the Fc region includes human IgG1, IgG2 or IgG4 sequence which increases the serum half-life to around 3 weeks. Serum half-life and effector functions (if desired) can be modulated by engineering the Fc region to increase or reduce its binding to FcRn, FcγRs and C1q respectively.

Increasing the serum persistence of a therapeutic antibody is one way to improve efficacy, allowing higher circulating levels, less frequent administration and reduced doses. This can be achieved by enhancing the binding of the Fc region to neonatal FcR (FcRn). FcRn, which is expressed on the surface of endothelial cells, binds the IgG in a pH-dependent manner and protects it from degradation. Several mutations located at the interface between the CH2 and CH3 domains have been shown to increase the half-life of IgG1 (Hinton P R. et al., 2004. J Biol. Chem. 279(8):6213-6; the contents of which is herein incorporated by reference, Vaccaro C. et al., 2005. Nat. Biotechnol. 23(10):1283-8; the contents of which is herein incorporated by reference).

In one form of the invention, the carrier region comprises sequence(s) of the wild type human Fc IgG1 region, as described by the following sequence, or functional equivalents thereof

THTCPPCPAPELLGGPSVFLFPPKPKDTLMISRTPEVTCVVVDVSHED
PQVKFNWYVDGVQVHNAKTKPREQQYNSTYRVVSVLTVLHQNWLDGKE
YKCKVSNKALPAPIEKTISKAKGQPREPQVYTLPPSREEMTKNQVSLT
CLVKGFYPSDIAVEWESNGQPENNYKTTPPVLDSDGSFFLYSKLTVDK
SRWQQGNVFSCSVMHEALHNHYTQKSLSLSPG

While the polypeptide may be a fusion protein such as that described supra, it will be appreciated that the polypeptide may take any form that is capable of achieving the aim of binding a steroid hormone such that the level of steroid hormone in the blood or a cell is decreased.

In one form of the invention the polypeptide is selected from the group consisting of a fusion protein, a monoclonal antibody, a polyclonal antibody, and a single chain antibody.

For example, the polypeptide may be a therapeutic antibody. Many methods are available to the skilled artisan to design therapeutic antibodies that are capable of binding to a predetermined target, persist in the circulation for a sufficient period of time, and cause minimal adverse reaction on the part of the host (Carter, Nature Reviews (Immunology) Volume 6, 2006; the contents of which is herein incorporated by reference).

In one embodiment, the therapeutic antibody is a single clone of a specific antibody that is produced from a cell line, including a hybridoma cell. There are four classifications of therapeutic antibodies: murine antibodies; chimeric antibodies; humanized antibodies; and fully human antibodies. These different types of antibodies are distinguishable by the percentage of mouse to human parts making up the antibodies. A murine antibody contains 100% mouse sequence, a chimeric antibody contains approximately 30% mouse sequence, and humanized and fully human antibodies contain only 5-10% mouse residues.

Fully murine antibodies have been approved for human use on transplant rejection and colorectal cancer. However, these antibodies are seen by the human immune system as foreign and may need further engineering to be acceptable as a therapeutic.

Chimeric antibodies are a genetically engineered fusion of parts of a mouse antibody with parts of a human antibody. Generally, chimericantibodies contain approximately 33% mouse protein and 67% human protein. They combine the specificity of the murine antibody with the efficient human immune system interaction of a human antibody. Chimeric antibodies can trigger an immune response and may require further engineering before use as a therapeutic. In one form of the invention, the polypeptides include approximately 67% human protein sequences.

Humanized antibodies are genetically engineered such that the minimum mouse part from a murine antibody is transplanted onto a human antibody. Typically, humanized antibodies are 5-10% mouse and 90-95% human, Humanized antibodies counter adverse immune responses seen in murine and chimeric antibodies. Data from marketed humanized antibodies and those in clinical trials show that humanized antibodies exhibit minimal or no response of the human immune system against them. Examples of humanized antibodies include Enbrel® and Remicade®. In one form of the invention, the polypeptides are based on the non-ligand specific sequences included in the Enbrel® or Remicade® antibodies.

Fully human antibodies are derived from transgenic mice carrying human antibody genes or from human cells. An example of this is the Humira® antibody. In one form of the invention, the polypeptide of the present invention is based on the non-ligand specific sequences included in the Humira® antibody.

The polypeptide may be a single chain antibody (scFv), which is an engineered antibody derivative that includes heavy- and lightchain variable regions joined by a peptide linker. ScFv antibody fragments are potentially more effective than unmodified IgG antibodies. The reduced size of 27-30 kDa allows penetration of tissues and solid tumors more readily (Huston et al. (1993). Int. Rev. Immunol. 10, 195-217; the contents of which is herein incorporated by reference). Methods are known in the art for producing and screening scFv libraries for activity, with exemplary methods being disclosed in is disclosed by Walter et al 2001, Comb Chem High Throughput Screen; 4(2):193-205; the contents of which is herein incorporated by reference.

The polypeptide may have greater efficacy as a therapeutic if in the form of a multimer. The polypeptide may be effective, or have improved efficacy when present as a homodimer, homotrimer, or homotetramer; or as a heterodimer, heterotrimer, or heterotetramer. In these cases, the polypeptide may require multimerisation sequences to facilitate the correct association of the monomeric units. Thus, in one embodiment the polypeptide comprises a multimerisation region. It is anticipated that where the steroid binding region of the polypeptide comprises sequences from SHBG, a multimerisation region may be included.

The present invention also provides a nucleic acid molecule capable of encoding a polypeptide as described herein, and a vector comprising a nucleic acid molecule as described herein. These nucleic acid molecules and vectors will be useful in methods for the recombinant production of the subject polypeptides as well as gene therapy methods for the treatment or prevention of cancer.

Further provided is a composition comprising a polypeptide as described herein and a pharmaceutically acceptable carrier. The skilled person will be enabled to select the appropriate carrier(s) to include in the composition. Potentially suitable carriers include a diluent, adjuvant, excipient, or vehicle with which the polypeptide is administered. Diluents include sterile liquids, such as water and oils, including those of pefroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin.

The polypeptides of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Furthermore, aqueous compositions useful for practicing the methods of the invention have physiologically compatible pH and osmolality. One or more physiologically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers are included in an amount required to maintain pH of the composition in a physiologically acceptable range. One or more physiologically acceptable salts can be included in the composition in an amount sufficient to bring osmolality of the composition into an acceptable range. Such salts include those having sodium, potassium or ammonium cations and chloride, citrate, ascorbate, borate, phosphate, bicarbonate, sulfate, thiosulfate or bisulfite anions.

In another aspect the present invention provides a method for treating or preventing an estrogen-related cancer or an androgen-related cancer in a subject, the method comprising administering to a subject in need thereof an effective amount of a ligand capable of binding estrogen or androgen in the subject, such that the level of biologically available estrogen or androgen in the subject is decreased as compared with the level of biologically available estrogen or androgen present in the subject prior to administration of the ligand.

As used herein, the term “estrogen-related cancer” is intended to include any cancer that includes a cell that demonstrates estrogen sensitive growth, proliferation or differentiation. In one form of the method, the estrogen-related cancer is selected from the group consisting of breast cancer and ovarian cancer.

As used herein, the term “androgen-related cancer” is intended to include any cancer that includes a cell that demonstrates androgen sensitive growth, proliferation or differentiation. In one form of the method, the androgen-related cancer is endometrial cancer.

As discussed supra in describing properties of the polypeptides, the level of biologically available hormone may be measured in the blood of the subject. Alternatively, the level of biologically available estrogen may be measured in a breast cell or an ovarian cell. The level of biologically available androgen may be measured in an endometrial cell.

In one form of the method the ligand is a polypeptide as described herein. The amount of the polypeptide that will be effective for its intended therapeutic use can be determined by standard techniques well known to clinicians. Generally, suitable dosage ranges for intravenous administration are generally about 20 to 500 micrograms of active compound per kilogram body weight. Effective doses may be extrapolated from dose-response curves derived from in vitro or animal model test systems.

For systemic administration; a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. In cases of local administration or selective uptake, the effective local concentration of the compounds may not be related to plasma concentration. One having skill in the art will be able to optimize therapeutically effective local dosages without undue experimentation.

The dosage regime could be arrived at by routine experimentation on the part of the clinician. Generally, the aim of therapy would be to bind all, or the majority of free estrogen or androgen in the blood to the polypeptide. In deciding an effective dose, the amount of polypeptide could be titrated from a low level up to a level whereby the level of biologically available hormone is undetectable. Methods of assaying biologically available estrogens and androgens are known in the art, as discussed elsewhere herein. Alternatively, it may be possible to theoretically estimate (for example on a molar basis) the amount of polypeptide required to neutralize substantially all free hormone. Alternatively, the amount could be ascertained empirically by performing a trial comparing the dosage with clinical effect. This may give an indicative mg/kg body weight dosage for successful therapy.

The duration of treatment and regularity of dosage could also be arrived at by theoretical methods, or by reference to the levels of biologically available hormone in the patient and/or clinical effect.

In one form of the method, the level of biologically available steroid hormone is measured in the blood of the subject, and/or in a cell of the subject.

The methods of treatment will be most efficacious where cancer has already been diagnosed. However, it will be appreciated that the polypeptides may be used prophylactically before cancer has been diagnosed. For example, women with a strong family history of breast cancer could have an estradiol-specific polypeptide infused on a regular basis as a preventative measure.

In another aspect the present invention provides a method for treating or preventing an estrogen-related cancer or an androgen-related cancer, the method comprising administering to a subject in need thereof an effective amount of a nucleic acid molecule or a vector according as described herein. Thus, present invention encompasses the use of nucleic acids encoding the polypeptides of the invention for transfection of cells in vitro and in vivo. These nucleic acids can be inserted into any of a number of well-known vectors for transfection of target cells and organisms. The nucleic acids are transfected into cells ex vivo and in vivo, through the interaction of the vector and the target cell. The compositions are administered (e.g., by injection into a muscle) to a subject in an amount sufficient to elicit a therapeutic response. An amount adequate to accomplish this is defined as “an effective amount.”

For gene therapy procedures in the treatment or prevention of human disease, see for example, Van Brunt (1998) Biotechnology 6:1149 1154, the contents of which is incorporated herein by reference. Methods of treatment or prevention including the aforementioned nucleic acid molecules and vectors may include treatment with other compounds useful in the treatment of cancer. The estrogen-related cancer may be selected from the group consisting of breast cancer and ovarian cancer, while the androgen-related cancer may be endometrial cancer.

In a further aspect the present invention provides a method for treating or preventing estrogen flare or testosterone flare in the treatment of a subject having estrogen-related cancer with an LHRH agonist or antagonist comprising administering to a subject in need thereof an effective amount of a polypeptide as described herein. LHRH drugs eventually result in suppression of testosterone and estradiol, however before this occurs production of these hormones actually increases for a period. During the first week of treatment with a LHRH agonist or antagonist, the vastly increased production of testosterone or estradiol may cause the cancer to flare.

Another aspect of the invention provides the use of a polypeptide as described herein in the manufacture of a medicament for the treatment or prevention of an estrogen-related cancer or an androgen-related cancer. The estrogen-related cancer may be selected from the group consisting of breast cancer and ovarian cancer, and the androgen-related cancer may be endometrial cancer.

In a further aspect the present invention provides the use of a polypeptide as described herein in the manufacture of a medicament for the treatment or prevention of estrogen flare or testosterone flare.

In a first aspect the present invention provides a polypeptide comprising an androgen binding region, the androgen binding region capable of binding to an androgen at a sufficient affinity or avidity such that upon administration of the polypeptide to a mammalian subject the level of biologically available androgen is decreased. Applicant proposes that polypeptides having the ability to bind to an androgen are useful in decreasing the level of hormones such as testosterone and dihydrotestosterone that are biologically available to stimulate the androgen receptor in prostate cancer cells. In the normal course of events, the androgen receptor binds testosterone or its active metabolite dihydrotestosterone. After dissociation of heat shock proteins the receptor enters the nucleus via an intrinsic nuclear localization signal. Upon steroid hormone binding, which may occur either in the cytoplasm or in the nucleus, the androgen receptor binds as homodimer to specific DNA elements present as enhancers in upstream promoter sequences of androgen target genes. The next step is recruitment of coactivators, which can form the communication bridge between receptor and several components of the transcription machinery. The direct and indirect communication of the androgen receptor complex with several components of the transcription machinery such as RNA-polymerase II, TATA box binding protein (TBP), TBP associating factors, and general transcription factors, are key events in nuclear signaling. This communication subsequently triggers mRNA synthesis and consequently protein synthesis, which finally results in an androgen response.

Activation of the androgen receptor in prostate epithelial cells stimulates cell proliferation by increasing the transcription of genes encoding proteins such as cdks 2 and 4 that drive progression through G1, ultimately leading to Rb hypophosphorylation and commitment to cell division. Androgen receptor activation has recently been shown to result in non-genomic activation of a number of mitogenic cascades, including src/raf/ERK and PI3K/AKT. Activation of these pathways occurs rapidly, is ligand dependent, and results from direct interaction between the receptor and upstream kinases. While this stimulation of cell proliferation is necessary to maintain homeostasis in the prostate (1-2% of luminal secretory cells are lost per week though attrition or injury) the growth response must be regulated to prevent the uncontrolled growth seen in the cancerous prostate. The polypeptides described herein are proposed to limit or prevent activation of the androgen receptor by androgen, thereby decreasing or substantially arresting proliferation of prostate cells.

The present invention is distinct from approaches of the prior art that aim to decrease the production of testosterone. As discussed in the Background section herein, this has been achieved by removal of the testes, or decreasing the production of testosterone by the testes using compounds such as GnRH/LHRH agonists, GnRH antagonists, and cyproterone acetate (CPA). Compounds such as ketoconazole and corticosteroids have been used in the prior art to decrease the production of testosterone precursors by the adrenal glands. By contrast, the polypeptides of the present invention do not directly interfere with the production of androgen by the testes or adrenal glands.

The present invention is also distinguished from prior art treatments that act to block 5-alpha-reductase, the enzyme present in prostate cells that converts testosterone to dihydrotestosterone. While both testosterone and dihydrotestosterone are able to bind the androgen receptor, dihydrotestosterone is the more potent ligand. Thus, while compounds such as finasteride and dutasteride can limit the level of dihydrotestosterone in a prostate cell, they are unable to affect the binding of testosterone directly to the androgen receptor. In one embodiment of the invention, the polypeptides of the present invention are proposed to bind both testosterone and dihydrotestosterone, thereby overcoming the problems of 5-alpha-reductase inhibitors.

The polypeptides of the present invention are also different to compounds of the prior art such as CPA, bicalutamide, nilutamide and flutamide that bind to the androgen receptor. While these compounds have some efficacy in blocking the receptor they are incapable (as a monotherapy) to sufficiently limit androgen signaling. As mentioned supra antiandrogen monotherapy has been demonstrated to be inferior to castration at prolonging survival in metastatic disease. In addition, about 10% of hormone refractory prostate cancer patients have one or more mutations in the androgen receptor gene such that compounds of the prior art may act as partial agonists of the androgen receptor.

By contrast, the polypeptides of the present invention bind to molecules that have a set chemical structure, and “escape” variants do not need to be accounted for.

In one form of the invention the polypeptide is capable of binding to testosterone present in the blood. The vast majority of testosterone in the blood is bound to proteins such as steroid hormone binding globulin (SHBG) and albumin. The remaining testosterone (only about 1-2%) is biologically available. It is this unbound or “free” testosterone that is available for activating the androgen receptor in prostate cells.

In another form of the invention the polypeptide is capable of entering a prostate cell, and particularly a prostate epithelial cell. As used herein, the term “prostate cell” is intended to include a cell within or associated with the actual prostate gland, or a cell that has metastasized from the gland and has lodged in a remote location to form a secondary tumour. The term is also intended to include a cell that is in transit from the prostate gland to the final site of lodgement at the secondary tumour. The advantage of a polypeptide capable of entering the cell is that the opportunity is increased to bind all testosterone and/or dihydrotestosterone. It is pertinent to note that although after androgen ablation therapy serum testosterone levels decrease by >90%, the concentration of dihydrotestosterone in the prostate declines by only 60% (Labrie, F et al., Treatment of prostate cancer with gonadotropin releasing hormone agonists. Endocr review, 1986. 7(1): 67-74). This failure to achieve more complete ablation of androgen in the prostate may be due to cells in the organ retaining a reservoir of androgen capable of acting in an autocrine manner. There is also evidence to suggest that hormone refractory prostate cancer cells are capable of synthesizing androgens from circulating precursor molecules. Given that androgen receptor blockers of the prior art are simple competitive inhibitors, it is likely that intraprostatic steroidogenesis leads to locally increased concentrations of androgens thereby contributing at least in part to the failure of these therapies. By directly targeting intracellular androgen, Applicants propose a more complete ablation of androgen is possible using the polypeptides described herein. Certain forms of the polypeptide including features that facilitate entry into prostate cells are disclosed infra.

In a further form of the invention the polypeptide is capable of binding to androgen present in both the blood and in cells of the prostate. Typically, a polypeptide that has the ability to enter a cell, will also be operable in the blood.

It is proposed that the polypeptide is capable of removing testosterone such that the level of androgen available to bind to its receptor is decreased such that the growth of a prostate cancer cell in the subject is decreased or substantially arrested.

Typically, the polypeptide has an affinity or avidity for androgen that is sufficiently high such that upon administration of the polypeptide to a mammalian subject, the polypeptide is capable of decreasing biologically available androgen in the blood or prostate cell of the subject to a level lower than that demonstrated in the subject prior to administration of the polypeptide. As used herein, the term “biologically available androgen” means androgen that is capable of exerting its biological activity. As will be understood, the present invention is directed to polypeptides that are capable of decreasing the level of androgen available to bind to an androgen receptor in a prostate cell of the subject. Thus, in the context of the present invention where the androgen is testosterone, the term “biologically available” means that the testosterone is free for conversion to dihydrotestosterone, which subsequently binds to the androgen receptor. Where the androgen is dihydrotestosterone (typically located intracellularly) the term “biologically available” means that the dihydrotestosterone is free to bind to an androgen receptor.

The vast majority of testosterone circulating in the blood is not biologically available in that about 98% is bound to serum protein. In men, approximately 40% of serum protein bound testosterone is associated with sex hormone binding globulin (SHBG),which has an association constant (Ka) of about 1×10⁹ L/mol. The remaining approximately 60% is bound weakly to albumin with a Ka of about 3×10⁴ L/mol.

As discussed supra, the polypeptide is capable of decreasing biologically available androgen. In this regard, androgen assays that measure levels of total testosterone in the blood (i.e. free testosterone in addition to bound testosterone) may not be relevant to an assessment of whether a polypeptide is capable of decreasing biologically available androgen. A more relevant assay would be one that measures free testosterone. These assays require determination of the percentage of unbound testosterone by a dialysis procedure, estimation of total testosterone, and the calculation of free testosterone. Free testosterone can also be calculated if total testosterone, SHBG, and albumin concentrations are known (Sødergard et al, Calculation of free and bound fractions of testosterone and estradiol-17β to human plasma proteins at body temperature. J Steroid Biochem. 16:801-810; the contents of which is herein incorporated by reference). Methods are also available for determination of free testosterone without dialysis. These measurements may be less accurate than those including a dialysis step, especially when the testosterone levels are low and SHBG levels are elevated (Rosner W. 1997 Errors in measurement of plasma free testosterone. J Clin Endocrinol Metabol. 82:2014-2015; the contents of which is herein incorporated by reference; Giraudi et al. 1988. Effect of tracer binding to serum proteins on the reliability of a direct free testosterone assay. Steroids. 52:423-424; the contents of which is herein incorporated by reference). However, these assays may nevertheless be capable of determining whether or not a polypeptide is capable of decreasing biologically available testosterone.

Another method of measuring biologically available testosterone is disclosed by Nankin et al 1986 (Decreased bioavailable testosterone in aging normal and impotent men. J Clin Endocrinol Metab. 63:1418-1423; the contents of which is herein incorporated by reference. This method determines the amount of testosterone not bound to SHBG and includes that which is nonprotein bound and weakly bound to albumin. The assay method relies on the fact SHBG is precipitated by a lower concentration of ammonium sulfate, 50%, than albumin. Thus by precipitating a serum sample with 50% ammonium sulfate and measuring the testosterone value in the supernate, non-SHBG bound or biologically available testosterone is measured. This fraction of testosterone can also be calculated if total testosterone, SHBG, and albumin levels are known.

Further exemplary methods of determining levels of biologically available testosterone are disclosed in de Ronde et al., 2006 (Calculation of bioavailable and free testosterone in men: a comparison of 5 published algorithms. Clin Chem 52(9):1777-1784; the contents of which is herein incorporated by reference).

In determining whether or not a polypeptide is capable of decreasing biologically available androgen, the skilled person will understand that it may be necessary to account for the natural variability of androgen levels that occur in an individual. It is known that androgen levels fluctuate in an individual according to many factors, including the time of day and the amount of exercise performed. For example, it is typically observed that testosterone levels are higher in the morning as compared with a sample taken in the evening. Even in consideration of these variables, by careful planning of sample withdrawal, or by adjusting a measurement obtained from the individual, it will be possible to ascertain whether the level of biologically available androgen in an individual (and the resultant effect on prostate cancer growth) has been affected by the administration of a polypeptide as described herein.

In one form of the invention the polypeptide has an affinity or avidity for androgen that is equal to or greater than that noted for natural carriers of androgen in the body. As discussed supra, natural carriers in the blood include SHBG and serum albumin. It will be appreciated that the binding of testosterone to these natural carriers is reversible, and an equilibrium exists between the bound and unbound form of testosterone. In one form of the invention, to decrease the level of biologically available testosterone to below that normally present (i.e. less than 1-2%) the polypeptide has an affinity or avidity for testosterone that is greater than that between SHBG and testosterone, or albumin and testosterone. Thus in one embodiment of the invention, the polypeptide has an association constant for testosterone that is greater than that for a natural carrier of testosterone such as SHBG or albumin.

In another form of the invention the polypeptide has an association constant for testosterone that is about equal or less than that for a natural carrier of testosterone such as SHBG or albumin. In this embodiment, while free testosterone may bind to SHBG or albumin in preference to the polypeptide, addition of polypeptide to the circulation may still be capable of decreasing the level of biologically available testosterone. Where the polypeptide has a low affinity or avidity for androgen, it may be necessary to administer the polypeptide in larger amounts to ensure that the level of androgen is sufficiently depleted.

In another form of the invention the polypeptide has an affinity or avidity for testosterone that is sufficiently high such that it is capable of maintaining decreased levels of testosterone levels within a prostate cell, and more particularly a prostate epithelial cell. Administration of the polypeptide can achieve this result by depleting the level of testosterone in the circulation such that little or no testosterone can therefore enter the prostate cell. Additionally, or alternatively, the polypeptide is capable of entering the prostate cell and binding to intracellular testosterone and or dihydrotestosterone.

Given that testosterone is converted into dihydrotestosterone in cells of the prostate, another form of the invention provides that the polypeptide has an affinity or avidity for dihydrotestosterone that is sufficiently high such that it is capable of maintaining decreased levels of dihydrotestosterone levels within a prostate cell. These forms of the polypeptide interfere with the binding of testosterone and/or dihydrotestosterone to the androgen receptor within the prostate cell. Testosterone and dihydrotestosterone are capable of binding to common targets (for example, the androgen receptor) and it is therefore proposed that the polypeptides described herein are capable of binding to both testosterone and dihydrotestosterone. As discussed supra the proliferation of cancerous prostate cells may be decreased or arrested by inhibiting the androgen response of the cells.

In a further form of the invention the polypeptide has an affinity or avidity for testosterone that is equal to or greater than that between testosterone and the 5-alpha-reductase enzyme present in prostate cells. As discussed supra upon entry of testosterone into the prostate cell, the steroid is typically converted to dihydrotestosterone by the enzyme 5-alpha-reductase. In order to decrease the opportunity for intracellular testosterone to associate with the enzyme the polypeptide has a greater affinity than the enzyme for testosterone. By virtue of the superior binding of testosterone with the polypeptide, the opportunity for conversion of testosterone to dihydrotestosterone is limited. However, given the potential for a reversible association of testosterone with the polypeptide, all testosterone may eventually be converted to the dihydro form. In that case it is desirable for the polypeptide to be capable of binding to testosterone and dihydrotestosterone, or for two polypeptide species to be used (one for binding testosterone, and the other for binding dihydrotestosterone). In this embodiment of the invention, the precursor and product of the 5-alpha-reductase catalyzed reaction are liable to be bound to polypeptide the end result being lowered concentrations of both molecules available for binding to the androgen receptor.

In a further embodiment, the polypeptide has an affinity or avidity for dihydrotestosterone that is equal to or greater than the affinity or avidity of the androgen receptor for dihydrotestosterone. In another embodiment, the polypeptide has an affinity or avidity for testosterone that is equal to or greater than the affinity or avidity of the androgen receptor for testosterone.

In one form of the invention the androgen binding region of the polypeptide includes a sequence or sequences derived from human androgen receptor. The gene encoding the receptor is more than 90 kb long and codes for a protein that has 3 major functional domains. The N-terminal domain, which serves a modulatory function, is encoded by exon 1 (1,586 bp). The DNA-binding domain is encoded by exons 2 and 3 (152 and 117 bp, respectively). The steroid-binding domain is encoded by 5 exons which vary from 131 to 288 bp in size. The amino acid sequence of the human androgen receptor protein is described by the following sequence (SEQ ID NO: 1)

mevqlglgrv yprppsktyr gafqnlfqsv reviqnpgpr
hpeaasaapp gasllllqqq qqqqqqqqqq qqqqqqqqet
sprqqqqqqg edgspqahrr gptgylvlde eqqpsqpqsa
lechpergcv pepgaavaas kglpqqlpap pdeddsaaps
tlsllgptfp glsscsadlk dilseastmq llqqqqqeav
segsssgrar easgaptssk dnylggtsti sdnakelcka
vsvsmglgve alehlspgeq lrgdcmyapl lgvppavrpt
pcaplaeckg sllddsagks tedtaeyspf kggytkgleg
eslgcsgsaa agssgtlelp stlslyksga ldeaaayqsr
dyynfplala gpppppppph phariklenp ldygsawaaa
aaqcrygdla slhgagaagp gsgspsaaas sswhtlftae
egqlygpcgg gggggggggg gggggggggg ggeagavapy
gytrppqgla gqesdftapd vwypggmvsr vpypsptcvk
semgpwmdsy sgpygdmrle tardhvlpid yyfppqktcl
icgdeasgch ygaltcgsck vffkraaegk qkylcasrnd
ctidkfrrkn cpscrlrkcy eagmtlgark lkklgnlklq
eegeasstts pteettqklt vshiegyecq piflnvleai
epgvvcaghd nnqpdsfaal lsslnelger qlvhvvkwak
alpgfrnlhv ddqmaviqys wmglmvfamg wrsftnvnsr
mlyfapdlvf neyrmhksrm ysqcvrmrhl sqefgwlqit
pqeflcmkal llfsiipvdg lknqkffdel rmnyikeldr
iiackrknpt scsrrfyqlt klldsvqpia relhqftfdl
likshmvsvd fpemmaeiis vqvpkilsgk vkpiyfhtq

The present invention also includes functional equivalents of sequences as described herein. As will be understood, bases or amino acid residues may be substituted, repeated, deleted or added without substantially affecting the biological activity of the polypeptide. It will therefore be understood that strict congruence with the above sequence is not necessarily required.

In one embodiment, the androgen binding region includes or consists of the steroid binding domain of the human androgen receptor, but is devoid of regions of the receptor that are not involved in steroid binding. The identity of the steroid binding domain of the androgen receptor has been the subject of considerable research (Ai et al, Chem Res Toxicol 2003, 16, 1652-1660; Bohl et al, J Biol Chem 2005, 280(45) 37747-37754; Duff and McKewan, Mol Endocrinol 2005, 19(12) 2943-2954; Ong et al, Mol Human Reprod 2002, 8(2) 101-108; Poujol et al, J Biol Chem 2000, 275(31) 24022-24031; Rosa et al, J Clin Endocrinol Metab 87(9) 4378-4382; Marhefka et al, J Med Chem 2001, 44, 1729-1740; Matias et al, J Biol Chem 2000, 275(34) 26164-26171; McDonald et al, Cancer Res 2000, 60, 2317-2322; Sack et al, PNAS 2001, 98(9) 4904-4909; Steketee et al, Int J Cancer 2002, 100, 309-317; the contents of all aforementioned publications are herein incorporated by reference). While the exact residues essential for steroid binding are not known, it is generally accepted that the region spanning the approximately 250 amino acid residues in the C-terminal end of the molecule is involved (Trapman et al (1988). Biochem Biophys Res Commun 153, 241-248, the contents of which is herein incorporated by reference).

In one embodiment of the invention the androgen binding region includes or consists of the sequence defined by the 230 C-terminal amino acids of SEQ ID NO:1 (i.e. the sequence dnnqpd . . . iyfhtq).

Some studies have considered the crystal structure of the steroid binding domain of the human androgen receptor in complex with a synthetic steroid. For example, Sack et al (ibid) propose that the 3-dimensional structure of the receptor includes a typical nuclear receptor ligand binding domain fold. Another study proposes that the steroid binding pocket has been consists of 18 (noncontiguous) amino acid residues that interact with the ligand (Matias et al, ibid). It is emphasized that this study utilized a synthetic steroid ligand (R1881) rather than actual dihydrotestosterone. The binding pocket for dihydrotestosterone may include the same residues as that shown for R1181 or different residues.

Further crystallographic data on the steroid binding domain complexed with agonist predict 11 helices (no helix 2) with two anti-parallel β-sheets arranged in a so-called helical sandwich pattern. In the agonist-bound conformation the carboxy-terminal helix 12 is positioned in an orientation allowing a closure of the steroid binding pocket. The fold of the ligand binding domain upon hormone binding results in a globular structure with an interaction surface for binding of interacting proteins like co-activators.

From the above, it will be understood that the identity of the minimum residues required for binding androgen has not been settled at the filing date of this application. Accordingly, the present invention is not limited to polypeptides including any specific region of the androgen receptor as discussed supra. It is therefore to be understood that the scope of the present invention is not necessarily limited to any specific residues as detailed herein.

In any event, while the steroid binding domain of the androgen receptor is generally well conserved, the skilled person understands that various alterations may be made without completely ablating the ability of the sequence to bind steroid. Indeed it may be possible to alter the sequence to improve the ability of the domain to bind androgen. Therefore, the scope of the invention extends to functional derivatives of the steroid binding domain of the androgen receptor. It is expected that certain alterations could be made to the ligand binding domain sequence of the androgen receptor without substantially affecting the ability of the domain to bind androgen. For example, the possibility exists that certain amino acid residues may be deleted, substituted, or repeated. Furthermore, the sequence may be truncated at the C-terminus and/or the N-terminus. Furthermore additional bases may be introduced within the sequence. Indeed, it may be possible to achieve a sequence having an increased affinity for androgen by trialing a number of alterations to the amino acid sequence. The skilled person will be able to ascertain the effect (either positive or negative) on the binding by way of standard association assay with androgen, as described supra.

In one form of the invention the androgen binding region of the polypeptide includes a sequence or sequences derived from the steroid binding domain of the human sex hormone binding protein. The sequence of human SHBG is described by the following sequence (SEQ ID NO: 2)

esrgplatsr llllllllll rhtrqgwalr pvlptqsahd
ppavhlsngp gqepiavmtf dltkitktss sfevrtwdpe
gvifygdtnp kddwfmlglr dgrpeiqlhn hwaqltvgag
prlddgrwhq vevkmegdsv llevdgeevl rlrqvsgplt
skrhpimria lggllfpasn lrlplvpald gclrrdswld
kqaeisasap tslrscdves npgiflppgt qaefnlrdip
qphaepwafs ldlglkqaag sghllalgtp enpswlslhl
qdqkvvlssg sgpgldlplv lglplqlkls msrvvlsqgs
kmkalalppl glapllnlwa kpqgrlflga lpgedsstsf
clnglwaqgq rldvdqalnr sheiwthscp qspgngtdas
h

The scope of the invention extends to fragments and functional equivalents of the above protein sequence.

As discussed supra, SHBG is responsible for binding the vast majority of testosterone in the serum. Accordingly, in one embodiment of the invention the steroid binding domain of the polypeptide includes the testosterone binding domain of SHBG. This domain comprises the region defined approximately by amino acid residues 18 to 177.

While the polypeptide may have more than one androgen binding region, in one form of the invention the polypeptide has only a single androgen binding region. This form of the polypeptide may be advantageous due to the potentially small size of the molecule. A smaller polypeptide may have a longer half life in the circulation, or may elicit a lower level of immune response in the body. A smaller polypeptide may also have a greater ability to enter a prostate cell to neutralize intracellular androgen.

It is emphasized that the steroid binding region of the polypeptide is not restricted to any specific sequence or sequences described herein. The domain may be determined by reference to any other molecule (natural or synthetic) capable of binding androgen including any carrier protein, enzyme, receptor, or antibody.

In one form of the invention, the polypeptide includes a carrier region. The role of the carrier region is to perform any one or more of the following functions: to generally improve a pharmacological property of the polypeptide including bioavailability, toxicity, and half life; limit rejection or destruction by an immune response; facilitate the expression or purification of the polypeptide when produced in recombinant form; all as compared with a polypeptide that does not include a carrier region.

In one form of the invention, the carrier region comprises sequence(s) of the Fc region of an IgG molecule. Methods are known in the art for generating Fc-fusion proteins, with a number being available in kit form by companies such as Invivogen (San Diego Calif.). The Invivogen system is based on the pFUSE-Fc range of vectors which include a collection of expression plasmids designed to facilitate the construction of Fc-fusion proteins. The plasmids include wild-type Fc regions from various species and isotypes as they display distinct properties

The plasmids include sequences from human wild type Fc regions of IgG1, IgG2, IgG3 and IgG4. Furthermore, engineered human Fc regions are available that exhibit altered properties.

pFUSE-Fc plasmids feature a backbone with two unique promoters: EF1 prom/HTLV 5′UTR driving the Fc fusion and CMV enh/FerL prom driving the selectable marker Zeocin. The plasmid may also contain an IL2 signal sequence for the generation of Fc-Fusions derived from proteins that are not naturally secreted.

The Fc region binds to the salvage receptor FcRn which protects the fusion protein from lysosomal degradation giving increased half-life in the circulatory system. For example, the serum half-life of a fusion protein including the human IgG3 Fc region is around one week. In another form of the invention the Fc region includes human IgG1, IgG2 or IgG4 sequence which increases the serum half-life to around 3 weeks. Serum half-life and effector functions (if desired) can be modulated by engineering the Fc region to increase or reduce its binding to FcRn, FcγRs and C1q respectively.

Increasing the serum persistence of a therapeutic antibody is one way to improve efficacy, allowing higher circulating levels, less frequent administration and reduced does. This can be achieved by enhancing the binding of the Fc region to neonatal FcR (FcRn). FcRn, which is expressed on the surface of endothelial cells, binds the IgG in a pH-dependent manner and protects it from degradation. Several mutations located at the interface between the CH2 and CH3 domains have been shown to increase the half-life of IgG1 (Hinton P R. et al., 2004. Engineered human IgG antibodies with longer serum half-lives in primates. J Biol. Chem. 279(8):6213-6; the contents of which is herein incorporated by reference, Vaccaro C. et al., 2005. Engineering the Fc region of immunoglobulin G to modulate in vivo antibody levels. Nat Biotechnol. 23(10):1283-8; the contents of which is herein incorporated by reference).

In one form of the invention, the carrier region comprises sequence(s) of the wild type human Fc IgG1 region, as described by the following sequence (SEQ ID NO: 3), or functional equivalents thereof

thtcppcpap ellggpsvfl fppkpkdtlm isrtpevtcv
vvdvshedpq vkfnwyvdgv qvhnaktkpr eqqynstyrv
vsvltvlhqn wldgkeykck vsnkalpapi ektiskakgq
prepqvytlp psreemtknq vsltclykgf ypsdiavewe
sngqpennyk ttppvldsdg sfflyskltv dksrwqqgnv
fscsvmheal hnhytqksls lspg

While the polypeptide may be a fusion protein such as that described supra, it will be appreciated that the polypeptide may take any form that is capable of achieving the aim of binding an androgen such that the level of androgen in the blood or prostate cell is decreased.

For example, the polypeptide may be a therapeutic antibody. Many methods are available to the skilled artisan to design therapeutic antibodies that are capable of binding to a predetermined target, persist in the circulation for a sufficient period of time, and cause minimal adverse reaction on the part of the host (Carter, Nature Reviews (Immunology) Volume 6, 2006; the contents of which is herein incorporated by reference).

In one embodiment, the therapeutic antibody is a single clone of a specific antibody that is produced from a cell line, including a hybridoma cell. There are four classifications of therapeutic antibodies: murine antibodies; chimeric antibodies; humanized antibodies; and fully human antibodies. These different types of antibodies are distinguishable by the percentage of mouse to human parts making up the antibodies. A murine antibody contains 100% mouse sequence, a chimeric antibody contains approximately 30% mouse sequence, and humanized and fully human antibodies contain only 5-10% mouse residues.

Fully murine antibodies have been approved for human use on transplant rejection and colorectal cancer. However, these antibodies are seen by the human immune system as foreign and may need further engineering to be acceptable as a therapeutic.

Chimeric antibodies are a genetically engineered fusion of parts of a mouse antibody with parts of a human antibody. Generally, chimeric antibodies contain approximately 33% mouse protein and 67% human protein. They combine the specificity of the murine antibody with the efficient human immune system interaction of a human antibody. Chimeric antibodies can trigger an immune response and may require further engineering before use as a therapeutic. In one form of the invention, the polypeptides include approximately 67% human protein sequences.

Humanized antibodies are genetically engineered such that the minimum mouse part from a murine antibody is transplanted onto a human antibody. Typically, humanized antibodies are 5-10% mouse and 90-95% human. Humanized antibodies counter adverse immune responses seen in murine and chimeric antibodies. Data from marketed humanized antibodies and those in clinical trials show that humanized antibodies exhibit minimal or no response of the human immune system against them. Examples of humanized antibodies include Enbrel® and Remicade®. In one form of the invention, the polypeptides are based on the non-ligand specific sequences included in the Enbrel® or Remicade® antibodies.

Fully human antibodies are derived from transgenic mice carrying human antibody genes or from human cells. An example of this is the Humira® antibody. In one form of the invention, the polypeptide of the present invention is based on the non-ligand specific sequences included in the Humira® antibody.

The polypeptide may be a single chain antibody (scFv), which is an engineered antibody derivative that includes heavy- and lightchain variable regions joined by a peptide linker. ScFv antibody fragments are potentially more effective than unmodified IgG antibodies. The reduced size of 27-30 kDa allows penetration of tissues and solid tumors more readily (Huston et al. (1993). Int. Rev. Immunol. 10, 195-217; the contents of which is herein incorporated by reference). Methods are known in the art for producing and screening scFv libraries for activity, with exemplary methods being disclosed in is disclosed by Walter et al 2001, High-throughput screening of surface displayed gene products Comb Chem High Throughput Screen; 4(2):193-205; the contents of which is herein incorporated by reference.

The polypeptide may have greater efficacy as a therapeutic if in the form of a multimer. The polypeptide may be effective, or have improved efficacy when present as a homodimer, homotrimer, or homotetramer; or as a heterodimer, heterotrimer, or heterotetramer. In these cases, the polypeptide may require multimerisation sequences to facilitate the correct association of the monomeric units. Thus, in one embodiment the polypeptide includes a multimerisation region. It is anticipated that where the steroid binding region of the polypeptide includes sequences from SHBG, a multimerisation region may be included.

In another aspect, the present invention provides a composition comprising a polypeptide of the present invention in combination with a pharmaceutically acceptable carrier. The skilled person will be enabled to select the appropriate carrier(s) to include in the composition. Potentially suitable carriers include a diluent, adjuvant, excipient, or vehicle with which the polypeptide is administered. Diluents include sterile liquids, such as water and oils, including those of petroleum, animal, vegetable or synthetic origin, such at peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin.

The polypeptides of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Furthermore, aqueous compositions useful for practicing the methods of the invention have physiologically compatible pH and osmolality. One or more physiologically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers are included in an amount required to maintain pH of the composition in a physiologically acceptable range. One or more physiologically acceptable salts can be included in the composition in an amount sufficient to bring osmolality of the composition into an acceptable range. Such salts include those having sodium, potassium or ammonium cations and chloride, citrate, ascorbate, borate, phosphate, bicarbonate, sulfate, thiosulfate or bisulfite anions.

In another aspect, the present invention includes a method for treating or preventing prostate cancer in a subject, the method comprising administering to a subject in need thereof an effective amount of a ligand capable of binding androgen in the subject, such that the level of biologically available androgen in the subject is decreased. In one form of the method, the ligand is a polypeptide as described herein.

The amount of the polypeptide that will be effective for its intended therapeutic use can be determined by standard clinical techniques well known to clinicians. Generally, suitable dosage ranges for intravenous administration are generally about 20 to 500 micrograms of active compound per kilogram body weight. Effective doses may be extrapolated from dose-response curves derived from in vitro or animal model test systems.

For systemic administration, a therapeutically effective dose can be estimated initially from in vitro assays. For example, a dose can be formulated in animal models to achieve a Circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. In cases of local administration or selective uptake, the effective local concentration of the compounds may not be related to plasma concentration. One having skill in the art will be able to optimize therapeutically effective local dosages without undue experimentation.

The dosage regime could be arrived at by routine experimentation on the part of the clinician. Generally, the aim of therapy would be to bind all, or the majority of free androgen in the blood and prostate cell to the polypeptide. In deciding an effective dose, the amount of polypeptide could be titrated from a low level up to a level whereby the level of biologically available testosterone is undetectable. Methods of assaying biologically available testosterone are known in the art, as discussed elsewhere herein. Alternatively, it may be possible to theoretically estimate (for example on a molar basis) the amount of polypeptide required to neutralize substantially all free testosterone. Alternatively, the amount could be ascertained empirically by performing a trial comparing the dosage with clinical effect. This may give an indicative mg/kg body weight dosage for successful therapy.

The duration of treatment and regularity of dosage could also be arrived at by theoretical methods, or by reference to the levels of biologically available testosterone in the patient and/or clinical effect.

In one form of the method, the level of biologically available androgen is measured in the blood of the subject, and/or in a prostate cell (and particularly a prostate epithelial cell) of the subject.

The methods of treatment will be most efficacious where the prostate cancer is in the androgen dependent phase. However, it will be appreciated that the polypeptides may be used prophylactically before the prostate, cancer has been diagnosed. Polypeptide may be administered in this way to a person with a strong family history of prostate cancer, or with any other predisposition to the disease.

It is contemplated that the methods of treatment and prophylaxis included the use a polypeptide as described herein as a monotherapy, or in combination with at least one other therapeutic used in the treatment of prophylaxis of prostate cancer. It is proposed that in some forms of the invention use of the polypeptides as described herein as part of a combination therapy provide advantages. An advantage may be due to the unique mechanism by which the polypeptides, of the present invention act as therapeutics. As discussed herein, the polypeptides act to bind androgen, such that the level of biologically available androgen in the blood and/or prostate cell is decreased. This is distinct from prior art therapeutics that typically act by decreasing the amount of androgen secreted by the body. It is therefore proposed that by the use of combination, and additive or synergistic effect may be realized.

As a non-limiting example of a combination therapy, an androgen agonist and a polypeptide of the present invention may be co-administered to patients in the early androgen dependent phase of the disease. Androgen agonist drugs (such as leuprolide) are typically administered with the aim of inducing castrate levels of androgens in the blood. This is typically defined as a 90% reduction in levels of serum testosterone. However, it is contemplated that an advantage is gained where low levels of androgen agonist drugs are administered such that serum testosterone is reduced to supra-castrate levels (for example, a reduction of from about 25% to about 75%). In this case, the polypeptide is administered with the aim of neutralizing the remaining testosterone. The advantage of this approach, is that for a given dose of polypeptide a longer half-life results since the polypeptide would not have neutralize all of the serum testosterone but only 25 to 50% of normal levels.

Combination treatment including a polypeptide of the present invention will further decrease the levels of serum testosterone by physically sequestering the remaining testosterone. In this example, the different, yet complementary mechanisms of action of the two therapeutic agents may result in a superior depletion of serum testosterone available for binding to the androgen receptor in prostate cancer cells. The combination therapy may also provide an improved side effect profile, or allow for the use of lower dosages of androgen agonist.

Combination therapy may also be useful where patients are administered a dosage of androgen agonist sufficient to provide castrate levels of serum testosterone, and the disease has progressed to an androgen refractory stage. In this situation, it is proposed that while serum testosterone levels are decreased to very low levels, androgen present within the prostate cancer cell is still capable of fuelling growth of the tumor. Given that the aim of this therapy is to decrease the level of biologically available androgen within the cancer cell, it will be advantageous for the polypeptide to have the ability to enter the cell cytoplasm.

In addition, some prostate cancer epithelial cells might also secrete testosterone which is taken up by surrounding prostate cancer epithelial cells and our polypeptide drug would be able to soak up this source of androgen, irrespective of whether the polypeptide drug is able postal enter a prostate cancer epithelial cell directly.

In one form of the invention, the method of treatment or prevention includes administrates of a polypeptide of the present invention in combination with at least one other chemotherapeutic drug useful in the treatment of prostate cancer. Suitable compounds include, but are not limited to a cytostatic agent or cytotoxic agent. Nonlimiting examples of cytostatic agents are selected from: (1) microtubule-stabilizing agents such as but not limited tataxanes, paclitaxel, docetaxel, epothilones and laulimalides; (2) kinase inhibitors, illustrative examples of which include Iressa®, Gleevec, Tarceva™, (Erlotinib HCl), BAY-43-9006, inhibitors of the split kinase domain receptor tyrosine kinase subgroup (for example, 15 PTK787/ZK 222584 and SU11248); (3) receptor kinase targeted antibodies, which include, but are not limited to, Trastuzumab (Herceptin®), Cetuximab (Erbitux®), Bevacizumab (Avastin™), Rituximab (Ritusan®), Pertuzumab (Omnitarg™); (4) mTOR pathway inhibitors, illustrative examples of which include rapamycin and CCl-778; (5) Apo2L/Trail, antiangiogenic agents such as but not limited to endostatin, combrestatin, angiostatin, 20 thrombospondin and vascular endothelial growth inhibitor (VEGI); (6) antineoplastic immunotherapy vaccines, representative examples of which include activated T-cells, non-specific immune boosting agents (i.e., interferons, interleukins); (7) antibiotic cytotoxic agents such as but not limited to doxorubicin, bleomycin, dactinomycin, daunorubicin, epirubicin, mitomycin and mitozantrone; (8) alkylating agents, illustrative examples of which include Melphalan, Carmustine, Lomustine, Cyclophosphamide, Ifosfamide, Chlorambucil, Fotemustine, Busulfan, Temozolomide and Thiotepa; (9) hormonal antineoplastic agents, nonlimiting examples of which include, Nilutamide, Cyproterone acetate, Anastrozole, Exemestane, Tamoxifen, Raloxifene, Bicalutamide, Aminoglutethimide, Leuprorelin acetate, Toremifene citrate, Letrozole, Flutamide, Megestrol acetate and Goserelin acetate; (10) gonadal hormones such as but not limited to Cyproterone acetate and Medoxyprogesterone acetate; (11) antimetabolites, illustrative examples of which include Cytarabine, Fluorouracil, Gemcitabine, Topotecan, Hydroxyurea, Thioguanine, Methotrexate, Colaspase, Raltitrexed and Capicitabine; (12) anabolic agents, such as but not limited to, Nandrolone; (13) adrenal steroid hormones, illustrative examples of which include Methylprednisolone acetate, Dexamethasone, Hydrocortisone, Prednisolone and Prednisone; (14) neoplastic agents such as but not limited to Irinotecan, Carboplatin, Cisplatin, Oxaliplatin, Etoposide and Dacarbazine; and (15) topoisomerase inhibitors, illustrative examples of which include topotecan and irinotecan.

In some embodiments, the cytostatic agent is a nucleic acid molecule, suitably an antisense or siRNA recombinant nucleic acid molecule. In other embodiments, the cytostatic agent is a peptide or polypeptide. In still other embodiments, the cytostatic agent is a small molecule. The cytostatic agent may be a cytotoxic agent that is suitably modified to enhance uptake or delivery of the agent. Non-limiting examples of such modified cytotoxic agents include, but are not limited to, pegylated or albumin-labelled cytotoxic drugs.

In specific embodiments, the cytostatic agent is a microtubule stabilizing agent, especially a taxane and preferably docetaxel. In some embodiments, the cytotoxic agent is selected from the anthracyclines such as idarubicin, doxorubicin, epirubicin, daunorubicin and mitozantrone, CMF agents such as cyclophosphamide, methotrexate and 5-fluorouracil or other cytotoxic agents such as cisplatin, carboplatin, bleomycin, topotecan, irinotecan, melphalan, chlorambucil, vincristine, vinblastine and mitomycin-C.

Illustrative agents for chemical hormone ablation therapy include GnRH agonists or antagonists such as Cetrorelix, agents that interfere with the androgen receptor including non-steroidal agents such as Bicalutamide and steroidal agents such as Cyproterone, and agents that interfere with steroid biosynthesis such as Ketoconazole. Chemical agents suitable for use in combination with the polypeptide and pharmaceutically acceptable salts as hormone ablation therapy for prostate cancer include, but are not limited to, non-steroidal anti-androgens such as Nilutamide, Bicalutamide and flutamide; GnRH agonists such as Goserelin acetate, leuprorelin and triptorelin; 5-alpha reductase inhibitors such as finasteride; and cyproterone acetate.

Given that the polypeptides of the present invention are proposed to be capable of decreasing the levels of biologically available androgen in the serum and/or in the prostate cancer cell, the combination therapy may provide an additive or synergistic effect.

In another aspect, the present invention provides a method for treating or preventing prostate cancer, the method comprising administering to a subject in need thereof an effective amount of a nucleic acid molecule or vector encoding a polypeptide as disclosed herein. The present invention encompasses the use of nucleic acids encoding the polypeptides of the invention for transfection of cells in vitro and in vivo. These nucleic acids can be inserted into any of a number of well-known vectors for transfection of target cells and organisms. The nucleic acids are transfected into cells ex vivo and in vivo, through the interaction of the vector and the target cell. The compositions are administered (e.g., by injection into a muscle) to a subject in an amount sufficient to elicit a therapeutic response. An amount adequate to accomplish this is defined as “a therapeutically effective dose or amount.” For gene therapy procedures in the treatment or prevention of human disease, see for example, Van Brunt (1998) Biotechnology 6:1149 1154, the contents of which is incorporated herein by reference. Methods of treatment or prevention including the aforementioned nucleic acid molecules and vectors may include treatment with other compounds useful in the treatment of prostate cancer. Suitable compounds include, but are not limited to those described supra.

In a further aspect, the present invention provides a method for treating or preventing testosterone flare comprising administering to a subject in need thereof an effective amount of a polypeptide as described herein. LHRH drugs eventually result in suppression of testosterone, however before this occurs production of testosterone actually increases for a period. During the first week of treatment with a LHRH agonist or antagonist, the vastly increased production of testosterone may cause the cancer to flare.

In yet a further aspect, the present invention provides the use of a polypeptide as described herein in the manufacture of a medicament for the treatment or prevention of prostate cancer or testosterone flare.

In another aspect, the present invention provides the use of a nucleic acid molecule as described herein in the manufacture of a medicament for the treatment or prevention of prostate cancer or testosterone flare.

Still a further aspect provides the use of a vector as described herein in the manufacture of medicament for the treatment or prevention of prostate cancer or testosterone flare.

The present invention will now be more fully described by reference to the following non-limiting Examples.

In a first aspect the present invention provides a polypeptide for regulating a reproductive physiology in an animal, the polypeptide comprising a steroid sex hormone binding region, the steroid sex hormone binding region capable of binding to a steroid sex hormone at a sufficient affinity or avidity such that upon administration of the polypeptide to the animal the level of biologically available steroid sex hormone is decreased. Administration of a polypeptide capable of binding to a steroid sex hormone is capable of regulating physiological processes involved in, for example, fertility, the timing of estrus, and parturition. The ability to regulate such processes allows for the better management of solitary animals, as well as animals that are part of a group.

Where the animal is part of a group, the method may be applied to the majority or the whole of the herd allowing for the more efficient management of the herd as a whole. Common to all uses of the polypeptide is the requirement for a modulation of the level of a sex steroid hormone in the animal

As used herein, the term “a reproductive physiology” is intended to include any physiological process associated with reproduction that is regulated directly or indirectly by a sex steroid hormone. The term includes for example, ovulation, conception, parturition, commencement of estrus, maintenance of estrus, termination of estrus, commencement of pregenancy, maintainance of pregnancy, termination of pregnancy, erection, and semen production, spermatogenesis. The term extends to physiological processes or behaviours that are associated with or are a result of a reproductive process. For example, it is known that certain behaviours are associated with or are the result of reproductive processes. A mare on heat may exhibit any one or more of the following behaviours: restlessness, agitation, hyperactivity, frequent urination, sniffing or licking a stallion, straddling posture, clitoral “winking”, or raising the tail. Likewise a stallion, particularly when in the presence of a mare on heat, may exhibit any one or more of the following reproductively-associated behaviours: dominance, aggression, Flehmen response, impatience, alertness, hyperactivity, restlessness, vocalization, nudging or smelling or biting a mare.

The use of a polypeptide to sequester sex hormones is a significant departure from prior art methods that rely on the administration of hormones and other compounds, or surgery. Depleting a target steroid sex hormone from the circulation may cause less disruption to the animal's hormonal balance, and therefore produce less side effects, or lower-level side effects.

In one form of the polypeptide, the polypeptide comprises a carrier region. The role of the carrier region is to perform any one or more of the following functions: to generally improve a pharmacological property of the polypeptide including bioavailability, toxicity, and half life; limit rejection or destruction by an immune response; facilitate the expression or purification of the polypeptide when produced in recombinant form; all as compared with a polypeptide that does not include a carrier region. Given that the polypeptide of the present invention may be administered to a broad range of species, and in order to optimise the usefulness of the polypeptide in any given animal, it may be necessary to pay particular attention to the species specificity of this region. However, it is emphasised that even carrier regions that are not optimised for the intended recipient animal will still be operable.

In one form of the invention, the carrier region comprises sequence(s) of the Fc region of an IgG molecule. The human Fc region is commonly used in polypeptides for human use, and it is proposed that equivalents from animal species will be useful in the context for the present invention. For example, the structure and sequence of canine immunoglobulin has been well investigated (see for example, Tang et al 2001. Vet. Immunol. Immunopath. 80:259-270; Patel et al 1995, Immunogenetics 41:282-286; Wasserman, R.L., and J.D. Capra. 1978, Science 200:1159-1161, the sequence held on National Center for Biotechnology Information (NCBI) database under the accession NM_{—}001002976), as well as horse (the sequence held on NCBI database under the accessions AAG01011.1 and AAG01010), cat (the sequence held on NCBI database under accession BAA24986), and pig (the sequence held on NCBI database under accession BAE20056).

The Fc region binds to the salvage receptor FcRn which protects the fusion protein from lysosomal degradation giving increased half-life in the circulatory system. For example, the serum half-life of a fusion protein including the human IgG3 Fc region is around one week. In another form of the invention the Fc region comprises an IgG1, IgG2 or IgG4 sequence which increases the serum half-life to around 3 weeks. Serum half-life and effector functions (if desired) can be modulated by engineering the Fc region to increase or reduce its binding to FcRn, FcγRs and C1q respectively.

Increasing the serum persistence of a therapeutic antibody is one way to improve efficacy, allowing higher circulating levels, less frequent administration and reduced doses. This can be achieved by enhancing the binding of the Fc region to neonatal FcR (FcRn). FcRn, which is expressed on the surface of endothelial cells, binds the IgG in a pH-dependent manner and protects it from degradation. Several mutations located at the interface between the CH2 and CH3 domains have been shown to increase the half-life of IgG1 (Hinton P R. et al., 2004. J Biol. Chem. 279(8):6213-6; the contents of which is herein incorporated by reference, Vaccaro C. et al., 2005. Nat. Biotechnol. 23(10):1283-8; the contents of which is herein incorporated by reference).

In one form of the polypeptide, the carrier region is a species-specific carrier region. While not absolutely necessary, it may be preferable to use a carrier region that is specifically designed for the species into which the polypeptide is to be administered. For example, where a horse is to be treated the carrier region is from a horse-derived molecule, such as equine IgG Fc.

Given the above discussion on carrier regions, it will be appreciated that certain circumstances exist where the inclusion of such a region would be detrimental. For example, where a short serum half-life is desired a carrier region may be contraindicated. A practical application of a short half-life polypeptide may be where short term inhibition of androgen activity is required to control aggression in an animal.

In one embodiment of the invention, the level of biologically available steroid sex hormone is measured in the blood of the animal. It is an aim of the invention that the polypeptide is capable of decreasing biologically available steroid sex hormone. In this regard, assays that measure levels of total steroid sex hormone in the blood (i.e. free hormone in addition to bound hormone) may not be relevant to an assessment of whether a polypeptide is capable of decreasing biologically available steroid sex hormone. A more relevant assay would be one that measures free steroid sex hormone. These assays require determination of the percentage of unbound steroid sex hormone by a dialysis procedure, estimation of total steroid, and the calculation of free steroid. Free steroid hormone can also be calculated if total steroid, SHBG, and albumin concentrations are known (Sødergard et al, Calculation of free and bound fractions of testosterone and estradiol-17β to human plasma proteins at body temperature. J Steroid Biochem. 16:801-810; the contents of which is herein incorporated by reference). Methods are also available for determination of free steroid without dialysis. These measurements may be less accurate than those including a dialysis step, especially when the steroid hormone levels are low and SHBG levels are elevated (Rosner W. 1997, J Clin Endocrinol Metabol. 82:2014-2015; the contents of which is herein incorporated by reference; Giraudi et al. 1988. Steroids. 52:423-424; the contents of which is herein incorporated by reference). However, these assays may nevertheless be capable of determining whether or not a polypeptide is capable of decreasing biologically available steroid hormone.

Another method of measuring biologically available sex steroid hormone is disclosed by Nankin et al 1986 (J Clin Endocrinol Metab. 63:1418-1423; the contents of which is herein incorporated by reference. This method determines the amount of steroid not bound to SHBG and includes that which is nonprotein bound and weakly bound to albumin. The assay method relies on the fact SHBG is precipitated by a lower concentration of ammonium sulfate, 50%, than albumin. Thus by precipitating a serum sample with 50% ammonium sulfate and measuring the steroid value in the supernate, non-SHBG bound or biologically available steroid is measured. This fraction of steroid can also be calculated if total steroid, SHBG, and albumin levels are known.

Further exemplary methods of determining levels of biologically available testosterone are disclosed in de Ronde et al., 2006 (Olin Chem 52(9):1777-1784; the contents of which is herein incorporated by reference). Methods for assaying free dihydrotestosterone (Horst et al Journal of Clinical Endocrinology and Metabolism 45: 522, 1977, the contents of which is herein incorporated by reference), dihydroepiandosterone (Parker and O'Dell Journal of Clinical Endocrinology and Metabolism 47: 600, 1978, the contents of which is herein incorporated by reference), estrogen (Blondeau and Robel (1975) Eur. J. Biochem. 55, 375-384, the contents of which is herein incorporated by reference), estradiol (Mounib et al Journal of Steroid Biochemistry 31: 861-865, 1988), and progesterone (Batra et al Journal of Clinical Endocrinology and Metabolism 42: 1041, 1976, the contents of which is herein incorporated by reference).

In determining whether or not a polypeptide is capable of decreasing biologically available steroid sex hormone, the skilled person will understand that it may be necessary to account for the natural variability of hormone levels that occur in an individual animal. It is known that hormone levels fluctuate in an individual animal according to many factors, including the time of day and the amount of physical activity. For example, it is typically observed that testosterone levels are higher in the morning as compared with a sample taken in the evening. Even in consideration of these variables, by careful planning of sample withdrawal, or by adjusting a measurement obtained from the individual, it will be possible to ascertain whether the level of biologically available steroid sex hormone in an individual has been affected by the administration of a polypeptide as described herein.

In one embodiment, the polypeptide has an affinity or avidity for the steroid sex hormone that is equal to or greater than the affinity or avidity between the steroid sex hormone and a natural carrier of the steroid sex hormone. Natural carriers in the blood include SHBG and serum albumin. It will be appreciated that the binding of a steroid sex hormone to these natural carriers is reversible, and an equilibrium exists between the bound and unbound form of the hormone. In one form of the invention, to decrease the level of biologically available steroid sex hormone to below that normally present (for example less than 1-2% in the case of testosterone) the polypeptide has an affinity or avidity for the steroid sex hormone that is greater than that between the cognate binding protein and the hormone. Thus in one embodiment of the invention, the polypeptide has an association constant for the steroid sex hormone that is greater than that for a natural carrier of the steroid such as SHBG or albumin.

In another form of the invention the polypeptide has an association constant for the steroid sex hormone that is about equal to or less than that for the cognate natural carrier. In this embodiment, while free steroid may bind to the natural carrier in preference to the polypeptide, addition of polypeptide to the circulation may still be capable of decreasing the level of biologically available steroid sex hormone. Where the polypeptide has a low affinity or avidity for hormone, it may be necessary to administer the polypeptide in larger amounts to ensure that the level of steroid sex hormone is sufficiently depleted.

Steroid hormones exert their biological activities via a common mechanism. In the absence of hormone, steroid hormone receptors exist as inactive oligomeric complexes with a number of other proteins including chaperon proteins, namely the heat shock proteins Hsp90 and Hsp70 and cyclophilin-40 and p23. The role of Hsp90 and other chaperons is to maintain the receptors folded in an appropriate conformation to respond rapidly to hormonal signals. Following hormone binding, the oligomeric complex dissociates allowing the receptors to function either directly as transcription factors by binding to DNA in the vicinity of target genes or indirectly by modulating the activity of other transcription factors.

In light of the above, all steroid hormones must have a cognate receptor which includes sequences capable of binding the steroid molecule. Steroid hormone receptors are all members of the nuclear receptor family, which function as transcription factors in many different mammalian species. The receptors are highly related in both primary amino acid sequence and the organisation of functional domains suggesting that many aspects of their mechanism of action are conserved. Indeed, progress in understanding of steroid hormone action has been facilitated by studies of many nuclear receptor family members.

Steroid hormone receptors share a modular structure in which six distinct structural and functional domains, A to F, are displayed (Evans, Science 240, 889-895, 1988, the contents of which is herein incorporated by reference). A nuclear hormone receptor is Characterized by a variabel N-terminal region (domain A/B), followed by a centrally located, highly conserved DNA-binding domain (hereinafter referred to as DBD; domain C), a variable hinge region (domain D), a conserved hormone binding domain; domain E) and a variable C-terminal region (domain F).

The N-terminal region, which is highly variable in size and sequence, is poorly conserved among the different members of the superfamily. This part of the receptor is involved in the modulation of transcription activation (Bocquel et al, Nucl. Acid Res., 17, 2581-2595, 1989; Tora et al, Cell 59, 477-487, 1989, the contents of which are herein incorporated by reference).

The DBD consists of approximately 66 to 70 amino acids and is responsible for DNA-binding activity: it targets the receptor to specific DNA sequences called hormone responsive elements within the transcription control unit of specific target genes on the chromatin (Martinez and Wahli, In ‘Nuclear Hormone Receptors’, Acad. Press, 125-153, 1991, the contents of which is herein incorporated by reference).

The hormone binding domain is located in the C-terminal part of the receptor and is primarily responsible for ligand binding activity. This domain is therefore required for recognition and binding of the hormone ligand thereby determining the specificity and selectivity of the hormone response of the receptor. In the context of the present invention, the hormone binding domain is the most important region since it affords the polypeptides of the present invention the ability to effectively sequester biologically available hormone.

In one embodiment of the invention the steroid sex hormone receptor is selected from the group consisting of an androgen receptor, a progesterone receptor, and an estrogen receptor.

In one form of the polypeptide, the nuclear hormone receptor agonist binding region includes sequences from the hormone binding domain of the progesterone receptor, or functional equivalent thereof. Like all nuclear hormone receptors, the progesterone receptor has a regulatory domain, a DNA binding domain, a hinge section, and a hormone binding domain. The progesterone receptor has two isoforms (A and B). The single-copy gene uses separate promoters and translational start sites to produce the two isoforms. Both are included in the scope of this invention:

Williams and Sigler have solved the atomic structure of progesterone complexed with its receptor (Nature. 1998 May 28; 393(6683):392-6, the contents of which is herein incorporated by reference). The authors report the 1.8 A crystal structure of a progesterone-bound ligand-binding domain of the progesterone receptor. The nature of this structure explains the receptor's selective affinity or avidity for progestins and establishes a common mode of recognition of 3-oxy steroids by the cognate receptors. The wild type sequence of the progesterone sequence is known:

MTELKAKGPRAPHVAGGPPSPEVGSPLLCRPAAGPFPGSQTSDTLPEV
SAIPISLDGLLFPRPCQGQDPSDEKTQDQQSLSDVEGAYSRAEATRGA
GGSSSSPPEKDSGLLDSVLDTLLAPSGPGQSQPSPPACEVTSSWCLFG
PELPEDPPAAPATQRVLSPLMSRSGCKVGDSSGTAAAHKVLPRGLSPA
RQLLLPASESPHWSGAPVKPSPQAAAVEVEEEDGSESEESAGPLLKGK
PRALGGAAAGGGAAAVPPGAAAGGVALVPKEDSRFSAPRVALVEQDAP
MAPGRSPLATTVMDFIHVPILPLNHALLAARTRQLLEDESYDGGAGAA
SAFAPPRSSPCASSTPVAVGDFPDCAYPPDAEPKDDAYPLYSDFQPPA
LKIKEEEEGAEASARSPRSYLVAGANPAAFPDFPLGPPPPLPPRATPS
RPGEAAVTAAPASASVSSASSSGSTLECILYKAEGAPPQQGPFAPPPC
KAPGASGCLLPRDGLPSTSASAAAAGAAPALYPALGLNGLPQLGYQAA
VLKEGLPQVYPPYLNYLRPDSEASQSPQYSFESLPQKICLICGDEASG
CHYGVLTCGSCKVFFKRAMEGQHNYLCAGRNDCIVDKIRRKNCPACRL
RKCCQAGMVLGGRKFKKFNKVRVVRALDAVALPQPVGVPNESQALSQR
FTFSPGQDIQLIPPLINLLMSIEPDVIYAGHDNTKPDTSSSLLTSLNQ
LGERQLLSVVKWSKSLPGFRNLHIDDQITLIQYSWMSLMVFGLGWRSY
KHVSGQMLYFAPDLILNEQRMKESSFYSLCLTMWQIPQEFVKLQVSQE
EFLCMKVLLLLNTIPLEGLRSQTQFEEMRSSYIRELIKAIGLRQKGVV
SSSQRFYQLTKLLDNLHDLVKQLHLYCLNTFIQSRALSVEFPEMMSEV
IAAQLPKILAGMVKPLLFHKK

In one embodiment of the polypeptide, the nuclear hormone receptor agonist binding region includes residues approximately 676 to 693 of the progesterone receptor.

In another embodiment of the polypeptide, the nuclear hormone receptor agonist binding region includes sequences from the hormone binding domain of the estrogen receptor, or functional equivalent thereof. Wurtz et al (J Med Chem. 1998 May 21; 41(11), the contents of which is herein incorporated by reference) published a three-dimensional model of the estrogen receptor hormone binding domain. The quality of the model was tested against mutants, which affect the binding properties. A thorough analysis of all published mutants was performed with Insight II to elucidate the effect of the mutations. 45 out of 48 mutants can be explained satisfactorily on the basis of the model. After that, the natural ligand estradiol was docked into the binding pocket to probe its interactions with the protein. Energy minimizations and molecular dynamics calculations were performed for various ligand orientations with Discover 2.7 and the CFF91 force field. The analysis revealed two favorite estradiol orientations in the binding niche of the binding domain forming hydrogen bonds with Arg394, Glu353 and His524. The crystal structure of the ER LBD in complex with estradiol has been published (Brzozowski et al. Nature 389, 753-758, 1997, the contents of which is herein incorporated by reference). The amino acid sequence of the estrogen receptor is as follows:

MTMTLHTKASGMALLHQIQGNELEPLNRPQLKIPLERPLGEVYLDSSK
PAVYNYPEGAAYEFNAAAAANAQVYGQTGLPYGPGSEAAAFGSNGLGG
FPPLNSVSPSPLMLLHPPPQLSPFLQPHGQQVPYYLENEPSGYTVREA
GPFAFYRPNSDNRRQGGRERLASTNDKGSMAMESAKETRYCAVCNDYA
SGYHYGVWSCEGCKAFFKRSIQGHNDYMCPATNQCTIDKNRRKSCQAC
RLRKCYEVGMMKGGIRKDRRGGRMLKHKRQRDDGEGRGEVGSAGDMRA
ANLWPSPLMIKRSKKNSLALSLTADQMVSALLDAEPPILYSEYDPTRP
FSEASMMGLLTNLADRELVHMINWAKRVPGFVDLTLHDQVHLLECAWL
EILMIGLVWRSMEHPGKLLFAPNLLLDRNQGKCVEGMVEIFDMLLATS
SRFRMMNLQGEEFVCLKSIILLNSGVYTFLSSTLKSLEEKDHIHRVLD
KITDTLIHLMAKAGLTLQQQHQRLAQLLLILSHIRHMSNKGMEHLYSM
KCKNVVPLYDLLLEMLDAHRLHAPTSRGGASVEETDQSHLATAGSTSS
HSLQKYYITGEAEGFPATV

In another embodiment of the polypeptide, the nuclear hormone receptor agonist binding region includes sequences from the hormone binding domain of the androgen receptor, or functional equivalent thereof. The gene encoding the receptor is more than 90 kb long and codes for a protein that has 3 major functional domains. The N-terminal domain, which serves a modulatory function, is encoded by exon 1 (1,586 bp). The DNA-binding domain is encoded by exons 2 and 3 (152 and 117 bp, respectively). The steroid-binding domain is encoded by 5 exons which vary from 131 to 288 bp in size. The amino acid sequence of the androgen receptor protein is described by the following sequence.

MEVQLGLGRVYPRPPSKTYRGAFQNLFQSVREVIQNPGPRHPEAASAA
PPGASLLLLQQQQQQQQQQQQQQQQQQQQQETSPRQQQQQQGEDGSPQ
AHRRGPTGYLVLDEEQQPSQPQSALECHPERGCVPEPGAAVAASKGLP
QQLPAPPDEDDSAAPSTLSLLGPTFPGLSSCSADLKDILSEASTMQLL
QQQQQEAVSEGSSSGRAREASGAPTSSKDNYLGGTSTISDNAKELCKA
VSVSMGLGVEALEHLSPGEQLRGDCMYAPLLGVPPAVRPTPCAPLAEC
KGSLLDDSAGKSTEDTAEYSPFKGGYTKGLEGESLGCSGSAAAGSSGT
LELPSTLSLYKSGALDEAAAYQSRDYYNFPLALAGPPPPPPPPHPHAR
IKLENPLDYGSAWAAAAAQCRYGDLASLHGAGAAGPGSGSPSAAASSS
WHTLFTAEEGQLYGPCGGGGGGGGGGGGGGGGGGGGGGGGEAGAVAPY
GYTRPPQGLAGQESDFTAPDVWYPGGMVSRVPYPSPTCVKSEMGPWMD
SYSGPYGDMRLETARDHVLPIDYYFPPQKTCLICGDEASGCHYGALTC
GSCKVFFKRAAEGKQKYLCASRNDCTIDKFRRKNCPSCRLRKCYEAGM
TLGARKLKKLGNLKLQEEGEASSTTSPTEETTQKLTVSHIEGYECQPI
FLNVLEAIEPGVVCAGHDNNQPDSFAALLSSLNELGERQLVHVVKWAK
ALPGFRNLHVDDQMAVIQYSWMGLMVFAMGWRSFTNVNSRMLYFAPDL
VFNEYRMHKSRMYSQCVRMRHLSQEFGWLQITPQEFLCMKALLLFSII
PVDGLKNQKFFDELRMNYIKELDRIIACKRKNPTSCSRRFYQLTKLLD
SVQPIARELHQFTFDLLIKSHMVSVDFPEMMAEIISVQVPKILSGKVK
PIYFHTQ

The identity of the steroid binding domain has been the subject of considerable research (Ai et al, Chem Res Toxicol 2003, 16, 1652-1660; Bohl et al, J Biol Chem 2005, 280(45) 37747-37754; Duff and McKewan, Mol Endocrinol 2005, 19(12) 2943-2954; Ong et al, Mol Human Reprod 2002, 8(2) 101-108; Poujol et al, J Biol Chem 2000, 275(31) 24022-24031; Rosa et al, J Clin Endocrinol Metab 87(9) 4378-4382; Marhefka et al, J Med Chem 2001, 44, 1729-1740; Matias et al, J Biol Chem 2000, 275(34) 26164-26171; McDonald et al, Cancer Res 2000, 60, 2317-2322; Sack et al, PNAS 2001, 98(9) 4904-4909; Steketee et al, Int J Cancer 2002, 100, 309-317; the contents of which are all herein incorporated by reference). While the exact residues essential for steroid binding are not known, it is generally accepted that the region spanning the approximately 250 amino acid residues in the C-terminal end of the molecule is involved (Trapman et al (1988). Biochem Biophys Res Commun 153, 241-248, the contents of which is herein incorporated by reference).

In one embodiment of the invention where the polypeptide is directed against testosterone, the steroid sex hormone binding region comprises or consists of the sequence defined by the 230 C-terminal amino acids of the sequence dnnqpd . . . iyfhtq.

Some studies have considered the crystal structure of the steroid binding domain of the androgen receptor in complex with a synthetic steroid. For example, Sack et al (ibid) propose that the 3-dimensional structure of the receptor includes a typical nuclear receptor ligand binding domain fold. Another study proposes that the steroid binding pocket has consists of 18 (noncontiguous) amino acid residues that interact with the ligand (Matias et al, ibid). It is emphasized that this study utilized a synthetic steroid ligand (R1881) rather than actual dihydrotestosterone. The binding pocket for dihydrotestosterone may include the same residues as that shown for R1181 or different residues.

Further crystallographic data on the steroid binding domain complexed with agonist predict 11 helices (no helix 2) with two anti-parallel 13-sheets arranged in a so-called helical sandwich pattern. In the agonist-bound conformation the carboxy-terminal helix 12 is positioned in an orientation allowing a closure of the steroid binding pocket. The fold of the ligand binding domain upon hormone binding results in a globular structure with an interaction surface for binding of interacting proteins like co-activators.

In one embodiment, the steroid sex hormone binding region, comprises or consists of the steroid hormone binding domain of the cognate receptor, but is devoid of regions of the receptor that are not involved in steroid hormone binding.

In another embodiment of the invention the steroid hormone binding region of the polypeptide comprises a sequence or sequences derived from the steroid binding domain of a sex hormone binding protein. The sequence of SHBG is described by the following sequence:

ESRGPLATSRLLLLLLLLLLRHTRQGWALRPVLPTQSAHDPPAVHLSN
GPGQEPIAVMTFDLTKITKISSSFEVRTWDPEGVIFYGDTNPKDDWFM
LGLRDGRPEIQLHNHWAQLTVGAGPRLDDGRWHQVEVKMEGDSVLLEV
DGEEVLRLRQVSGPLTSKRHPIMRIALGGLLFPASNLRLPLVPALDGC
LRRDSWLDKQAEISASAPTSLRSCDVESNPGIFLPPGTQAEFNLRDIP
QPHAEPWAFSLDLGLKQAAGSGHLLALGTPENPSWLSLHLQDQKVVLS
SGSGPGLDLPLVLGLPLQLKLSMSRVVLSQGSKMKALALPPLGLAPLL
NLWAKPQGRLFLGALPGEDSSTSFCLNGLWAQGQRLDVDQALNRSHEI
WTHSCPQSPGNGTDASH

The scope of the invention extends to fragments and functional equivalents of the above protein sequence.

From the above, it will be understood that the identity of the minimum residues required for binding any given steroid sex hormone may not have been settled at the filing date of this application. Accordingly, the present invention is not limited to polypeptides comprising any specific region of the receptor. It is therefore to be understood that the scope of the present invention is not necessarily limited to any specific residues as detailed herein.

In any event, the skilled person understands that various alterations may be made to the steroid sex hormone binding sequence without completely ablating the ability of the sequence to bind steroid. Indeed it may be possible to alter the sequence to improve the ability of the domain to bind a steroid sex hormone. Therefore, the scope of the invention extends to functional equivalents of the steroid binding domain of the cognate receptor. It is expected that certain alterations could be made to the ligand binding domain sequence of the receptor without substantially affecting the ability of the domain to bind steroid. For example, the possibility exists that certain amino acid residues may be deleted, substituted, or repeated. Furthermore, the sequence may be truncated at the C-terminus and/or the N-terminus. Furthermore additional bases may be introduced within the sequence. Indeed, it may be possible to achieve a sequence having an increased affinity or avidity for steroid hormone by trialling a number of alterations to the amino acid sequence. The skilled person will be able to ascertain the effect (either positive or negative) on the binding by way of standard association assay with steroid, as described herein.

It is emphasized that the steroid sex hormone binding region of the polypeptide is not restricted to any specific sequence or sequences described herein. The domain may be determined by reference to any other molecule (natural or synthetic) capable of binding steroid sex hormone including any carrier protein, enzyme, receptor, or antibody.

The scope of the present invention includes all steroid sex hormones found in any animal species. However, in one form of the invention the steroid sex hormone is selected from the group consisting of androstenedione (4-androstene-3,17-dione); 4-hydroxy-androstenedione; 11β-hydroxyandrostenedione (11beta-4-androstene-3,17-dione); androstanediol (3-beta,17-beta-Androstanediol); androsterone (3alpha-hydroxy-5alpha-androstan-17-one); epiandrosterone (3beta-hydroxy-5alpha-androstan-17-one); adrenosterone (4-androstene-3,11,17-trione); dehydroepiandrosterone (3beta-hydroxy-5-androsten-17-one); dehydroepiandrosterone sulphate (3beta-sulfoxy-5-androsten-17-one); testosterone (17beta-hydroxy-4-androsten-3-one); epitestosterone (17alpha-hydroxy-4-androsten-3-one); 50-dihydrotestosterone (17beta-hydroxy-5alpha-androstan-3-one 5β-dihydrotestosterone; 5-beta-dihydroxy testosterone (17beta-hydroxy-5beta-androstan-3-one); 11β-hydroxytestosterone (11beta,17beta-dihydroxy-4-androsten-3-one); 11-ketotestosterone (17beta-hydroxy-4-androsten-3,17-dione), estrone (3-hydroxy-1,3,5(10)-estratrien-17-one); estradiol (1,3,5(10)-estratriene-3,17beta-diol); estriol 1,3,5(10)-estratriene-3,16alpha,17beta-triol; pregnenolone (3-beta-hydroxy-5-pregnen-20-one); 17-hydroxypregnenolone (3-beta,17-dihydroxy-5-pregnen-20-one); progesterone (4-pregnene-3,20-dione); 17-hydroxyprogesterone (17-hydroxy-4-pregnene-3,20-dione) and progesterone (pregn-4-ene-3,20-dione).

While the polypeptide may have more than one steroid hormone binding region, in one form of the invention the polypeptide has a single steroid hormone binding region. This form of the polypeptide may be advantageous due to the potentially small size of the molecule. A smaller polypeptide may have a longer half life in the circulation, or may elicit a lower level of immune response in the body. A smaller polypeptide may also have a greater ability to enter a cell to neutralize intracellular steroid.

While the polypeptide may be a fusion protein such as that described supra, it will be appreciated that the polypeptide may take any form that is capable of achieving the aim of binding a steroid sex hormone such that the level of hormone in the blood or a cell is decreased.

For example, the polypeptide may be a therapeutic antibody. Many methods are available to the skilled artisan to design therapeutic antibodies that are capable of binding to a predetermined target, persist in the circulation for a sufficient period of time, and cause minimal adverse reaction on the part of the host (Carter, Nature Reviews (Immunology) Volume 6, 2006; the contents of which is herein incorporated by reference).

In one embodiment, the therapeutic antibody is a single clone of a specific antibody that is produced from a cell line, including a hybridoma cell. There are four classifications of therapeutic antibodies: murine antibodies; chimeric antibodies; antibodies tailored for use in a target species; and antibodies that are completely derived from a target species. These different types of antibodies are distinguishable by the percentage of mouse to target species parts making up the antibodies. A murine antibody contains 100% mouse sequence, a chimeric antibody contains approximately 30% mouse sequence, and antibodies that are tailored for use in, or completely derived from a target species contain only 5-10% mouse residues.

The polypeptide may be a single chain antibody (scFv), which is an engineered antibody derivative that includes heavy- and lightchain variable regions joined by a peptide linker. ScFv antibody fragments are potentially more effective than unmodified IgG antibodies. The reduced size of 27-30 kDa allows penetration of tissues and solid tumors more readily (Huston et al. (1993). Int. Rev. Immunol. 10, 195-217; the contents of which is herein incorporated by reference). Methods are known in the art for producing and screening scFv libraries for activity, with exemplary methods being disclosed in is disclosed by Walter et al 2001, Comb Chem High Throughput Screen; 4(2):193-205; the contents of which is herein incorporated by reference.

The polypeptide may have greater efficacy as a therapeutic if in the form of a multimer. The polypeptide may be effective, or have improved efficacy when present as a homodimer, homotrimer, or homotetramer; or as a heterodimer, heterotrimer, or heterotetramer. In these cases, the polypeptide may require multimerisation sequences to facilitate the correct association of the monomeric units. Thus, in one embodiment the polypeptide comprises a multimerisation region. It is anticipated that where the steroid binding region of the polypeptide comprises sequences from SHBG, a multimerisation domain may be included.

In another aspect, the present invention provides a composition comprising a polypeptide of the present invention in combination with a pharmaceutically acceptable carrier. The skilled person is adequately enabled to select the appropriate carrier(s) to include in the composition. Potentially suitable carriers include a diluent, adjuvant, excipient, or vehicle with which the polypeptide is administered. Diluents include sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin.

The polypeptides of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Furthermore, aqueous compositions useful for practicing the methods of the invention have physiologically compatible pH and osmolality. One or more physiologically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers are included in an amount required to maintain pH of the composition in a physiologically acceptable range. One or more physiologically acceptable salts can be included in the composition in an amount sufficient to bring osmolality of the composition into an acceptable range. Such salts include those having sodium, potassium or ammonium cations and chloride, citrate, ascorbate, borate, phosphate, bicarbonate, sulfate, thiosulfate or bisulfite anions.

It is anticipated that gene therapy methods could be used to manufacture the polypeptides of the invention within the animal's body. Accordingly a further aspect of the present invention provides a nucleic acid molecule capable of encoding a polypeptide as described herein. Another aspect of the present invention provides a vector comprising a nucleic acid molecule as described herein.

In a further aspect, the present invention provides a method for regulating a reproductive physiology of an animal, the method comprising administering to an animal in need thereof an effective amount of a polypeptide as described herein.

As will be appreciated by the skilled person, the method could be used for any circumstance where it is desired to modulate fertility by depleting the level of a steroid sex hormone. An exemplary use of the method is for the temporary sterilisation of animals. For example, a male dog could be temporarily sterilised by administration of a testosterone-specific polypeptide at sufficient dosage to bind substantially all biologically available testosterone. This would have the effect of shutting down sperm production, such that after a sufficient treatment period the dog would become sterile. Similarly, a bitch could be sterilised by the administration of an estrogen-specific polypeptide.

For the control of estrus, a polypeptide capable of binding any one of the sex steroid hormones associated with the estrus cycle could be administered. As an example, progesterone may be used to induce estrus, and so sequestration of progesterone by the administration of a progesterone-specific polypeptide would lead to a delay in estrus, or to a prevention of estrus.

In one form of the method, the polypeptide is administered in the form of a composition as described herein.

In a further aspect, the present invention provides a method for regulating a reproductive physiologyof an animal, the method comprising administering to a subject in need thereof an effective amount of a nucleic acid molecule as described herein, or a vector as described herein.

The present invention encompasses the use of nucleic acids encoding the polypeptides of the invention for transfection of cells in vitro and in vivo. These nucleic acids can be inserted into any of a number of well-known vectors for transfection of target cells and organisms. The nucleic acids are transfected into cells ex vivo and in vivo, through the interaction of the vector and the target cell. The compositions are administered (e.g., by injection into a muscle) to an animal in an amount sufficient to elicit a therapeutic response. An amount adequate to accomplish this is defined as “a therapeutically effective dose or amount.” For gene therapy procedures in the treatment or prevention of disease, see for example, Van Brunt (1998) Biotechnology 6:1149 1154, the contents of which is incorporated herein by reference. Methods of treatment or prevention including the aforementioned nucleic acid molecules and vectors may include treatment with other compounds useful in the regulation of a reproductive physiology.

The present invention further provides the use of a polypeptide according to as described herein in the manufacture of a medicament for regulating a reproductive physiology in an animal. Also provided is the use of a nucleic acid molecule as described herein in the manufacture of a medicament for the regulating a reproductive physiologyin an animal. The present invention still further provides the use of a vector as described herein in the manufacture of a medicament for the regulating a reproductive physiologyin an animal.

It is to be understood that the present invention is not limited in application to any given non-human animal(s). In one form of the invention the animal is selected from the group consisting of a horse, a pig, a cow, a goat, a sheep, an alpaca, a dog, and a cat

The invention will now be further described by reference to the following non-limiting examples.

### EXAMPLES

### Example 1

### Construction of Androgen-Binding Polypeptide

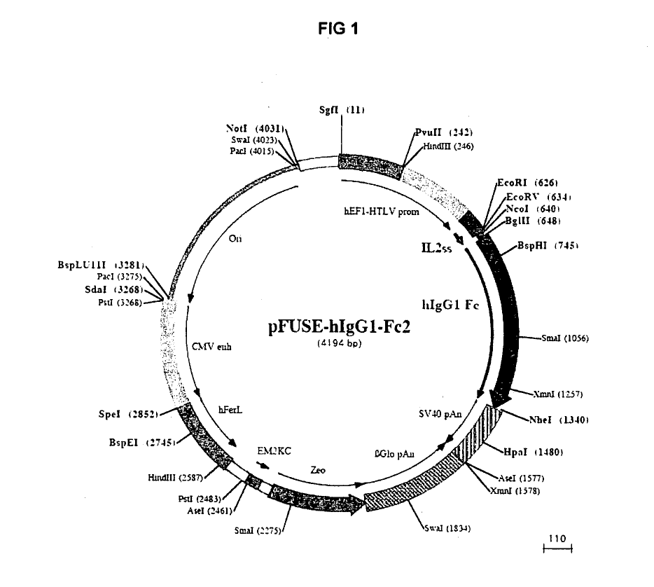

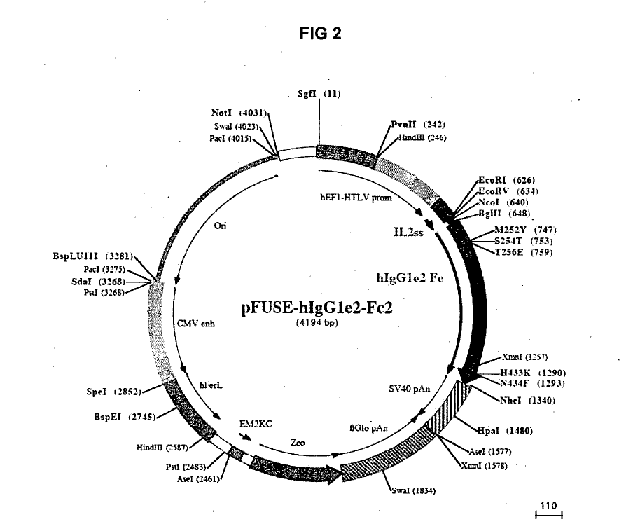

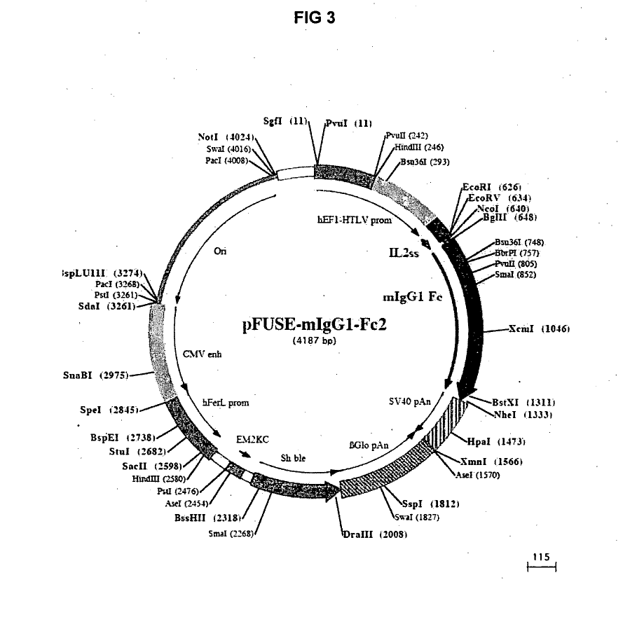

The following coding region for human androgen receptor ligand binding domain (690 bp) is subcloned into various vectors (pFUSE-hIgGi-Fc2, pFUSE-hIgGie2-Fc2, pFUSE-mlgG1-Fc2 from Invivogen) using EcoRI and BglII RE sites (see FIGS. 1 to 3).

GACAACAACCAGCCCGACAGCTTCGCCGCCCTGCTGTCCAGCCTGAAC
GAGCTGGGCGAGAGGCAGCTGGTGCACGTGGTGAAGTGGGCCAAGGCC
CTGCCCGGCTTCAGAAACCTGCACGTGGACGACCAGATGGCCGTGATC
CAGTACAGCTGGATGGGCCTGATGGTGTTCGCTATGGGCTGGCGGAGC
TTCACCAACGTGAACAGCAGGATGCTGTACTTCGCCCCCGACCTGGTG
TTCAACGAGTACAGGATGCACAAGAGCAGGATGTACAGCCAGTGCGTG
AGGATGAGGCACCTGAGCCAGGAATTTGGCTGGCTGCAGATCACCCCC
CAGGAATTTCTGTGCATGAAGGCCCTGCTGCTGTTCAGCATCATCCCC
GTGGACGGCCTGAAGAACCAGAAGTTCTTCGACGAGCTGCGGATGAAC
TACATCAAAGAGCTGGACAGGATCATCGCCTGCAAGAGGAAGAACCCC
ACCTCCTGCAGCAGAAGGTTCTACCAGCTGACCAAGCTGCTGGACAGC
GTGCAGCCCATCGCCAGAGAGCTGCACCAGTTCACCTTCGACCTGCTG
ATCAAGAGCCACATGGTGTCCGTGGACTTCCCCGAGATGATGGCCGAG
ATCATCAGCGTGCAGGTGCCCAAGATCCTGAGCGGCAAGGTCAAGCCC
ATCTACTTCCACACCCAG

This sequence encodes the 230 C-terminal residues of the human androgen receptor protein disclosed herein.

The various vectors are separately transfected into CHO cells and secreted protein collected. The cell culture supernatant after various times of incubation is spun at 10,000-13,000 rpm for 15 min at 4° C. and filtered prior to purification.

The supernatant is diluted 50:50 with a binding buffer (PBS, pH 7.4, containing 500 mM Glycine) before injection on to the Protein G affinity chromatography column (Mo Bi Tech, Molecular Biotechnology), which is pre-equilibrated with 5 column volumes of the binding buffer. The column is washed with 10 column volumes of binding buffer. The sample is then eluted off the column with 100 mM Glycine-HCl, pH 3.0 and collected in eppendorf tubes containing a 15% final fraction volume of 2.0M Tris-HCl, pH 7.4.

### Cell Line

Mammalian CHO cell cultures are maintained in a Form a Scientific Incubator with 10% carbon dioxide at 37° C. in Dulbecco's Modified Eagle Medium (DMEM) (Gibco). Penicillin (100 U/ml), streptomycin (100 μg/ml) and amphotericin B (25 ng/ml) (Gibco Invitrogen #15240-062) are added to media as standard. As a routine, cells are maintained in the presence of 5% or 10% fetal bovine serum (Gibco Invitrogen #10099-141) unless otherwise stated. Subconfluent cells are passaged with 0.5% trypsin-EDTA (Gibco Invitrogen #15400-054).

### Propagation of DNA Constructs

DNA expression constructs are propagated in supercompetent DH5α E. Coli (Stratagene). To transform bacteria, 1 μg of plasmid DNA is added to 200 μl of bacteria in a microfuge tube and placed on ice for 20 min. Bacteria are heat shocked at 42° C. for 1.5 min, then replaced on ice for a further 5 min. 1 ml of Luria-Bertani broth (LB) without antibiotics is then added, and the bacteria incubated at 37° C. on a heat block for 1 h. This is then added to 200 ml of LB with penicillin 50 μg/ml and incubated overnight at 37° C. with agitation in a Bioline Shaker (Edwards Instrument Company, Australia). The following morning the bacterial broth are transferred to a large centrifuge tube and spun at 10,000 rpm for 15 min. The supernatant is removed and the pellet dried by inverting the tube on blotting paper. Plasmid DNA is recovered using the Wizard® Plus Midipreps DNA purification system (Promega #A7640). The pellet is resuspended in 3 ml of Cell Resuspension Solution (50 mM Tris-HCl pH 7.5, 10 mM EDTA, 100 μg/ml RNase A) and an equal volume of Cell Lysis Solution added (0.2 M NaOH, 1% SDS). This is mixed by inversion four times. 3 ml of neutralization solution (1.32 M potassium acetate pH 4.8) is then added, and the solution again mixed by inversion. This is centrifuged at 14,000 g for 15 min at 4° C. The supernatant is then carefully decanted to a new tube by straining through muslin cloth. 10 ml of resuspended DNA purification resin is added to the DNA solution and mixed thoroughly. The Midi column tip is inserted into a vacuum pump, the DNA solution/resin mixture added to the column, and the vacuum applied. Once the solution is passed through the column it is washed twice by adding 15 ml of Column Wash Solution and applying the vacuum until the solution had drawn through. After the last wash the column is sharply incised to isolate the column reservoir which is transferred to a microfuge tube and spun at 13,000 rpm for 2 min to remove any residual wash solution. 100 μl of pre-heated nuclease-free water is added and the DNA eluted by centrifuging at 13,000 rpm for 20 sec in a fresh tube. DNA concentration is measured by absorbance spectroscopy (Perkin Elmer MBA2000).

### Examination of DNA Products by Gel Electrophoresis

The DNA products of polymerase chain reactions or restriction enzyme digests of plasmid DNA are analysed by agarose gel electrophoresis. Agarose (1-1.2%) is dissolved in TAE buffer (40 mM Tris acetate, 2 mM EDTA pH 8.5) containing 0.5 μg/ml ethidium bromide. A DNA loading dye consisting of 0.2% w/v xylene cyanol, 0.2% bromophenol blue, 40 mM Tris acetate, 2 mM EDTA pH 8.5 and 50% glycerol is added to the samples before electrophoresis. Electrophoresis is conducted at approximately 100V in 1×TAE. DNA samples are visualized under ultraviolet light (254 nm).

Polypeptide Fusion Protein Transfection and Expression in CHO cells

Plasmids encoding polypeptide fusion proteins are transfected into CHO cells using calcium phosphate. Cells are seeded in 6-well plates to be ˜40-50% confluent on the day of transfection. Growth media is changed 3 h prior to transfection. 2 μg of plasmid DNA is mixed with 37 μl of 2 M calcium phosphate in a microfuge tube and the final volume made up to 300 μl with dH₂O. This is added dropwise to an equal volume of 2×HBS with continuous vortexing, and incubated at RT for 30 min. This solution is then added dropwise to the plate. Cells are incubated for 6 h, cells washed twice with TBS, and fresh media added. Transfection efficiency is determined by spiking a control sample with 0.2 μg of pcDNA3.GFP.

### Example 2

### Construction of Estrogen-Binding Polypeptide

The following coding region for human estrogen receptor ligand binding domain (723 bp) is subcloned into various vectors (pFUSE-hIgG1-Fc2, pFUSE-hIgGle2-Fc2, pFUSE-mlgG1-Fc2 from Invivogen) using EcoRI and BglII RE sites (see FIGS. 1 to 3).

ACCGCCGACC AGATGGTGTC CGCCCTGCtG GACGCCGAGC
CCCCCATCCT GTACAGCGAG TACGACCCCA CCAGGCCCTT
CTCCGAGGCT AGCATGATGG GCCTGCTGAC CAACCTGGCC
GACCGGGAGC TGGTGCACAT GATCAACTGG GCCAAGAGGG
TGCCCGGCTT CGTCGACCTG ACACTGCACG ATCAGGTCCA
CCTGCTGGAA TGCGCCTGGC TGGAAATCCT GATGATCGGC
CTGGTCTGGC GGAGCATGGA ACACCCCGGC AAGCTGCTGT
TCGCCCCCAA CCTGCTGCTG GACAGGAACC AGGGCAAGTG
CGTCGAGGGC ATGGTGGAGA TTTTCGACAT GCTGCTGGCC
ACCTCCAGCA GGTTCAGGAT GATGAACCTG CAGGGCGAGG
AATTTGTGTG CCTGAAGAGC ATCATCCTGC TGAACAGCGG
CGTGTACACC TTCCTGAGCA GCACCCTGAA GAGCCTGGAA
GAGAAGGACC ACATCCACAG GGTGCTGGAC AAGATCACCG
ACACCCTGAT CCACCTGATG GCCAAGGCCG GCCTGACACT
CCAGCAGCAG CACCAGAGGC TGGCCCAGCT GCTGCTGATC
CTGAGCCACA TCAGGCACAT GAGCAACAAG GGGATGGAAC
ACCTGTACAG CATGAAGTGC AAGAACGTGG TGCCCCTGTA
CGATCTGCTC CTGGAAATGC TGGACGCCCA CAGGCTGCAC
GCC

The above DNA sequence encodes the 241 C-terminal residues of the human estrogen receptor protein disclosed herein. The 241 amino acid residues are as follows.

TADQMVSALL DAEPPILYSE YDPTRPFSEA SMMGLLTNLA
DRELVHMINW AKRVPGFVDL TLHDQVHLLE CAWLEILMIG
LVWRSMEHPG KLLFAPNLLL DRNQGKCVEG MVEIFDMLLA
TSSRFRMMNL QGEEFVCLKS IILLNSGVYT FLSSTLKSLE
EKDHIHRVLD KITDTLIHLM AKAGLTLQQQ HQRLAQLLLI
LSHIRHMSNK GMEHLYSMKC KNVVPLYDLL LEMLDAHRLH
A

The various vectors are separately transfected into CHO cells and secreted protein collected. The cell culture supernatant after various times of incubation is spun at 10,000-13,000 rpm for 15 min at 4° C. and filtered prior to purification.

The supernatant is diluted 50:50 with a binding buffer (PBS, pH 7.4, containing 500 mM Glycine) before injection on to the Protein G affinity chromatography column (Mo Bi Tech, Molecular Biotechnology), which is pre-equilibrated with 5 column volumes of the binding buffer. The column is washed with 10 column volumes of binding buffer. The sample is then eluted off the column with 100 mM Glycine-HCl, pH 3.0 and collected in eppendorf tubes containing a 15% final fraction volume of 2.0M Tris.HCl, pH 7.4.

### Cell Line

Mammalian CHO cell cultures are maintained in a Form a Scientific Incubator with 10% carbon dioxide at 37° C. in Dulbecco's Modified Eagle Medium (DMEM) (Gibco). Penicillin (100 U/ml), streptomycin (100 μg/ml) and amphotericin B (25 ng/ml) (Gibco Invitrogen #15240-062) are added to media as standard. As a routine, cells are maintained in the presence of 5% or 10% fetal bovine serum (Gibco Invitrogen #10099-141) unless otherwise stated. Subconfluent cells are passaged with 0.5% trypsin-EDTA (Gibco Invitrogen #15400-054).

### Propagation of DNA Constructs

DNA expression constructs are propagated in supercompetent DH5α E. Coli (Stratagene). To transform bacteria, 1 μg of plasmid DNA is added to 200 μl of bacteria in a microfuge tube and placed on ice for 20 min. Bacteria are heat shocked at 42° C. for 1.5 min, then replaced on ice for a further 5 min. 1 ml of Luria-Bertani broth (LB) without antibiotics is then added, and the bacteria incubated at 37° C. on a heat block for 1 h. This is then added to 200 ml of LB with penicillin 50 μg/ml and incubated overnight at 37° C. with agitation in a Bioline Shaker (Edwards Instrument Company, Australia). The following morning the bacterial broth are transferred to a large centrifuge tube and spun at 10,000 rpm for 15 min. The supernatant is removed and the pellet dried by inverting the tube on blotting paper. Plasmid DNA is recovered using the Wizard@ Plus Midipreps DNA purification system (Promega #A7640). The pellet is resuspended in 3 ml of Cell Resuspension Solution (50 mM Tris-HCl pH 7.5, 10 mM EDTA, 100 μg/ml RNase A) and an equal volume of Cell Lysis Solution added (0.2 M NaOH, 1% SDS). This is mixed by inversion four times. 3 ml of neutralization solution (1.32 M potassium acetate pH 4.8) is then added, and the solution again mixed by inversion. This is centrifuged at 14,000 g for 15 min at 4° C. The supernatant is then carefully decanted to a new tube by straining through muslin cloth. 10 ml of resuspended DNA purification resin is added to the DNA solution and mixed thoroughly. The Midi column tip is inserted into a vacuum pump, the DNA solution/resin mixture added to the column, and the vacuum applied. Once the solution is passed through the column it is washed twice by adding 15 ml of Column Wash Solution and applying the vacuum until the solution had drawn through. After the last wash the column is sharply incised to isolate the column reservoir which is transferred to a microfuge tube and spun at 13,000 rpm for 2 min to remove any residual wash solution. 100 μl of pre-heated nuclease-free water is added and the DNA eluted by centrifuging at 13,000 rpm for 20 sec in a fresh tube. DNA concentration is measured by absorbance spectroscopy (Perkin Elmer MBA2000).

### Examination of DNA Products by Gel Electrophoresis

The DNA products of polymerase chain reactions or restriction enzyme digests of plasmid DNA are analysed by agarose gel electrophoresis. Agarose (1-1.2%) is dissolved in TAE buffer (40 mM Tris acetate, 2 mM EDTA pH 8.5) containing 0.5 μg/ml ethidium bromide. A DNA loading dye consisting of 0.2% w/v xylene cyanol, 0.2% bromophenol blue, 40 mM Tris acetate, 2 mM EDTA pH 8.5 and 50% glycerol is added to the samples before electrophoresis. Electrophoresis is conducted at approximately 100V in 1×TAE. DNA samples are visualized under ultraviolet light (254 nm).

Polypeptide Fusion Protein Transfection and Expression in CHO cells

Plasmids encoding polypeptide fusion proteins are transfected into CHO cells using calcium phosphate. Cells are seeded in 6-well plates to be ˜40-50% confluent on the day of transfection. Growth media is changed 3 h prior to transfection. 2 μg of plasmid DNA is mixed with 37 μl of 2 M calcium phosphate in a microfuge tube and the final volume made up to 300 μl with dH₂O. This is added dropwise to an equal volume of 2×HBS with continuous vortexing, and incubated at RT for 30 min. This solution is then added dropwise to the plate. Cells are incubated for 6 h, cells washed twice with TBS, and fresh media added. Transfection efficiency is determined by spiking a control sample with 0.2 μg of pcDNA3.GFP.

### Example 3

### Efficacy of Androgen-Binding Polypeptide by In Vitro Assay

A human hormone sensitive prostate cancer cell line, LNCaP, is exposed to a polypeptide as described in Example 1. The effects on of the polypeptide on the growth and proliferation of the cells is then assessed.

As a control for hormone ablation therapy, the cells are cultured in hormone depleted serum (Charcoal stripped serum) as well as in normal serum to demonstrate growth in normal levels of androgens.

### Cell Culture.

The human prostate cancer cell line, LNCaP is obtained from American Type Tissue Collection (ATCC) and is routinely cultured in growth medium containing phenol red RPMI 1640 (Invitrogen, Auckland, New Zealand) supplemented with 10% fetal bovine serum (FBS, GIBCO) and 1% antibiotic/antimycotic mixture (Invitrogen, Auckland, New Zealand). Cells are maintained at 37° C. in 5% CO₂. Serial dilutions are made for the polypeptide (0.001 ng/ml-100 ug/ml) in either 5% FBS or 5% charcoal strip serum (CSS, HyClone) for in vitro experiments.

### In Vitro—Growth Proliferation Study.

5×10³ LNCaP cells are plated per well in a Falcon 96-well plate and allowed to attach overnight at 5% CO₂/37° C. in growth medium (as indicated above). The medium is replaced with fresh complete growth medium containing various concentrations (0.001 ng/ml-100 ug/ml) of polypeptide in RPMI medium supplemented either with 5% FBS (normal serum, NS) or 5% CSS. After between 96-168 hours in culture, cells are washed once with PBS and labelled with calcein (C1430, Molecular Probes, Oregon, USA) at 1 mM final concentration in PBS. Calcein positive cells are detected using a FLUOstar OPTIMA plate reader (BMG Labtech, Victoria, Australia). Experiments are performed in 6 replicates per polypeptide concentration for each condition: serum (containing NS) and serum-free (containing charcoal strip serum).

### Statistical Analysis

Data are presented as mean±SD unless otherwise indicated. Differences between treatment groups are analyzed using Fisher's least significant difference test with significance assumed at 99% confidence interval, for p>0.01, One-Way ANOVA. All statistical analysis is performed using STATGRAPHICS statistical software (Virginia, USA). The proliferative effect of the polypeptide at different concentrations in combination with either normal serum or charcoal strip serum is calculated according to the method of Romanelli S et al (Cancer Chemother Pharmacol. 1998:41(5):385-90).

### Example 4

### Efficacy of Estrogen-Binding Polypeptide by In Vivo Assay

A human hormone sensitive breast cancer cell line, MCF-7, is exposed to a polypeptide as described in Example 2. The effects on of the polypeptide on the growth and proliferation of the cells is then assessed.

As a control for hormone ablation therapy, the cells are cultured in hormone depleted serum (Charcoal stripped serum) as well as in normal serum to demonstrate growth in normal levels of estrogens.

### Cell Culture.

The human breast cancer cell line, MCF-7 is obtained from American Type Tissue Collection (ATCC) and is routinely cultured in growth medium containing phenol red RPMI 1640 (Invitrogen, Auckland, New Zealand) supplemented with 10% fetal bovine serum (FBS, GIBCO) and 1% antibiotic/antimycotic mixture (Invitrogen, Auckland, New Zealand). Cells are maintained at 37° C. in 5% CO₂. Serial dilutions are made for the polypeptide (0.001 ng/ml-100 ug/ml) in either 5% FBS or 5% charcoal strip serum (CSS, HyClone) for in vitro experiments.

### In Vitro—Growth Proliferation Study.

5×10³ MCF-7 cells are plated per well in a Falcon 96-well plate and allowed to attach overnight at 5% CO₂/37° C. in growth medium (as indicated above). The medium is replaced with fresh complete growth medium containing various concentrations (0.001 ng/ml-100 ug/ml) of polypeptide in RPMI medium supplemented either with 5% FBS (normal serum, NS) or 5% CSS. After between 96-168 hours in culture, cells are washed once with PBS and labelled with calcein (C1430, Molecular Probes, Oregon, USA) at 1 mM final concentration in PBS. Calcein positive cells are detected using a FLUOstar OPTIMA plate reader (BMG Labtech, Victoria, Australia). Experiments are performed in 6 replicates per polypeptide concentration for each condition: serum (containing NS) and serum-free (containing charcoal strip serum).

### Statistical Analysis

Data are presented as mean±SD unless otherwise indicated. Differences between treatment groups are analyzed using Fisher's least significant difference test with significance assumed at 99% confidence interval, for p>0.01, One-Way ANOVA. All statistical analysis is performed using STATGRAPHICS statistical software (Virginia, USA). The proliferative effect of the polypeptide at different concentrations in combination with either normal serum or charcoal strip serum is calculated according to the method of Romanelli S et al (Cancer Chemother Pharmacol. 1998:41(5):385-90).

### Example 5

### Efficacy of Polypeptide by In Vivo Assay

4-6 week old female balb/c mice housed under standard conditions. All mice are ovariectomised and a controlled amount of oestradiol (up to 30-100 micrograms per day) is delivered by subcutaneous hormone pellets or via acute tail vein injection. Each group will comprise eight mice per group.

### Treatment Arms

Polypeptide capable of binding estrogen is given as alternate tail vein injection once a week (maximum of 200 μl injection, up to 3 mg/kg) for the duration of the experiment.

Pellets for either oestradiol replacement are implanted either using a stainless steel reusable precision trochar (for pellets 0.3 cm in diameter or smaller), supplied from Innovative Research of America or via surgery (with the maximum size of under 0.5 cm). Pellets are implanted on the back of the mice. Animals receiving surgery for implantation are administered an anaesthetic of isoflurane, and the incision is closed with 4/0 silk.

### Monitoring and Collection of Samples

Blood is sampled at specific time points after oestrogen dosing to monitor free and total estrogen and polypeptide levels. Blood (maximum of 200 μL) is collected via alternating mandibular or tail vein bleeds, procedures carried out by animal house staff experienced in this technique.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as broadly described herein.

Future patent applications may be filed in Australia or overseas on the basis of or claiming priority from the present application. It is to be understood that the following provisional claims are provided by way of example only, and are not intended to limit the scope of what may be claimed in any such future application. Features may be added to or omitted from the provisional claims at a later date so as to further define or re-define the invention or inventions.

The present invention will now be more fully described by reference to the following non-limiting Examples.

### Example 6

### Construction of Androgen-Binding or Estrogen-Binding Bi-Functional Molecule Including Elastin-Like Polypeptide Sequences

This example refers to the following sequences:

```
AR - ELP DNA (Open Reading Frame)
      1 ATGGGCAGCA GCCATCACCA TCATCACCAC AGCCAGGATC CGAATTCACC
    51 ATGGGTCGAC AACAACCAGC CGGATAGCTT CGCGGCGCTG CTGTCTAGCC
  101 TGAACGAACT GGGCGAACGT CAGCTGGTGC ATGTGGTGAA ATGGGCGAAA
  151 GCGCTGCCGG GCTTTCGTAA CCTGCATGTG GATGATCAGA TGGCGGTGAT
  201 TCAGTATAGC TGGATGGGCC TGATGGTGTT TGCGATGGGC TGGCGCAGCT
  251 TTACCAACGT GAACAGCCGT ATGCTGTATT TTGCGCCGGA TCTGGTGTTT
  301 AACGAATACC GCATGCATAA AAGCCGTATG TATAGCCAGT GCGTGCGTAT
  351 GCGTCATCTG AGCCAGGAAT TTGGCTGGCT GCAGATTACC CCGCAAGAAT
  401 TTCTGTGCAT GAAAGCGCTG CTGCTGTTTA GCATTATTCC GGTGGATGGC
  451 CTGAAAAACC AGAAATTTTT CGATGAACTG CGCATGAACT ACATCAAAGA
  501 ACTGGATCGT ATTATTGCGT GCAAACGCAA AAATCCGACC AGCTGCAGCC
  551 GTCGTTTTTA TCAGCTGACC AAACTGCTGG ATAGCGTGCA GCCGATTGCG
  601 CGTGAACTGC ATCAGTTTAC CTTTGATCTG CTGATCAAAA GCCATATGGT
  651 GAGCGTGGAT TTTCCGGAAA TGATGGCGGA AATTATTAGC GTGCAGGTGC
  701 CGAAAATTCT GAGCGGCAAA GTGAAACCGA TCTATTTTCA TACCCAGCTC
  751 GAGGGCCACG GCGTGGGTGT TCCGGGTGTT GGTGTGCCGG GTGTGGGCGT
  801 TCCGGGCGTT GGCGTTCCGG GCGTGGGTGT GCCGGGCGTT GGTGTTCCGG
  851 GTGTTGGCGT TCCGGGTGTT GGTGTGCCGG GCGTTGGCGT GCCGGGTGTG
  901 GGCGTGCCGG GCGGGCAGTA TGGTACCCTC GAGTCTGGTA AAGAAACCGC
  951 TGCTGCGAAA TTTGAACGCC AGCACATGGA CTCGTCTACT AGCGCAGCTT
1001 AA
AR - ELP Amino Acid Sequence
    1 MGSSHHHHHH SQDPNSPWVD NNQPDSFAAL LSSLNELGER QLVHVVKWAK
  51 ALPGFRNLHV DDQMAVIQYS WMGLMVFAMG WRSFTNVNSR MLYFAPDLVF
101 NEYRMHKSRM YSQCVRMRHL SQEFGWLQIT PQEFLCMKAL LLFSIIPVDG
151 LKNQKFFDEL RMNYIKELDR IIACKRKNPT SCSRRFYQLT KLLDSVQPIA
201 RELHQFTFDL LIKSHMVSVD FPEMMAEIIS VQVPKILSGK VKPIYFHTQL
251 EGHGVGVPGV GVPGVGVPGV GVPGVGVPGV GVPGVGVPGV GVPGVGVPGV
301 GVPGGQYGTL ESGKETAAAK FERQHMDSST SAA*
ER - ELP DNA (Open Reading Frame)
    1 ATGGGCAGCA GCCATCACCA TCATCACCAC AGCCAGGATC CGAATTCGCC
    51 ATGGGTCGAC ACCGCGGATC AGATGGTGAG CGCGCTGCTG GATGCGGAAC
  101 CGCCGATTCT GTATAGCGAA TATGATCCGA CCCGTCCGTT TAGCGAAGCG
  151 AGCATGATGG GCCTGCTGAC CAACCTGGCC GATCGTGAAC TGGTGCATAT
  201 GATTAACTGG GCGAAACGTG TGCCGGGCTT TGTGGATCTG ACCCTGCATG
  251 ATCAGGTGCA TCTGCTGGAA TGCGCGTGGC TGGAAATTCT GATGATTGGC
  301 CTGGTGTGGC GCAGCATGGA ACATCCGGGC AAACTGCTGT TTGCGCCGAA
  351 CCTGCTGCTG GATCGTAACC AGGGCAAATG CGTGGAAGGC ATGGTGGAAA
  401 TTTTTGATAT GCTGCTGGCG ACGTCTAGCC GTTTCCGTAT GATGAACCTG
  451 CAGGGCGAAG AATTTGTGTG CCTGAAAAGC ATTATTCTGC TGAACAGCGG
  501 CGTGTATACC TTTCTGAGCA GCACCCTGAA AAGCCTGGAA GAAAAAGATC
  551 ATATTCACCG CGTGCTGGAT AAAATTACCG ATACCCTGAT TCATCTGATG
  601 GCGAAAGCCG GCCTGACCCT GCAGCAGCAG CATCAGCGTC TGGCCCAGCT
  651 GCTGCTGATT CTGAGCCATA TTCGTCACAT GAGCAACAAA GGTATGGAAC
  701 ACCTGTATAG CATGAAATGC AAAAACGTGG TGCCGCTGTA TGATCTGCTG
  751 CTGGAAATGC TGGATGCGCA TCGTCTGCAT GCCTCGAGCC ACGGCGTGGG
  801 TGTTCCGGGT GTTGGTGTGC CGGGTGTGGG CGTTCCGGGC GTTGGCGTTC
  851 CGGGCGTGGG TGTGCCGGGC GTTGGTGTTC CGGGTGTTGG CGTTCCGGGT
  901 GTTGGTGTGC CGGGCGTTGG CGTGCCGGGT GTGGGCGTGC CGGGCGGGCA
  951 GTATGGTACC CTCGAGTCTG GTAAAGAAAC CGCTGCTGCG AAATTTGAAC
1001 GCCAGCACAT GGACTCGTCT ACTAGCGCAG CTTAA
ER - ELP Amino Acid Sequence
    1 MGSSHHHHHH SQDPNSPWVD TADQMVSALL DAEPPILYSE YDPTRPFSEA
  51 SMMGLLTNLA DRELVHMINW AKRVPGFVDL TLHDQVHLLE CAWLEILMIG
101 LVWRSMEHPG KLLFAPNLLL DRNQGKCVEG MVEIFDMLLA TSSRFRMMNL
151 QGEEFVCLKS IILLNSGVYT FLSSTLKSLE EKDHIHRVLD KITDTLIHLM
201 AKAGLTLQQQ HQRLAQLLLI LSHIRHMSNK GMEHLYSMKC KNVVPLYDLL
251 LEMLDAHRLH ASSHGVGVPG VGVPGVGVPG VGVPGVGVPG VGVPGVGVPG
301 VGVPGVGVPG VGVPGGQYGT LESGKETAAA KFERQHMDSS TSAA*
```

Maps of the above ORFs and polypeptides are shown in FIGS. 1 to 4 herein.

A synthetic cassette encoding the human androgen receptor (AR) ligand binding domain (690 bp) fused N-terminal to ELP[V5A2G3]-10, an ELP encoding 10 Val-Pro-Gly-Xaa-Gly repeats where Xaa is Val, Ala, and Gly in a 5:2:3 ratio, respectively, is subcloned into a modified E. coli cloning vector PUC18 utilising EcoR1 and Bgl II restriction sites. The modified PUC18 vector has the two Bgl I sites (at positions 245 and 1813) in the parental PUC18 vector mutated via silent site directed mutagenesis so that both Bgl I sites are destroyed. The sequence of the human AR ligand binding region are also modified to utilise optimal E. coli expression codons to optimise expression in prokaryotic systems, whilst the AA sequence is identical to the human AR protein sequence.

The 10 repeats of the elastin like peptide sequence, ELP, optimised for expression in E. coli is as follows:

GGCCACGGCG TGGGTGTTCC GGGTGTTGGT GTGCCGGGTG
TGGGCGT TCCGGGCGTT GGCGTTCCGG GCGTGGGTGT
GCCGGGCGTT GGTGTTCCGG GTGTTGGCGT TCCGGGTGTT
GGTGTGCCGG GCGTTGGCGT GCCGGGTGTG GGCGTGCCGG
GCGGGCAG

### Plasmid Construction.

ELP[V5A2G3]-90, an ELP encoding 90 Val-Pro-Gly-Xaa-Gly repeats where Xaa is Val, Ala, and Gly in a 5:2:3 ratio, respectively, was fused downstream and 3′ to either the AR or ER LBD. An ELP protein fused with AR-LBD (AR-LBD-ELP) is synthesized by inserting the AR LBD gene 5′ to the ELP-[V5A2G3]-90 gene in pET DUET vector with a T-7 promoter (Novagen, Madison, Wis.)

A synthetic gene with EcoR1 and Bgl II restriction sites encoding for AR-LBD-ELP[V5A2G3]-90 is synthesized by recursive directional ligation in a modified pUC-18 vector. ELP repeats of varying lengths are then oligomerized and selected using standard restriction digestion, fragment purification and ligation techniques. For a typical oligomerization, the vector is linearized with PfIMI and enzymatically dephosphorylated. The insert is doubly digested with PfIMI and BglI, purified by agarose gel electrophoresis (QIAquick Gel Extraction Kit, Qiagen), and ligated into the linearized vector. This is performed sequentially so that a range of AR-ELP repeat protein lengths are synthesized.

The PUC18 vector containing the AR-LBD-ELP construct is then digested with EcoRI and KpnI, followed by enzymatic dephosphorylation with calf intestinal phosphatase and purification from a low melting point agarose gel. The expression fragment is then cloned into a doubly digested EcoRI and KpnI pET DUET vector with a T-7 promoter (Novagen, Madison, Wis.).

The pET DUET expression vector is then transformed into E. coli strain BLR(DE3) (Novagen), which is commonly used for expressing recombinant proteins with tandem repeats due to its deficiency in homologous recombination.

### Protein Expression.

Terrific Broth (TB) (for 1 L, 12 g tryptone and 24 g yeast extract (TB basal, TBB). Phosphate buffer (2.31 g potassium phosphate monobasic and 12.54 g potassium phosphate dibasic) and glycerol (4 mL) (PBG) are added separately as supplements, where noted. Stock solutions of the 20 amino acid supplements (Sigma, St. Louis, Mo.) are prepared in deionized water at 200 mM and were sterilized separately using 0.2 □m filters before being added to the medium. The initial pH values of cultures supplemented with various amino acids ranged from 7 to 7.2, except those with aspartic acid, glutamic acid, and histidine, which are adjusted to this range by adding an appropriate amount of 1 M sodium hydroxide. A 2 mL culture of E. coli BLR(DE3) harboring a plasmid for the AR-LBD-ELP or ER-LBD-ELP protein is inoculated with a single colony from a freshly streaked agar plate supplemented with 100 □g/mL ampicillin. After overnight incubation at 37° C. with orbital agitation at 300 rpm, the optical density (OD600) of the culture is determined on a spectrophotometer.

E. coli cells are pelleted by centrifugation (2000×g, 4° C., 15 min), resuspended in fresh medium, and used to inoculate 50 mL of medium in a 250-mL Erlenmeyer flask, unless otherwise stated. The inoculum volume is adjusted to obtain an initial OD600 of 0.1. The culture is incubated at 37° C. with orbital agitation at 300 rpm. For the IPTG induction protocol, isopropyl □-thiogalactopyranoside (IPTG) is added to a final concentration of 1 mM to induce protein expression when OD600. Cultures are then continued for an additional 4 h postinduction, which is the typical incubation duration for induced cultures. For the hyperexpression protocol, no IPTG is added to the 50 mL cultures, which are allowed to grow for 24 h after inoculation. Cells are harvested from the cultures by centrifugation (2000×g, 4° C., 15 min), resolubilized in low-ionic-strength buffer (˜ 1/30 culture volume), and lysed by ultrasonic disruption at 4° C. The lysate is centrifuged at ˜20,000 g at 4° C. for 15 min to remove insoluble matter. Nucleic acids are precipitated by the addition of polyethylenimine (0.5% final concentration), followed by centrifugation at ˜20,000 g at 4° C. for 15 min.

### Fusion Protein Purification.

The AR-LBD-ELP fusion proteins, are purified by inverse transition cycling. For purification by inverse transition cycling, ELP fusion proteins are aggregated by increasing the temperature of the cell lysate to □45° C. and/or by adding NaCl to a concentration □2 M. The aggregated fusion protein is separated from solution by centrifugation at 35-45° C. at 10,000-15,000 g for 15 min. The supernatant is decanted and discarded, and the pellet containing the fusion protein is resolubilized by agitation in cold, low-ionic-strength buffer. The resolubilized pellet is then centrifuged at 4° C. to remove any remaining insoluble matter.

### Characterization of ELP Fusion Proteins.

The optical absorbance at 350 nm of ELP fusion solutions is monitored in the 4-80° C. range on a Cary 300 ultraviolet-visible spectrophotometer equipped with a multicell thermoelectric temperature controller (Varian Instruments). The Tt is determined from the midpoint of the transition-induced change at a heating or cooling rate of 1.5° C. min-1. The SDS-PAGE analysis uses precast Mini-PROTEAN 10-20% gradient gels (Bio-Rad, Hercules, Calif.) with a discontinuous buffer system, staining with Coomassie brilliant blue. The concentration of the fusion proteins is determined spectrophotometrically using calculated extinction coefficients. Total protein concentrations are determined by bicinchonic acid assay (Pierce Chemical Co.).

### Example 7

### Use of Bi-Functional Protein to Deplete Androgen from Fetal Bovine Serum

Specific Depletion of Testosterone from Fetal Calf Serum:

Total testosterone levels in fetal or newborn calf serum is typically around the 20 ng/dl level as determined by the Coat-A-Count solid-phase radioimmunoassay. To deplete testosterone from serum 1 ml of serum is incubated with a range of different AR-LBD-ELP fusion protein concentrations ranging from 10 ng, 25 ng, 50 ng, and 100 ng at 37° C. for 30 min to 1 hr to permit binding of endogenous testosterone present in the serum to the fusion protein.

The AR-LBD-ELP fusion proteins with bound testosterone, are then purified from the serum by inverse transition cycling. For purification by inverse transition cycling, ELP fusion proteins are aggregated by increasing the temperature of the serum to □55° C. The aggregated fusion protein is separated from solution by microfiltration by passing the heated serum solution with aggregated fusion protein through a 0.2 □m syringe pore filter (Corning Incorporated). The filtrate is collected and then total testosterone levels in the filtered serum determined by competitive radioimmunoassay procedures.

### Quantification of Testosterone Levels in Serum:

The Coat-A-Count (DPC Corporation, 5210 Pacific Concourse Drive Los Angeles, Calif., TKTT1 (100 tubes) procedure is a solid-phase radioimmunoassay, based on testosterone-specific antibody immobilized to the wall of a polypropylene tube. ¹²⁵I-labeled testosterone competes for a fixed time with testosterone in the serum sample for antibody sites. The tube is then decanted, to separate bound from free, and counted in a gamma counter. The amount of testosterone present in the serum sample is determined from a calibration curve.

### Radioimmunoassay Procedure

A single calibration curve provides the basis for determining testosterone concentrations in serum. All components are at room temperature (15-28° C.) before use.

### 1 Plain Tubes:

Label four plain (uncoated) 12×75 mm polypropylene tubes T (total counts) and NSB (nonspecific binding) in duplicate.

### Coated Tubes:

Label twelve Total Testosterone Ab-Coated Tubes A (maximum binding) and B through F in duplicate. Label additional antibody-coated tubes, also in duplicate, for controls and serum samples.

| Calibrators | ng/dL | nmol/L |
|---|---|---|
| A (MB) | 0 | 0 |
| B | 20 | 0.7 |
| C | 100 | 3.5 |
| D | 400 | 14 |
| E | 800 | 28 |
| F | 1,600 | 55 |

2 Pipet 50 μL of the zero calibrator A into the NSB and A tubes, and 50 μL of each remaining calibrator, control and serum sample into the tubes prepared.

3 Add 1.0 mL of ¹²⁵I Total Testosterone to every tube. Vortex. Set the T tubes aside for counting (at step 6); they require no further processing.

4 Incubate for 3 hours at 37° C.

5 Decant thoroughly and allow them to drain for 2 or 3 minutes. Then strike the tubes sharply on absorbant paper to shake off all residual droplets.

6 Count for 1 minute in a gamma counter.

### Calculation and Quality Control

To calculate total testosterone concentrations from a logit-log representation of the calibration curve, first calculate for each pair of tubes the average NSB-corrected counts per minute: Net Counts=(Average CPM) minus (Average NSB CPM). Then determine the binding of each pair of tubes as a percent of maximum binding (MB), with the NSB-corrected counts of the A tubes taken as 100%:

Percent Bound=(Net Counts/Net MB Counts)×100

The calculation can be simplified by omitting the correction for non-specific binding; samples within range of the calibrators yield virtually the same results when Percent Bound is calculated directly from Average CPM. Using logit-log graph paper, plot Percent Bound on the vertical (probability) axis against Concentration on the horizontal (logarithmic) axis for each of the nonzero calibrators, and draw a straight line approximating the path of these points. Results for the unknowns may then be read from the line by interpolation.

### Example 8

### Assessment of Bi-Functional Protein Using Androgen-Dependant Cell Line

A human hormone sensitive prostate cancer cell line, LNCaP, is cultured in a depleted serum as prepared in Example 2. The effects of the depleted serum on the growth and proliferation of the cells is then assessed. As a control replicate cells are cultured in normal serum.

### Cell Culture.

The human prostate cancer cell line, LNCaP is obtained from American Type Tissue Collection (ATCC) and is routinely cultured in growth medium containing phenol red RPMI 1640 (Invitrogen, Auckland, New Zealand) supplemented with 10% fetal bovine serum (FBS, GIBCO) and 1% antibiotic/antimycotic mixture (Invitrogen, Auckland, New Zealand). Cells are maintained at 37° C. in 5% CO₂.

### In Vitro—Growth Proliferation Study.

5×10³ LNCaP cells are plated per well in a Falcon 96-well plate and allowed to attach overnight at 5% CO₂/37° C. in androgen depleted medium. After between 96-168 hours in culture, cells are washed once with PBS and labelled with calcein (C1430, Molecular Probes, Oregon, USA) at 1 mM final concentration in PBS. Calcein positive cells are detected using a FLUOstar OPTIMA plate reader (BMG Labtech, Victoria, Australia).

### Statistical Analysis

Data are presented as mean±SD unless otherwise indicated. Differences between treatment groups are analyzed using Fisher's least significant difference test with significance assumed at 99% confidence interval, for p>0.01, One-Way ANOVA. All statistical analysis is performed using STATGRAPHICS statistical software (Virginia, USA). The proliferative effect of normal serum or hormone depleted serum is calculated according to the method of Romanelli S et al (Cancer Chemother Pharmacol. 1998:41(5):385-90).

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as broadly described herein.

Future patent applications may be filed in Australia or overseas on the basis of or claiming priority from the present application. It is to be understood that the following provisional claims are provided by way of example only, and are not intended to limit the scope of what may be claimed in any such future application. Features may be added to or omitted from the provisional claims at a later date so as to further define or re-define the invention or inventions.

### Example 9

### Construction of Estrogen-Binding Polypeptide

The following coding region for human estrogen receptor ligand binding domain (723 bp) is subcloned into various vectors (pFUSE-hIgG1-Fc2, pFUSE-hIgG1e2-Fc2, pFUSE-mIgG1-Fc2 from Invivogen) using EcoRI and BglII RE sites (see FIGS. 1 to 3).

ACCGCCGACC AGATGGTGTC CGCCCTGCTG GACGCCGAGC
CCCCCATCCT GTACAGCGAG TACGACCCCA CCAGGCCCTT
CTCCGAGGCT AGCATGATGG GCCTGCTGAC CAACCTGGCC
GACCGGGAGC TGGTGCACAT GATCAACTGG GCCAAGAGGG
TGCCCGGCTT CGTCGACCTG ACACTGCACG ATCAGGTCCA
CCTGCTGGAA TGCGCCTGGC TGGAAATCCT GATGATCGGC
CTGGTCTGGC GGAGCATGGA ACACCCCGGC AAGCTGCTGT
TCGCCCCCAA CCTGCTGCTG GACAGGAACC AGGGCAAGTG
CGTCGAGGGC ATGGTGGAGA TTTTCGACAT GCTGCTGGCC
ACCTCCAGCA GGTTCAGGAT GATGAACCTG CAGGGCGAGG
AATTTGTGTG CCTGAAGAGC ATCATCCTGC TGAACAGCGG
CGTGTACACC TTCCTGAGCA GCACCCTGAA GAGCCTGGAA
GAGAAGGACC ACATCCACAG GGTGCTGGAC AAGATCACCG
ACACCCTGAT CCACCTGATG GCCAAGGCCG GCCTGACACT
CCAGCAGCAG CACCAGAGGC TGGCCCAGCT GCTGCTGATC
CTGAGCCACA TCAGGCACAT GAGCAACAAG GGGATGGAAC
ACCTGTACAG CATGAAGTGC AAGAACGTGG TGCCCCTGTA
CGATCTGCTC CTGGAAATGC TGGACGCCCA CAGGCTGCAC
GCC

This sequence encodes the 241 C-terminal residues of the human estrogen receptor protein disclosed as follows:

TADQMVSALL DAEPPILYSE YDPTRPFSEA SMMGLLTNLA
DRELVHMINW AKRVPGFVDL TLHDQVHLLE CAWLEILMIG
LVWRSMEHPG KLLFAPNLLL DRNQGKCVEG MVEIFDMLLA
TSSRFRMMNL QGEEFVCLKS IILLNSGVYT FLSSTLKSLE
EKDHIHRVLD KITDTLIHLM AKAGLTLQQQ HQRLAQLLLI
LSHIRHMSNK GMEHLYSMKC KNVVPLYDLL LEMLDAHRLH
A

The various vectors are separately transfected into CHO cells and secreted protein collected. The cell culture supernatant after various times of incubation is spun at 10,000-13,000 rpm for 15 min at 4° C. and filtered prior to purification.

The supernatant is diluted 50:50 with a binding buffer (PBS, pH 7.4, containing 500 mM Glycine) before injection on to the Protein G affinity chromatography column (Mo Bi Tech, Molecular Biotechnology), which is pre-equilibrated with 5 column volumes of the binding buffer. The column is washed with 10 column volumes of binding buffer. The sample is then eluted off the column with 100 mM Glycine-HCl, pH 3.0 and collected in eppendorf tubes containing a 15% final fraction volume of 2.0M Tris-HCl, pH 7.4.

### Cell Line

Mammalian CHO cell cultures are maintained in a Form a Scientific Incubator with 10% carbon dioxide at 37° C. in Dulbecco's Modified Eagle Medium (DMEM) (Gibco). Penicillin (100 U/ml), streptomycin (100 μg/ml) and amphotericin B (25 ng/ml) (Gibco Invitrogen #15240-062) are added to media as standard. As a routine, cells are maintained in the presence of 5% or 10% fetal bovine serum (Gibco Invitrogen #10099-141) unless otherwise stated. Subconfluent cells are passaged with 0.5% trypsin-EDTA (Gibco Invitrogen #15400-054).

### Propagation of DNA Constructs

DNA expression constructs are propagated in supercompetent DH5α E. Coli (Stratagene). To transform bacteria, 1 μg of plasmid DNA is added to 200 μl of bacteria in a microfuge tube and placed on ice for 20 min. Bacteria are heat shocked at 42° C. for 1.5 min, then replaced on ice for a further 5 min. 1 ml of Luria-Bertani broth (LB) without antibiotics is then added, and the bacteria incubated at 37° C. on a heat block for 1 h. This is then added to 200 ml of LB with penicillin 50 μg/ml and incubated overnight at 37° C. with agitation in a Bioline Shaker (Edwards Instrument Company, Australia). The following morning the bacterial broth are transferred to a large centrifuge tube and spun at 10,000 rpm for 15 min. The Supernatant is removed and the pellet dried by inverting the tube on blotting paper. Plasmid DNA is recovered using the Wizard® Plus Midipreps DNA purification system (Promega #A7640). The pellet is resuspended in 3 ml of Cell Resuspension Solution (50 mM Tris-HCl pH 7.5, 10 mM EDTA, 100 μg/ml RNase A) and an equal volume of Cell Lysis Solution added (0.2 M NaOH, 1% SDS). This is mixed by inversion four times. 3 ml of neutralization solution (1.32 M potassium acetate pH 4.8) is then added, and the solution again mixed by inversion. This is centrifuged at 14,000 g for 15 min at 4° C. The supernatant is then carefully decanted to a new tube by straining through muslin cloth. 10 ml of resuspended DNA purification resin is added to the DNA solution and mixed thoroughly. The Midi column tip is inserted into a vacuum pump, the DNA solution/resin mixture added to the column, and the vacuum applied. Once the solution is passed through the column it is washed twice by adding 15 ml of Column Wash Solution and applying the vacuum until the solution had drawn through. After the last wash the column is sharply incised to isolate the column reservoir which is transferred to a microfuge tube and spun at 13,000 rpm for 2 min to remove any residual wash solution. 100 μl of pre-heated nuclease-free water is added and the DNA eluted by centrifuging at 13,000 rpm for 20 sec in a fresh tube. DNA concentration is measured by absorbance spectroscopy (Perkin Elmer MBA2000).

### Examination of DNA Products by Gel Electrophoresis

The DNA products of polymerase chain reactions or restriction enzyme digests of plasmid DNA are analysed by agarose gel electrophoresis. Agarose (1-1.2%) is dissolved in TAE buffer (40 mM Tris acetate, 2 mM EDTA pH 8.5) containing 0.5 μg/ml ethidium bromide. A DNA loading dye consisting of 0.2% w/v xylene cyanol, 0.2% bromophenol blue, 40 mM Tris acetate, 2 mM EDTA pH 8.5 and 50% glycerol is added to the samples before electrophoresis. Electrophoresis is conducted at approximately 100V in 1×TAE. DNA samples are visualized under ultraviolet light (254 nm).

### Polypeptide Fusion Protein Transfection and Expression in CHO Cells

Plasmids encoding polypeptide fusion proteins are transfected into CHO cells using calcium phosphate. Cells are seeded in 6-well plates to be ˜40-50% confluent on the day of transfection. Growth media is changed 3 h prior to transfection. 2 μg of plasmid DNA is mixed with 37 μl of 2 M calcium phosphate in a microfuge tube and the final volume made up to 300 μl with dH₂O. This is added dropwise to an equal volume of 2×HBS with continuous vortexing, and incubated at RT for 30 min. This solution is then added dropwise to the plate. Cells are incubated for 6 h, cells washed twice with TBS, and fresh media added. Transfection efficiency is determined by spiking a control sample with 0.2 μg of pcDNA3.GFP.

### Example 10

### Construction of Androgen-Binding Polypeptide

The following coding region for human androgen receptor ligand binding domain (690 bp) is subcloned into various vectors (pFUSE-hIgG1-Fc2, pFUSE-hIgG1e2-Fc2, pFUSE-mIgG1-Fc2 from Invivogen) using EcoRI and BglII RE sites (see FIGS. 1 to 3).

GACAACAACCAGCCCGACAGCTTCGCCGCCCTGCTGTCCAGCCTGAAC
GAGCTGGGCGAGAGGCAGCTGGTGCACGTGGTGAAGTGGGCCAAGGCC
CTGCCCGGCTTCAGAAACCTGCACGTGGACGACCAGATGGCCGTGATC
CAGTACAGCTGGATGGGCCTGATGGTGTTCGCTATGGGCTGGCGGAGC
TTCACCAACGTGAACAGCAGGATGCTGTACTTCGCCCCCGACCTGGTG
TTCAACGAGTACAGGATGCACAAGAGCAGGATGTACAGCCAGTGCGTG
AGGATGAGGCACCTGAGCCAGGAATTTGGCTGGCTGCAGATCACCCCC
CAGGAATTTCTGTGCATGAAGGCCCTGCTGCTGTTCAGCATCATCCCC
GTGGACGGCCTGAAGAACCAGAAGTTCTTCGACGAGCTGCGGATGAAC
TACATCAAAGAGCTGGACAGGATCATCGCCTGCAAGAGGAAGAACCCC
ACCTCCTGCAGCAGAAGGTTCTACCAGCTGACCAAGCTGCTGGACAGC
GTGCAGCCCATCGCCAGAGAGCTGCACCAGTTCACCTTCGACCTGCTG
ATCAAGAGCCACATGGTGTCCGTGGACTTCCCCGAGATGATGGCCGAG
ATCATCAGCGTGCAGGTGCCCAAGATCCTGAGCGGCAAGGTCAAGCCC
ATCTACTTCCACACCCAG

This sequence encodes the 230 C-terminal residues of the human androgen receptor protein.

The various vectors are separately transfected into CHO cells and secreted protein collected. The cell culture supernatant after various times of incubation is spun at 10,000-13,000 rpm for 15 min at 4° C. and filtered prior to purification.

The supernatant is diluted 50:50 with a binding buffer (PBS, pH 7.4, containing 500 mM Glycine) before injection on to the Protein G affinity chromatography column (Mo Bi Tech, Molecular Biotechnology), which is pre-equilibrated with 5 column volumes of the binding buffer. The column is washed with 10 column volumes of binding buffer. The sample is then eluted off the column with 100 mM Glycine-HCl, pH 3.0 and collected in eppendorf tubes containing a 15% final fraction volume of 2.0M Tris-HCl, pH 7.4.

### Cell Line

Mammalian CHO cell cultures are maintained in a Form a Scientific Incubator with 10% carbon dioxide at 37° C. in Dulbecco's Modified Eagle Medium (DMEM) (Gibco). Penicillin (100 U/ml), streptomycin (100 μg/ml) and amphotericin B (25 ng/ml) (Gibco Invitrogen #15240-062) are added to media as standard. As a routine, cells are maintained in the presence of 5% or 10% fetal bovine serum (Gibco Invitrogen #10099-141) unless otherwise stated. Subconfluent cells are passaged with 0.5% trypsin-EDTA (Gibco Invitrogen #15400-054).

### Propagation of DNA Constructs

DNA expression constructs are propagated in supercompetent DH5α E. Coli (Stratagene). To transform bacteria, 1 μg of plasmid DNA is added to 200 μl of bacteria in a microfuge tube and placed on ice for 20 min. Bacteria are heat shocked at 42° C. for 1.5 min, then replaced on ice for a further 5 min. 1 ml of Luria-Bertani broth (LB) without antibiotics is then added, and the bacteria incubated at 37° C. on a heat block for 1 h. This is then added to 200 ml of LB with penicillin 50 μg/ml and incubated overnight at 37° C. with agitation in a Bioline Shaker (Edwards Instrument Company, Australia). The following morning the bacterial broth are transferred to a large centrifuge tube and spun at 10,000 rpm for 15 min. The supernatant is removed and the pellet dried by inverting the tube on blotting paper. Plasmid DNA is recovered using the Wizard® Plus Midipreps DNA purification system (Promega #A7640). The pellet is resuspended in 3 ml of Cell Resuspension Solution (50 mM Tris-HCl pH 7.5, 10 mM EDTA, 100 μg/ml RNase A) and an equal volume of Cell Lysis Solution added (0.2 M NaOH, 1% SDS). This is mixed by inversion four times. 3 ml of neutralization solution (1.32 M potassium acetate pH 4.8) is then added, and the solution again mixed by inversion. This is centrifuged at 14,000 g for 15 min at 4° C. The supernatant is then carefully decanted to a new tube by straining through muslin cloth. 10 ml of resuspended DNA purification resin is added to the DNA solution and mixed thoroughly. The Midi column tip is inserted into a vacuum pump, the DNA solution/resin mixture added to the column, and the vacuum applied. Once the solution is passed through the column it is washed twice by adding 15 ml of Column Wash Solution and applying the vacuum until the solution had drawn through. After the last wash the column is sharply incised to isolate the column reservoir which is transferred to a microfuge tube and spun at 13,000 rpm for 2 min to remove any residual wash solution. 100 μl of pre-heated nuclease-free water is added and the DNA eluted by centrifuging at 13,000 rpm for 20 sec in a fresh tube. DNA concentration is measured by absorbance spectroscopy (Perkin Elmer MBA2000).

### Examination of DNA Products by Gel Electrophoresis

The DNA products of polymerase chain reactions or restriction enzyme digests of plasmid DNA are analysed by agarose gel electrophoresis. Agarose (1-1.2%) is dissolved in TAE buffer (40 mM Tris acetate, 2 mM EDTA pH 8.5) containing 0.5 μg/ml ethidium bromide. A DNA loading dye consisting of 0.2% w/v xylene cyanol, 0.2% bromophenol blue, 40 mM Tris acetate, 2 mM EDTA pH 8.5 and 50% glycerol is added to the samples before electrophoresis. Electrophoresis is conducted at approximately 100V in 1×TAE. DNA samples are visualized under ultraviolet light (254 nm).

### Polypeptide Fusion Protein Transfection and Expression in CHO Cells

Plasmids encoding polypeptide fusion proteins are transfected into CHO cells using calcium phosphate. Cells are seeded in 6-well plates to be ˜40-50% confluent on the day of transfection. Growth media is changed 3 h prior to transfection. 2 μg of plasmid DNA is mixed with 37 μl of 2 M calcium phosphate in a microfuge tube and the final volume made up to 300 μl with dH2O. This is added dropwise to an equal volume of 2×HBS with continuous vortexing, and incubated at RT for 30 min. This solution is then added dropwise to the plate. Cells are incubated for 6 h, cells washed twice with TBS, and fresh media added. Transfection efficiency is determined by spiking a control sample with 0.2 μg of pcDNA3.GFP.

### Example 11

### Efficacy of Estrogen-Binding Polypeptide by In Vitro Assay

A human hormone sensitive breast cancer cell line, MCF-7, is exposed to a polypeptide as described in Example 2. The effects on of the polypeptide on the growth and proliferation of the cells is then assessed.

As a control for hormone ablation therapy, the cells are cultured in hormone depleted serum (Charcoal stripped serum) as well as in normal serum to demonstrate growth in normal levels of estrogens.

### Cell Culture.

The human breast cancer cell line, MCF-7 is obtained from American Type Tissue Collection (ATCC) and is routinely cultured in growth medium containing phenol red RPMI 1640 (Invitrogen, Auckland, New Zealand) supplemented with 10% fetal bovine serum (FBS, GIBCO) and 1% antibiotic/antimycotic mixture (Invitrogen, Auckland, New Zealand). Cells are maintained at 37° C. in 5% CO₂. Serial dilutions are made for the polypeptide (0.001 ng/ml-100 ug/ml) in either 5% FBS or 5% charcoal strip serum (CSS, HyClone) for in vitro experiments.

### In Vitro—Growth Proliferation Study.

5×10³ MCF-7 cells are plated per well in a Falcon 96-well plate and allowed to attach overnight at 5% CO₂/37° C. in growth medium (as indicated above). The medium is replaced with fresh complete growth medium containing various concentrations (0.001 ng/ml-100 ug/ml) of polypeptide in RPMI medium supplemented either with 5% FBS (normal serum, NS) or 5% CSS. After between 96-168 hours in culture, cells are washed once with PBS and labelled with calcein (C1430, Molecular Probes, Oregon, USA) at 1 mM final concentration in PBS. Calcein positive cells are detected using a FLUOstar OPTIMA plate reader (BMG Labtech, Victoria, Australia). Experiments are performed in 6 replicates per polypeptide concentration for each condition: serum (containing NS) and serum-free (containing charcoal strip serum).

### Statistical Analysis

Data are presented as mean±SD unless otherwise indicated. Differences between treatment groups are analyzed using Fisher's least significant difference test with significance assumed at 99% confidence interval, for p>0.01, One-Way ANOVA. All statistical analysis is performed using STATGRAPHICS statistical software (Virginia, USA). The proliferative effect of the polypeptide at different concentrations in combination with either normal serum or charcoal strip serum is calculated according to the method of Romanelli S et al (Cancer Chemother Pharmacol. 1998:41(5):385-90).

### Example 12

### Efficacy of Androgen-Binding Polypeptide by In Vitro Assay

A human hormone sensitive prostate cancer cell line, LNCaP, is exposed to a polypeptide as described in Example 1. The effects on of the polypeptide on the growth and proliferation of the cells is then assessed.

As a control for hormone ablation therapy, the cells are cultured in hormone depleted serum (Charcoal stripped serum) as well as in normal serum to demonstrate growth in normal levels of androgens.

### Cell Culture.

The human prostate cancer cell line, LNCaP is obtained from American Type Tissue Collection (ATCC) and is routinely cultured in growth medium containing phenol red RPMI 1640 (Invitrogen, Auckland, New Zealand) supplemented with 10% fetal bovine serum (FBS, GIBCO) and 1% antibiotic/antimycotic mixture (Invitrogen, Auckland, New Zealand). Cells are maintained at 37° C. in 5% CO2. Serial dilutions are made for the polypeptide (0.001 ng/ml-100 ug/ml) in either 5% FBS or 5% charcoal strip serum (CSS, HyClone) for in vitro experiments.

### In Vitro—Growth Proliferation Study.

5×103 LNCaP cells are plated per well in a Falcon 96-well plate and allowed to attach overnight at 5% CO2/37° C. in growth medium (as indicated above). The medium is replaced with fresh complete growth medium containing various concentrations (0.001 ng/ml-100 ug/ml) of polypeptide in RPMI medium supplemented either with 5% FBS (normal serum, NS) or 5% CSS. After between 96-168 hours in culture, cells are washed once with PBS and labelled with calcein (C1430, Molecular Probes, Oregon, USA) at 1 mM final concentration in PBS. Calcein positive cells are detected using a FLUOstar OPTIMA plate reader (BMG Labtech, Victoria, Australia). Experiments are performed in 6 replicates per polypeptide concentration for each condition: serum (containing NS) and serum-free (containing charcoal strip serum).

### Statistical Analysis

Data are presented as mean±SD unless otherwise indicated. Differences between treatment groups are analyzed using Fisher's least significant difference test with significance assumed at 99% confidence interval, for p>0.01, One-Way ANOVA. All statistical analysis is performed using STATGRAPHICS statistical software (Virginia, USA). The proliferative effect of the polypeptide at different concentrations in combination with either normal serum or charcoal strip serum is calculated according to the method of Romanelli S et at (Cancer Chemother Pharmacol. 1998:41(5):385-90).

### Example 13

### Efficacy of Estrogen-Binding Polypeptide by In Vivo Assay

### Breast Cancer Models

4-6 week old female balb/c nude or SCID, SCIDNOD mice are housed under sterile conditions in micro-isolators. Antibiotics (Baytril 25) is given via drinking water to all mice.

All mice are ovariectomised and require a controlled amount of oestradiol (up to 30 micrograms per day) that will be delivered by subcutaneous hormone pellets. Each group comprise eight mice. One control group has no tumour injected while another is injected with tumour cells but receive no treatment.

Subcutaneous models comprise subcutaneous flank injection of the animals with up to 1×10⁷ cells in up to 200 μl at each site, of sterile culture medium containing 100 μl of sterile Matrigel or sterile culture medium. The injections are carried out in the animal facility under sterile conditions.

Orthotopic Breast cancer is established by injection into the mammary fat pad, with up to 1×10⁷ cells (i.e. MCF-7) in up to 200 μl at each site, of sterile culture medium containing 100 μl of sterile Matrigel or sterile culture medium. The injections are carried out in the animal facility under sterile conditions.

### Treatment Arms

Er tarp binding protein is given as alternate tail vein injection once a week (maximum of 200 μl injection, up to 3 mg/kg) for the duration of the experiment.

Pellets for either oestradiol replacement or hormone therapy are implanted either using a stainless steel reusable precision trochar (for pellets 0.3 cm in diameter or smaller), supplied from Innovative Research of America or via surgery (with the maximum size of under 0.5 cm). Pellets are implanted on the back of the mice. Animals receiving surgery for implantation are administered an anaesthetic of isoflurane. The incision is closed with 4/0 silk.

### Monitoring and Collection of Samples

In both subcutaneous and orthotopic models blood is sampled at distinct time points after tumour implantation to monitor free and total estrogen levels. Blood (maximum of 200 μL) is collected via alternating mandibular or tail vein bleeds.

The end of the experiment is defined as a humane endpoints in the experimental induction of neoplasia in all tumour cohorts apart from the orthotopic prostate model, when tumours in the untreated control animal groups approach 10% of the animal's normal body weight. This represents a subcutaneous flank tumour diameter of 17 mm in a 25 g mouse. Tumours are monitored and the hair of the SCID/SCIDNOD mice removed. Mice are euthanased with carbon dioxide, tumours removed, weighed and the dimensions recorded. Specimens are fixed and embedded for future analysis.

### Example 14

### Efficacy of Androgen-Binding Polypeptide by In Vivo Assay

A xenograft animal model of an androgen dependent tumor is used to assess efficacy in vivo. 5-7 week old SCID (severe-combined immunodeficiency) or athymic balb/c nude male mice are purchased from the Animal Resources Centre, Perth, Western Australia, and housed in microisolator. Mice are given free access to standard rodent chow and drinking water throughout all experiments.

### Orthotopic Model of Hormone Dependent Prostate Cancer

Orthotopic tumours are established as follows. Mice (between 6-10 per treatment group) are anaesthetized with a mixture of ketamine 10.0 mg/kg and xylazine 20 mg/kg injected intraperitoneally to allow a small transverse lower abdominal incision to be made. The bladder, seminal vesicles and prostate are delivered into the wound and 1×10⁶ LNCaP cells in 20 μl of cell culture medium with Matrigel injected into the dorsolateral prostate with a 29 gauge needle. Injections are performed with the aid of an operating microscope at ×10 magnification. A technically satisfactory injection is confirmed by the formation of a subcapsular bleb and the absence of visible leak. The lower urinary tract is replaced and the anterior abdominal wall closed with 4/0 silk. The skin is apposed with surgical staples. Postoperatively the animals are given an intraperitoneal injection of normal saline at a calculated volume of 3-5% of the pre-anaesthetic weight. Mice are recovered under radiant heating lamps until fully mobile.

Animals are divided into treatment groups of 6-10 mice and after different time periods following tumour cell injection are administered IV tail vein Injections of the polypepetide at different concentrations (optimised from in vitro experimental results). At the end of the experiment mice are sacrificed by carbon dioxide narcosis. The prostate, seminal vesicles and bladder are removed en bloc, and appendages carefully dissected from the tumour containing prostate if not grossly involved. The tumour containing prostate gland is weighed, and diameter measured in three dimensions with Vernier calipers. The retroperitoneum is explored under magnification cephadally to the level of the renal veins. Lymph nodes found in the para-aortic and para-iliac areas are dissected free and their long axis measured. Tissue for Immunohistochemical staining is embedded in OCT and frozen in liquid nitrogen cooled isopentane. Tumours are stored at −70° C. until analysis.

### Subcutaneous Tumour Models

To establish androgen responsive tumours, 5×106 washed LNCaPs cells are resuspended in 50 μl PBS, mixed with an equal volume of Matrigel (BD #354234) and injected subcutaneously into the right flank of 0.6-8 week old male nude mice with a 23G needle. To establish androgen insensitive tumours, 1×106 PC3 cells are similarly injected, but no Matrigel is used.

### Surgical Castration

As controls for hormone ablation therapy, Mice are anaesthetized with a mixture of ketamine 100 mg/kg and xylazine 20 mg/kg injected intraperitoneally to allow a small transverse lower abdominal incision to be made. The lower genitourinary organs are delivered into the wound, the vas deferens and vascular pedicle ligated with 4/0 silk, and the testes excised. The abdomen is closed with 4/0 silk with clips to skin. Mice are recovered on a heating pad until fully recovered.

### Local Tumour Growth in Orthotopic Models of ADPC

At specified times post inoculation (from days 25-42), mice are euthanased by carbon monoxide narcosis and a necroscopy performed. The abdomen is opened in the midline from sternum to pubis and retracted, and the abdominal organs inspected. Under magnification, the urethra is transected at the prostatic apex and the ureters and vas deferentia are identified bilaterally and divided close to the prostate. The specimen is then removed en bloc and the seminal vesicles and bladder dissected free under magnification. The tumour containing prostate gland is then weighed and its dimensions measured in 3 axes with Vernier calipers. Where a discrete nodule is found this is dissected away and weighed separately.

After these measurements, the prostate or tumour is embedded in OCT, snap frozen in liquid nitrogen cooled isopentane and stored at −70° C. until use. Prostate glands without macroscopic tumours are serially sectioned and analysed histologically to confirm the presence of tumour.

Volume of the tumour containing prostate gland is calculated using the formula a*b*c, where a, b and c represent maximum length of the gland measured with Vernier calipers in three dimensions at right angles to one another.

### Example 15

### A Study to Determine the Efficacy and Safety of Estrogen-Specific Polypeptide in Patients with Metastatic Breast Cancer Who have Failed Previous Hormonal Therapy

This study includes up to 15 post-menopausal women with hormone-sensitive (ER+ or PgR+) metastatic breast cancer, who progress on prior hormone therapy. The purpose of this study is to evaluate the safety and efficacy of estrogen-specific polypeptide in patients who progress on prior hormone therapy for breast cancer. Study participants remain on treatment until disease progression or until other treatment discontinuation criteria are met.

This Example is directed to patients who fail primary hormone therapy. While it would be possible (and desirable) to trial the polypeptide in patients with hormone dependent tumours, patients with advanced breast cancer who fail first line hormone therapy are used at first instance for ethical reasons. This approach allows an assessment of whether the polypeptide is well tolerated, and also permits assessment of the effects on levels of biologically available estrogen levels.

### Objectives

The primary objectives of this study are to determine the safety and tolerability of intra venous infusions of the polypeptide binding protein in patients with advanced breast cancer, and to evaluate its pharmacokinetic profile when given as a single IV infusion once every three weeks. Secondary objectives include: to determine whether treatment with polypeptide binding protein can lead to clinical responses; to estimate progression-free survival; to determine whether treatment with polypeptide binding protein can lead to biological responses in patients with advanced breast cancer.

### Study Design

This study describes an open label phase I dose escalation study. After signing informed consent, patients undergo baseline testing to confirm eligibility. Patients then commence treatment with polypeptide binding protein, administered as a single intravenous infusion once every three weeks (one cycle). After four cycles of therapy (12 weeks), patients with stable or responding disease, and who wish to continue on study, are offered treatment extension for up to another four cycles. All patients are assessed for safety 28 days after the last dose of study drug, and where possible, are evaluated three months after their final treatment of study drug. In total, 12-15 patients (4-patients per dose level) are recruited from a variety of multidisciplinary breast-oncology clinics.

### Patient Eligibility

Patients are screened for study eligibility based on the following inclusion and exclusion criteria. To participate in the study a patient should meet the following criteria:
provide written informed consent
be female with histological/cytological confirmation of hormone sensitive breast cancer with evidence of metastatic disease
have one or more measureable lesions

Any of the following is regarded as a criterion for exclusion from the trial:
1. Prior cytotoxic chemotherapy for advanced breast cancer
2. had radiation therapy within 4 weeks prior to provision of consent
3. Treatment with an investigational agent in the last 4 weeks
4. Other co-existing malignancies or malignancies diagnosed within the last 5 years with the exception of non-melanomatous skin cancer
5. Any unresolved chronic toxicity greater than CTC grade 2 from previous anticancer therapy
6. Incomplete healing from previous surgery
7. Absolute neutrophil counts <1×10⁹/l or platelets <100×10⁹/l
8. Serum bilirubin >1.25 times the upper limit of reference range (ULRR)
9. In the opinion of the investigator, any evidence of severe or uncontrolled systemic disease (e.g. unstable or uncompensated respiratory, cardiac, hepatic or renal disease)
10. Serum creatinine >1.5 times the ULRR
11. Alanine aminotransferase (ALT) or aspartate aminotransferase (AST)>2.5 times the ULRR
12. Evidence of any other significant clinical disorder or laboratory finding that makes it undesirable for the patient to participate in the trial
13. Patients may not use unapproved or herbal remedies for breast cancer
14. A history of alcoholism, drug addiction, or any psychiatric condition which in the opinion of the investigator would impair the patient's ability to comply with study procedures.

### Study Agent

The polypeptide is produced in accordance with Example 1. All formulation and packing of the study agent is in accordance with applicable current Good Manufacturing Practice (GMP) for Investigation Medicinal Products as specified by the Therapeutic Goods Administration (Australia) and meet applicable criteria for use in humans.

### Treatment Plan

Three dose levels of polypeptide binding protein are investigated (0.3, 1.0, and 3.0 mg/kg). After enrollment in the 0.3-mg/kg cohort is complete, there is a 2-week waiting period before the 1.0-mg/kg cohort is begun. There is also a 2-week waiting period after the 1.0-mg/kg cohort is enrolled before enrollment of the 3.0-mg/kg cohort is begun.

Individual patient doses are prepared by diluting the appropriate volume of polypeptide binding protein (25 mg/ml) with 0.9% sodium chloride to yield a final concentration of 4 mg/ml. The volume of solution prepared is 25 to 150 ml, depending on the patient's dose and body weight. The polypeptide is infused over a period of no less than 1 hour by a registered nurse or physician's assistant under the guidance of one of the trial investigators. In addition, internists or anesthesiologists are present to oversee the administration of the study agent and aid in the management of adverse events.

All adverse events are graded according to the Common Terminology Criteria for Adverse Events Version 3.0 (Cancer Therapy Evaluation Program, DCTD, NCl, NIH, DHHS, Mar. 31, 2003, http://ctep.cancer.gov). DRT and DLT is based on the first three weeks of treatment. DRT is defined as any Grade 2 non-haematological or Grade 3 haematological toxicity. DLT is defined as any Grade 3/4 non-haematological or Grade 4 haematological toxicity. Patients who require other treatment for progressive breast cancer, such as radiotherapy to new metastatic lesions, surgery or chemotherapy, are removed from the study and are not replaced. Treatment will not be administered if there is ≧Grade 2 haematological and/or non-haematological toxicity. Treatment may be re-initiated once the toxicity is ≦Grade 1, with treatment delayed for up to two weeks. In the absence of treatment delays, treatment may continue for up to four cycles or until there is disease progression; intercurrent illness prevents further administration of treatment; unacceptable adverse events occur; the patient decides to withdraw from the study; or general or specific changes in the patient's condition render the patients unacceptable for further treatment in the judgment of the trial investigator.

### Pre-Treatment and Treatment Evaluation

At study entry, patients are screened for measurable disease by radionuclide bone scintigraphy and computed tomography of the chest, abdomen and pelvis. In patients with measurable disease, tumour response is assessed according to the Response Evaluation Criteria in Solid Tumours (Therasse, P., et al., J Natl Cancer. Inst, 2000. 92(3): p. 205-16). Given the stage of disease at which patients are enrolled, it is anticipated that the majority will have measurable disease at the time of study entry. Toxicity is evaluated according to the Common Terminology Criteria for Adverse Events Version 3.0.

### Sample Collection

Sample collection to determine population pharmacokinetic parameters for polypeptide binding protein is performed in patients accrued to the study. Serial blood samples (10 ml/sample) are collected at the following times: pre-dose (within 60 min prior to study drug administration) and post-dose at 30 min, 1, 2, 4, 6, 24, 48 and 72 h. In addition, trough samples are taken at days 7, 14 and 21, weeks. Blood samples are collected into heparinised vacutainers for assessment of sodium selenate status. The plasma is separated by centrifugation (2000 g at 4° C. for 15 min). Following centrifugation, the plasma is separated into three aliquots (each approximately 1 ml) and placed in identically labelled polypropylene tubes. Samples are frozen at −80° C. until analysis.

### Study Completion

A patient is considered to have completed the study following the evaluations for the primary endpoint after 4 cycles of treatment. However, patients continuing on study and, receiving further treatment are followed and data collected. Where possible, all patients are evaluated every three months. The study is closed when the final patient has undergone this last review. Patients who have received at least 1 cycle of study agent are evaluable for safety and for clinical and biological response. Proportions and durations of progression-free survival are summarised by Kaplan-Meier methods. Toxicity is summarised according to Common Terminology Criteria for Adverse Events Version 3.0.

### Example 16

### Construction of Androgen-Binding Polypeptide

The following coding region (SEQ ID NO: 4) for human androgen receptor ligand binding domain (690 bp) is subcloned into various vectors (pFUSE-hIgG1-Fc2, pFUSE-hIgG1e2-Fc2, pFUSE-mIgG1-Fc2 from Invivogen) using EcoRI and BglII RE sites (see FIGS. 1 to 3).

gacaacaaccagcccgacagcttcgccgccctgctgtccagcctgaac
gagctgggcgagaggcagctggtgcacgtggtgaagtgggccaaggcc
ctgcccggcttcagaaacctgcacgtggacgaccagatggccgtgatc
cagtacagctggatgggcctgatggtgttcgctatgggctggcggagc
ttcaccaacgtgaacagcaggatgctgtacttcgcccccgacctggtg
ttcaacgagtacaggatgcacaagagcaggatgtacagccagtgcgtg
aggatgaggcacctgagccaggaatttggctggctgcagatcaccccc
caggaatttctgtgcatgaaggccctgctgctgttcagcatcatcccc
gtggacggcctgaagaaccagaagttcttcgacgagctgcggatgaac
tacatcaaagagctggacaggatcatcgcctgcaagaggaagaacccc
acctcctgcagcagaaggttctaccagctgaccaagctgctggacagc
gtgcagcccatcgccagagagctgcaccagttcaccttcgacctgctg
atcaagagccacatggtgtccgtggacttccccgagatgatggccgag
atcatcagcgtgcaggtgcccaagatcctgagcggcaaggtcaagccc
atctacttccacacccag

This sequence encodes the 230 C-terminal residues of the human androgen receptor protein disclosed herein as SEQ ID NO: 1.

The various vectors were separately transfected into CHO cells and secreted protein collected. The cell culture supernatant after various times of incubation was spun at 10,000-13,000 rpm for 15 min at 4° C. and filtered/concentrated prior to use.

### Cell Line

Mammalian CHO cell cultures were maintained in a Form a Scientific Incubator with 10% carbon dioxide at 37° C. in Dulbecco's Modified Eagle Medium (DMEM) (Gibco). Penicillin (100 U/ml), streptomycin (100 μg/ml) and amphotericin B (25 ng/ml) (Gibco Invitrogen #15240-062) were added to media as standard. As a routine, cells were maintained in the presence of 5% or 10% fetal bovine serum (Gibco Invitrogen #10099-141) unless otherwise stated. Subconfluent cells were passaged with 0.5% trypsin-EDTA (Gibco Invitrogen #15400-054).

### Propagation of DNA Constructs

DNA expression constructs were propagatfed in supercompetent DH5α E. Coli (Stratagene). To transform bacteria, 1 μg of plasmid DNA was added to 200 μl of bacteria in a microfuge tube and placed on ice for 20 min. Bacteria were heat shocked at 42° C. for 1.5 min, then replaced on ice for a further 5 min. 1 ml of Luria-Bertani broth (LB) without antibiotics was then added, and the bacteria incubated at 37° C. on a heat block for 1 h. This was then added to 200 ml of LB with penicillin 50 μg/ml and incubated overnight at 37° C. with agitation in a Bioline Shaker (Edwards Instrument Company, Australia). The following morning the bacterial broth were transferred to a large centrifuge tube and spun at 10,000 rpm for 15 min. The supernatant was removed and the pellet dried by inverting the tube on blotting paper. Plasmid DNA was then recovered using the Wizards Plus Midipreps DNA purification system (Promega #A7640). The pellet was resuspended in 3 ml of Cell Resuspension Solution (50 mM Tris-HCl pH 7.5, 10 mM EDTA, 100 μg/ml RNase A) and an equal volume of Cell Lysis Solution added (0.2 M NaOH, 1% SDS). This was mixed by inversion four times. 3 ml of neutralization solution (1.32 M potassium acetate pH 4.8) then added, and the solution again mixed by inversion. This was centrifuged at 14,000 g for 15 min at 4° C. The supernatant was then carefully decanted to a new tube by straining through muslin cloth. 10 ml of resuspended DNA purification resin was added to the DNA solution and mixed thoroughly. The Midi column tip was inserted into a vacuum pump, the DNA solution/resin mixture added to the column, and the vacuum applied. Once the solution was passed through the column it was washed twice by adding 15 ml of Column Wash Solution and applying the vacuum until the solution had drawn through. After the last wash the column was sharply incised to isolate the column reservoir which was transferred to a microfuge tube and spun at 13,000 rpm for 2 min to remove any residual wash solution. 100 μl of pre-heated nuclease-free water was added and the DNA eluted by centrifuging at 13,000 rpm for 20 sec in a fresh tube. DNA concentration was measured by absorbance spectroscopy (Perkin Elmer MBA2000).

### Examination of DNA Products by Gel Electrophoresis

The DNA products of polymerase chain reactions or restriction enzyme digests of plasmid DNA were analysed by agarose gel electrophoresis. Agarose (1-1.2%) was dissolved in TAE buffer (40 mM Tris acetate, 2 mM EDTA pH 8.5) containing 0.5 μg/ml ethidium bromide. A DNA loading dye consisting of 0.2% w/v xylene cyanol, 0.2% bromophenol blue, 40 mM Tris acetate, 2 mM EDTA pH 8.5 and 50% glycerol was added to the samples before electrophoresis. Electrophoresis was conducted at approximately 100V in 1×TAE. DNA samples were visualized under ultraviolet light (254 nm).

### Polypeptide Fusion Protein Transfection and Expression in CHO Cells

The pFUSE-AR-hIgG1e2-Fc2 plasmid encoding the AR-LBD-IgG1FC polypeptide fusion protein was transfected into CHO cells (ATCC) using Fugene HD (Roche, Cat No: 04709691001) and selected with Zeocin (Invitrogen, Cat No:R250-01). 2-5×10⁶ cells were then grown in 100-250 ml CHO-S-SFM II serum free suspension medium (Invitrogen, Cat No:12052-062) for 4-7 days. The cell culture was spun and the supernatant concentrated (using Amicon Ultra 15-50 kDa concentrators, Millipore Cat No:UFC905024).

### Analysis of Fusion Protein Expression Levels

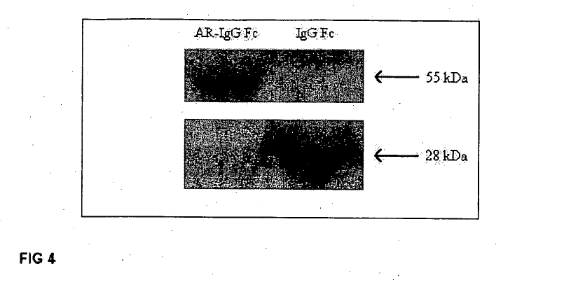

8 μl of concentrated AR or ER-LBD IgG Fc supernatant concentrates and 1 μl of concentrated IgG Fc control supernatants were loaded on to a 12% SDS page gel, and run at 170V for 70 min. The electrophoresed proteins were transferred on to nitrocellulose (100V for 90 min) using standard techniques. The nitrocellulose membranes were then probed with an Anti-Hu IgG Fc HRP conjugate (Pierce, cat no:31413) at 1:20,000 dilution and developed using the Super Signal West Femto developing kit (Pierce, Cat No: 34094) according to the manufacturers specifications. The results are depicted in FIG. 4.

Clear expression of a single predominant polypeptide of size approx 55 kD was observed for both a AR-IgG1 Fc fusion protein as well as a ER-IgG1 Fc fusion protein. The control IgG1 Fc control protein of the correct size (28 kD) was also clearly apparent (FIG. 4).

### Example 17

### Efficacy of Polypeptide by In Vitro Assay

A human hormone sensitive prostate cancer cell line, LNCaP, was exposed to the AR-LBD-IgG1 FC fusion protein as described in Example 1. The effects of the polypeptide on the growth and proliferation of the cells was then assessed.

As a control for hormone ablation therapy, the cells were cultured in hormone depleted serum (Charcoal stripped serum, CSS) as well as in normal serum to demonstrate growth in normal levels of androgens. In addition, LNCaP cells were also cultured in the presence of the non-steroidal antiandrogen nilutamide

### Cell Culture.

The human prostate cancer cell line, LNCaP was obtained from American Type Tissue Collection (ATCC) and was routinely cultured in growth medium containing phenol red RPMI 1640 (Invitrogen, Auckland, New Zealand) supplemented with 10% fetal bovine serum (FBS, GIBCO) and 1% antibiotic/antimycotic mixture (Invitrogen, Auckland, New Zealand). Cells were maintained at 37° C. in 5% CO2.

### In Vitro—Growth Proliferation Study.

2×103 LNCaP cells were plated per well in a Falcon 96-well plate in 5% CO2/37° C. in growth medium in growth medium containing phenol red RPMI 1640 (Invitrogen, Auckland, New Zealand) supplemented with 10% fetal bovine serum (FBS, GIBCO) and 1% antibiotic/antimycotic mixture (Invitrogen, Auckland, New Zealand). Cells were treated with either AR-LBD IgG1 Fc fusion protein (12 ng/ml) or IgG1 Fc control protein (12 ng/ml). In addition as control, 6 wells were treated with the nonsteroidal antiandrogen nilutamide (0.10M) as well as 6 wells with 10% charcoal stripped serum, to simulate steroid free conditions. After 120 hours in culture, cells were washed once with PBS and labelled with calcein (C1430, Molecular Probes, Oregon, USA) at 1 mM final concentration in PBS. Calcein positive cells were detected using a FLUOstar OPTIMA plate reader (BMG Labtech, Victoria, Australia). Experiments were performed in 6 replicates for each treatment condition.

### Statistical Analysis

Data are presented as mean±SEM unless otherwise indicated.

### Results

Treatment of the human hormone sensitive prostate cancer LNCaP cells with the AR IgG1 Fc fusion protein produced a dramatic effect on growth after 5 days exposure as assessed by the fluorescent calcein uptake assay. A 94% reduction in viable LNCaP cells was observed in wells treated with the AR IgG1 Fc fusion protein compared to LNCaP cells grown in media with complete 10% serum (FBS) (FIG. 5, Table 1). In comparison, the control IgG1 Fc protein lacking the AR LBD region had only a negligible effect on growth of the LNCaP cells with only a 6% decline in total cell number (FIG. 5, Table 1), indicating that the growth suppression effect is mediated via the androgen binding domain of the fusion protein. Growth of the LNCaP cells in media devoid of steroids, in the charcoal stripped serum (CSS) had only a modest effect on reducing LNCaP cell proliferation in the assay time frame, with a 18% decline observed (FIG. 5, Table 1). Interestingly, the AR IgG1 Fc fusion protein showed superior efficacy to the antiandrogen nilutamide in reducing LNCaP cell proliferation, with nilutamide reducing prostate cancer cell proliferation by 80% (FIG. 5, Table 1).

These results indicate that the AR IgG1 Fc fusion protein is able to suppress androgen mediated growth of prostate cancer cells. However, this suppression is occurring not only via depleting free androgen levels in the exogenous media, as growth of the LNCaP cells in media totally devoid of steroids had only a modest effect on the cellular proliferation. This superior effect of the AR IgG1Fc protein compared to growth in steroid stripped serum indicates that the fusion protein is able to sequester endogenous androgens either internally or externally produced by the LNCaP cells.

### Example 18

### Efficacy of Polypeptide by In Vivo Assay. Rapid Reduction in, Circulating Free Testosterone Levels

Athymic balb/c nude male mice, 6 weeks of age, were purchased from the Animal Resources Centre, Perth, Western Australia, and housed in a microisolator. Mice were given free access to standard rodent chow and drinking water throughout all experiments.

5 animals were administered IV tail vein injections of the AR-LBD IgG1 Fc fusion protein (25 ng in 200 □l of PBS). Three hours after injection the blood of all 5 mice was collected/pooled via mandibular bleeds (approx 100 □L blood per animal) in Lithium/heparin tubes. In addition, 5 control athymic balb/c nude male mice of the same sex and age were similarly bled at the same time and samples pooled. The unclotted blood was then spun at 2500 rpm for 5 min to separate the red blood cells from the serum. 10001 samples of pooled serum were then run according to the manufacturers specification of the Coat-a-count Free testosterone kit (Siemens, Cat No: TKTF1).

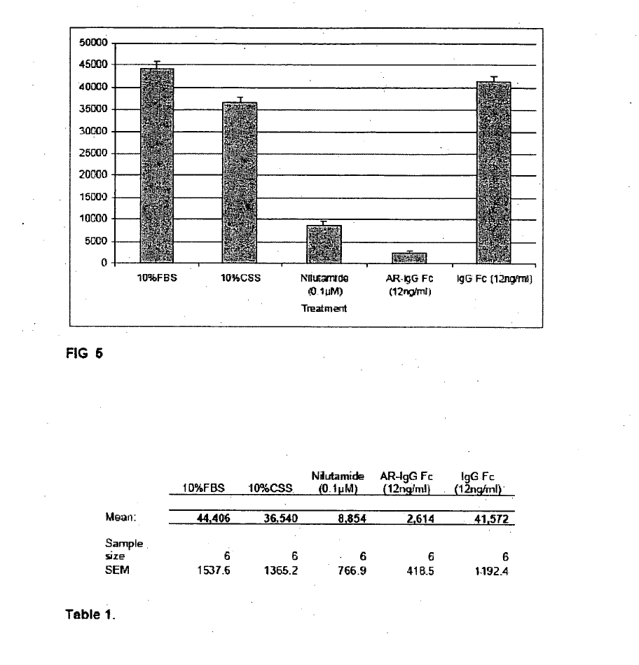

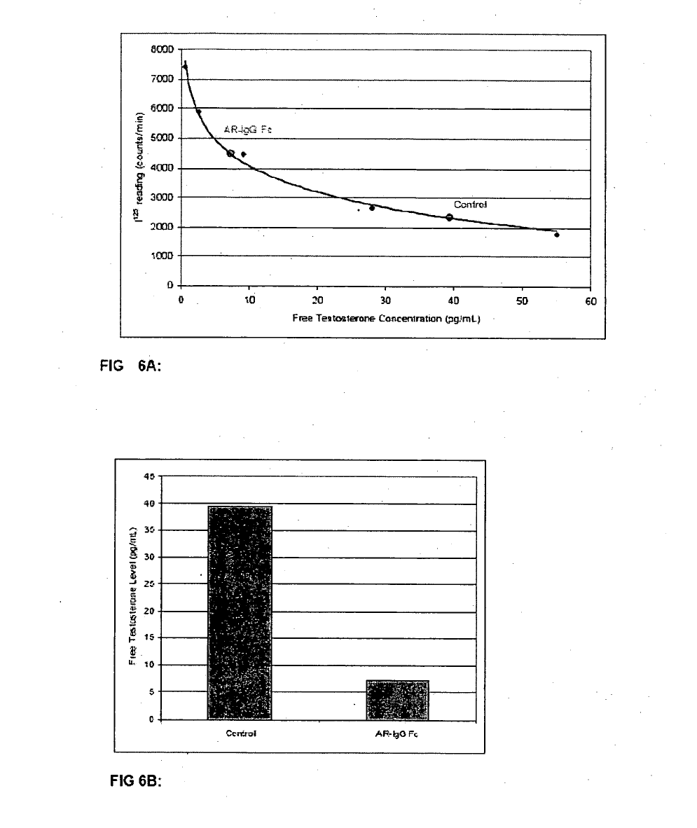

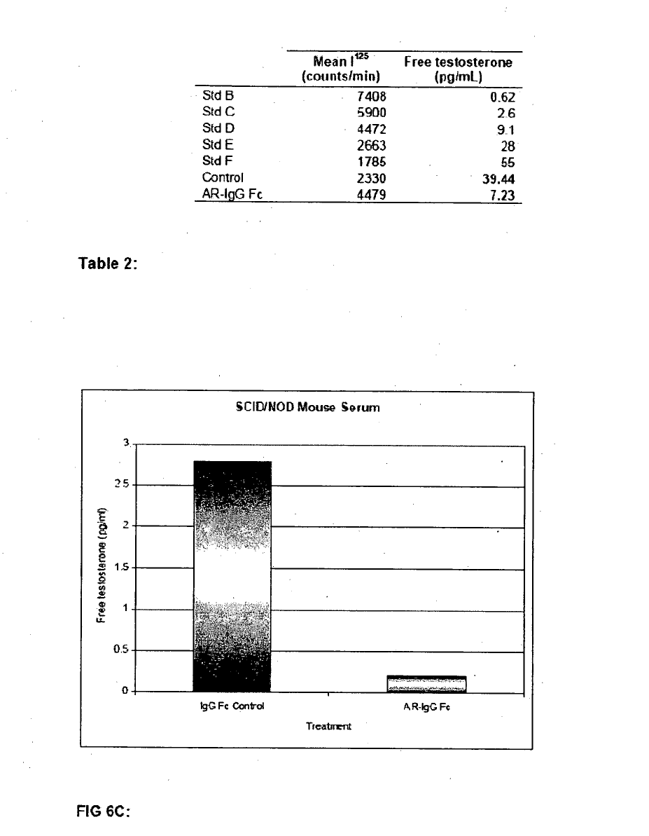

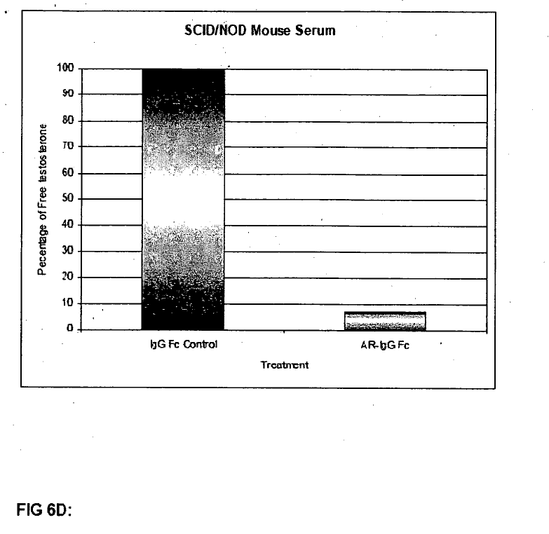

The results are depicted in FIG. 6A, B and Table 2. The free testosterone levels in the serum of the control mice averaged 39.44 pg/ml. However, the free testosterone levels of the mice injected with the AR IgG1 Fc fusion protein was only 7.23 pg/ml. This represents a dramatic 82% decline in bioavailable testosterone levels in only 3 hours after injection.

In a further experiment, 6 SCID/NOD male mice, 5 weeks of age were purchased from the Animal Resources Centre, Perth, Western Australia, and housed in a microisolator. Mice were given free access to standard rodent chow and drinking water throughout all experiments. The animals were then separated into two groups of 3 mice. Three animals in one group were administered IV tail vein injections of the AR-LBD IgG1 Fc fusion protein (200 μl of 1 ng/μl of PBS). Three mice in the other control group, were then administered IV tail vein injections of the control IgG1 Fc protein (200 μl of 1 ng/μl of PBS). Four hours after injection the blood of all 6 mice was collected via mandibular bleeds (approx 100 □l blood per animal) in Lithium/heparin tubes. The unclotted blood was then spun at 2500 rpm for 5 min to separate the red blood cells from the serum. 100 □l samples of pooled serum were then run according to the manufacturers specification of the Coat-a-count Free testosterone kit (Siemens, Cat No: TKTFI).

The results are depicted in FIGS. 6C and D. The free testosterone levels in the serum of the control mice injected with the control IgG1 Fc protein averaged 2.8 pg/ml. However, the free testosterone levels of the mice injected with the AR-LBD IgG1 Fc fusion protein was only 0.2 pg/ml. This represents a dramatic 93% decline in bioavailable testosterone levels only 4 hours after injection.

### Example 19

### Efficacy of Polypeptide by In Vivo Assay

A xenograft animal model of an androgen dependent tumor is used to assess efficacy in vivo. 5-7 week old SCID (severe combined immunodeficiency) or athymic balb/c nude male mice are purchased from the Animal Resources Centre, Perth, Western Australia, and housed in microisolators. Mice are given free access to standard rodent chow and drinking water throughout all experiments.

### Subcutaneous Tumour Models

To establish flank prostate tumours, 4×105 washed LNCaP cells were resuspended in 50 □l PBS, mixed with an equal volume of Matrigel (BD #354234) and injected subcutaneously into the right flank of 6 week old male nude mice with a 23G needle. Following tumour cell injection, 100 μl of 1 ng/μl control IgG1 Fc was injected into the flanks of three mice and 100 μl of 1 ng/μl AR-LBD IgG1 Fc fusion protein injected into the flanks of the three remaining mice. Seven days later, a second flank injection of 200 μl of 1 ng/μl IgG1 Fc was administered to the three animals in the control group and 200 μl of 1 ng/μl AR-LBD IgG1 Fc fusion protein was administered to the three animals in the active treatment group. No further treatment was given and the animals were monitored and tumour sizes measured regularly. The experiment was terminated 5 weeks after the initial tumour cell injection, and final tumour volumes and weight were recorded.

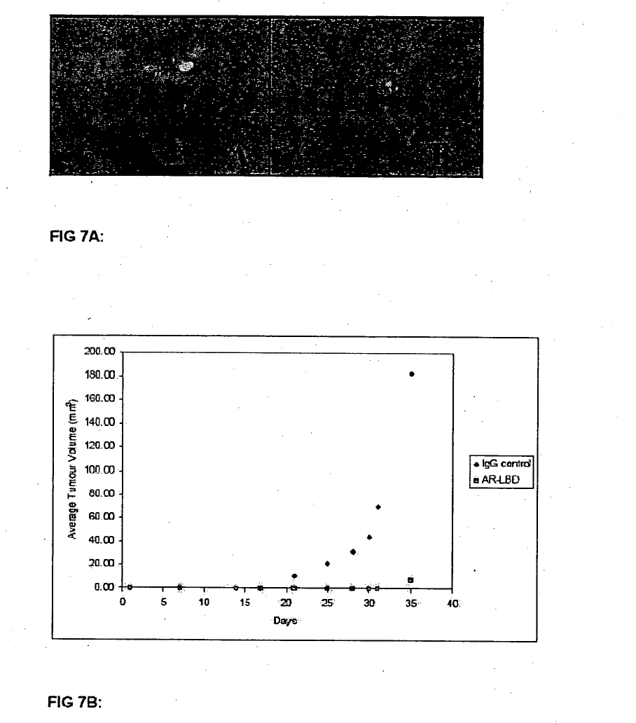

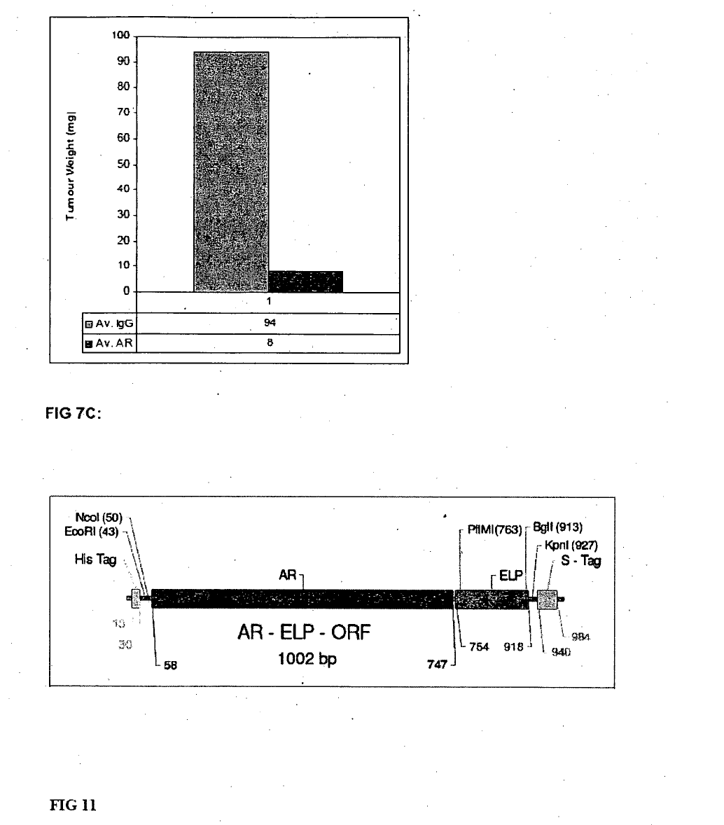

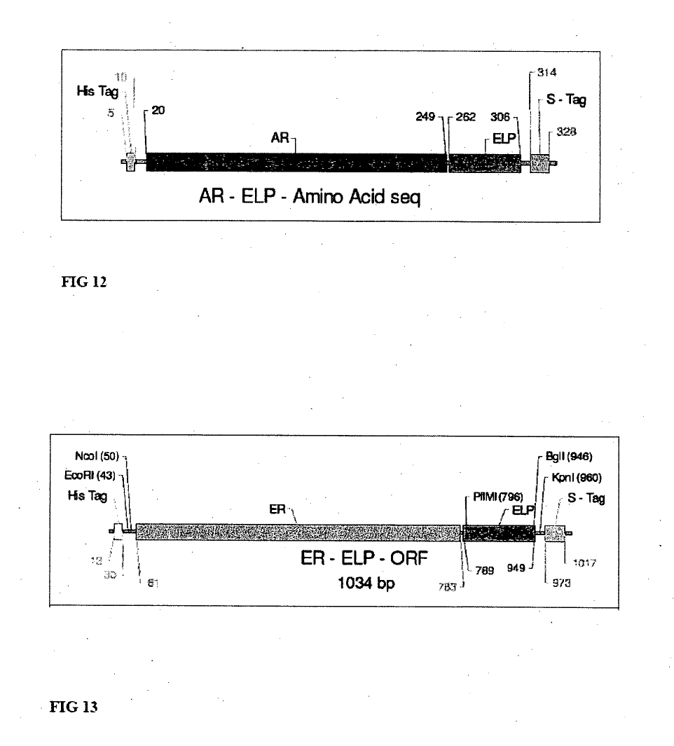

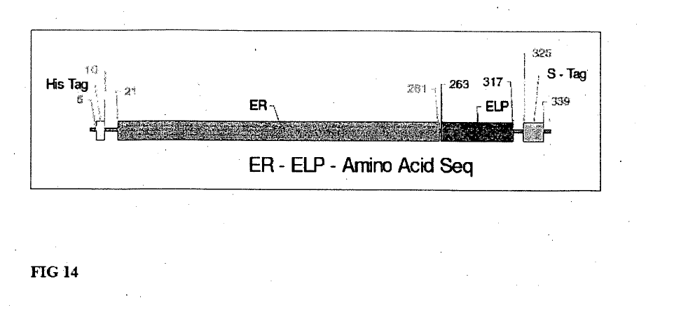

The results are depicted in FIGS. 7A, B and C. The final tumour volume of the control mice injected with the IgG1 Fc protein averaged 182.9 mm3. However, the final tumour volume of the mice injected with the AR-LBD IgG1 Fc fusion protein was only 7.3 mm3 (FIGS. 7A and B). There was also a significant effect of the AR-LBD IgG1 Fc fusion protein in inhibiting prostate tumour growth throughout the experiment with animals treated with the androgen binding fusion protein only developing very small tumours at the end of the experiment (FIG. 7B). This was in marked contrast with animals injected with the control IgG1 protein which developed tumours much earlier and which were much larger at the end of the experiment (FIG. 7B).

There was similarly a very large effect of the AR-LBD IgG1 Fc fusion protein on final tumour weights with average weight being only 8 mg whilst control mice injected with the IgG1 Fc protein averaged 94 mg (FIG. 7C).

### Orthotopic Model of Hormone Dependent Prostate Cancer

Orthotopic tumours are established as follows. Mice (between 6-10 per treatment group) are anaesthetized with a mixture of ketamine 100 mg/kg and xylazine 20 mg/kg injected intraperitoneally to allow a small transverse lower abdominal incision to be made. The bladder, seminal vesicles and prostate are delivered into the wound and 1×10⁶ LNCaPcells in 20 μl of cell culture medium with Matrigel injected into the dorsolateral prostate with a 29 gauge needle. Injections are performed with the aid of an operating microscope at ×10 magnification: A technically satisfactory injection is confirmed by the formation of a subcapsular bleb and the absence of visible leak. The lower urinary tract is replaced and the anterior abdominal wall closed with 4/0 silk. The skin is apposed with surgical staples. Postoperatively the animals are given an intraperitoneal injection of normal saline at a calculated volume of 3-5% of the pre-anaesthetic weight. Mice are recovered under radiant heating lamps until fully mobile.

Animals are divided into treatment groups of 6-10 mice and after different time periods following tumour cell injection are administered IV tail vein injections of the polypepetide at different concentrations (optimised from in vitro experimental results). At the end of the experiment mice are sacrificed by carbon dioxide narcosis. The prostate, seminal vesicles and bladder are removed en bloc, and appendages carefully dissected from the tumour containing prostate if not grossly involved. The tumour containing prostate gland is weighed, and diameter measured in three dimensions with Vernier calipers. The retroperitoneum is explored under magnification cephadally to the level of the renal veins. Lymph nodes found in the para-aortic and para-iliac areas are dissected free and their long axis measured. Tissue for Immunohistochemical staining is embedded in OCT and frozen in liquid nitrogen cooled isopentane. Tumours are stored at −70° C. until analysis.

### Surgical Castration

As controls for hormone ablation therapy, Mice are anaesthetized with a mixture of ketamine 100 mg/kg and xylazine 20 mg/kg injected intraperitoneally to allow a small transverse lower abdominal incision to be made. The lower genitourinary organs are delivered into the wound, the vas deferens and vascular pedicle ligated with 4/0 silk, and the testes excised. The abdomen is closed with 4/0 silk with clips to skin. Mice are recovered on a heating pad until fully recovered.

### Local Tumour Growth in Orthotopic Models of ADPC

At specified times post inoculation (from days 25-42), mice are euthanased by carbon monoxide narcosis and'a necroscopy performed. The abdomen is opened in the midline from sternum to pubis and retracted, and the abdominal organs inspected. Under magnification, the urethra is transected at the prostatic apex and the ureters and vas deferentia are identified bilaterally and divided close to the prostate. The specimen is then removed en bloc and the seminal vesicles and bladder dissected free under magnification. The tumour containing prostate gland is then weighed and its dimensions measured in 3 axes with Vernier calipers. Where a discrete nodule is found this is dissected away and weighed separately.

After these measurements, the prostate or tumour is embedded in OCT, snap frozen in liquid nitrogen cooled isopentane and stored at −70° C. until use. Prostate glands without macroscopic tumours are serially sectioned and analysed histologically to confirm the presence of tumour.

Volume of the tumour containing prostate gland is calculated using the formula a*b*c, where a, b and c represent maximum length of the gland measured with Verniers calipers in three dimensions at right angles to one another.

### Example 20

### Safety and Efficacy of Polypeptide in Human Subjects

This Example is directed to patients with early hormone refractory prostate cancer (HRPC). While it would be possible (and desirable) to trial the polypeptide in patients with hormone dependent tumours, patients with HRPC are used at first instance for ethical reasons. HRPC patients have failed their first line hormone ablation therapy and have no other treatment options until they progress to metastases, when chemotherapy becomes an option. Furthermore, these patients have low levels of circulating testosterone (as they typically remain on androgen ablation therapy, but not on androgen antagonist drugs) and their PSA levels would be just starting to rise. This approach allows an assessment of whether the polypeptide is well tolerated, the effects on levels of biologically available testosterone levels, and also levels PSA.

### Objectives

The primary objectives of this study are to determine the safety and tolerability of intra venous infusions of the polypeptide binding protein in patients with HRPC, and to evaluate its pharmacokinetic profile when given as a single IV infusion once every three weeks. Secondary objectives include: to determine whether treatment with polypeptide binding protein can lead to clinical responses as determined by serum PSA in patients with HRPC; to estimate the duration of PSA response (decline); to estimate progression-free survival; to determine whether treatment with polypeptide binding protein can lead to biological responses in patients with HRPC; and to evaluate the PSA slope before and during polypeptide binding protein therapy.

### Study Design

This study describes an open label phase I dose escalation study. After signing informed consent, patients undergo baseline testing to confirm eligibility. Patients then commence treatment with polypeptide binding protein, administered as a single intravenous infusion once every three weeks (one cycle). After four cycles of therapy (12 weeks), patients with stable or responding disease, and who wish to continue on study, are offered treatment extension for up to another four cycles. All patients are assessed for safety 28 days after the last dose of study drug, and where possible, are evaluated three months after their final treatment of study drug. In total, 12-15 patients (4-patients per dose level) are recruited from a variety of multidisciplinary uro-oncology clinics.

### Patient Eligibility

Patients are screened for study eligibility based on the following inclusion and exclusion criteria.
To be eligible for enrolment, patients must fulfil the following criteria:
1. Provision of written informed consent
2. Male, aged 18 years or older
3. Hormone refractory prostate cancer confirmed by castrate serum testosterone levels and at least three elevated and rising PSA levels, with at least two weeks between measurements
4. The PSA level must be greater than 5 μg/l at study entry
5. Patients may be asymptomatic or have only minor symptoms due to prostate cancer
6. WHO performance status≦2
7. Anti-androgen therapy must have been stopped at least 4 weeks before entry into the trial, with evidence of continuing PSA rises after this time. LHRH agonists or antagonists should be continued and are allowed concurrently
8. Life expectancy of at least six months
   Any of the following is regarded as a criterion for exclusion from the trial:
15. Prior cytotoxic chemotherapy for hormone refractory prostate cancer
16. Prior strontium therapy
17. Treatment with an investigational agent in the last 4 weeks
18. Other co-existing malignancies or malignancies diagnosed within the last 5 years with the exception of non-melanomatous skin cancer
19. Any unresolved chronic toxicity greater than CTC grade 2 from previous anticancer therapy
20. Incomplete healing from previous surgery
21. Absolute neutrophil counts <1×10⁹/l or platelets <100×10⁹/l
22. Serum bilirubin >1.25 times the upper limit of reference range (ULRR)
23. In the opinion of the investigator, any evidence of severe or uncontrolled systemic disease (e.g. unstable or uncompensated respiratory, cardiac, hepatic or renal disease)
24. Serum creatinine >1.5 times the ULRR
25. Alanine aminotransferase (ALT) or aspartate aminotransferase (AST)>2.5 times the ULRR
26. Evidence of any other significant clinical disorder or laboratory finding that makes it undesirable for the patient to participate in the trial
27. Patients may not use unapproved or herbal remedies for prostate cancer
28. A history of alcoholism, drug addiction, or any psychiatric condition which in the opinion of the investigator would impair the patient's ability to comply with study procedures.

### Study Agent

The polypeptide is produced in accordance with Example 1. All formulation and packing of the study agent is in accordance with applicable current Good Manufacturing Practice (GMP) for Investigation Medicinal Products as specified by the Therapeutic Goods Administration (Australia) and meet applicable criteria for use in humans.

### Treatment Plan

Three dose levels of polypeptide binding protein are investigated (0.3, 1.0, and 3.0 mg/kg). After enrollment in the 0.3-mg/kg cohort is complete, there is a 2-week waiting period before the 1.0-mg/kg cohort is begun. There is also a 2-week waiting period after the 1.0-mg/kg cohort is enrolled before enrollment of the 3.0-mg/kg cohort is begun.

Individual patient doses are prepared by diluting the appropriate volume of polypeptide binding protein (25 mg/ml) with 0.9% sodium chloride to yield a final concentration of 4 mg/ml. The volume of solution prepared is 25 to 150 ml, depending on the patient's dose and body weight. The polypeptide is infused over a period of no less than 1 hour by a registered nurse or physician's assistant under the guidance of one of the trial investigators. In addition, internists or anesthesiologists are present to oversee the administration of the study agent and aid in the management of adverse events.

All adverse events are graded according to the Common Terminology Criteria for Adverse Events Version 3.0 (Cancer Therapy Evaluation Program, DCTD, NCI, NIH, DHHS, Mar. 31, 2003, http://ctep.cancer.gov). DRT and DLT is based on the first three weeks of treatment. DRT is defined as any Grade 2 non-haematological or Grade 3 haematological toxicity. DLT is defined as any Grade 3/4 non-haematological or Grade 4 haematological toxicity. Patients who require other treatment for progressive prostate cancer, such as radiotherapy to new metastatic lesions, surgery or chemotherapy, are removed from the study and are not replaced. Treatment will not be administered if there is ≧Grade 2 haematological and/or non-haematological toxicity. Treatment may be re-initiated once the toxicity is ≦Grade 1, with treatment delayed for up to two weeks. In the absence of treatment delays, treatment may continue for up to four cycles or until there is disease progression; intercurrent illness prevents further administration of treatment; unacceptable adverse events occur; the patient decides to withdraw from the study; or general or specific changes in the patient's condition render the patients unacceptable for further treatment in the judgment of the trial investigator.

### Pre-Treatment and Treatment Evaluation

At study entry, patients are screened for measurable disease by radionuclide bone scintigraphy and computed tomography of the chest, abdomen and pelvis. In patients with measurable disease, tumour response is assessed according to the Response Evaluation Criteria in Solid Tumours (Therasse, P., et al., J Natl Cancer Inst, 2000. 92(3): p. 205-16). Given the stage of disease at which patients are enrolled, it is anticipated that the majority will not have measurable disease at the time of study entry. However, patients will have a rising PSA, which is measured every three weeks for the duration of the study. Therefore in patients with no radiologically evaluable disease, PSA response is used as a surrogate marker of tumour response, defined as a reduction in PSA of at least 50% below the level measured at study entry, documented on at least two separate occasions at least four weeks apart. PSA progression is defined as the time from the first PSA decline ≦50% of baseline until an increase in PSA above that level. Toxicity is evaluated according to the Common Terminology Criteria for Adverse Events Version 3.0.

### Sample Collection

Sample collection to determine population pharmacokinetic parameters for polypeptide binding protein is performed in patients accrued to the study. Serial blood samples (10 ml/sample) are collected at the following times: pre-dose (within 60 min prior to study drug administration) and post-dose at 30 min, 1, 2, 4, 6, 24, 48 and 72 h. In addition, trough samples are taken at days 7, 14 and 21, weeks. Blood samples are collected into heparinised vacutainers for assessment of sodium selenate status. The plasma is separated by centrifugation (2000 g at 4° C. for 15 min). Following centrifugation, the plasma is separated into three aliquots (each approximately 1 ml) and placed in identically labelled polypropylene tubes. Samples are frozen at −80° C. until analysis.

### Study Completion

A patient is considered to have completed the study following the evaluations for the primary endpoint after 4 cycles of treatment. However, patients continuing on study and receiving further treatment are followed and data collected. Where possible, all patients are evaluated every three months. The study is closed when the final patient has undergone this last review. Patients who have received at least 1 cycle of study agent are evaluable for safety and for clinical and biological response. PSA response rates are summarised by proportions together with 95% confidence intervals. Proportions and durations of progression-free survival are summarised by Kaplan-Meier methods. Toxicity is summarised according to Common Terminology Criteria for Adverse Events Version 3.0.

Finally, it is to be understood that various other modifications and/or alterations may be made without departing from the spirit of the present invention as outlined herein.

Future patent applications may be filed in Australia or overseas on the basis of or claiming priority from the present application. It is to be understood that the following provisional claims are provided by way of example only, and are not intended to limit the scope of what may be claimed in any such future application. Features may be added to or omitted from the provisional claims at a later date so as to further define or re-define the invention or inventions.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as broadly described herein.

Future patent applications may be filed in Australia or overseas on the basis of or claiming priority from the present application. It is to be understood that the following provisional claims are provided by way of example only, and are not intended to limit the scope of what may be claimed in any such future application. Features may be added to or omitted from the provisional claims at a later date so as to further define or re-define the invention or inventions.

### Example 21

### Control of Estrus in a Bitch

Use polypeptide capable of binding Estrogen to control estrus in a greyhound bitch. Absence of estrogen means that she will not cycle and so can race.

### Example 22

### Chemical Sterilisation to Change Meat Characteristics in Pigs

Administer anti-androgen so that male pigs can be grown to an older age before slaughter without ‘boar taint’. This increases efficiency as more meat per animal will be produced.

1. A polypeptide comprising an androgen binding region, the androgen binding region capable of binding to an androgen at a sufficient affinity or avidity such that upon administration of the polypeptide to a mammalian subject the level of biologically available androgen is decreased.

2. A method for treating or preventing prostate cancer in a subject, the method comprising administering to a subject in need thereof an effective amount of a ligand capable of binding androgen in the subject, such that the level of biologically available androgen in the subject is decreased as compared with the level of biologically available androgen present in the subject prior to administration of the polypeptide.

34. A polypeptide comprising an estrogen or androgen binding region, the binding region capable of binding to an estrogen or androgen at a sufficient affinity or avidity such that upon administration of the polypeptide to a mammalian subject the level of biologically available estrogen or androgen is decreased.

4. A method for treating or preventing an estrogen-related cancer or an androgen-related cancer in a subject, the method comprising administering to a subject in need thereof an effective amount of a ligand capable of binding estrogen or androgen in the subject, such that the level of biologically available estrogen or androgen in the subject is decreased as compared with the level of biologically available estrogen or androgen present in the subject prior to administration of the ligand.

5. A polypeptide comprising a nuclear hormone receptor agonist binding region, the nuclear hormone receptor agonist binding region capable of binding to a nuclear hormone receptor agonist at a sufficient affinity or avidity such that upon administration of the polypeptide to a mammalian subject the level of biologically available nuclear hormone receptor agonist is decreased.

6. A method for treating or preventing a condition related to excess nuclear hormone receptor agonist in a subject, the method comprising administering to a subject in need thereof an effective amount of a ligand capable of binding a nuclear hormone receptor agonist in the subject, such that the level of biologically available nuclear hormone receptor agonist in the subject is decreased as compared with the level of biologically available nuclear hormone receptor agonist present in the subject prior to administration of the polypeptide.

7. A polypeptide for regulating a reproductive physiology of an animal, the polypeptide comprising a steroid sex hormone binding region, the steroid sex hormone binding region capable of binding to a steroid sex hormone at a sufficient affinity or avidity such that upon administration of the polypeptide to the animal the level of biologically available steroid sex hormone is decreased.
